SEQUENCE LISTING (63 sequences)

SEQ ID NO: 1 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 2 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTAPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 3 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 4 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTTPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 5 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 6 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 7 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGCIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 8 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVICIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 9 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYATWLLTCPVILIHLSNLTGLSNDYSRRTMGLLVSDIGCIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 10 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 11 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAAWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 12 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYATWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 13 - protein, 344 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 14 - protein, 285 aa, Artificial sequence
  Synthetic polypeptide
NNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 15 - protein, 344 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 16 - protein, 344 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 17 - protein, 357 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPATLWLSSGNGVVWMRYGTWLLTCPVLLIHLSNLTGLKDDYSKRTMGLLVSDIACIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDDTVKQSTAKYASR

SEQ ID NO: 18 - protein, 361 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPATLWLSSGNGVVWMRYGEWLLTCPVLLIHLSNLTGLKDDYSKRTMGLLVSDIACIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDDTVKQSNPHRTAKYASR

SEQ ID NO: 19 - protein, 357 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPATLWLSSGNGVVWMRYGTWLLTCPVLLIHLSNLTGLKDDYSKRTMGLLVSDIACIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDDTVKQSTAKYASR

SEQ ID NO: 20 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 21 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTSPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 22 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTSPVLLIHLSNLTGLKDDYSKRTMGLLVSAVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 23 - protein, 350 aa, Chlamydomonas rheinhardtii
MVSRRPWLLALALAVALAAGSAGASTGSDATVPVATQDGPDYVFHRAHERMLFQTSYTLENNGSVICIPNNGQCFCLAWLKSNGTNAEKLAANILQWVVFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 24 - protein, 350 aa, Artificial sequence
  Synthetic polypeptide
MVSRRPWLLALALAVALAAGSAGASTGSDATVPVATQDGPDYVFHRAHERMLFQTSYTLENNGSVICIPNNGQCFCLAWLKSNGTNAEKLAANILQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 25 - protein, 258 aa, Halorubrum sodomense
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 26 - protein, 248 aa, Artificial sequence
  Synthetic polypeptide
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 27 - protein, 534 aa, Artificial sequence
  Synthetic polypeptide
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAVSKAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 28 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 29 - protein, 262 aa, Halobacterium salinarum
MLELLPTAVEGVSQAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATSD

SEQ ID NO: 30 - protein, 365 aa, Dunaliella salina
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 31 - protein, 270 aa, Guillardia theta
MDYGGALSAVGRELLFVTNPVVVNGSVLVPEDQCYCAGWIESRGTNGASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 32 - protein, 291 aa, Natromonas pharaonis
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 33 - protein, 559 aa, Artificial sequence
  Synthetic polypeptide
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 34 - protein, 542 aa, Artificial sequence
  Synthetic polypeptide
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 35 - protein, 434 aa, Artificial sequence
  Synthetic polypeptide
MNGTEGPNFYVPFSNKTGVVRSPFEAPQYYLAEPWQFSMLAAYMFLLIMLGFPINFLTLYVIAKFERLQTVLNYILLNLAVADLFMVFGGFTTTLYTSLHGYFVFGPTGCNLEGFFATLGGEIALWSLVVLAIERYVVVTSPFKYQSLLTKNKAIMGVAFTWVMALACAAPPLVGWSRYIPEGMQCSCGIDYYTPHEETNNESFVIYMFVVHFIIPLIVIFFCYGRVFQVAKRQLQKIDKSEGRFHSPNLGQVEQDGRSGHGLRRSSKFCLKEHKALRMVIIMVIAFLICWLPYAGVAFYIFTHQGSDFGPIFMTIPAFFAKTSAVYNPVIYIMMNKQFRIAFQELLCLRRSSSKAYGNGYSSNSNGKTDYMGEASGCQLGQEKESERLCEDPPGTESFVNCQGTVPSLSLDSQGRNCSTNDSPLTETSQVAPA

SEQ ID NO: 36 - protein, 495 aa, Artificial sequence
  Synthetic polypeptide
MNGTEGPNFYVPFSNKTGVVRSPFEAPQYYLAEPWQFSMLAAYMFLLIMLGFPINFLTLYVVACHRHLHSVLNYILLNLAVADLFMVFGGFTTTLYTSLHGYFVFGPTGCNLEGFFATLGGEIALWSLVVLAIERYVVVSYPLRYPTIVTQRRAIMGVAFTWVMALACAAPPLVGWSRYIPEGMQCSCGIDYYTPHEETNNESFVIYMFVVHFIIPLIVIFFCYGRVYVVAKRESRGLKSGLKTDKSDSEQVTLRIHRKNAPAGGSGMASAKTKTHFSVRLLKFSREKKAARMVIIMVIAFLICWLPYAGVAFYIFTHQGSDFGPIFMTIPAFFAKTSAVYNPVIYIMMNKQFRKAFQNVLRIQCLCRKQSSKHALGYTLHPPSQAVEGQHKDMVRIPVGSRETFYRISKTDGVCEWKFFSSMPRGSARITVSKDQSSCTTARVRSKSFLQVCCCVGPSTPSLDKNHQVPTIKVHTISLSENGEEVTETSQVAPA

SEQ ID NO: 37 - protein, 20 aa, Artificial sequence
  Synthetic polypeptide
KSRITSEGEYIPLDQIDINV

SEQ ID NO: 38 - protein, 26 aa, Artificial sequence
  Synthetic polypeptide
MDYGGALSAVGRELLFVTNPVVVNGS

SEQ ID NO: 39 - protein, 27 aa, Artificial sequence
  Synthetic polypeptide
MAGHSNSMALFSFSLLWLCSGVLGTEF

SEQ ID NO: 40 - protein, 23 aa, Artificial sequence
  Synthetic polypeptide
MGLRALMLWLLAAAGLVRESLQG

SEQ ID NO: 41 - protein, 18 aa, Artificial sequence
  Synthetic polypeptide
MRGTPLLLVVSLFSLLQD

SEQ ID NO: 42 - protein, 5 aa, Artificial sequence
  Synthetic polypeptide
  MISC_FEATURE (2)..(3): Xaa can be any naturally occurring amino acid
VXXSL

SEQ ID NO: 43 - protein, 5 aa, Artificial sequence
  Synthetic polypeptide
VKESL

SEQ ID NO: 44 - protein, 5 aa, Artificial sequence
  Synthetic polypeptide
VLGSL

SEQ ID NO: 45 - protein, 16 aa, Artificial sequence
  Synthetic polypeptide
NANSFCYENEVALTSK

SEQ ID NO: 46 - protein, 6 aa, Artificial sequence
  Synthetic polypeptide
  MISC_FEATURE (2)..(2): Xaa can be any naturally occurring amino acid
FXYENE

SEQ ID NO: 47 - protein, 7 aa, Artificial sequence
  Synthetic polypeptide
FCYENEV

SEQ ID NO: 48 - protein, 18 aa, Artificial sequence
  Synthetic polypeptide
MTETLPPVTESAVALQAE

SEQ ID NO: 49 - protein, 357 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPATLWLSSGNGVVWMRYGEWLLTCPVLLIHLSNLTGLKDDYSKRTMGLLVSDIACIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDDTVKQSTAKYASR

SEQ ID NO: 50 - DNA, 7811 bp, Artificial sequence
  Synthetic oligonucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggaccccatcgctctgcaggctggttacgacctgctgggtgacggcagacctgaaactctgtggctgggcatcggcactctgctgatgctgattggaaccttctactttctggtccgcggatggggagtcaccgataaggatgcccgggaatattacgctgtgactatcctggtgcccggaatcgcatccgccgcatatctgtctatgttctttggtatcgggcttactgaggtgaccgtcgggggcgaaatgttggatatctattatgccaggtacgccgactggctgtttaccaccccacttctgctgctggatctggcccttctcgctaaggtggatcgggtgaccatcggcaccctggtgggtgtggacgccctgatgatcgtcactggcctcatcggagccttgagccacacggccatagccagatacagttggtggttgttctctacaatttgcatgatagtggtgctctattttctggctacatccctgcgatctgctgcaaaggagcggggccccgaggtggcatctacctttaacaccctgacagctctggtcttggtgctgtggaccgcttaccctatcctgtggatcataggcactgagggcgctggcgtggtgggcctgggcatcgaaactctgctgtttatggtgttggacgtgactgccaaggtcggctttggctttatcctgttgagatcccgggctattctgggcgacaccgaggcaccagaacccagtgccggtgccgatgtcagtgccgccgacaagagcaggatcaccagcgagggcgagtacatccccctggaccagatcgacatcaacgtgggcgcgcccggctccggagccacgaacttctctctgttaaagcaagcaggagacgtggaagaaaaccccggtcccatggacctgaaggagtcaccaagcgagggatcactgcagccatcaagcattcagattttcgctaatacaagcacactgcacggcatccggcatatcttcgtgtacggcccactgaccattcggagagtcctgtgggcagtggcctttgtcggaagcctgggactgctgctggtggagagctccgaaagagtcagttactatttctcatatcagcacgtgactaaggtggacgaggtggtcgctcagtccctggtgtttcccgcagtcaccctgtgcaacctgaatgggttcaggttttctcgcctgaccacaaacgacctgtaccacgccggagagctgctggctctgctggatgtgaatctgcagatcccagacccccatctggccgatccaaccgtgctggaagcactgaggcagaaggccaacttcaaacactacaagcccaaacagttcagcatgctggagtttctgcaccgcgtgggacatgacctgaaagatatgatgctgtattgcaagttcaaaggccaggagtgtgggcatcaggacttcactaccgtgtttacaaagtacggcaaatgttacatgttcaactccggggaagatggaaaacctctgctgacaactgtgaagggcgggacagggaatggactggagatcatgctggacattcagcaggatgagtacctgccaatctggggagaaactgaggaaaccacattcgaggccggcgtgaaggtccagatccactcacagagcgagccccctttcattcaggaactgggatttggagtggcaccaggattccagacatttgtcgctactcaggagcagcgcctgacctatctgccacccccttggggcgagtgccgatctagtgaaatggggctggacttctttcctgtgtactctatcaccgcctgccgaattgattgtgagacacggtatatcgtggaaaactgcaattgtaggatggtccacatgcctggcgacgccccattctgcactcccgaacagcataaagagtgtgctgaacctgcactggggctgctggctgagaaggatagtaactactgcctgtgtagaacaccctgtaacctgactaggtataataaggaactgagcatggtgaagatcccttccaaaacatctgcaaagtacctggagaagaagttcaacaagtctgagaagtacatcagtgaaaacattctggtgctggacatcttctttgaagctctgaattacgagaccattgaacagaagaaagcatatgaggtggccgctctgctgggggatattggaggccagatgggactgttcatcggcgccagcctgctgacaattctggagctgtttgactacatctatgagctgattaaggaaaaactgctggatctgctggggaaggaggaagaggaaggatcacacgacgaaaacatgagcacttgcgataccatgcctaatcacagcgagaccatctcccatacagtgaatgtcccactgcagactgcactgggcaccctggaggaaattgcctgtgcggccgccaagagcaggatcaccagcgagggcgagtacatccccctggaccagatcgacatcaacgtggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcagggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagttctgctacgagaacgaggtgtaatgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 51 - protein, 348 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 52 - protein, 350 aa, Artificial sequence
  Synthetic polypeptide
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIRLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 53 - protein, 356 aa, Artificial sequence
  Synthetic polypeptide
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTTPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVSSEDLYFQ

SEQ ID NO: 54 - protein, 309 aa, Artificial sequence
  Synthetic polypeptide
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTAPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 55 - protein, 309 aa, Artificial sequence
  Synthetic polypeptide
MDYGGALSAVGLFQTSYTLENNGSVICIPNNGQCFCLAWLKSNGTNAEKLAANILQWISFALSALCLMFYGYQTWKSTCGWENIYVATIQMIKFIIEYFHSFDEPAVIYSSNGNKTRWLRYASWLLTAPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSRYGSNVGHTIIDLMSKQCWGLLGHYLRVLIHSHILIHGDIRKTTKLNIGGTEIEVETLVEDEAEAGAV

SEQ ID NO: 56 - protein, 309 aa, Artificial sequence
  Synthetic polypeptide
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 57 - protein, 309 aa, Artificial sequence
  Synthetic polypeptide
MDYGGALSAVGLFQTSYTLENNGSVICIPNNGQCFCLAWLKSNGTNAEKLAANILQWISFALSALCLMFYGYQTWKSTCGWENIYVATIQMIKFIIEYFHSFDEPAVIYSSNGNKTRWLRYASWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSRYGSNVGHTIIDLMSKQCWGLLGHYLRVLIHSHILIHGDIRKTTKLNIGGTEIEVETLVEDEAEAGAV

SEQ ID NO: 58 - protein, 296 aa, Artificial sequence
  Synthetic polypeptide
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAVSKAAAKSRITSEGEYIPLDQIDINVFCYENEV

SEQ ID NO: 59 - protein, 248 aa, Artificial sequence; identical to SEQ ID NO: 26
  Synthetic polypeptide

SEQ ID NO: 60 - DNA, 1293 bp, Mus musculus
ttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 61 - protein, 359 aa, Artificial sequence
  Synthetic polypeptide
MVSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGKYESS

SEQ ID NO: 62 - protein, 325 aa, Artificial sequence
  Synthetic polypeptide
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 63 - protein, 276 aa, Artificial sequence
  Synthetic polypeptide
MTAVSTTATTVLQATQSDVLQEIQSNFLLNSSIWVNIALAGVVILLFVAMGRDLESPRAKLIWVATMLVPLVSISSYAGLASGLTVGFLQMPPGHALAGQEVLSPWGRYLTWTFSTPMILLALGLLADTDIASLFTAITMDIGMCVTGLAAALITSSHLLRWVFYGISCAFFVAVLYVLLVQWPADAEAAGTSEIFGTLRILTVVLWLGYPILFALGSEGVALLSVGVTSWGYSGLDILAKYVFAFLLLRWVAANEGTVSGSGMGIGSGGAAPADD

(19) United States
(12) Patent Application Publication
(10) Pub. No.: US 2016/0030765 A1
(43) Pub. Date: Feb. 4, 2016

(54) SYSTEM AND METHOD FOR OPTOGENETIC THERAPY

(71) Applicant: Circuit Therapeutics, Inc., Menlo Park, CA (US)

(72) Inventors: Christopher L. Towne, San Francisco, CA (US); Michael Kaplitt, Mountain View, CA (US); Scott Delp, Stanford, CA (US); Karl Deisseroth, Stanford, CA (US); David Angeley, Charottesville, VA (US); Greg Stahler, Belmont, CA (US); Dan Andersen, Menlo Park, CA (US); David C. Lundmark, Los Altos, CA (US)

(73) Assignee: Circuit Therapeutics, Inc., Menlo Park, CA (US)

(21) Appl. No.: 14/813,106

(22) Filed: Jul. 29, 2015

Related U.S. Application Data
(60) Provisional application No. 62/030,467, filed on Jul. 29, 2014.

(51) Int. Cl. A61N 5/06

(52) U.S. Cl. CPC A61N 5/0622; A61N 5/0601; A61N 2005/0626; A61N 2005/0651; A61N 2005/0662

(57) ABSTRACT

One embodiment is directed to a system for controllably managing pain in the afferent nervous system of a patient having a targeted tissue structure that has been genetically modified to have light sensitive protein, comprising a light delivery element configured to direct radiation to at least a portion of a targeted tissue structure; a light source configured to provide light to the light delivery element; and a controller operatively coupled to light source; wherein the targeted tissue structure comprises a sensory neuron of the patient; and wherein the controller is configured to be automatically operated to illuminate the targeted tissue structure with radiation such that a membrane potential of cells comprising the targeted tissue structure is modulated at least in part due to exposure of the light sensitive protein to the radiation.

### RELATED APPLICATION DATA

The present application claims priority to U.S. Provisional Application Ser. No. 62/030,467, filed Jul. 29, 2014. The foregoing application is hereby incorporated by reference into the present application in its entirety.

### INCORPORATION-BY-REFERENCE OF MATERIAL SUBMITTED ELECTRONICALLY

Incorporated by reference in its entirety is a computer-readable nucleotide/amino acid sequence listing submitted concurrently herewith, and identified as follows: One 156 KiloByte ASCII (Text) file named “20041_SeqList_ST25.txt” created on Jun. 11, 2015.

### FIELD OF THE INVENTION

The present invention relates generally to systems, devices, and processes for facilitating various levels of control over cells and tissues in vivo, and more particularly to systems and methods for physiologic intervention wherein light may be utilized as an input to tissues which have been modified to become light sensitive.

### BACKGROUND

An estimated 70 million people are affected by chronic pain. It is responsible for an estimated $100 billion a year in medical costs, lost working days, and workers compensation, and is a major risk factor for depression and suicide.

Pain can be divided into two general categories: nociceptive and neuropathic. In the former, mechanical, thermal, or chemical damage to tissue causes nociceptor response and initiates action potentials in nerve fibers. Afferent fibers terminate directly or indirectly on transmission cells in the spinal cord that convey information to the brainstem and midbrain. Neuropathic pain, in contrast, involves a miscoding of afferent input; mild inputs yield dramatic pain responses, through mechanisms that are not well understood. Often this is the result of an initial nociceptive pain that, instead of resolving with healing of the initial stimulus, proceeds to spontaneous pain and low-threshold for light touch to evoke pain.

Treatment of pain depends on many factors, including type, cause, and location. There are myriad options, most notably topical agents, acetaminophen and nsaids, antidepressants, anticonvulsant drugs, sodium and calcium channel antagonists, opioids, epidural and intrathecal analgesia, acupuncture and other alternative techniques, botulinum toxin injections, neurolysis, cryoneurolysis, spinal cord stimulation, neurosurgical techniques, radiofrequency ablation, peripheral nerve stimulation, transcutaneous electrical nerve stimulation, and rehabilitation therapy.

So many treatments exist because each has important limitations. For example, local anesthetic drugs block sodium channels, preventing neurons from achieving action potentials. However, effectiveness of this treatment is limited by the degree to which specificity for pain neurons can be maintained, avoiding the side effects of numbness or paralysis from blocking other sensory or motor fibers (as well as potential cardiac effects should the drug travel further through the circulatory system). In order to achieve this, low dosages are needed, requiring frequent administration of the drug. Additionally, not all kinds of pain react to local anesthetic treatment, and some cases become refractory over time, or require ever increasing doses.

Surgical treatments, including dorsal or cranial nerve rhizotomy, ganglionectomy, sympathectomy, or thalomatomy, are more drastic options, appropriate in certain severe cases. However, relief from these is unpredictable; notably, it is sometimes only temporary, and may involve complications. Spinal cord stimulation (SCS) is also used in some cases, attempting to limit chronic pain through placement of electrodes in the epidural space adjacent to a targeted spinal cord area thought to be causing pain; however, there is limited evidence of the effectiveness of this technique. In addition, because electrical stimulation is not selective, the stimulation excites motor nerves that produce twitching. Because spinal cord stimulation is excitatory patients often feel a tingling sensation.

While each of these traditional methods is effective in some cases, chronic pain remains a largely intractable problem. Thus, there is a clear need for new way to treat pain such as is described herein which offers the possibility to selectively interrupt or alter neurotransmission and even to interfere with the plastic changes in the nervous system underlying the development or persistence of chronic pain.

Pharmacological and direct electrical neuromodulation techniques have been employed in various interventional settings to address challenges such as prolonged orthopedic pain, epilepsy, and hypertension. Pharmacological manipulations of the neural system may be targeted to certain specific cell types, and may have relatively significant physiologic impacts, but they typically act on a time scale of minutes, whereas neurons physiologically act on a time scale of milliseconds. Electrical stimulation techniques, on the other hand, may be more precise from an interventional time scale perspective, but they generally are not cell type specific and may therefore involve significant clinical disadvantages. A new neurointerventional field termed “Optogenetics” is being developed which involves the use of light-sensitive proteins, configurations for delivering related genes in a very specific way to targeted cells, and targeted illumination techniques to produce interventional tools with both low latency from a time scale perspective, and also high specificity from a cell type perspective.

For example, optogenetic technologies and techniques recently have been utilized in laboratory settings to change the membrane voltage potentials of excitable cells, such as neurons, and to study the behavior of such neurons before and after exposure to light of various wavelengths. In neurons, membrane depolarization leads to the activation of transient electrical signals (also called action potentials or “spikes”), which are the basis of neuronal communication. Conversely, membrane hyperpolarization leads to the inhibition of such signals. By exogenously expressing light-activated proteins that change the membrane potential in neurons, light can be utilized as a triggering means to induce inhibition or excitation.

One approach is to utilize naturally-occurring genes that encode light-sensitive proteins, such as the so-called “opsins”. These light-sensitive transmembrane proteins may be covalently bonded to chromophore retinal, which upon absorption of light, isomerizes to activate the protein. Notably, retinal compounds are found in most vertebrate cells in sufficient quantities, thus eliminating the need to administer exogenous molecules for this purpose. The first genetically encoded system for optical control in mammalian neurons using light-sensitive signaling proteins was established in Drosophila melanogaster, a fruit fly species, and neurons expressing such proteins were shown to respond to light exposure with waves of depolarization and spiking. More recently it has been discovered that opsins from microorganisms which combine the light-sensitive domain with an ion pump or ion channel in the same protein may also modulate neuronal signaling to facilitate faster control in a single, easily-expressed, protein. In 2002, it was discovered that a protein that causes green algae (Chlamydomonas reinhardtii) to move toward areas of light exposure is a light-sensitive channel; exposure to light of a particular wavelength (maximum results at blue light spectrum 480 nm for the opsin ChR2, also known as “channelrhodopsin”) causes the membrane channel to open, allowing positive ions, such as sodium ions, to flood into the cell, much like the influx of ions that cause nerve cells to fire. Various other excitatory opsins, such as Volvox Channelrhodopsin (“VChR1”), Step Function Opsins (or “SFO”; ChR2 variants which can produce prolonged, stable, excitable states with blue-wavelength light exposure, and be reversed with exposure to green-wavelength light), or red-shifted optical excitation variants, such as “C1V1”, have been described by Karl Deisseroth and others, such as at the opsin sequence information site hosted at the URL: http://www.stanford.edu/group/dlab/optogenetics/sequence_info.html, the content of which is incorporated by reference herein in its entirety. Examples of opsins are described in U.S. patent application Ser. Nos. 11/459,638, 12/988,567, 12/522,520, and 13/577,565, and in Yizhar et al. 2011, Neuron 71:9-34 and Zhang et al. 2011, Cell 147:1446-1457, all of which are incorporated by reference herein in their entirety.

While excitation is desirable in some clinical scenarios, such as to provide a perception of a sensory nerve stimulation equivalent, relatively high-levels of excitation may also be utilized to provide the functional equivalent of inhibition in an “overdrive” or “hyperstimulation” configuration. For example, a hyperstimulation configuration has been utilized with capsaicin, the active component of chili peppers, to essentially overdrive associated pain receptors in a manner that prevents pain receptors from otherwise delivering pain signals to the brain (i.e., in an analgesic indication). An example of clinical use of hyperstimulation is the Brindley anterior sacral nerve root stimulator for electrical stimulation of bladder emptying (Brindley et al. Paraplegia 1982 20:365-381; Brindley et al. Journal of Neurology, Neurosurgery, and Psychiatry 1986 49:1104-1114; Brindley Paraplegia 1994 32:795-805; van der Aa et al. Archives of Physiology and Biochemistry 1999 107:248-256; Nosseir et al. Neurourology and Urodynamics 2007 26:228-233; Martens et al. Neurourology and Urodynamics 2011 30:551-555). In a parallel manner, hyperstimulation or overdriving of excitation with an excitatory opsin configuration may provide inhibitory functionality.

Other opsin configurations have been found to directly inhibit signal transmission without hyperstimulation or overdriving. For example, light stimulation of halorhodopsin (“NpHR”), a chloride ion pump, hyperpolarizes neurons and directly inhibits spikes in response to yellow-wavelength (˜589 nm) light irradiation. Other more recent variants (such as those termed “eNpHR2.0” and “eNpHR3.0”) exhibit improved membrane targeting and photocurrents in mammalian cells. Light driven proton pumps such as archaerhodopsin-3 (“Arch”), Mac, bacteriorhodopsin (“eBR”), and Guillardia theta rhodopsin-3 (“GtR3) may also be utilized to hyperpolarize neurons and block signaling. A new class of channel, recently described by Karl Deisseroth et al, such as in Science. April 2014. 344(6182):420-4, and Jonas Weitek, et al, in Science. April 2014. 344(6182):409-12, in which are incorporated by reference in their entirety, that is based on ChR but is modified to permit cations to pass through the “inhibitory” channel (which may be termed, by way of non-limiting examples; “iChR”, “iC1C2”, “ChloC”, or “SwiChR”) will open and permit large amounts of Cl-ions to pass, thereby hyperpolarizing the neuron more effectively and thus inhibiting the cell with greater efficiency and sensitivity. Thus this new class of channel, which is based on ChR (channel rhodopsin) but is modified to permit cations to pass through the channel rather than anions, provides yet further options. In response to blue light, this new “inhibitory” channel (iChR) will open and permit large amounts of Cl− ions to pass, thereby hyperpolarizing the neuron more effectively and thus inhibiting the cell with greater efficiency and sensitivity. When these opsins are transferred into neurons in the nervous system, those neurons can be activated or inactivated at will and with great efficiency and temporal control in response to specific wavelengths of light delivered by a light emitting device. Optogenetics therefore provides opportunities to regulate circuits with great biological specificity, so that only specific populations of neurons are activated or inhibited, without influencing nearby axons which are passing by and serve functions which are not intended targets of the therapy. This also provides opportunities for greater degree of restoration of broader circuit function by specific activating and/or inactivating multiple populations of neurons in a fashion that cannot be achieved with existing therapies. Direct hyperpolarization is a specific and physiological intervention that mimics normal neuronal inhibition. Suitable inhibitory opsins are also described in the aforementioned incorporated by reference resources.

Further, a ChR2 variant known as a Stabilized Step Function Opsin (or “SSFO”) provides light-activated ion channel functionality that can inhibit neural activity by depolarization block at the level of the axon. This occurs when the depolarization results in a depolarized membrane potential such that sodium channels are inactivated and no action potential of spikes can be generated.

We have demonstrated in animal models that NpHR can inhibit pain after the generation of neuropathic pain using intaneural AAV6 delivery i.e. viral delivery after onset of mechanical allodynia. That is, our optogenetic approach can inhibit pain when virus is delivered after nerve injury. We have also demonstrated in animal models that inhibitory chloride channels iC1C2167C and iC1C2167T (SwiChR) can reduce mechanical allodynia following intraneural AAV6 delivery. We have further demonstrated in animal models that intrathecal delivery is also a promising route by showing that the delivery of AAV8 expressing iC1C2 can transduce multiple dorsal root ganglion (DRG) and result in inhibition of neuropathic pain due to a Chronic Constrictive Injury (CCI). That is, inhibitory channels have been shown to inhibit pain using any of the herein described intraneural, intrathecal and direct DRG delivery approaches. Furthermore, light-mediated increases in pain tolerance were observed in the contralateral foot. This demonstrates this therapeutic delivery approach and the ability to affect multiple dermatomes following a single injection. That is, intrathecal delivery of AAV8:iC1C2 have been shown to result in more widespread transduction and inhibit pain in multiple dermatomes in response to light following a single injection. We have still further demonstrated in animal models that the present inventive optogenetic approach can reduce pain in at least two different neuropathic pain models, Chronic Constrictive Injury (CCI) and Complex Regional Pain Syndrome (CRPS). That is, our inventive optogenetic approach have been shown to inhibit pain in at least two different neuropathic pain models. We have also demonstrated in animal models that direct DRG injections of AAV5 expressing iC1C2 can lead to more restricted expression and result in inhibition of neuropathic pain in both rat CCI and CRPS models. That is, delivery of AAV5:iC1C2 directly to the DRG have been shown to result in opsin expression restricted to relevant neurons and inhibit pain in response to light in at least two different species utilizing the present invention. All of this supporting evidence strongly points to the present invention's clinical potentiality.

With a variety of opsins available for optogenetic experimentation in the laboratory, there is a need to bring such technologies to the stage of medical intervention, which requires not only a suitable selection of opsin-based tools for excitation and/or inhibition, but also a means for delivering the genetic material to the subject patient and a means for controllably illuminating the subject tissue within the patient to utilize the light-driven capabilities which may address the need for improved pain therapies.

### SUMMARY

One embodiment is directed to a system for controllably managing pain in the afferent nervous system of a patient having a targeted tissue structure that has been genetically modified to have light sensitive protein, comprising a light delivery element configured to direct radiation to at least a portion of a targeted tissue structure; a light source configured to provide light to the light delivery element; and a controller operatively coupled to light source; wherein the targeted tissue structure comprises a sensory neuron of the patient; and wherein the controller is configured to be automatically operated to illuminate the targeted tissue structure with radiation such that a membrane potential of cells comprising the targeted tissue structure is modulated at least in part due to exposure of the light sensitive protein to the radiation. The portion of the targeted tissue structure of the patient may be selected from the group consisting of: a spinal cord, a nerve cell body, a ganglion, a dorsal root ganglion, an afferent nerve fiber, an afferent nerve bundle, an afferent nerve ending, a sensory nerve fiber, a sensory nerve bundle, a sensory nerve ending, a sensory receptor, a free nerve ending, a mechanoreceptor, and a nociceptor. An applicator may be disposed to illuminate the target tissue structure, the applicator being comprised of at least a light delivery element and a sensor, wherein the sensor is configured to: produce an electrical signal representative of the state of the target tissue or its environment; and deliver the signal to the controller, wherein the controller is further configured to interpret the signal from the sensor and adjust at least one light source output parameter such that the signal is maintained within a desired range, wherein the light source output parameter may be chosen from the group containing of; current, voltage, optical power, irradiance, pulse duration, pulse interval time, pulse repetition frequency, and duty cycle. The sensor may be selected from the group consisting of: an optical sensor, a temperature sensor, a chemical sensor, and an electrical sensor. The controller further may be configured to drive the light source in a pulsatile fashion. The current pulses may be of a duration within the range of 1 millisecond to 100 seconds. The duty cycle of the current pulses may be within the range of 99% to 0.1%. The controller may be responsive to a patient input. The system may be configured such that patient input may trigger the delivery of current. The current controller further may be configured to control one or more variables selected from the group consisting of: the current amplitude, the pulse duration, the duty cycle, and the overall energy delivered. The light delivery element may be placed about at least 60% of circumference of a nerve or nerve bundle. The light delivery element may be placed inside of the body of a patient. The light delivery element may be placed outside of a body of a patient. The light sensitive protein may be an opsin protein. The opsin protein may be selected from the group consisting of: a depolarizing opsin, a hyperpolarizing opsin, a stimulatory opsin, an inhibitory opsin, a chimeric opsin, and a step-function opsin. The opsin protein may be selected from the group consisting of: NpHR, eNpHR 1.0, eNpHR 2.0, eNpHR 3.0, SwiChR, SwiChR 2.0, SwiChR 3.0, Mac, Mac 3.0, Arch, ArchT, Arch 3.0, ArchT 3.0, iChR, ChR2, C1V1-T, C1V1-TT, Chronos, Chrimson, ChrimsonR, CatCh, VChR1-SFO, ChR2-SFO, ChR2-SSFO, ChEF, ChIEF, Jaws, ChloC, Slow ChloC, iC1C2, iC1C2 2.0, and iC1C2 3.0. The light sensitive protein may be delivered to the target tissue using a virus. The virus may be selected from the group consisting of: AAV1, AAV2, AAV4, AAV5, AAV6, AAV7, AAV8, AAV9, lentivirus, and HSV. The virus may contain a polynucleotide that encodes for the opsin protein. The polynucleotide may encode for a transcription promoter. The transcription promoter may be selected from the group consisting of: CaMKIIa, hSyn, CMV, Hb9Hb, Thy1, and Ef1a. The viral construct may be selected from the group consisting of: AAV5-hSyn-eNpHR3.0, AAV5-CAG-eNpHR3.0, AAV5-hSyn-Arch3.0, AAV5-CAG-Arch3.0, AAV5-hSyn-iC1C23.0, AAV5-CAG-iC1C23.0, AAV5-hSyn-SwiChR3.0, AAV5-CAG-SwiChR3.0, AAV6-hSyn-eNpHR3.0, AAV6-CAG-eNpHR3.0, AAV6-hSyn-Arch3.0, AAV6-CAG-Arch3.0, AAV6-hSyn-iC1C23.0, AAV6-CAG-iC1C23.0, AAV6-hSyn-SwiChR3.0, AAV6-CAG-SwiChR3.0, AAV8-hSyn-eNpHR3.0, AAV8-CAG-eNpHR3.0, AAV8-hSyn-Arch3.0, AAV8-CAG-Arch3.0, AAV8-hSyn-iC1C23.0, AAV8-CAG-iC1C23.0, AAV8-hSyn-SwiChR3.0, and AAV8-CAG-SwiChR3.0. The light source may be configured to emit light having a wavelength that is within a wavelength range that is selected from the group consisting of: 440 nm to 490 nm, 491 nm to 540 nm, 541 nm to 600 nm, 601 nm to 650 nm, and 651 nm to 700 nm. The light delivery element may comprise a light emitting diode (LED). The virus may be delivered to an anatomical location that is different than that of the target tissue structure. Such anatomical location may be selected from the group consisting of: a spinal cord, a nerve cell body, a ganglion, a dorsal root ganglion, an afferent nerve fiber, an afferent nerve bundle, an afferent nerve ending, a sensory nerve fiber, a sensory nerve bundle, a sensory nerve ending, and a sensory receptor.

### BRIEF DESCRIPTION OF THE DRAWINGS

FIG. 1 depicts an embodiment of a technique for optogenetic treatment of a human in accordance with the present invention.

FIGS. 2A and 2B depict an embodiment of an injection configuration for optogenetic treatment of a human in accordance with the present invention.

FIG. 3 depicts an embodiment of a system level componentry configuration for optogenetic treatment of a human in accordance with the present invention.

FIGS. 4A and 4B depict activation wavelength and timing charts for various opsin proteins that may be utilized in embodiments of the present invention.

FIG. 4C depicts an LED specification table for various LEDs that may be utilized in embodiments of the present invention.

FIG. 5 depicts an embodiment of one portion of an illumination configuration for optogenetic treatment of a human in accordance with the present invention.

FIG. 6 depicts a light power density chart that may be applied in embodiments of the present invention.

FIG. 7 depicts an irradiance versus geometry chart that may be applied in embodiments of the present invention.

FIGS. 8-28 depict various aspects of embodiments of light delivery configurations which may be utilized for optogenetic treatment of a human in accordance with the present invention.

FIGS. 29A and 29B illustrate system level deployments of optogenetic treatment systems for nerve root intervention in accordance with the present invention.

FIGS. 30A-37 depict various aspects of embodiments of light delivery configurations and related issues and data, which may be utilized for optogenetic treatment of a human in accordance with the present invention.

FIGS. 38A-48Q depict various amino acid sequences of exemplary opsins, signal peptides, signal sequences, ER export sequences, and a trafficking sequence, as well as a polynucleotide sequence encoding Champ.

FIGS. 49-50C depict various aspects of embodiments of light delivery configurations and related issues and data, which may be utilized for optogenetic treatment of a human in accordance with the present invention.

FIGS. 51A-52D depict various aspects of embodiments related to intraneural injection, which may be utilized for optogenetic treatment of a human in accordance with the present invention.

FIGS. 53A-53J depict various aspects of embodiments related to device implantation, which may be utilized for optogenetic treatment of a human in accordance with the present invention.

FIGS. 54A-54J depict tables and charts containing descriptions of at least some of the opsins described herein.

FIGS. 55-76 illustrate various aspects of embodiments pertinent to optogenetic therapy embodiments, which may be utilized for optogenetic treatment of a human in accordance with the present invention.

FIGS. 77 to 84 illustrate various aspects of embodiments of optogenetic therapy for pain intervention.

FIG. 85 depicts a schematic representation of the pain pathway in a typical patient.

FIG. 86 shows the different types of pain, their classifications, and some exemplary clinical indications.

FIG. 87 depicts a schematic representation of the mechanisms of peripheral neuropathic pain.

FIG. 88 depicts a schematic representation of the means of light delivery to the target tissue.

FIG. 89 depicts the location and distribution of nerves for hairy and glabrous skin.

FIG. 90 depicts an optical solid-model of the skin.

FIG. 91 depicts the fluence rate through the depth of the skin for two different exposure diameters.

FIG. 92 depicts the fluence rate through the depth of the skin for two different optical configurations.

FIGS. 93-96 depict the fluence rate through different skin types for two different treatment wavelengths.

FIG. 97 depicts the fluence rate through a depth of darkly pigmented skin for two different treatment wavelengths.

FIGS. 98 and 99 illustrate exemplary system level deployment of an optogenetic treatment system for pain intervention in accordance with the present invention.

FIGS. 100A through 100D illustrate means to illuminate a surface.

FIGS. 101 through 103 illustrate exemplary system level deployments of optogenetic treatment systems for pain intervention in accordance with the present invention.

FIGS. 104A through 108G depict various aspects of preclinical testing of the embodiments of the present invention.

### DETAILED DESCRIPTION

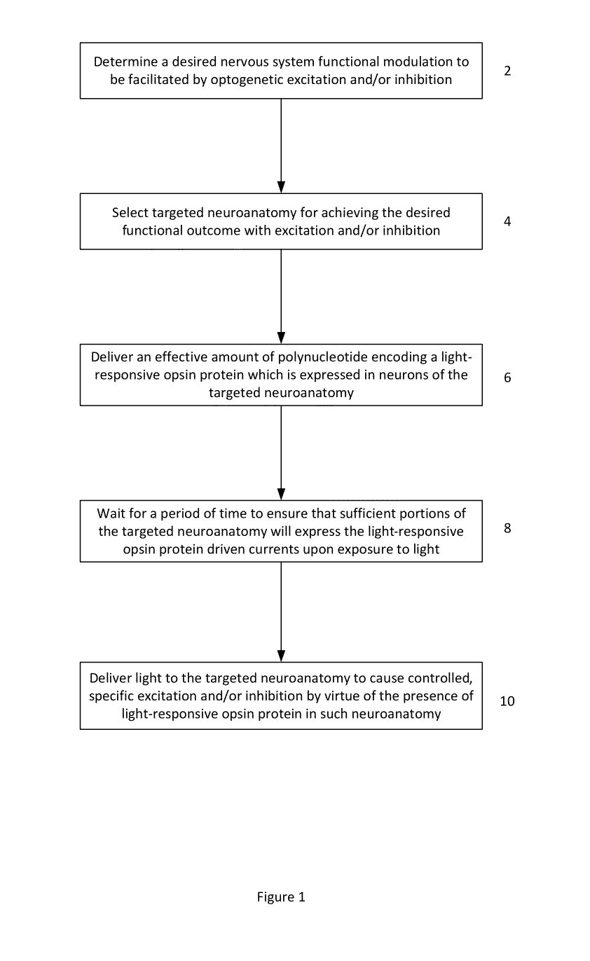

Referring to FIG. 1, from a high-level perspective, an optogenetics-based neuromodulation intervention involves determination of a desired nervous system functional modulation which can be facilitated by optogenetic excitation and/or inhibition (2), followed by a selection of neuroanatomic resource within the patient to provide such outcome (4), delivery of an effective amount of polynucleotide encoding a light-responsive opsin protein which is expressed in neurons of the targeted neuroanatomy (6), waiting for a period of time to ensure that sufficient portions of the targeted neuroanatomy will indeed express the light-responsive opsin protein-driven currents upon exposure to light (8), and delivering light to the targeted neuroanatomy to cause controlled, specific excitation and/or inhibition of such neuroanatomy by virtue of the presence of the light-responsive opsin protein therein (10).

As noted above, an optogenetics-based neuromodulation intervention involves determination of a desired nervous system functional modulation which can be facilitated by optogenetic excitation and/or inhibition, followed by a selection of neuroanatomic resource within the patient to provide such outcome, delivery of an effective amount of polynucleotide encoding a light-responsive opsin protein which is expressed in neurons of the targeted neuroanatomy, waiting for a period of time to ensure that sufficient portions of the targeted neuroanatomy will indeed express the light-responsive opsin protein-driven currents upon exposure to light, and delivering light to the targeted neuroanatomy to cause controlled, specific excitation and/or inhibition of such neuroanatomy by virtue of the presence of the light-responsive opsin protein therein.

While the development and use of transgenic animals has been utilized to address some of the aforementioned challenges, such techniques are not suitable in human medicine. Means to deliver the light-responsive opsin to cells in vivo are required; there are a number of potential methodologies that can be used to achieve this goal. These include viral mediated gene delivery, electroporation, optoporation, ultrasound, hydrodynamic delivery, or the introduction of naked DNA either by direct injection or complemented by additional facilitators such as cationic lipids or polymers.

Viral expression systems have the dual advantages of fast and versatile implementation combined with high copy number for robust expression levels in targeted neuroanatomy. Cellular specificity may be obtained with viruses by virtue of promoter selection if the promoters are small and specific, by localized targeting, and by restriction of opsin activation (i.e., via targeted illumination) of particular cells or projections of cells. In an embodiment, an opsin is targeted by methods described in Yizhar et al. 2011, Neuron 71:9-34. In addition, different serotypes of the virus (conferred by the viral capsid or coat proteins) will show different tissue tropism. Lenti- and adeno-associated (“AAV”) viral vectors have been utilized successfully to introduce opsins into the mouse, rat and primate brain. Other vectors include but are not limited to equine infectious anemia virus pseudotyped with a retrograde transport protein (e.g., Rabies G protein), and herpes simplex virus (“HSV”).

Additionally, these have been well tolerated and highly expressed over relatively long periods of time with no reported adverse effects, providing the opportunity for long-term treatment paradigms. Lentivirus, for example, is easily produced using standard tissue culture and ultracentrifuge techniques, while AAV may be reliably produced either by individual laboratories or through core viral facilities. AAV is a preferred vector due to its safety profile, and AAV serotypes 1 and 6 have been shown to infect motor neurons following intramuscular injection in primates. Additionally, AAV serotype 2 has been shown to be expressed and well tolerated in human patients.

AAV6 may be a preferred serotype for intraneural injections as it has been demonstrated to preferentially infect nociceptive fibers following nerve injection in rodents.

AAV8 may be a preferred serotype for intrathecal injections as it has been demonstrated to efficiently transduce DRG neurons following lumbar puncture in rodents, dogs and pigs.

AAV5 may be a preferred serotype for direct DRG injections as it has high neural tropism when injected into rodent and primate brains, but also, has low tropism for axons of passage, which may be important to restrict expression from motor neurons which have axons of passage adjacent to the DRG. AAV2 may also be a preferred serotype for direct DRG injections as it has experience in neural parenchyma injections in the humans, and also, has limited tropism for axons of passage.

Viral expression techniques, generally comprising delivery of DNA encoding a desired opsin and promoter/catalyst sequence packaged within a recombinant viral vector have been utilized with success in mammals to effectively transfect targeted neuroanatomy and deliver genetic material to the nuclei of targeted neurons, thereby inducing such neurons to produce light-sensitive proteins which are migrated throughout the neuron cell membranes where they are made functionally available to illumination components of the interventional system. Typically a viral vector will package what may be referred to as an “opsin expression cassette”, which will contain the opsin (e.g., ChR2, NpHR, etc.) and a promoter that will be selected to drive expression of the particular opsin within a targeted set of cells. In the case of adeno-associated virus (or AAV), the gene of interest (opsin) can be in a single stranded configuration with only one opsin expression cassette or in a self-complementary structure with two copies of opsin expression cassette complimentary in sequence with one another and connected by hairpin loops. The self-complementary AAVs are thought to be more stable and show higher expression levels and shows faster expression. The promoter may confer specificity to a targeted tissue, such as in the case of the human synapsin promoter (“hSyn”) or the human Thy1 promoter (“hThy1”) which allow protein expression of the gene under its control in neurons. Another example is the calcium/calmodulin-dependent kinase II promoter (“CAMKII”), which allows protein expression of the gene under its control only in excitatory neurons, a subset of the neuron population. Alternatively, a ubiquitous promoter may be utilized, such as the human cytomegalovirus (“CMV”) promoter, or the chicken beta-actin (“CBA”) promoter, each of which is not particularly neural specific, and each of which has been utilized safely in gene therapy trials for neurodegenerative disease. Viral constructs carrying opsins are optimized for specific neuronal populations and are not limited to such illustrative examples.

Viral expression systems have the dual advantages of fast and versatile implementation combined with high infective/copy number for robust expression levels in targeted neuroanatomy. Cellular specificity may be obtained with viruses by virtue of promoter selection if the promoters are small, specific, and strong enough, by localized targeting of virus injection, as discussed in further detail below, and by restriction of opsin activation (i.e., via targeted illumination) of particular cells or projections of cells, also as described in further detail below. In an embodiment, an opsin is targeted by methods described in Yizhar et al. 2011, Neuron 71:9-34. In addition, different serotypes of the virus (conferred by the viral capsid or coat proteins) will show different tissue trophism. Lenti- and adeno-associated (“AAV”) viral vectors have been utilized successfully to introduce opsins into the mouse, rat and primate brain. Additionally, these have been well tolerated and highly expressed over relatively long periods of time with no reported adverse effects, providing the opportunity for long-term treatment paradigms. Lentivirus, for example, is easily produced using standard tissue culture and ultracentrifuge techniques, while AAV may be reliably produced either by individual laboratories or through core viral facilities. Viruses have been utilized to target many tissue structures and systems, including but not limited to hypocretin neurons in the hypothalamus, excitatory pyramidal neurons, basal ganglia dopaminergic neurons, striatal GABAergic neurons, amygdala glutamatergic neurons, prefrontal cortical excitatory neurons and others, as well as astroglia. For example, it has been shown that the use of AAV-delivered ChR2 to control astroglial activity in the brainstem of mice and create a mechanism by which astroglia can transfer systemic information from the blood to neurons underlying homeostasis, in this case directly modulating neurons that manipulate the rate of respiration. AAV is a preferred vector due to its safety profile, and AAV serotypes 1 and 6 have been shown to infect motor neurons following intramuscular injection in primates. Other vectors include but are not limited to equine infectious anemia virus pseudotyped with a retrograde transport protein (e.g., Rabies G protein), and herpes simplex virus (“HSV”).

Delivery of the virus comprising the light-responsive opsin protein to be expressed in neurons of the targeted neuroanatomy may involve injection, infusion, or instillation in one or more configurations. By way of nonlimiting example, in a Pain therapy configuration, delivery means may include tissue structure injection (or infusion) (i.e., directly into the DRG, and/or the intrathecal space, and/or the targeted nerve or bundle thereof). Each of these injection configurations is explored in further detail below.

In one embodiment, nerve fibers may be targeted by direct injection (i.e., injection into the nerve itself). This approach, which may be termed “intrafascicular” or “intraneural” injection, involves placing a needle into the fascicle of a nerve bundle. Intrafascicular injections are an attractive approach because they allow specific targeting those neurons which may innervate a relatively large target (e.g., fibers across entire kidney, fibers across entire dermatome of skin, fibers across entire stomach wall) with one injection (e.g., before the fibers enter the tissue and anatomically bifurcate). The pertinent vector solution may be injected through the needle where it may diffuse throughout the entire nerve bundle (10 to 1000's of axon fibers). The vector may then enter the individual axon fibers through active (receptor-mediated) or passive (diffusion across intact membranes or transiently disrupted membranes) means. Once it has entered the axon, the vector may be delivered to the cell body via retrograde transport mechanisms, as described above. The number of injections and dose of virus injected to the nerve are dependent upon the size of the nerve, and can be extrapolated from successful transduction studies. For example, injection of the sciatic nerve of mice (approximately 0.3 mm diameter) with 0.002 mL saline containing 1×10⁹ vg of AAV has been shown to result in efficient transgene delivery to sensory neurons involved in pain sensing. Likewise, injection of the sciatic nerve of rats (1 mm diameter) with 0.010 mL saline containing 1-4×10¹⁰ vg of AAV has also achieved desirable transfection results. The trigeminal nerve in humans is 2 mm in diameter, and through extrapolation of the data from these pertinent studies, the trigeminal nerve may be transfected to efficiently deliver a transgene to these pertinent pain neurons using a direct injection of 0.05 mL saline containing 4×10¹⁰×10¹⁴ vg of AAV into the trigeminal bundle. These titers and injection volumes are illustrative examples and are specifically determined for each viral construct-target neuron pairing.

The protocol for nerve injections will vary depending upon the target. Superficial nerves may be targeted by making an incision through the skin, and then exposing the nerve through separation of muscles, fascia and tendons. Deeper nerves (i.e., outside of the abdominal and thoracic cavity—such as the pudendal nerve) may be targeted through ultrasound-guided surgical intervention. Nerves in the abdominal cavity may be targeted through laparoscopic surgical approaches wherein one or more small incisions may be made through the skin and other structures (such as the abdominal wall) to allow insertion of the surgical apparatus (camera, needle, tools, etc.) to a position adjacent the anatomy of interest. The needle may be guided into the nerve (as visualized through the camera and other available imaging systems, such as ultrasound, fluoroscopy, radiography, etc.). In all cases, the vector solution may be injected as a single bolus dose, or slowly through an infusion pump (0.001 to 0.1 mL/min).

In another particular example of intraneural injection, nociceptive fibers of the trigeminal nerve may be directly injected to address neuropathic pain symptoms, as briefly described above. In one embodiment, the trigeminal nerve may be directly injected with an AAV vector solution either through exposure of the nerve or through the skin via ultrasound guidance. Once in the nerve fascicle, the vector is configured to preferentially enter the non-myelinated or poorly-myelinated fibers that correspond to those cells mediating pain.

In another particular example of intraneural injection, the sciatic nerve may be injected with an AAV vector solution either through exposure of the nerve or through the skin via ultrasound guidance. The vector may be configured such that once it accesses the nerve fascicle, it preferentially enters the sensory neurons or motor neurons responsible for the symptoms of spasticity.

In another particular example of intraneural injection, the cervical vagus nerve may be injected with an AAV vector solution through exposure of the nerve in the neck. Once in the nerve fascicle, the vector may be configured to preferentially enter the relevant nerve fibers that are the mediators of the therapeutic effect of electrical vagus nerve stimulation for epilepsy.

In another particular example of intraneural injection, the cervical vagus nerve may be injected with an AAV vector solution through exposure of the nerve in the neck. Once in the nerve fascicle, the vector may be configured to preferentially enter the relevant nerve fibers that are the mediators of the therapeutic effect of vagus electric nerve stimulation for depression.

As mentioned above, injection into the ganglion may be utilized to target the neural cell bodies of peripheral nerves. Ganglia consist of sensory neurons of the peripheral nervous system, as well as autonomic neurons of the parasympathetic and sympathetic nervous system. A needle may be inserted into the ganglion which contains the cell bodies and a vector solution injected through the needle, where it may diffuse throughout the tissue and be taken up by the cell bodies (100s to 1000s of cells). In one embodiment, a dose of approximately 0.1 mL saline containing from 1×10¹¹ vg to 1×10¹⁴ vg of AAV may be used per ganglion. There are different types of ganglia that may be targeted. Dorsal root ganglion of the spinal cord may be injected in a similar method that is used during selective dorsal rhizotomy (i.e. injection via the intrathecal subarachnoid space of the spinal cord), except rather than cutting the nerves, the dorsal root ganglia may be injected. Other ganglia not in the abdominal cavity, such as the nodose ganglion of the vagus nerve, may be targeted by making an incision through the skin, and then exposing the ganglia through separation of muscles, fascia and tendons. Ganglia in the abdominal cavity, such as the ganglia of the renal plexus, may be injected through laparoscopic techniques, wherein one or more small incisions may be made through the skin and abdominal wall to allow insertion of the surgical apparatus (camera, needle, tools, etc.) to locations facilitating access and imaging of the pertinent targeted tissue. The needle may be guided into the ganglia (as visualized through a camera or other imaging device, such as ultrasound or fluoroscopy). In all cases, the vector solution may be injected as a single bolus dose, or slowly through an infusion pump (0.001 to 0.1 mL/min). These ranges are illustrative, and doses are tested for each virus-promoter-opsin construct pairing them with the targeted neurons.

In one particular example of ganglion injection, the dorsal root ganglia mediating clinical neuropathic pain may be injected with an AAV vector solution, preferably containing an AAV vector that has tropism for cell body.

In another particular example of ganglion injection, the dorsal root ganglia mediating undesired muscular spasticity may be injected with an AAV vector solution. An AAV vector that has tropism for cell body may be used towards this goal, as is described elsewhere herein.

In addition to the method described previously for direct ganglion injection (i.e. enter through the route used for dorsal rhizotomy, however, rather than cutting the nerve we will inject the viral solution) we propose an alternative method wherein a myelogram may be obtained by administering contrast medium into the dorsal subarachnoid space. A guide needle may then be passed through the skin lateral to the midline and progressed ventromedially toward the DRG under CT guidance. Upon the needle being directly adjacent to the dorsal aspect of the DRG, the stylet of the guide needle may be withdrawn and more contrast medium may be injected to verify the tip has reached the lateral recess of the intrathecal space without penetrating the DRG. A second stepped cannula may then be inserted through the guide needle such that it may puncture the DRG by a predetermined length (by way of non-limiting example, for between 1 and 2 mm). Then a higher gauge needle (32 to 34 G) may be put through the second cannula to penetrate further into the DRG. The virus may then be delivered through this needle at a rate between 50 nL and 1 μL per minute. Volumes between 5 and 100 μL may be delivered containing between 5×10⁹ vg and 1×10¹⁴ vg of AAV.

Finally, topical injection or application to a tissue structure surface may be utilized to deliver genetic material for optogenetic therapy. Recombinant vectors are capable of diffusing through membranes and infecting neural nerve endings following such topical application or exposure. Examples are the infection of sensory fibers following topical application on skin, which has been shown in pain treatment studies. Likewise, efficacy of topical application of viral vectors has been increased using vector solutions suspended in gels. In one embodiment, a vector may be suspended in a gel and applied (e.g., swabbed, painted, injected, or sprayed) to the surface of tissues that have high densities of targeted superficial nerve fibers. With such embodiment, vectors will diffuse through the gel and infect nerve fibers via diffusion across intact neural fiber membranes. Internal topical application may be achieved using laparoscopic techniques, wherein one or more small incisions may be made through the skin and other pertinent tissue structures (such as the abdominal wall) to allow insertion of the surgical apparatus (camera, needle, tools, etc.). A needle may be guided into the target tissue (as visualized through the camera or other imaging devices). In all cases, the vector may be mixed with the gel (e.g. the product sold under the tradename “KY Jelly” by Johnson & Johnson Corporation) and then sprayed onto, painted onto, or injected out upon the surface of the pertinent tissue. A dose of approximately 0.1 mL saline containing 1×10¹⁰ vg to 1×10¹⁴ vg of AAV may be used to cover each 1 cm² area. These ranges are illustrative, and doses are tested for each virus-promoter-opsin construct pairing them with the targeted neurons.

In one particular example of topical application, a solution or gel may be applied to infect the targeted afferent nerve fibers of the skin, such as, but not limited to, the free nerve endings which reside in the upper dermis and epidermis.

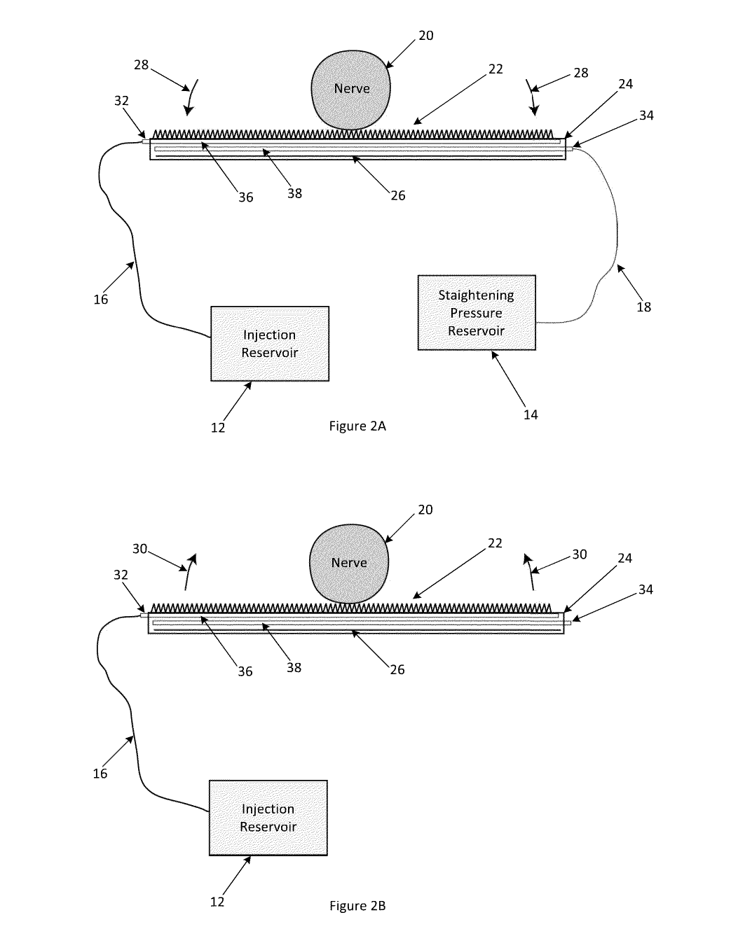

Alternately, the micropuncture device shown in FIGS. 2A and 2B may also be used on a tissue surface to introduce a genetic material and/or viral vector.

Referring back to FIG. 1, after delivery of the polynucleotide to the targeted neuroanatomy (6), an expression time period generally is required to ensure that sufficient portions of the targeted neuroanatomy will express the light-responsive opsin protein-driven currents upon exposure to light (8). This waiting period may, for example, comprise a period of between about 1 month and 6 months. After this period of time, light may be delivered to the targeted neuroanatomy to facilitate the desired therapy. Such delivery of light may take the form of many different configurations, including transcutaneous configurations, implantable configurations, configurations with various illumination wavelengths, pulsing configurations, tissue interfaces, etc., as described below in further detail.

Referring to FIGS. 2A and 2B, both of which are end views showing a cross sectional anatomical face (N) and a cross sectional view of a treatment assembly which in orthogonal view may, for example, be rectangular, trapezoidal, or elliptical (i.e., so that it may provide a sufficient area of exposure to the anatomy N when in contact), a matrix of needles or needle-like injection structures (22) may be utilized to inject a vector solution or gel in a circumferential manner around a nerve (20), nerve bundle, vessel surrounded by nerve fibers, or other somewhat cylindrical targeted anatomic structure into which injection is desired. As shown in FIG. 2A, a flexible or deformable housing (24) may feature a bending spine member (26) configured to bias the housing into a cylindrical (i.e., like a cuff), arcuate, helical, or spiral shape without other counterbalancing loads, for example an angled stylette. For example, the bending spine member may comprise a superalloy such as Nitinol, which may be configured through heat treatment to be pre-biased to assume such cylindrical, arcuate, helical, or spiral shape. The depicted embodiment of the housing (24) also features two embedded bladders—an injection bladder (36) which is fluidly coupled between the matrix of injection members (22) and an injection reservoir by a fluid conduit (16) such as a tube or flexible needle, and a mechanical straightening bladder (38), which is fluidly coupled to a straightening pressure reservoir (14) by a fluid conduit (18) such as a tube or flexible needle. Preferably both fluid conduits (16, 18) are removably coupled to the respective bladders (36, 38) by a removable coupling (32, 34) which may be decoupled by manually pulling the conduits (16, 18) away from the housing (24). The housing (24) may be inserted, for example, through a port in a laparoscopic tool, cannula, or catheter and inserted to a position as shown in FIG. 2A with the straightening bladder (38) fully pressurized to bias the housing into the shown flat condition with the ends rotated downward (28) due to the pressure applied through the straightening pressure reservoir (14), for example using an operatively coupled syringe or controllable pump, and functionally delivered through the associated conduit (18). With the straightened housing (24) in a desirable position relative to the targeted anatomic structure (20), preferably as confirmed using one or more visualization devices such as a laparoscopic camera, ultrasound transducer, fluoroscopy, or the like, the pressure within the straightening pressure reservoir (14) may be controllably decreased (for example, in one embodiment, the associated conduit 18 may simply be disconnected from the coupling 34) to allow the ends of the housing (24) to flex and rotate (30) up and around the anatomical structure (20) due to the now un-counterbalanced bending loads applied by the pre-bending-biased bending spine member (26). FIG. 2B depicts the ends starting to rotate up and around (30) the anatomical structure (20). With complete rotation, the flexible housing preferably will substantially surround at least a portion of the anatomical structure (20) in an arcuate, cuff, helical, or spiral configuration with the matrix of needles (22) interfaced directly against the outer surface of the anatomical structure (20), after which the pressure within the injection reservoir (12) may be controllably increased, for example using an infusion pump or syringe, to inject the anatomical structure (20) with the desired solution or gel. In one embodiment, it may be desirable to leave the housing in place as a prosthesis; in another embodiment it may be desirable to remove the housing after successful injection. In the former scenario, in one variation, the housing may also comprise a light delivery interface, such as is described below (i.e., in addition to a bending spine 26, a straightening bladder 38, an injection bladder 36, and a matrix of needles 22, the housing 24 may also comprise one or more light delivery fibers, lenses, and the like, as described below, to facilitate light therapy after injection of the pertinent genetic material). In the latter scenario, wherein the housing is to be removed after injection, the straightening pressure conduit (18) will remain coupled to the straightening bladder (38) so that after injection has been completed, the pressure within the straightening reservoir (14) may again be controllably increased, thereby rotating (28) the housing back out into a flat configuration as shown in FIG. 2A such that it may then be removed away from the subject anatomy (20). In one embodiment, the matrix of needles (22) may reside upon a movable or flexible membrane or layer relative to the supporting housing (24), and may be biased to recede inward toward the housing (24) when the injection pressure is not heightened, and to become more prominent relative to the supporting housing (24) when the injection pressure is increased; in other words, to assist with delivery and retraction (i.e., so that the housing 24 may be moved around relative to other nearby tissues without scratching, scraping, injuring, or puncturing such tissues without intention), when the injection pressure is relatively low, the injection structures may be configured to become recessed into the housing. It may also be desirable to have the matrix of needles (22) retract subsequent to injection to generally prevent tissue trauma upon exit of the housing (24) in the event that the housing (24) is to be removed, or to prevent fibrous tissue encapsulation of the targeted tissue structure (4) which may be associated with or accelerated by relatively abrasive or indwelling foreign body presence. Indeed, in one embodiment wherein the housing (24) is to remain in place (for example, as an illumination/light applicator platform), the matrix of needles (22) may comprise a bioresorbable material such as PLGA, which is commonly utilized in surgery for its resorbable qualities and may be configured to dissolve and/or resorb away within a short time period after injection has been completed.

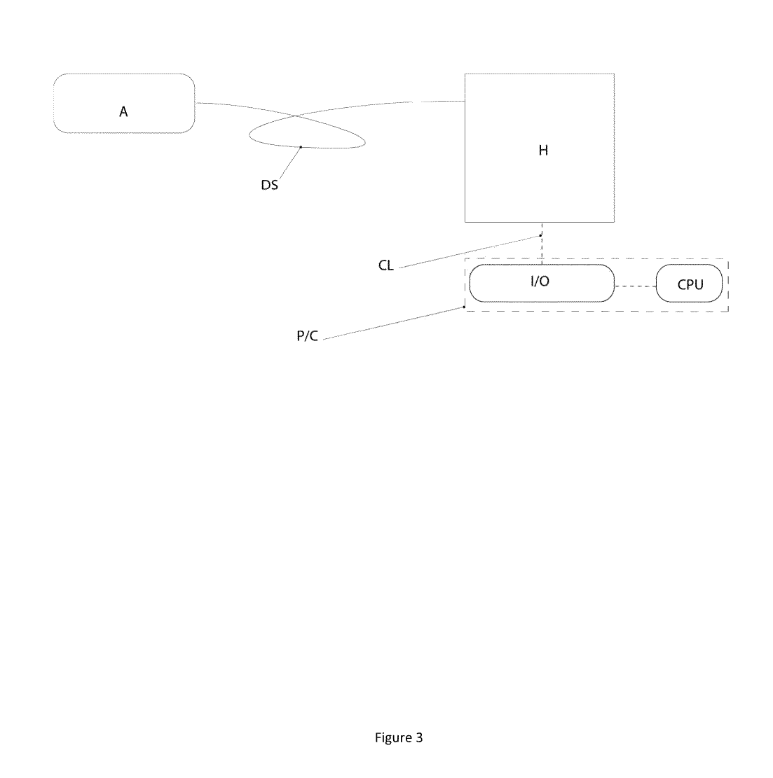

Referring to FIG. 3, a suitable light delivery system comprises one or more applicators (A) configured to provide light output to the targeted tissue structures. The light may be generated within the applicator (A) structure itself, or within a housing (H) that is operatively coupled to the applicator (A) via one or more delivery segments (DS), or at a location between the housing (H) and the applicator (A). The one or more delivery segments (DS) serve to transport, or guide, the light to the applicator (A) when the light is not generated in the applicator itself. In an embodiment wherein the light is generated within the applicator (A), the delivery segment (DS) may simply comprise an electrical connector to provide power to the light source and/or other components which may be located distal to, or remote from, the housing (H). The one or more housings (H) preferably are configured to serve power to the light source and operate other electronic circuitry, including, for example, telemetry, communication, control and charging subsystems. External programmer and/or controller (P/C) devices may be configured to be operatively coupled to the housing (H) from outside of the patient via a communications link (CL), which may be configured to facilitate wireless communication or telemetry, such as via transcutaneous inductive coil configurations, between the programmer and/or controller (P/C) devices and the housing (H). The programmer and/or controller (P/C) devices may comprise input/output (I/O) hardware and software, memory, programming interfaces, and the like, and may be at least partially operated by a microcontroller or processor (CPU), which may be housed within a personal computing system which may be a standalone system, or be configured to be operatively coupled to other computing or storage systems.

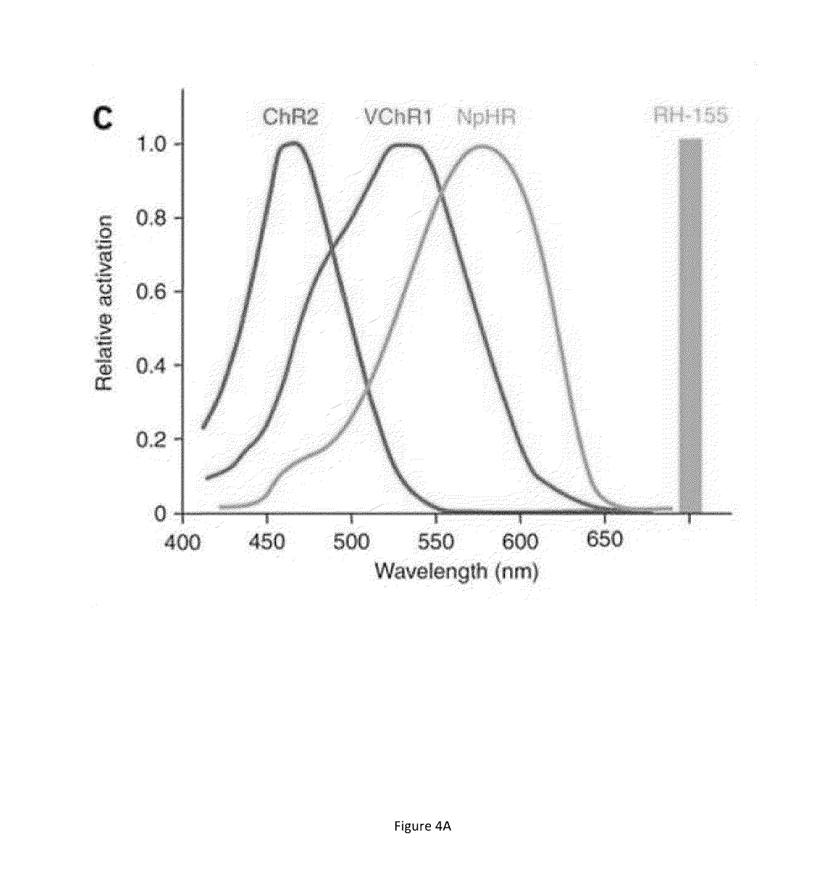

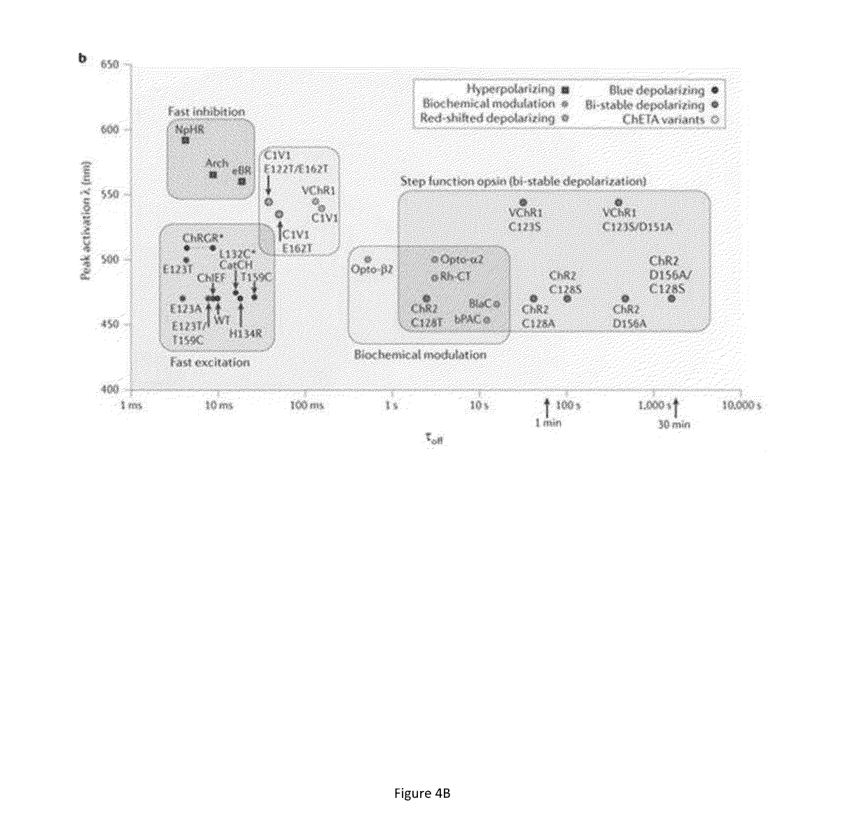

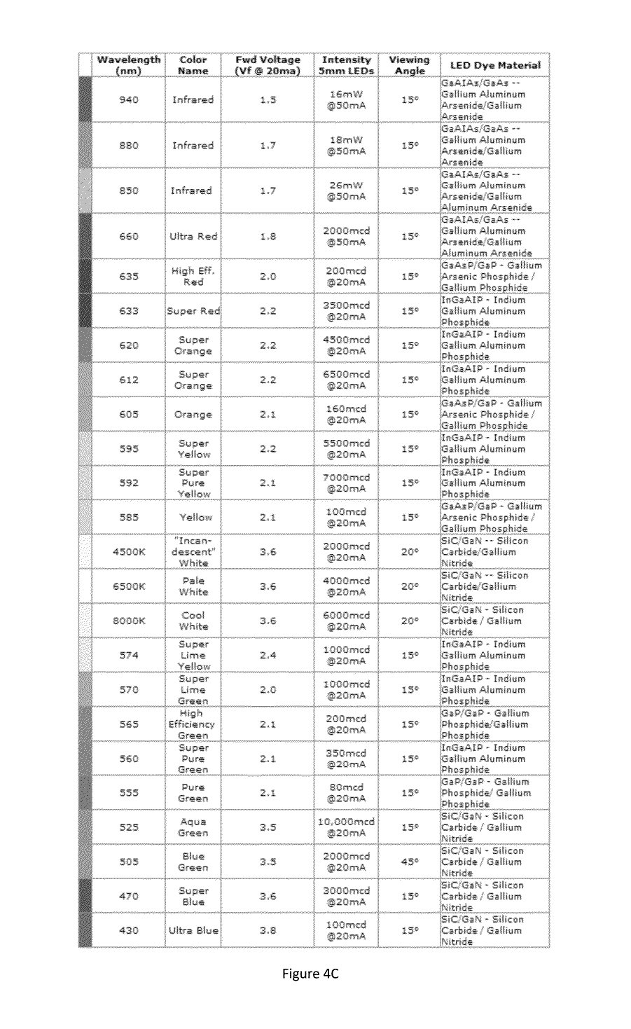

Referring to FIGS. 4A and 4B, as described above, various opsin protein configurations are available to provide excitatory and inhibitory functionality in response to light exposure at various wavelengths. FIG. 4A depicts wavelength vs activation for three different opsins; FIG. 4B emphasizes that various opsins also have time domain activation signatures that may be utilized clinically; for example, certain step function opsins (“SFO”) are known to have activations which last into the range of 30 minutes after stimulation with light.

Referring to FIG. 4C, a variety of light emitting diodes (LED) are commercially available to provide illumination at relatively low power with various wavelengths. As described above in reference to FIG. 3, in one embodiment, light may be generated within the housing (H) and transported to the applicator (A) via the delivery segment (DS). Light may also be produced at or within the applicator (A) in various configurations. The delivery segments (DS) may consist of electrical leads or wires without light transmitting capability in such configurations. In other embodiments, light may be delivered using the delivery segments (DS) to be delivered to the subject tissue structures at the point of the applicator (A), or at one or more points along the deliver segment (DS) itself (for example, in one case the DS may be a fiber laser). Referring again to FIG. 4C, an LED (or alternatively, “ILED”, to denote the distinction between this inorganic system and Organic LEDs) typically is a semiconductor light source, and versions are available with emissions across the visible, ultraviolet, and infrared wavelengths, with relatively high brightness. When a light-emitting diode is forward-biased (switched on), electrons are able to recombine with electron holes within the device, releasing energy in the form of photons. This effect is called electroluminescence and the color of the light (corresponding to the energy of the photon) is determined by the energy gap of the semiconductor. An LED is often small in area (less than 1 mm²), and integrated optical components may be used to shape its radiation pattern. In one embodiment, for example, an LED variation manufactured by Cree Inc. and comprising a Silicon Carbide device providing 24 mW at 20 mA may be utilized as an illumination source.

Organic LEDs (or “OLED”s) are light emitting diodes wherein the emissive electroluminescent layer is a film of organic compound that emits light in response to an electric current. This layer of organic semiconductor material is situated between two electrodes, which can be made to be flexible. At least one of these electrodes may be made to be transparent. The nontransparent electrode may be made to serve as a reflective layer along the outer surface on an optical applicator, as will be explained later. The inherent flexibility of OLEDs provides for their use in optical applicators such as those described herein that conform to their targets or are coupled to flexible or movable substrates, as described above in reference to FIGS. 2A-2B, and in further detail below. It should be noted, however, due to their relatively low thermal conductivity, OLEDs typically emit less light per area than an inorganic LED.

Other suitable light sources for embodiments of the inventive systems described herein include polymer LEDs, quantum dots, light emitting electrochemical cells, laser diodes, vertical cavity surface-emitting lasers, and horizontal cavity surface-emitting lasers.

Polymer LEDs (or “PLED”s), and also light-emitting polymers (“LEP”), involve an electroluminescent conductive polymer that emits light when connected to an external voltage. They are used as a thin film for full-spectrum color displays. Polymer OLEDs are quite efficient and require a relatively small amount of power for the amount of light produced.

Quantum dots (or “QD”) are semiconductor nanocrystals that possess unique optical properties. Their emission color may be tuned from the visible throughout the infrared spectrum. They are constructed in a manner similar to that of OLEDs.

A light-emitting electrochemical cell (“LEC” or “LEEC”) is a solid-state device that generates light from an electric current (electroluminescence). LECs may be usually composed of two electrodes connected by (e.g. “sandwiching”) an organic semiconductor containing mobile ions. Aside from the mobile ions, their structure is very similar to that of an OLED. LECs have most of the advantages of OLEDs, as well as a few additional ones, including:
The device does not depend on the difference in work function of the electrodes. Consequently, the electrodes can be made of the same material (e.g., gold). Similarly, the device can still be operated at low voltages;
Recently developed materials such as graphene or a blend of carbon nanotubes and polymers have been used as electrodes, eliminating the need for using indium tin oxide for a transparent electrode;
The thickness of the active electroluminescent layer is not critical for the device to operate, and LECs may be printed with relatively inexpensive printing processes (where control over film thicknesses can be difficult).

Semiconductor Lasers are available in a variety of output colors, or wavelengths. There are a variety of different configurations available that lend themselves to usage in the present invention, as well. Indium gallium nitride (InₓGa₁₋ₓN, or just InGaN) laser diodes have high brightness output at both 405, 445, and 485 nm, which are suitable for the activation of ChR2. The emitted wavelength, dependent on the material's band gap, can be controlled by the GaN/InN ratio; violet-blue 420 nm for 0.2In/0.8Ga, and blue 440 nm for 0.3In/0.7Ga, to red for higher ratios and also by the thickness of the InGaN layers which are typically in the range of 2-3 nm.

A laser diode (or “LD”) is a laser whose active medium is a semiconductor similar to that found in a light-emitting diode. The most common type of laser diode is formed from a p-n junction and powered by injected electric current. The former devices are sometimes referred to as injection laser diodes to distinguish them from optically pumped laser diodes. A laser diode may be formed by doping a very thin layer on the surface of a crystal wafer. The crystal may be doped to produce an n-type region and a p-type region, one above the other, resulting in a p-n junction, or diode. Laser diodes form a subset of the larger classification of semiconductor p-n junction diodes. Forward electrical bias across the laser diode causes the two species of charge carrier—holes and electrons—to be “injected” from opposite sides of the p-n junction into the depletion region. Holes are injected from the p-doped, and electrons from the n-doped, semiconductor. (A depletion region, devoid of any charge carriers, forms as a result of the difference in electrical potential between n- and p-type semiconductors wherever they are in physical contact.) Due to the use of charge injection in powering most diode lasers, this class of lasers is sometimes termed “injection lasers” or “injection laser diodes” (“ILD”). As diode lasers are semiconductor devices, they may also be classified as semiconductor lasers. Either designation distinguishes diode lasers from solid-state lasers. Another method of powering some diode lasers is the use of optical pumping. Optically Pumped Semiconductor Lasers (or “OPSL”) use a III-V semiconductor chip as the gain media, and another laser (often another diode laser) as the pump source. OPSLs offer several advantages over ILDs, particularly in wavelength selection and lack of interference from internal electrode structures. When an electron and a hole are present in the same region, they may recombine or “annihilate” with the result being spontaneous emission—i.e., the electron may re-occupy the energy state of the hole, emitting a photon with energy equal to the difference between the electron and hole states involved. (In a conventional semiconductor junction diode, the energy released from the recombination of electrons and holes is carried away as phonons, i.e., lattice vibrations, rather than as photons.) Spontaneous emission gives the laser diode below lasing threshold similar properties to an LED. Spontaneous emission is necessary to initiate laser oscillation, but it is one among several sources of inefficiency once the laser is oscillating. The difference between the photon-emitting semiconductor laser and conventional phonon-emitting (non-light-emitting) semiconductor junction diodes lies in the use of a different type of semiconductor, one whose physical and atomic structure confers the possibility for photon emission. These photon-emitting semiconductors are the so-called “direct bandgap” semiconductors. The properties of silicon and germanium, which are single-element semiconductors, have bandgaps that do not align in the way needed to allow photon emission and are not considered “direct.” Other materials, the so-called compound semiconductors, have virtually identical crystalline structures as silicon or germanium but use alternating arrangements of two different atomic species in a checkerboard-like pattern to break the symmetry. The transition between the materials in the alternating pattern creates the critical “direct bandgap” property. Gallium arsenide, indium phosphide, gallium antimonide, and gallium nitride are all examples of compound semiconductor materials that may be used to create junction diodes that emit light.

Vertical-cavity surface-emitting lasers (or “VCSEL”s) have the optical cavity axis along the direction of current flow rather than perpendicular to the current flow as in conventional laser diodes. The active region length is very short compared with the lateral dimensions so that the radiation emerges from the surface of the cavity rather than from its edge as shown in the figure. The reflectors at the ends of the cavity are dielectric mirrors made from alternating high and low refractive index quarter-wave thick multilayer. VCSELs allow for monolithic optical structures to be produced.

Horizontal cavity surface-emitting lasers (or “HCSEL”s) combine the power and high reliability of a standard edge-emitting laser diode with the low cost and ease of packaging of a vertical cavity surface-emitting laser (VCSEL). They also lend themselves to use in integrated on-chip optronic, or photonic packages.

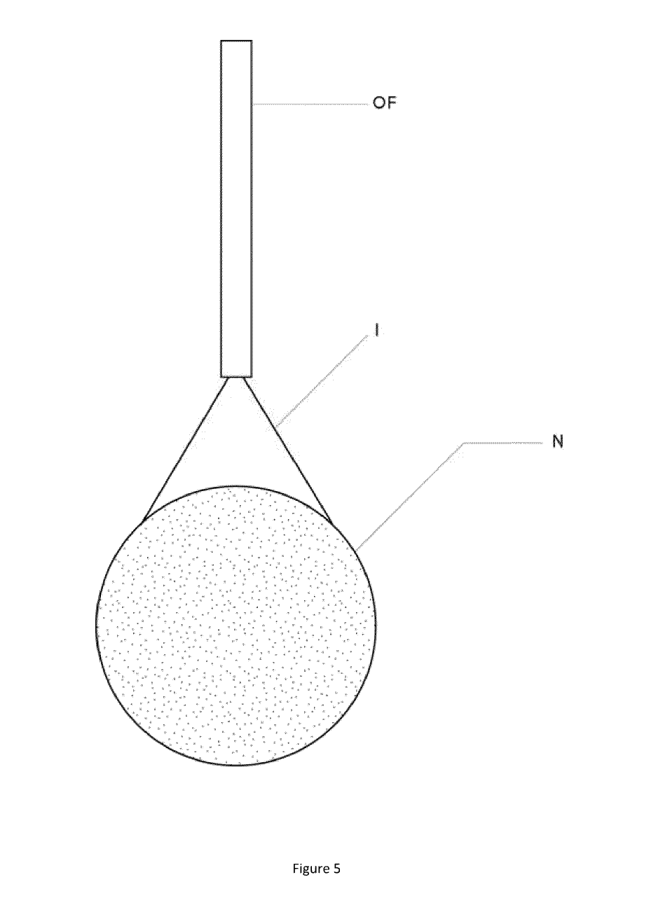

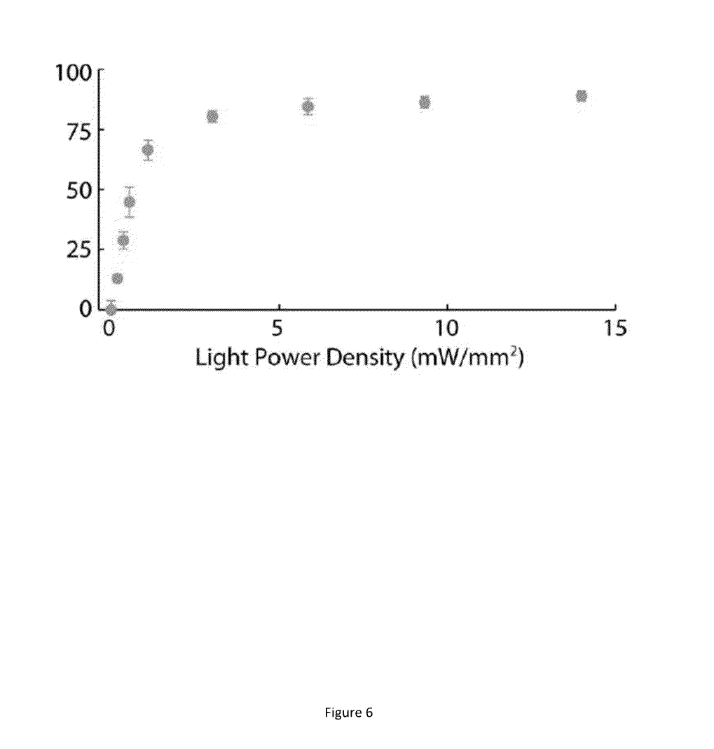

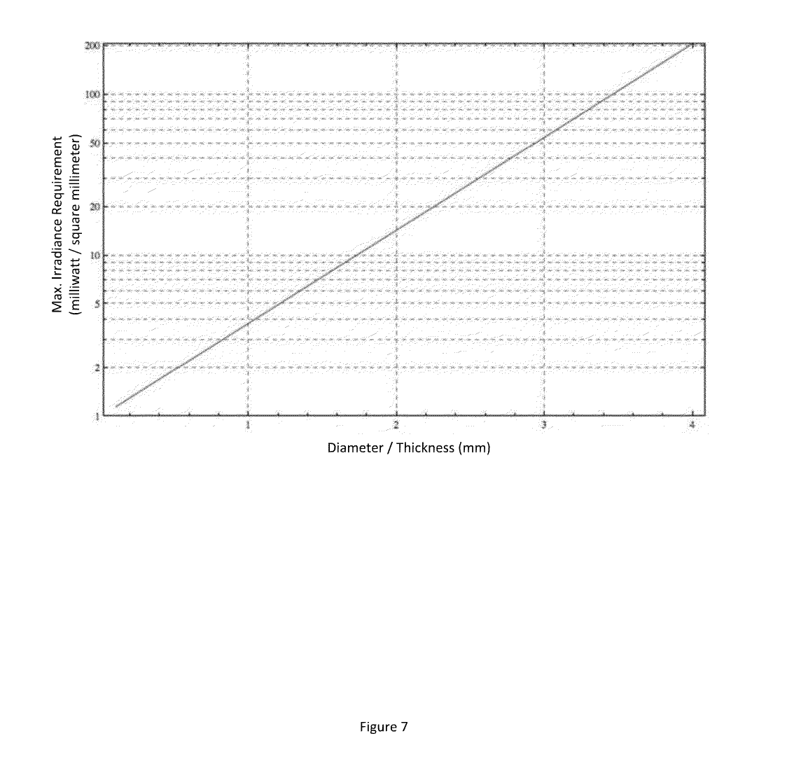

The irradiance required at the neural membrane in which the optogenetic channels reside is on the order of 0.05-2 mW/mm² and depends upon numerous elements, such as opsin channel expression density, activation threshold, etc. A modified channelrhodopsin-2 resident within a neuron may be activated by illumination of the neuron with green or blue light having a wavelength of between about 400 nm and about 550 nm, and in one example about 473 nm, with an intensity of between about 0.5 mW/mm² and about 10 mW/mm², such as between about 1 mW/mm² and about 5 mW/mm², and in one example about 2.4 mW/mm². Although the excitation spectrum may be different, similar exposure values hold for other opsins, such as NpHR and iC1C2, as well. Because most opsin-expressing targets are contained within a tissue or other structure, the light emitted from the applicator may need to be higher in order to attain the requisite values at the target itself. Light intensity, or irradiance, is lost predominantly due to optical scattering in tissue, which is a turbid medium. There is also parasitic absorption of endogenous chromophores, such as blood, that may also diminish the target exposure. Because of these effects, the irradiance range required at the output of an applicator is, for most of the cases described herein, between 1-100 mW/mm². Referring to FIG. 5, experiments have shown, for example, that for the single sided exposure of illumination (I) from an optical fiber (OF) of a 1 mm diameter nerve bundle (N), the measured response (in arbitrary units) vs. irradiance (or Light Power Density, in mW/mm²) is asymptotic, as shown in the graph depicted in FIG. 6. There is not appreciable improvement beyond 20 mW/mm² for this specific configuration of opsin protein, expression density, illumination geometry, and pulse parameters. However, we may use this result to scale the irradiance requirements to other targets with similar optical properties and opsin protein expression densities. The data in FIG. 6 may be used in a diffusion approximation optical model for neural materials, where the irradiance (I) obeys the following relation, I=Iₒe−^{(Qμz)}. The resulting expression fits well with the following experimental data, and the result of this is given in the plot of FIG. 7. The details are further discussed below.

The optical penetration depth, δ, is the tissue thickness that causes light to attenuate to e⁻¹ (˜37%) of its initial value, and is given by the following diffusion approximation.

$$δ = \frac{1}{\sqrt{3 {μ}_{a} {u}_{s}^{′}}} ,$$

where μₐ is the absorption coefficient, and μₛ, is the reduced scattering coefficient. The reduced scattering coefficient is a lumped property incorporating the scattering coefficient μₛ and the anisotropy g: μₛ′=μₛ(1−g) [cm⁻¹]. The purpose of μₛ′ is to describe the diffusion of photons in a random walk of step size of 1/μₛ′ [cm] where each step involves isotropic scattering. Such a description is equivalent to description of photon movement using many small steps 1/μₛ that each involve only a partial deflection angle θ, if there are many scattering events before an absorption event, i.e., μₐ<<μₛ′. The anisotropy of scattering, g, is effectively the expectation value of the scattering angle, θ. Furthermore, the “diffusion exponent,” μ_{eff}, is a lumped parameter containing ensemble information regarding the absorption and scattering of materials, μ_{eff}=Sqrt(3μₐ(μₐ+μ_{s′}). The cerebral cortex constitutes a superficial layer of grey matter (high proportion of nerve cell bodies) and internally the white matter, which is responsible for communication between axons. The white matter appears white because of the multiple layers formed by the myelin sheaths around the axons, which are the origin of the high, inhomogeneous and anisotropic scattering properties of brain, and is a suitable surrogate for use in neural tissue optics calculations with published optical properties, such as those below for feline white matter.

| λ [nm] | μₛ [cm⁻¹] | μₐ [cm⁻¹] | g | μₛ, [cm⁻¹] | μ_{eff} [cm⁻¹] | δ [cm] |
|---|---|---|---|---|---|---|
| 633 | 52.6 | 1.58 | 0.80 | 10.52 | 7.5 | 0.14 |
| 514 | — | — | — | — | 10.9 | 0.091 |
| 488 | — | — | — | — | 13.3 | 0.075 |

As was described earlier, the one-dimensional irradiance profile in tissue, I, obeys the following relation, I=Iₒe−^{(Qμz)}, where Q is the volume fraction of the characterized material that is surrounded by an optically neutral substance such as interstitial fluid or physiologic saline. In the case of most nerves, Q=0.45 can be estimated from cross-sectional images. The optical transport properties of tissue yield an exponential decrease of the irradiance (ignoring temporal spreading, which is inconsequential for this application) through the target, or the tissue surrounding the target(s). The plot above contains good agreement between theory and model, validating the approach. It can be also seen that the optical penetration depth, as calculated by the above optical parameters agrees reasonably well with the experimental observations of measured response vs. irradiance for the example described above.

Furthermore, the use of multidirectional illumination, as has been described herein, may serve to reduce this demand, and thus the target radius may be considered as the limiting geometry, and not the diameter. For instance, if the abovementioned case of illuminating a 1 mm nerve from 2 opposing sides instead of just the one, we can see that we will only need an irradiance of ˜6 mW/mm² because the effective thickness of the target tissue is now ½ of what it was. It should be noted that this is not a simple linear system, or the irradiance value would have been 20/2=10 mW/mm². The discrepancy lies in the exponential nature of the photon transport process, which yields the severe diminution of the incident power at the extremes of the irradiation field. Thus, there is a practical limit to the number of illuminations directions that provide an efficiency advantage for deep, thick, and/or embedded tissue targets.

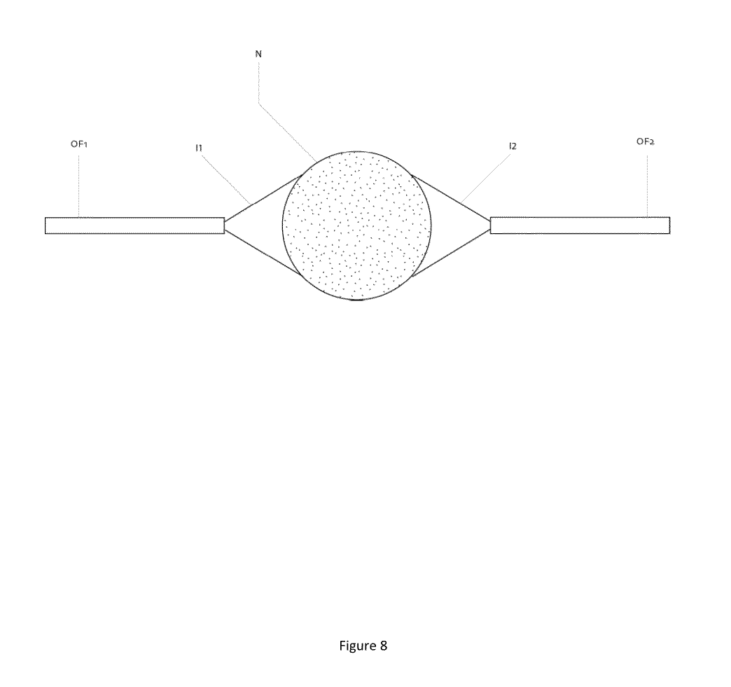

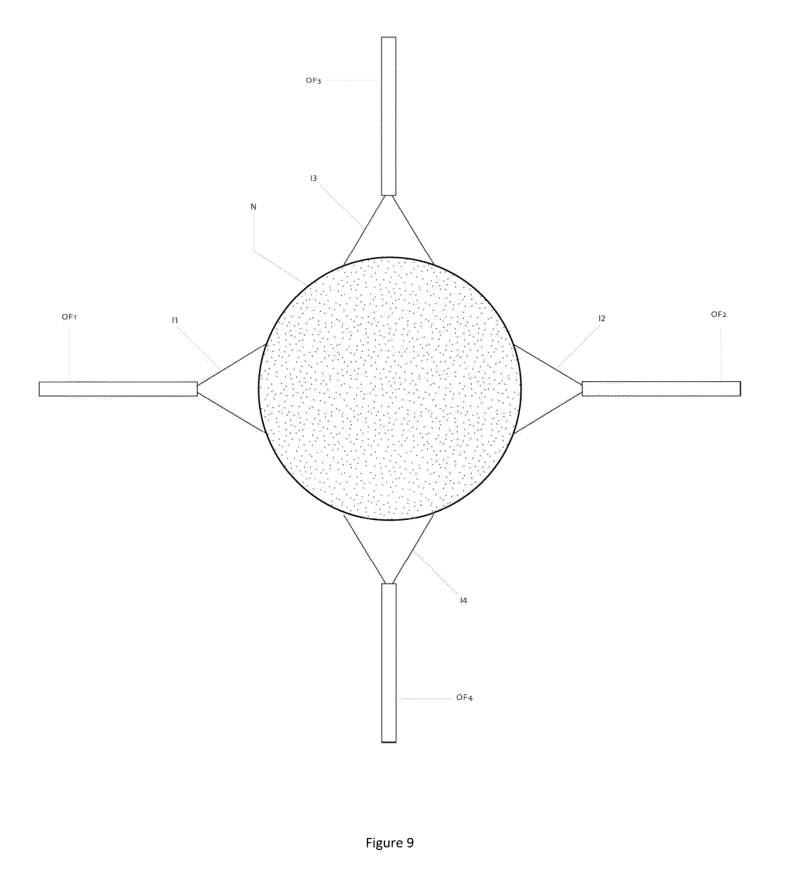

By way of non-limiting example, a 2 mm diameter nerve target may be considered a 1 mm thick target when illuminated circumferentially. Values of the sizes of a few key nerves follow as a set of non-limiting examples. The diameter of the main trunk of the pudendal nerve is 4.67±1.17 mm, whereas the branches of the ulnar nerve range in diameter from about 0.7-2.2 mm and the vagus nerve in the neck between 1.5-2.5 mm. Circumferential, and/or broad illumination may be employed to achieve electrically and optically efficient optogenetic target activation for larger structures and/or enclosed targets that cannot be addressed directly. This is illustrated in FIG. 8, where Optical Fibers OF1 and OF2 now illuminate the targeted tissue structure (N) from diametrically opposing sides with Illumination Fields I1 & I2, respectively. Alternately, the physical length of the illumination may be extended to provide for more photoactivation of expressed opsin proteins, without the commensurate heat build up associated with intense illumination limited to smaller area. That is, the energy may be spread out over a larger area to reduce localized temperature rises. In a further embodiment, the applicator may contain a temperature sensor, such as an RTD, thermocouple, or thermistor, etc. to provide feedback to the processor in the housing to assure that temperature rises are not excessive, as is discussed in further detail below.

From the examples above, activation of a neuron, or set(s) of neurons within a 2.5 mm diameter vagus nerve may be nominally circumferentially illuminated by means of the optical applicators described later using an external surface irradiance of ≧5.3 mW/mm², as can be seen using the above curve when considering the radius as the target tissue thickness, as before. However, this is greatly improved over the 28 mW/mm² required for a 2.5 mm target diameter, or thickness. In this case, 2 sets of the opposing illumination systems from the embodiment above may be used, as the target surface area has increased, configuring the system to use Optical Fibers OF3 & OF4 to provide Illumination Fields I3 & I4, as shown in FIG. 9. There are also thermal concerns to be understood and accounted for in the design of optogenetic systems, and excessive irradiances will cause proportionately large temperature rises. Thus, it may be beneficial to provide more direct optical access to targets embedded in tissues with effective depths of greater than ˜2 mm because of the regulatory limit applied to temperature rise allowed by conventional electrical stimulation, or “e-stim”, devices of ΔT≦2.0° C.

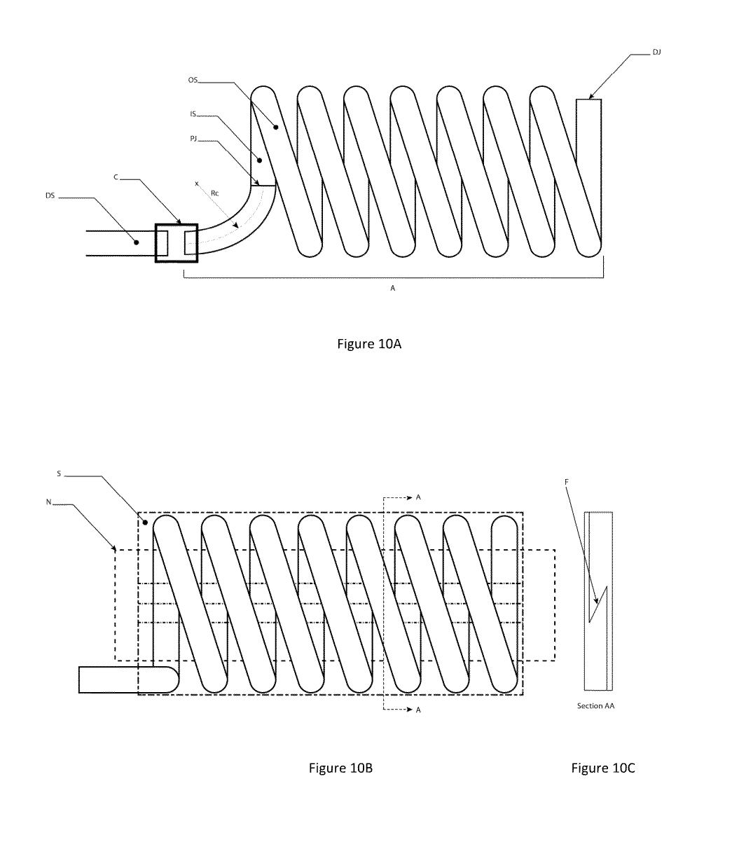

As described above, optical applicators suitable for use with the present invention may be configured in a variety of ways. Referring to FIGS. 10A-10C, a helical applicator with a spring-like geometry is depicted. Such a configuration may be configured to readily bend with, and/or conform to, a targeted tissue structure (N), such as a nerve, nerve bundle, vessel, or other structure to which it is temporarily or permanently coupled. Such a configuration may be coupled to such targeted tissue structure (N) by “screwing” the structure onto the target, or onto one or more tissue structures which surround or are coupled to the target. As shown in the embodiment of FIG. 10A, a waveguide may be connected to, or be a contiguous part of, a delivery segment (DS), and separable from the applicator (A) in that it may be connected to the applicator via connector (C). Alternately, it may be affixed to the applicator portion without a connector and not removable. Both of these embodiments are also described with respect to the surgical procedure described herein. Connector (C) may be configured to serve as a slip-fit sleeve into which both the distal end of Delivery Segment (DS) and the proximal end of the applicator are inserted. In the case where the delivery segment is an optical conduit, such an optical fiber, it preferably should be somewhat undersized in comparison to the applicator waveguide to allow for axial misalignment. For example, a 50 μm core diameter fiber may be used as delivery segment (DS) to couple to a 100 μm diameter waveguide in the applicator (A). Such 50 μm axial tolerances are well within the capability of modern manufacturing practices, including both machining and molding processes. The term waveguide is used herein to describe an optical conduit that confines light to propagate nominally within it, albeit with exceptions for output coupling of the light, especially to illuminate the target.

Biocompatible adhesive may be applied to the ends of connector (C) to ensure the integrity of the coupling. Alternately, connector (C) may be configured to be a contiguous part of either the applicator or the delivery device. Connector (C) may also provide a hermetic electrical connection in the case where the light source is located at the applicator. In this case, it may also serve to house the light source, too. The light source may be made to butt-couple to the waveguide of the applicator for efficient optical transport. Connector (C) may be contiguous with the delivery segment or the applicator. Connector (C) may be made to have cross-sectional shape with multiple internal lobes such that it may better serve to center the delivery segment to the applicator.

The applicator (A) in this embodiment also comprises a Proximal Junction (PJ) that defines the beginning of the applicator segment that is in optical proximity to the target nerve. That is, PJ is the proximal location on the applicator optical conduit (with respect to the direction the light travels into the applicator) that is well positioned and suited to provide for light output onto the target. The segment just before PJ is curved, in this example, to provide for a more linear aspect to the overall device, such as might be required when the applicator is deployed along a nerve, and is not necessarily well suited for target illumination. Furthermore, the applicator of this exemplary embodiment also comprises a Distal Junction (DJ), and Inner Surface (IS), and an Outer Surface (OS). Distal Junction (DJ) represents the final location of the applicator still well positioned and suited to illuminate the target tissue(s). However, the applicator may extend beyond DJ, no illumination is intended beyond DJ. DJ may also be made to be a reflective element, such as a mirror, retro-reflector, diffuse reflector, a diffraction grating, A Fiber Bragg Grating (“FBG”—further described below in reference to FIG. 12), or any combination thereof. An integrating sphere made from an encapsulated “bleb” of BaSO₄, or other such inert, non-chromophoric compound may serve a diffuse reflector when positioned, for example, at the distal and of the applicator waveguide. Such a scattering element should also be placed away from the target area, unless light that is disallowed from waveguiding due to its spatial and/or angular distribution is desired for therapeutic illumination.

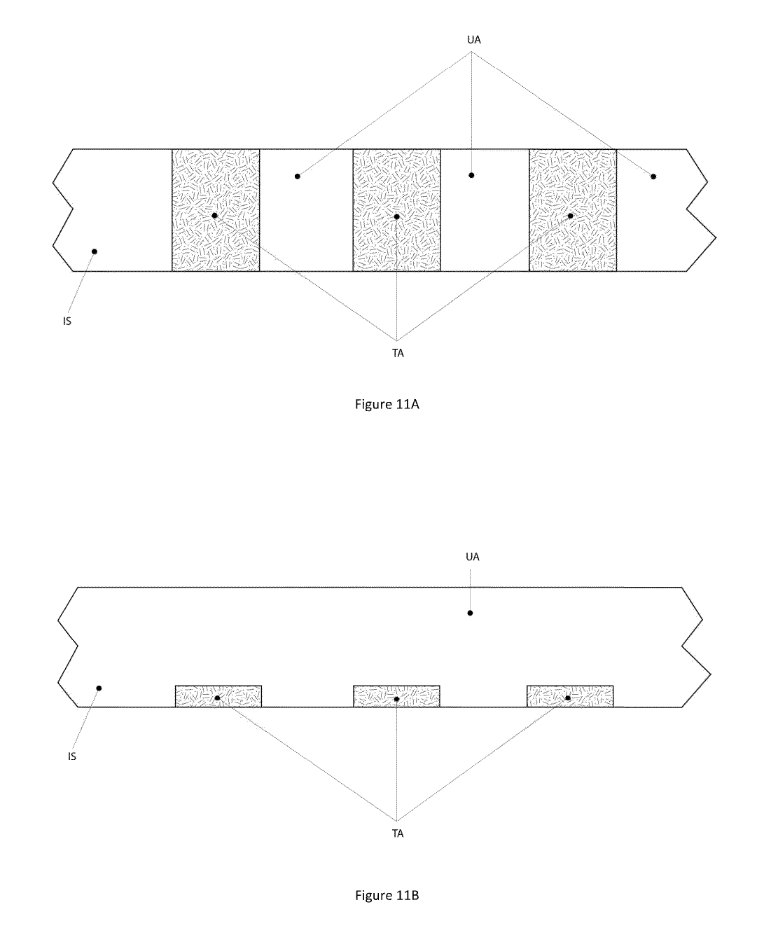

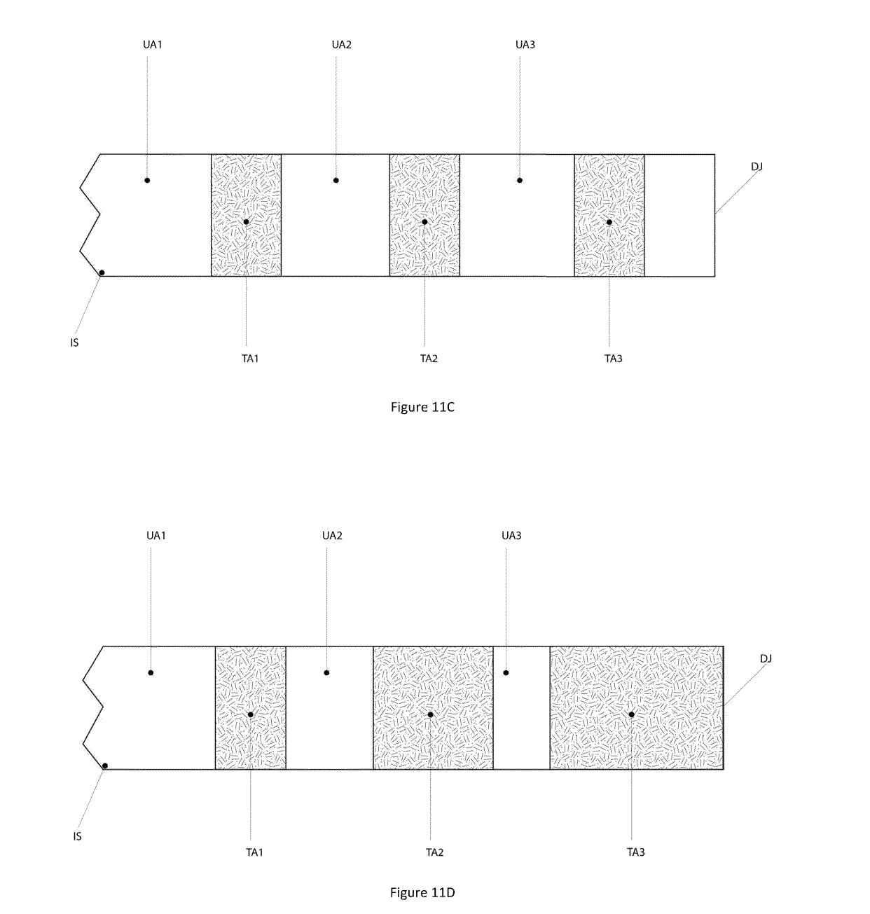

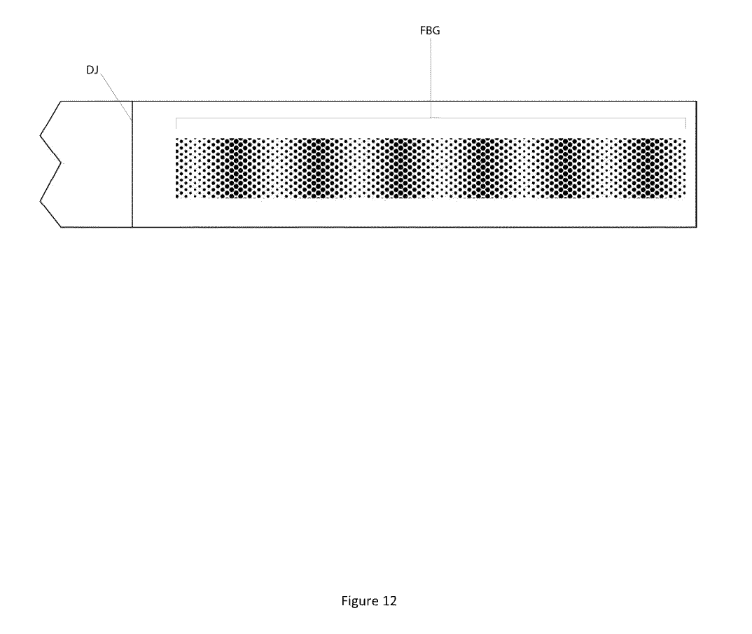

Inner Surface (IS) describes the portion of the applicator that “faces” the target tissue, shown here as Nerve (N). That is, N lies within the coils of the applicator and is in optical communication with IS. That is, light exiting IS is directed towards N. Similarly, Outer Surface (OS) describes that portion of the applicator that is not in optical communication with the target. That is, the portion that faces outwards, away from the target, such a nerve that lies within the helix. Outer Surface (OS) may be made to be a reflective surface, and as such will serve to confine the light within the waveguide and allow for output to the target via Inner Surface (IS). The reflectivity of OS may be achieved by use of a metallic or dielectric reflector deposited along it, or simply via the intrinsic mechanism underlying fiber optics, total internal reflection (“TIR”). Furthermore, Inner Surface (IS) may be conditioned, or affected, such that it provides for output coupling of the light confined within the helical waveguide. The term output coupling is used herein to describe the process of allowing light to exit the waveguide in a controlled fashion, or desired manner. Output coupling may be achieved in various ways. One such approach may be to texture IS such that light being internally reflected no longer encounters a smooth TIR interface. This may be done along IS continuously, or in steps. The former is illustrated in FIG. 11A in a schematic representation of such a textured applicator, as seen from IS. Surface texture is synonymous with surface roughness, or rugosity. It is shown in the accompanying figure as being isotropic, and thus lacking a definitive directionality. The degree of roughness is proportional to the output coupling efficiency, or the amount of light removed form the applicator in proportion to the amount of light encountering the Textured Area. One may envision this as being like a matte finish, whereas OS will be like a gloss finish. A Textured Area may be an area along or within a waveguide that is more than a simple surface treatment. It might also comprise a depth component that either diminishes the waveguide cross sectional area, or increases it to allow for output coupling of light for target illumination.

In this non-limiting example, IS contains areas textured with Textured Areas TA correspond to output couplers (OCs), and between them are Untextured Areas (UA). Texturing of textured Areas (TA) may be accomplished by, for example, mechanical means (such as abrasion) or chemical means (such as etching). In the case where optical fiber is used as the basis for the applicator, one may first strip the buffer and cladding layers to expose the core for texturing. The waveguide may lay flat (with respect to gravity) for more uniform depth of surface etching, or may be tilted to provide for a more wedge-shaped etch.

Referring to the schematic representation of FIG. 11B, an applicator is seen from the side with IS facing downward, and TA that do not wrap around the applicator to the outer surface (OS). Indeed, in such embodiment, they need not wrap even halfway around: because the texture may output couple light into a broad solid angle, Textured Areas (TA) need not be of large radial angular extent.

In either case, the proportion of light coupled out to the target should may also be controlled to be a function of the location along the applicator to provide more uniform illumination output coupling from IS to the target, as shown below. This may be done to account for the diminishing proportion of light encountering later (or distal) output coupling zones. For example, if we consider the three (3) output coupling zones represented by Textured Areas (TA) in the present non-limiting example schematically illustrated in FIG. 11B, we now have TA1, TA2, and TA3. In order to provide equal distribution of the output coupled energy (or power) the output coupling efficiencies would be as follows: TA1=33%, TA2=50%, TA3=100%. Of course, other such portioning schemes may be used for different numbers of output coupling zones TAx, or in the case where there is directionality to the output coupling efficiency and a retro-reflector is used in a two-pass configuration, as is described in further detail below.

Referring to FIG. 11C, in the depicted alternate embodiment, distal junction (DJ) is identified to make clear the distinction of the size of TA with respect to the direction of light propagation.

In another embodiment, as illustrated in FIG. 11D, Textured Areas TA1, TA2 and TA3 are of increasing size because they are progressively more distal with the applicator. Likewise, Untextured Areas UA1, UA2 and UA3 are shown to become progressively smaller, although they also may be made constant. The extent (or separation, size, area, etc.) of the Untextured Areas (UAx) dictates the amount of illumination zone overlap, which is another means by which the ultimate illumination distribution may be controlled and made to be more homogeneous in ensemble. Note that Outer Surface (OS) may be made to be reflective, as described earlier, to prevent light scattered from a TA to escape the waveguide via OS and enhance the overall efficiency of the device.

In a similar manner, the surface roughness of the Textured Areas (TA) may be changed as a function of location along the applicator. As described above, the amount of output coupling is proportional to the surface rugosity, or roughness. In particular, it is proportional to the first raw moment (“mean”) of the distribution characterizing the surface rugosity. The uniformity in both it spatial and angular emission are proportional to the third and forth standardized moments (or “skewness” and “kurtosis”), respectively. These are values that may be adjusted, or tailored, to suit the clinical and/or design need in a particular embodiment. Also, the size, extent, spacing and surface roughness may each be employed for controlling the amount and ensemble distribution of the target illumination.

Alternately, directionally specific output coupling maybe employed that preferentially outputs light traveling in a certain direction by virtue of the angle it makes with respect to IS. For example, a wedge-shaped groove transverse to the waveguide axis of IS will preferentially couple light encountering it when the angle incidence is greater than that required for TIR. If not, the light will be internally reflected and continue to travel down the applicator waveguide.

Furthermore, in such a directionally specific output coupling configuration, the applicator may utilize the abovementioned retro-reflection means distal to DJ. FIG. 12 illustrates an example comprising a FBG retro-reflector.

A waveguide, such as a fiber, can support one or even many guided modes. Modes are the intensity distributions that are located at or immediately around the fiber core, although some of the intensity may propagate within the fiber cladding. In addition, there is a multitude of cladding modes, which are not restricted to the core region. The optical power in cladding modes is usually lost after some moderate distance of propagation, but can in some cases propagate over longer distances. Outside the cladding, there is typically a protective polymer coating, which gives the fiber improved mechanical strength and protection against moisture, and also determines the losses for cladding modes. Such buffer coatings may consist of acrylate, silicone or polyimide. For long-term implantation in a body, moisture must be kept away from the waveguide to prevent refractive index changes that will alter the target illumination distribution and yield other commensurate losses. Therefore, for long-term implantation, a buffer layer (or region) may be applied to the Textured Areas TAx of the applicator waveguide. Long-term is herein defined as greater than or equal to 2 years. The predominant deleterious effect of moisture absorption on optical waveguides is the creation of hydroxyl absorption bands that cause transmission losses in the system. This is a negligible for the visible spectrum, but an issue for light with wavelengths longer than about 850 nm. Secondarily, moisture absorption may reduce the material strength of the waveguide itself and lead to fatigue failure. Thus, while it is a concern, it is more of a concern for the delivery segments, which may likely undergo more motion and cycles of motion than the applicator.

Furthermore, the applicator maybe enveloped or partially enclosed by a jacket, such as Sleeve S shown in the figure. Sleeve S may be made to be a reflector, as well, and serve to confine light to the intended target. Reflective material(s), such as Mylar, metal foils, or sheets of multilayer dielectric thin films may be located within the bulk of Sleeve S, or along its inner or outer surfaces. While the outer surface of Sleeve S may also be utilized reflective purposes, it is not preferred, as it is in more intimate contact with the surrounding tissue than the inner surface. Such a jacket may be fabricated from polymeric material to provide the necessary compliance required for a tight fit around the applicator. Sleeve S, or an adjunct or alternative to, may be configured such that its ends slightly compress the target over a slight distance, but circumferentially to prevent axial migration, infiltration along the target surface. Sleeve S may also be made to be highly scattering (white, high albedo) to serve as diffusive retro-reflector to improve overall optical efficiency by redirecting light to the target.

Fluidic compression may also be used to snug the sleeve over the applicator and provide for a tighter fit to inhibit proliferation of cells and tissue ingrowth that may degrade the optical delivery to the target. Fluidic channels may be integrated into Sleeve S and filled at the time of implantation. A valve or pinch-off may be employed to seal the fluidic channels. Further details are described in a subsequent section.

Furthermore, Sleeve S may also be made to elute compounds that inhibit scar tissue formation. This may provide for increased longevity of the optical irradiation parameters that might otherwise be altered by the formation of a scar, or the infiltration of tissue between the applicator and the target. Such tissues may scatter light and diminish the optical exposure. However, the presence of such infiltrates could also be detected by means of an optical sensor placed adjacent to the target or the applicator. Such a sensor could serve to monitor the optical properties of the local environment for system diagnostic purposes. Sleeve S may also be configured to utilize a joining means that is self-sufficient, such as is illustrated in the cross-section of FIG. 10C, wherein at least a part of the applicator is shown enclosed in cross-section A-A. Alternately, Sleeve S may be joined using sutures or such mechanical or geometric means of attachment, as illustrated by element F in the simplified schematic of FIG. 10C.

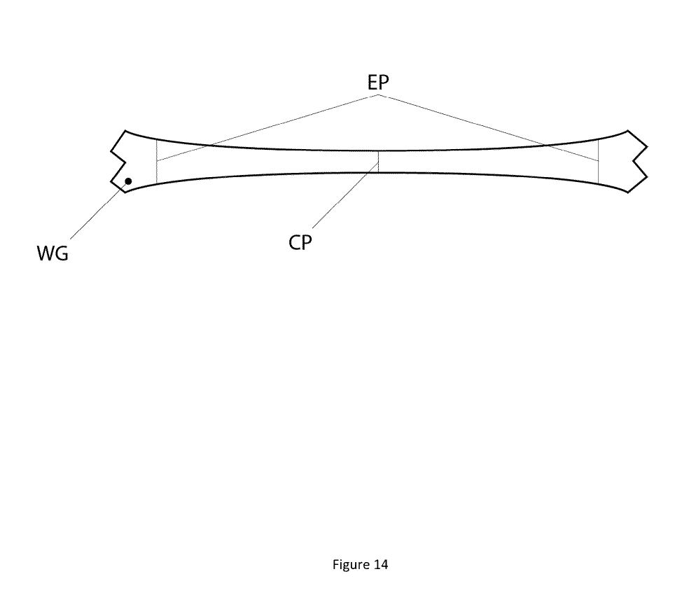

In a further embodiment, output coupling may be achieved by means of localized strain-induced effects with the applicator waveguide that serve to alter the trajectory of the light within it, or the bulk refractive index on the waveguide material itself, such as the use of polarization or modal dispersion. For example, output coupling may be achieved by placing regions (or areas, or volumes) of form-induced refractive index variation and/or birefringence that serve to alter the trajectory of the light within the waveguide beyond the critical angle required for spatial confinement and/or by altering the value of the critical angle, which is refractive-index-dependent. Alternately, the shape of the waveguide may be altered to output couple light from the waveguide because the angle of incidence at the periphery of the waveguide has been modified to be greater than that of the critical angle required for waveguide confinement. These modifications may be accomplished by heating, and/or twisting, and/or pinching the applicator in those regions where output coupling for target illumination is desired. A non-limiting example is shown in FIG. 14, where a truncated section of Waveguide WG has been modified between Endpoints (EP) and Centerpoint (CP). The cross-sectional area and/or diameter of CP<EP. Light propagating through Waveguide WG will encounter a higher angle of incidence at the periphery of the waveguide due to the mechanical alteration of the waveguide material, resulting in light output coupling near CP in this exemplary configuration. It should be noted that light impinging upon the relatively slanted surface provided by the taper between EP and CP may output couple directly from the WG when the angle is sufficiently steep, and may require more than a single interaction with said taper before its direction is altered to such a degree that is ejected from the WG. As such, consideration may be given to which side of the WG is tapered, if it is not tapered uniformly, such that the output coupled light exiting the waveguide is directed toward the target, or incident upon an alternate structure, such as a reflector to redirect it to the target.

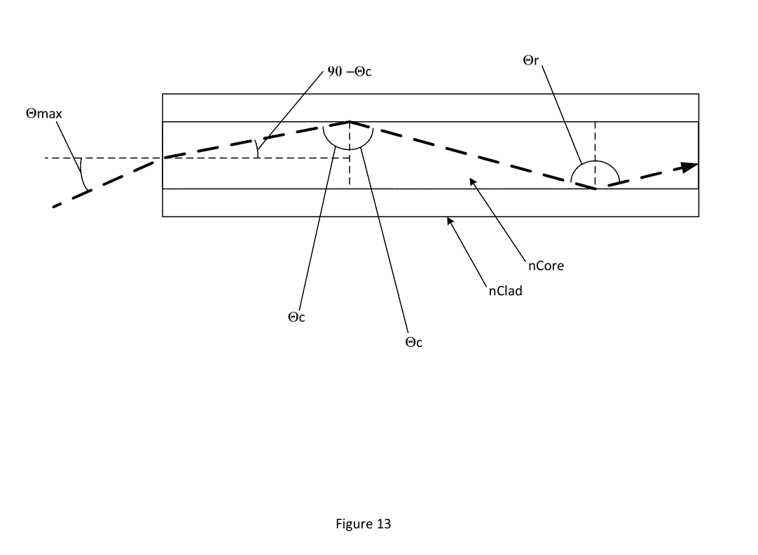

Referring to FIG. 13 and the description that follows, for contextual purposes an exemplary scenario is described wherein a light ray is incident from a medium of refractive index “n” upon a core of index “n_{core}” at a maximum acceptance angle, θₘₐₓ, with Snell's law at the medium-core interface being applied. From the geometry illustrated in FIG. 13, we have:

From the geometry of the above figure we have:

sin θᵣ=sin(90°−θ_{c})=cos θ_{c}

where

$${θ}_{c} = {sin}^{- 1} \frac{{n}_{clad}}{{n}_{core}}$$

is the critical angle for total internal reflection.

Substituting cos θc for sin θr in Snell's law we get:

$$\frac{n}{{n}_{core}} sin {θ}_{max} = cos {θ}_{c} .$$

By squaring both sides we get:

$$\frac{{n}^{2}}{{n}_{core}^{2}} {sin}^{2} {θ}_{max} = {cos}^{2} {θ}_{c} = 1 - {sin}^{2} {θ}_{c} = 1 - \frac{{n}_{clad}^{2}}{{n}_{core}^{2}} .$$

Solving, we find the formula stated above:

n sin θₘₐₓ=\sqrt{n_{core}²−n_{clad}²},

This has the same form as the numerical aperture (NA) in other optical systems, so it has become common to define the NA of any type of fiber to be

NA=\sqrt{n_{core}²−n_{clad}²},

It should be noted that not all of the optical energy impinging at less than the critical angle will be coupled out of the system.

Alternately, the refractive index may be modified using exposure to ultraviolet (UV) light, such might be done to create a Fiber Bragg Grating (FBG). This modification of the bulk waveguide material will cause the light propagating through the waveguide to refractive to greater or lesser extent due to the refractive index variation. Normally a germanium-doped silica fiber is used in the fabrication of such refractive index variations. The germanium-doped fiber is photosensitive, which means that the refractive index of the core changes with exposure to UV light.

Alternately, and/or in combination with the abovementioned aspects and embodiments of the present invention, “whispering gallery modes” may be utilized within the waveguide to provide for enhanced geometric and/or strain-induced output coupling of the light along the length of the waveguide. Such modes of propagation are more sensitive to small changes in the refractive index, birefringence and the critical confinement angle than typical waveguide-filling modes because they are concentrated about the periphery of a waveguide. Thus, they are more susceptible to such means of output coupling and provide for more subtle means of producing a controlled illumination distribution at the target tissue.

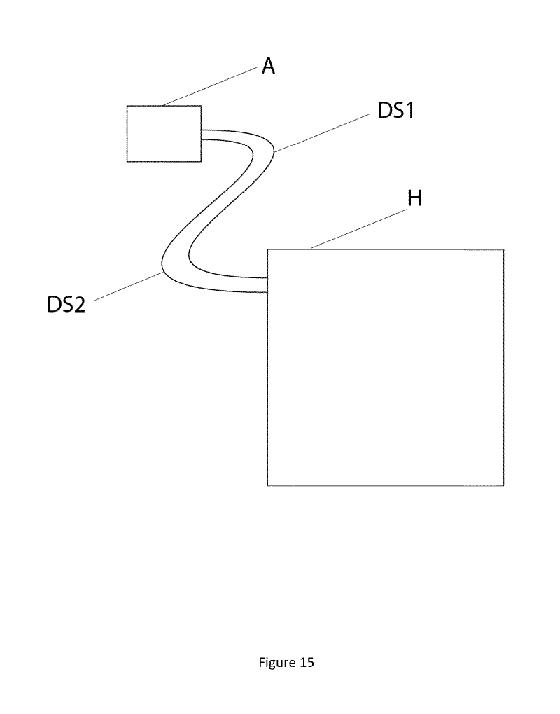

Alternately, more than a single Delivery Segment DS may be brought from the housing (H) to the applicator (A), as shown in FIG. 15. Here Delivery Segments DS1 and DS2 are separate and distinct. They may carry light from different sources (and of different color, or wavelength, or spectra) in the case where the light is created in housing (H), or they may be separate wires (or leads, or cables) in the case where the light is created at or near applicator (A).

In either case, the applicator may alternately further comprise separate optical channels for the light from the different Delivery Segments DSx (where x denotes the individual number of a particular delivery segment) in order to nominally illuminate the target area. A further alternate embodiment may exploit the inherent spectral sensitivity of the retro-reflection means to provide for decreased output coupling of one channel over another. Such would be the case when using a FBG retro-reflector, for instance. In this exemplary case, light of a single color, or narrow range of colors will be acted on by the FBG. Thus, it will retro-reflect only the light from a given source for bi-directional output coupling, while light form the other source will pass through largely unperturbed and be ejected elsewhere. Alternately, a chirped FBG may be used to provide for retro-reflection of a broader spectrum, allowing for more than a single narrow wavelength range to be acted upon by the FBG and be utilized in bi-directional output coupling. Of course, more than two such channels and/or Delivery Segments (DSx) are also within the scope of the present invention, such as might be the case when selecting to control the directionality of the instigated nerve impulse, as will be described in a subsequent section.

Alternately, multiple Delivery Segments may also provide light to a single applicator, or become the applicator(s) themselves, as is described in further detail below.

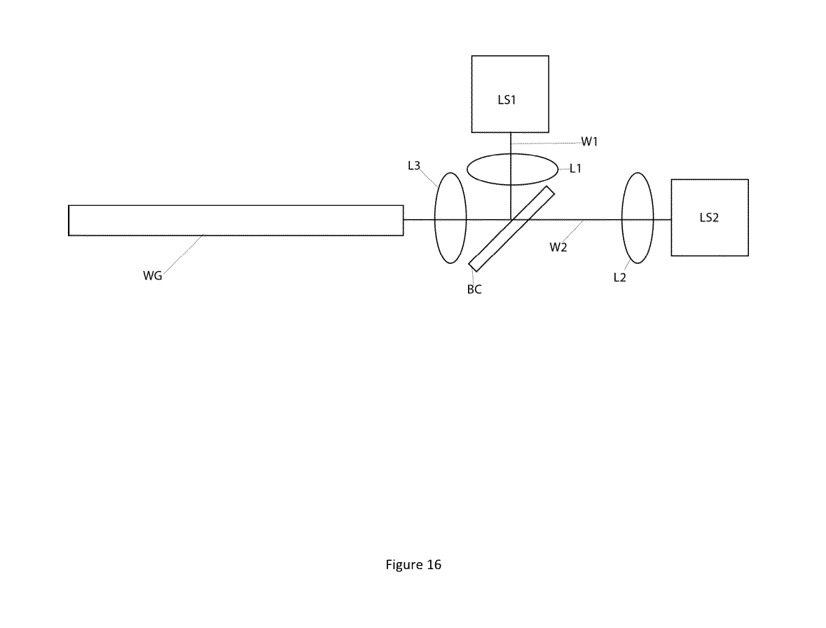

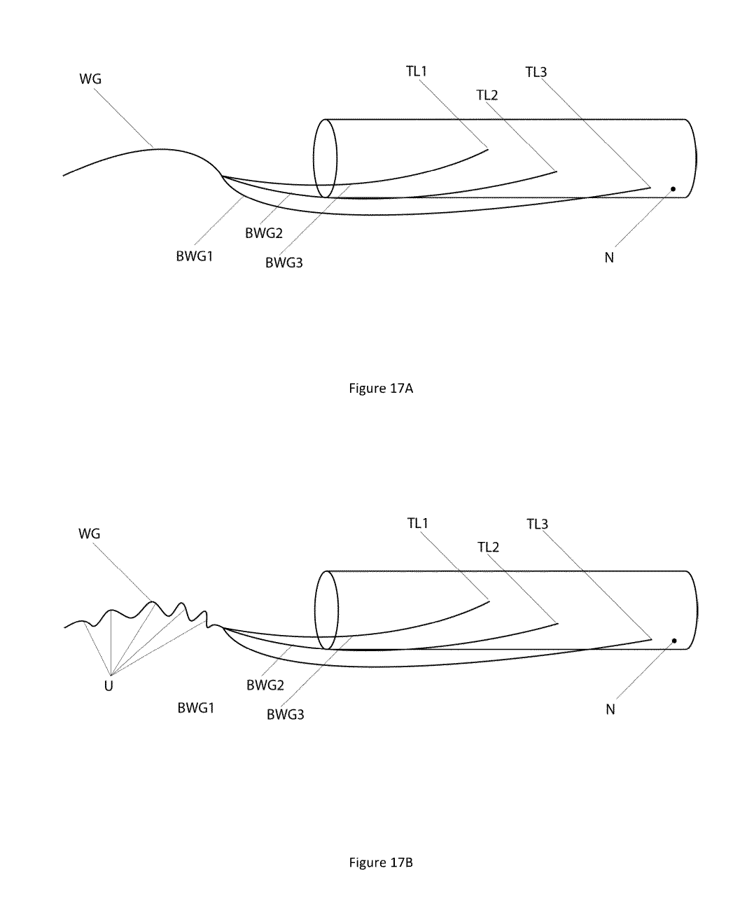

Alternately, a single delivery device may used to channel light from multiple light sources to the applicator. This may be achieved through the use of spliced, or conjoined, waveguides (such as optical fibers), or by means of a fiber switcher, or a beam combiner prior to initial injection into the waveguide, as shown in FIG. 16.

In this embodiment, Light Sources LS1 & LS2 output light along paths W1 & W2, respectively. Lenses L1 & L2 may be used to redirect the light toward Beam Combiner (BC), which may serve to reflect the output of one light source, while transmitting the other. The output of LS1 & LS2 may be of different color, or wavelength, or spectral band, or they may be the same. If they are different, BC may be a dichroic mirror, or other such spectrally discriminating optical element. If the outputs of Light Sources LS1 & LS2 are spectrally similar, BC may utilize polarization to combine the beams. Lens L3 may be used to couple the W1 & W2 into Waveguide (WG). Lenses L1 & L2 may also be replaced by other optical elements, such as mirrors, etc. This method is extensible to greater numbers of light sources.

The type of optical fiber that may be used as either delivery segments or within the applicators is varied, and may be selected from the group consisting of: Step-index, GRIN, Power-Law index, etc. Alternately, hollow-core waveguides, photonic crystal fiber (PCF), and/or fluid filled channels may also be used as optical conduits. PCF is meant to encompass any waveguide with the ability to confine light in hollow cores or with confinement characteristics not possible in conventional optical fiber. More specific categories of PCF include photonic-bandgap fiber (PBG, PCFs that confine light by band gap effects), holey fiber (PCFs using air holes in their cross-sections), hole-assisted fiber (PCFs guiding light by a conventional higher-index core modified by the presence of air holes), and Bragg fiber (PBG formed by concentric rings of multilayer film). These are also known as “microstructured fibers”. End-caps or such enclosure means should be used with open, hollow waveguides such as tubes and PCF to prevent fluid infill that would spoil the waveguide.

PCF and PBG intrinsically support higher numerical aperture (NA) than standard glass fibers, as do plastic and plastic-clad glass fibers. These provide for the delivery of lower brightness sources, such as LEDs, OLEDs, etc. This is important to note because such lower brightness sources are typically more electrically efficient than laser light sources, which is important for implantable device embodiments in accordance with the present invention that utilize battery power sources. Configurations for to creating high-NA waveguide channels are described in greater detail below.

Alternately, a bundle of small and/or single mode (SM) optical fibers/waveguides may be used to transport light as delivery segments, and/or as an applicator structure, such as is shown in a non-limiting exemplary embodiment in FIG. 17A. In this embodiment, Waveguide (WG) may be part of the Delivery Segment(s) (DS), or part of the applicator (A) itself. As shown in the embodiment of FIG. 17A, the waveguide (WG) bifurcates into a plurality of subsequent waveguides, BWGx. The terminus of each BWGx is Treatment Location (TLx). The terminus may be the area of application/target illumination, or may alternately be affixed to an applicator for target illumination. Such a configuration is appropriate for implantation within a distributed body tissue, such as, by way of non-limiting example, the liver, pancreas, or to access cavernous arteries of the corpora cavernosa (to control the degree of smooth muscle relaxation in erection inducement).

Referring to FIG. 17B, the waveguide (WG) may also be configured to include Undulations (U) in order to accommodate possible motion and/or stretching/constricting of the target tissues, or the tissues surrounding the target tissues. Undulations (U) may be pulsed straight during tissue extension and/or stretching. Alternately, Undulations (U) may be integral to the applicators itself, or it may be a part of the Delivery Segments (DS) supplying the applicator (A). The Undulations (U) may be made to areas of output coupling in embodiments when the Undulations (U) are in the applicator. This may be achieved by means of similar processes to those described earlier regarding means by which to adjust the refractive index and/or the mechanical configuration(s) of the waveguide for fixed output coupling in an applicator. However, in this case, the output coupling is achieved by means of tissue movement that causes such changes. Thus, output coupling is nominally only provided during conditions of tissue extension and/or contraction and/or motion. The Undulations (U) may be configured of a succession of waves, or bends in the waveguide, or be coils, or other such shapes. Alternately, DS containing Undulations (U) may be enclosed in a protective sheath or jacket to allow DS to stretch and contract without encountering tissue directly.

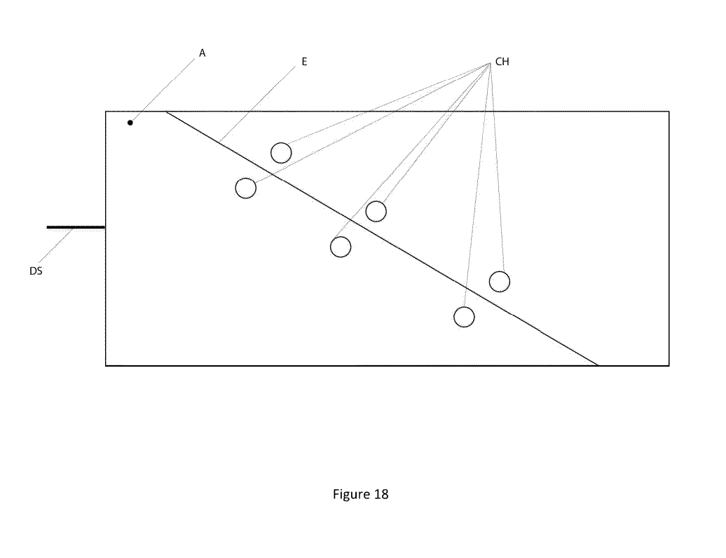

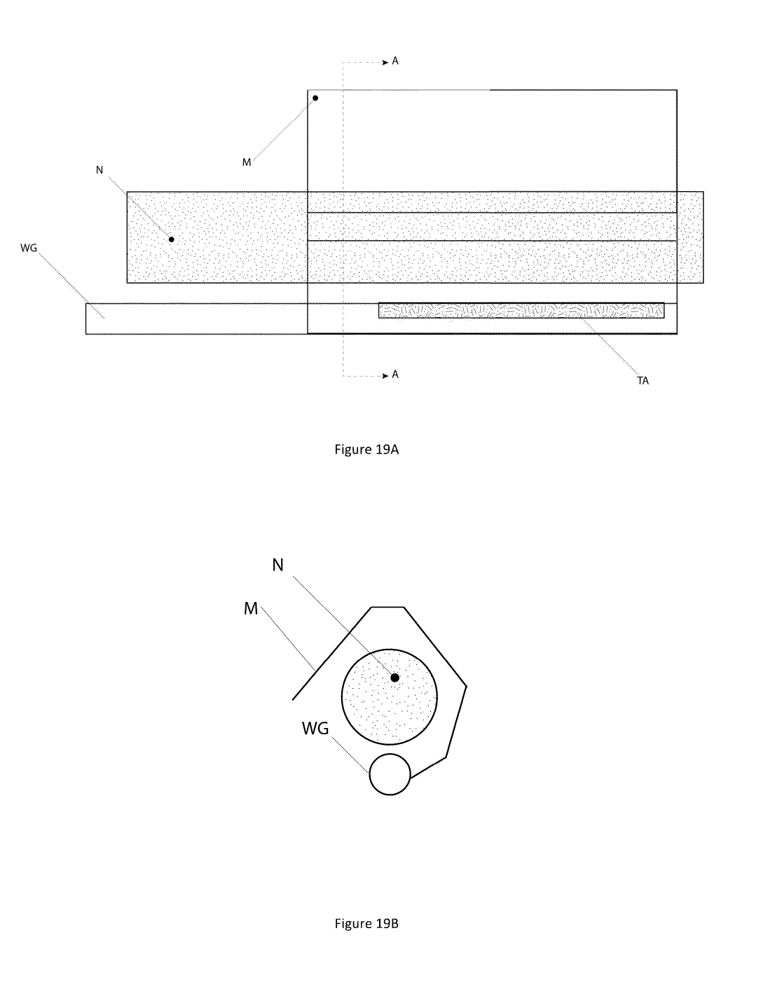

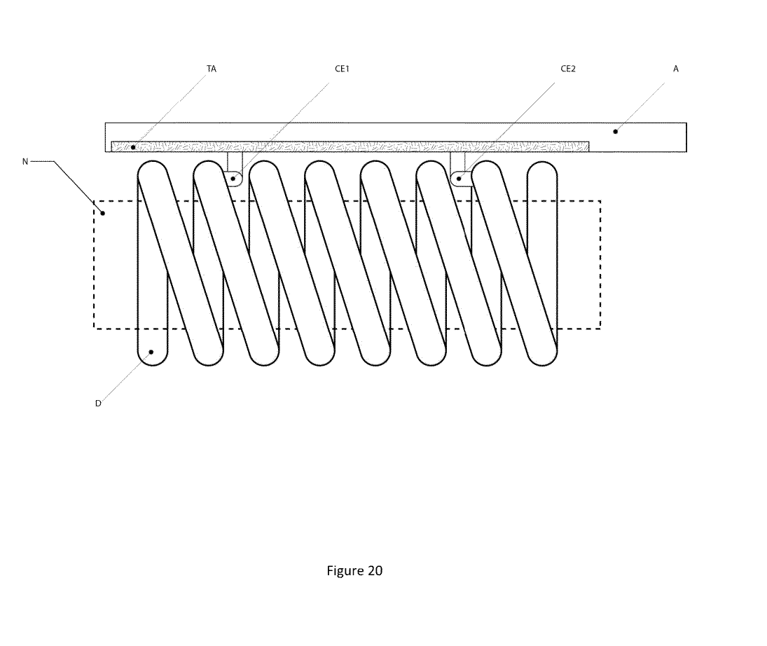

A rectangular slab waveguide may be configured to be like that of the aforementioned helical-type, or it can have a permanent waveguide (WG) attached/inlaid. For example, a slab may be formed such that is a limiting case of a helical-type applicator, such as is illustrated in FIG. 18 for explanatory purposes and to make the statement that the attributes and certain details of the aforementioned helical-type applicators are suitable for this slab-type as well and need not be repeated.

In the exemplary embodiment, Applicator (A) is fed by Delivery Segment (DS) and the effectively half-pitch helix is closed along the depicted edge ∈, with closure holes (CH) provided, but not required. Of course, this is a reduction of the geometries discussed previously, and meant to convey the abstraction and interchangeability of the basic concepts therein and between those of the slab-type waveguides to be discussed.

It should also be understood that the helical-type applicator described herein may also be utilized as a straight applicator, such as may be used to provide illumination along a linear structure like a nerve, etc. A straight applicator may also be configured as the helical-type applicators described herein, such as with a reflector to redirect stray light toward the target, as is illustrated in FIG. 19A by way of non-limiting example.

Here Waveguide (WG) contains Textured Area (TA), and the addition of Reflector (M) that at least partially surrounds target anatomy (N). This configuration provides for exposure of the far side of the target by redirecting purposefully exposed and scattered light toward the side of the target opposite the applicator. FIG. 19B illustrates the same embodiment, along cross-section A-A, showing schematically the use a mirror (as Reflector M) surrounding Target (N.) Although not shown, WG and M may be affixed to a common casing (not shown) that forms part of the applicator. Reflector (M) is shown as being comprised of a plurality of linear faces, but need not be. In one embodiment it may be made to be a smooth curve, or in another embodiment, a combination of the two.

In another alternate embodiment, a straight illuminator may be affixed to the target, or tissue surrounding or adjacent or nearby to the target by means of the same helix-type applicator. However, in this case the helical portion is not the illuminator, it is the means to position and maintain another illuminator in place with respect to the target. The embodiment illustrated in FIG. 20 utilizes the target-engaging feature(s) of the helical-type applicator to locate straight-type Applicator (A) in position near Target (N) via Connector Elements CE1 & CE2, which engage the Support Structure (D) to locate and maintain optical output. Output illumination is shown as being emitted via Textured Area (TA), although, as already discussed, alternate output coupling means are also within the scope of the present invention. The generality of the approach and the interchangeability of the different target-engaging means described herein (even subsequent to this section) are also applicable to serve as such Support Structures (D), and therefore the combination of them is also within the scope of the present invention.

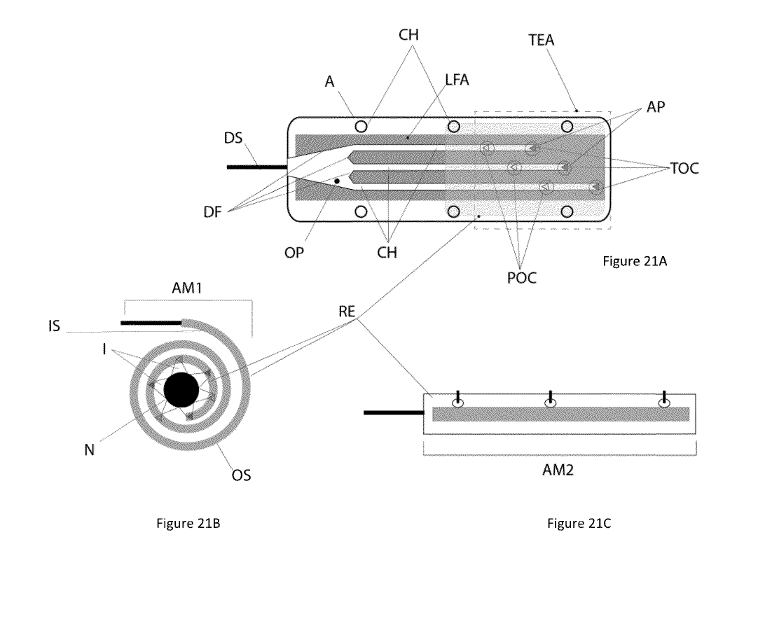

Slab-type geometries of Applicator A, such as thin, planar structures, can be implanted, or installed at, near, or around the tissue target or tissue(s) containing the intended target(s). An embodiment of such a slab-type applicator configuration is illustrated in FIGS. 21A-21C. It may be deployed near or adjacent to a target tissue, and it may also be rolled around the target tissue, or tissues surrounding the target(s). It may be rolled axially, as illustrated by element AM1 in FIG. 21B, (i.e. concentric with the long axis of the targeted tissue structure N), or longitudinally, as illustrated by element AM2 in FIG. 21C (i.e. along the long axis of target N), as required by the immediate surgical situation, as shown in the more detailed figure below. The lateral edges that come into contact with each other once deployed at the target location could be made with complementary features to assure complete coverage and limit the amount of cellular infiltrate (i.e. limit scar tissue or other optical perturbations over time to better assure an invariant target irradiance, as was described in the earlier section pertaining to the helical-type applicator). Closure Holes (CH) are provided for this purpose in the figure of this non-limiting example. The closure holes (CH) may be sutured together, of otherwise coupled using a clamping mechanism (not specifically called-out). It may also provide different output coupling mechanisms than the specific helical-type waveguides described above, although, it is to be understood that such mechanisms are fungible, and may be used generically. And vice-versa, that elements of output coupling, optical recirculation and waveguiding structures, as well as deployment techniques discussed in the slab-type section maybe applicable to helical-type, and straight waveguides, too.

The slab-type applicator (A) illustrated in FIGS. 21A-21C is comprised of various components, as follows. In the order “seen” by light entering the applicator, first is an interface with the waveguide of the delivery segment (DS). Alternately, the waveguide may be replaced by electrical wires, in the case where the emitter(s) is(are) included near or within the applicator. An Optical Plenum (OP) structure may be present after the interface to allow to segment and direct light propagation to different channels CH using distribution facets (DF), whether it comes from the delivery segments (DS), or from a local light source (not shown for simplicity). The optical plenum (OP) may also be configured to redirect all of the light entering the light entering it, such as might be desirable when the delivery segment (DS) should lie predominantly along the same direction as the applicator (A). Alternately, it may be made to predominantly redirect the light at angle to provide for the applicator to be directed differently than the delivery segment(s) (DS). Light propagating along the channel(s) (CH) may encounter an output coupling means, such as Partial Output Coupler (POC) & Total Output Coupler (TOC). The proximal output couplers (POC) redirect only part of the channeled light, letting enough light pass to provide adequate illumination to more distal targets, as was discussed previously. The final, or distal-most, output coupler (TOC) may be made to redirect nominally all of the impinging light to the target. The present embodiment also contains provisions for outer surface reflectors to redirect errant light to the target. It is also configured to support a reflector (RE) on or near the inner surface (IS) of applicator (A), with apertures (AP) to allow for the output coupled light to escape, that serves to more readily redirect any errant or scattered light back toward the target (N). Alternately, such a reflector (RE) may be constructed such that it is not covering the output coupler area, but proximal to it in the case of longitudinally rolled deployment such that it nominally covers the intended target engagement area (TEA). Reflector (RE) may be made from biocompatible materials such as Platinum, or Gold if they are disposed along the outside of the applicator (A). Alternately, such metallic coatings may be functionalized in order to make them bioinert, as is discussed below. The output couplers POC and TOC are shown in the accompanying exemplary figure as being located in the area of the applicator (A) suitable for longitudinal curling about the target (N), or tissues surrounding the target (N), but need not be, as would be the case for deployments utilizing the unrolled and axially rolled embodiments (AM1). Any such surface (or subsurface) reflector (RE) should be present along (or throughout) a length sufficient to provide at least complete circumferential coverage once the applicator is deployed.

The current embodiment utilizes PDMS, or some other such well-qualified polymer, as a substrate (SUB) that forms the body of the applicator (A). For example, biological materials such as hyaluronan, elastin, and collagen, which are components of the native extracellular matrix, may also be used alone or in combination with inorganic compounds to form the substrate (SUB).

A material with a refractive index lower than that of the substrate (SUB) (PDMS in this non-limiting example) may used as filling (LFA) to create waveguide cladding where the PDMS itself acts as the waveguide core. In the visible spectrum, the refractive index of PDMS is ˜1.4. Water, and even PBS & Saline have indices of ˜1.33, making them suitable for cladding materials. They are also biocompatible and safe for use in an illumination management system as presented herein, even if the integrity of the applicator (A) is compromised and they are released into the body.

Alternately, a higher index filling may be used as the waveguide channel. This may be thought of as the inverse of the previously described geometry, where in lieu of the polymer comprising substrate (SUB), you have a liquid filling (LFA) acting as the waveguide core medium, and the substrate (SUB) material acting as the cladding. Many oils have refractive indices of ˜1.5 or higher, making them suitable for core materials.

Alternately, a second polymer of differing refractive index may be used instead of the aforementioned liquid fillings. A high-refractive-index polymer (HRIP) is a polymer that has a refractive index greater than 1.50. The refractive index is related to the molar refractivity, structure and weight of the monomer. In general, high molar refractivity and low molar volumes increase the refractive index of the polymer. Sulfur-containing substituents including linear thioether and sulfone, cyclic thiophene, thiadiazole and thianthrene are the most commonly used groups for increasing refractive index of a polymer in forming a HRIP. Polymers with sulfur-rich thianthrene and tetrathiaanthrene moieties exhibit n values above 1.72, depending on the degree of molecular packing. Such materials may be suitable for use as waveguide channels within a lower refractive polymeric substrate. Phosphorus-containing groups, such as phosphonates and phosphazenes, often exhibit high molar refractivity and optical transmittance in the visible light region. Polyphosphonates have high refractive indices due to the phosphorus moiety even if they have chemical structures analogous to polycarbonates. In addition, polyphosphonates exhibit good thermal stability and optical transparency; they are also suitable for casting into plastic lenses. Organometallic components also result in HRIPs with good film forming ability and relatively low optical dispersion. Polyferrocenylsilanes and polyferrocenes containing phosphorus spacers and phenyl side chains show unusually high n values (n=1.74 and n=1.72), as well, and are also candidates for waveguides.

Hybrid techniques which combine an organic polymer matrix with highly refractive inorganic nanoparticles may be employed to produce polymers with high n values. As such, PDMS may also be used to fabricate the waveguide channels that may be integrated to a PDMS substrate, where native PDMS is used as the waveguide cladding. The factors affecting the refractive index of a HRIP nanocomposite include the characteristics of the polymer matrix, nanoparticles, and the hybrid technology between inorganic and organic components. Linking inorganic and organic phases is also achieved using covalent bonds. One such example of hybrid technology is the use of special bifunctional molecules, such as MEMO, which possess a polymerisable group as well as alkoxy groups. Such compounds are commercially available and can be used to obtain homogeneous hybrid materials with covalent links, either by simultaneous or subsequent polymerization reactions.

The following relation estimates the refractive index of a nanocomposite,

n_{comp}=φₚnₚ+φ_{org}n_{org}

where, n_{comp}, nₚ and n_{org} stand for the refractive indices of the nanocomposite, nanoparticle and organic matrix, respectively, while φp and φorg represent the volume fractions of the nanoparticles and organic matrix, respectively.

The nanoparticle load is also important in designing HRIP nanocomposites for optical applications, because excessive concentrations increase the optical loss and decrease the processability of the nanocomposites. The choice of nanoparticles is often influenced by their size and surface characteristics. In order to increase optical transparency and reduce Rayleigh scattering of the nanocomposite, the diameter of the nanoparticle should be below 25 nm. Direct mixing of nanoparticles with the polymer matrix often results in the undesirable aggregation of nanoparticles—this is may be avoided by modifying their surface, or thinning the viscosity of the liquid polymer with a solvent such as Xylenes; which may later be removed by vacuum during ultrasonic mixing of the composite prior to curing. Nanoparticles for HRIPs may be chosen from the group consisting of: TiO2 (anatase, n=2.45; rutile, n=2.70), ZrO2 (n=2.10), amorphous silicon (n=4.23), PbS (n=4.20) and ZnS (n=2.36). Further materials are given in the table below. The resulting nanocomposites may exhibit a tunable refractive index range, per the above relation.

| Substance | n (413.3 nm) | n (619.9 nm) |
|---|---|---|
| Os | 4.05 | 3.98 |
| W | 3.35 | 3.60 |
| Si crystalline | 5.22 | 3.91 |
| Si amorphous | 4.38 | 4.23 |
| Ge | 4.08 | 5.59-5.64 |
| GaP | 4.08 | 3.33 |
| GaAs | 4.51 | 3.88 |
| InP | 4.40 | 3.55 |
| InAs | 3.20 | 4.00 |
| InSb | 3.37 | 4.19 |
| PbS | 3.88 | 4.29 |
| PbSe | 1.25-3.00 | 3.65-3.90 |
| PbTe | 1.0-1.8 | 6.40 |
| Ag | 0.17 | 0.13 |
| Au | 1.64 | 0.19 |
| Cu | 1.18 | 0.27 |

In one exemplary embodiment, a HRIP preparation based on PDMS and PbS, the volume fraction of particles needs to be around 0.2 or higher to yield ncomp≧1.96, which corresponds to a weight fraction of at least 0.8 (using the density of PbS of 7.50 g cm⁻³ and of PDMS of 1.35 g cm⁻³). Such a HRIP can support a high numerical aperture (NA), which is useful when coupling light from relatively low brightness sources such as LEDs. The information given above allows for the recipe of other alternate formulations to be readily ascertained.

There are many synthesis strategies for nanocomposites. Most of them can be grouped into three different types. The preparation methods are all based on liquid particle dispersions, but differ in the type of the continuous phase. In melt processing particles are dispersed into a polymer melt and nanocomposites are obtained by extrusion. Casting methods use a polymer solution as dispersant and solvent evaporation yields the composite materials, as described earlier. Particle dispersions in monomers and subsequent polymerization result in nanocomposites in the so-called in situ polymerization route.

In a similar way, low refractive index composite materials have may also be prepared. As suitable filler materials, metals with low refractive indices below 1, such as gold (shown in the table above) may be chosen, and the resulting low index material used as the waveguide cladding.

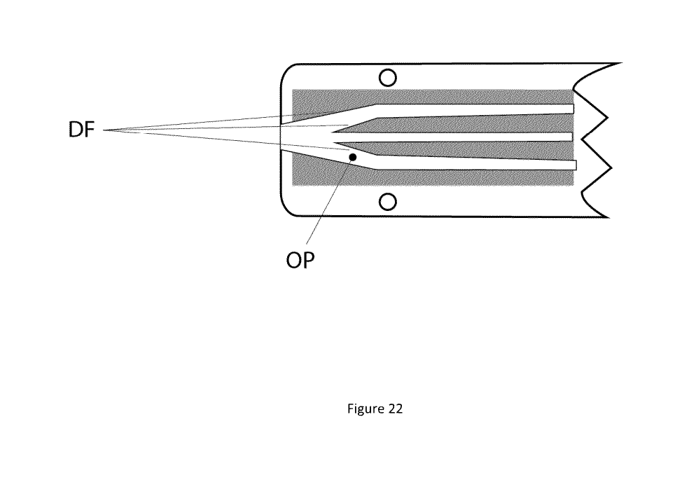

There are a variety of optical plenum configurations for capturing light input and creating multiple output channels. As shown in the figure, the facets are comprised of linear faces. The angle of the face with respect to the input direction of the light dictates the numerical aperture (NA). Alternately, curved faces may be employed for nonlinear angular distribution and intensity homogenization. A parabolic surface profile may be used, for example. Furthermore, the faces need be planar. A three-dimensional surface may similarly be employed. The position of these plenum distribution facets DF may be used to dictate the proportion of power captured as input to a channel, as well. Alternately, the plenum distribution facets DF may spatially located in accordance with the intensity/irradiance distribution of the input light source. As a non-limiting example, an input with a lambertian irradiance distribution, such as may be output by an LED, the geometry of the distribution facets DF may be tailored to limit the middle channel to have ⅓ of the emitted light, and the outer channels evenly divide the remaining ⅔, such as is shown in FIG. 22 by way on non-limiting example.

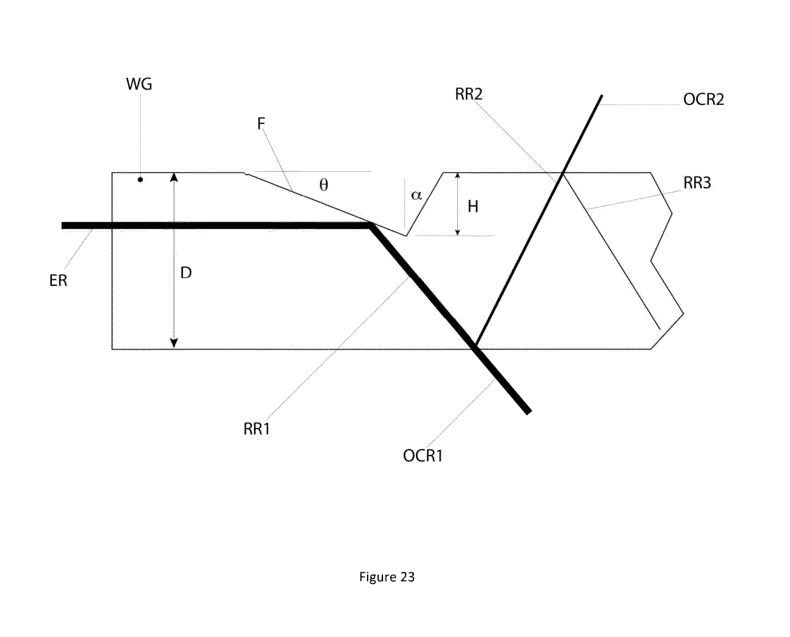

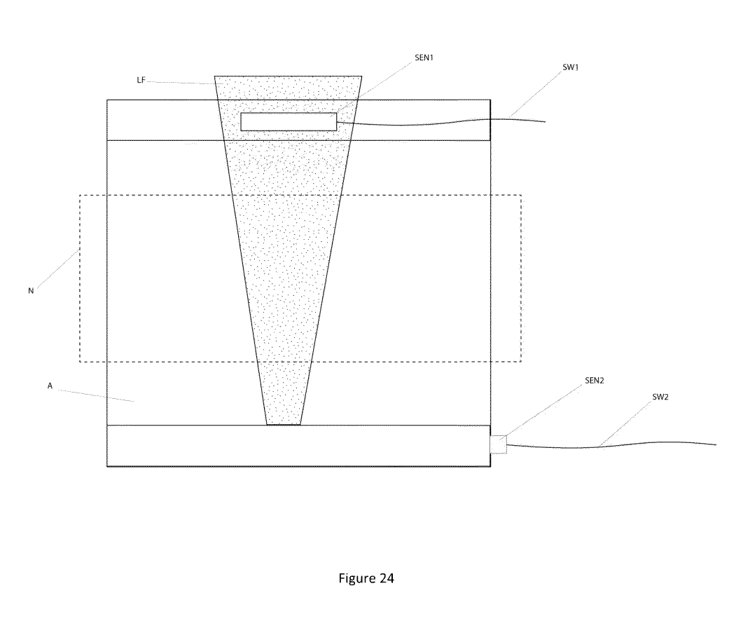

Output Coupling may be achieved many ways, as discussed earlier. Furthering that discussion, and to be considered as part thereof, scattering surfaces in areas of intended emission may be utilized. Furthermore, output coupling facets, such as POC and TOC shown previously, may also be employed. These may reflective, refractive, scattering, etc. The height of facet may be configured to be in proportion to the amount or proportion of light intercepted, while the longitudinal position dictates the output location. As was also discussed previously, for systems employing multiple serial OCs, the degree of output coupling of each may be made to be proportional to homogenize the ensemble illumination. A single-sided facet within the waveguide channel may be disposed such that it predominantly captures light traveling one way down the waveguide channel (or core). Alternately, a double-sided facet that captures light traveling both ways down the waveguide channel (or core) to provide both forward and backward output coupling. This would be used predominantly with distal retroreflector designs. Such facets may be shaped as, by way of non-limiting example; a pyramid, a ramp, an upward-curved surface, a downward-curved surface, etc. FIG. 23 illustrates output coupling for a ramp-shaped facet.

Light Ray ER enters (or is propagation within) Waveguide Core WG. It impinges upon Output Coupling Facet F and is redirected to the opposite surface. It becomes Reflected Ray RR1, from which Output Coupled Ray OCR1 is created, as is Reflected Ray RR2. OCR1 is directed at the target. OCR2 and RR3 are likewise created from RR2. Note that OCR2 is emitted from the same surface of WG as the facet. If there is no target or reflector on that side, the light is lost. The depth of F is H, and the Angle θ. Angle θ dictates the direction of RR1, and its subsequent rays. Angle α may be provided in order to allow for mold release for simplified fabrication. It may also be used to output couple light traversing in the opposite direct as ER, such as might be the case when distal retro-reflectors are used.

Alternately, Output Coupling Facet F may protrude from the waveguide, allowing for the light to be redirected in an alternate direction, but by similar means.

The waveguide channel(s) may be as described above. Use of fluidics may also be employed to expand (or contract) the applicator to alter the fit or “snugness”, as was described above regarding Sleeve S. When used with the applicator (A), it may serve to decrease infiltrate permeability as well as to increase optical penetration via pressure-induced tissue clearing. Fluidic channels incorporated into the applicator substrate may also be used to tune the output coupling facets. Small reservoirs beneath the facets may be made to swell and in turn distend the location and/or the angle of the facet in order to adjust the amount of light and/or the direction of that light.

Captured light may also be used to assess efficiency or functional “health” of the applicator and/or system by providing information regarding the optical transport efficiency of the device/tissue states. The detection of increased light scattering may be indicative of changes in the optical quality or character of the tissue and or the device. Such changes may be evidenced by the alteration of the amount of detected light collected by the sensor. It may take the form of an increase or a decrease in the signal strength, depending upon the relative positions of the sensor and emitter(s). An opposing optical sensor may be employed to more directly sample the output, as is illustrated in FIG. 24. In this non-limiting embodiment, Light Field LF is intended to illuminate the Target (N) via output coupling from a waveguide within Applicator A, and stray light is collected by Sensor SEN1. SEN1 may be electrically connected to the Housing (not shown) via Wires SW1 to supply the Controller with information regarding the intensity of the detected light. A second Sensor SEN2 is also depicted. Sensor SEN2 may be used to sample light within a (or multiple) waveguides of Applicator A, and its information conveyed to a controller (or processor) via Wires SW2. This provides additional information regarding the amount of light propagating within the Waveguide(s) of the Applicator. This additional information may be used to better estimate the optical quality of the target exposure by means of providing a baseline indicative of the amount of light energy or power that is being emitted via the resident output coupler(s), as being proportional to the conducted light within the Waveguide(s).

Alternately, the temporal character of the detected signals may be used for diagnostic purposes. For example, slower changes may indicate tissue changes or device aging, while faster changes could be strain, or temperature dependent fluctuations. Furthermore, this signal may be used for closed loop control by adjusting power output over time to assure more constant exposure at the target. The detected signal of a Sensor such as SEN1 may also be used to ascertain the amount of optogenetic absorbers present in the target. If such detection is difficult to the proportionately small effects on the signal, a heterodyned detection scheme may be employed for this purpose. Such an exposure may be of insufficient duration or intensity to cause a therapeutic effect, but made solely for the purposes of overall system diagnostics.

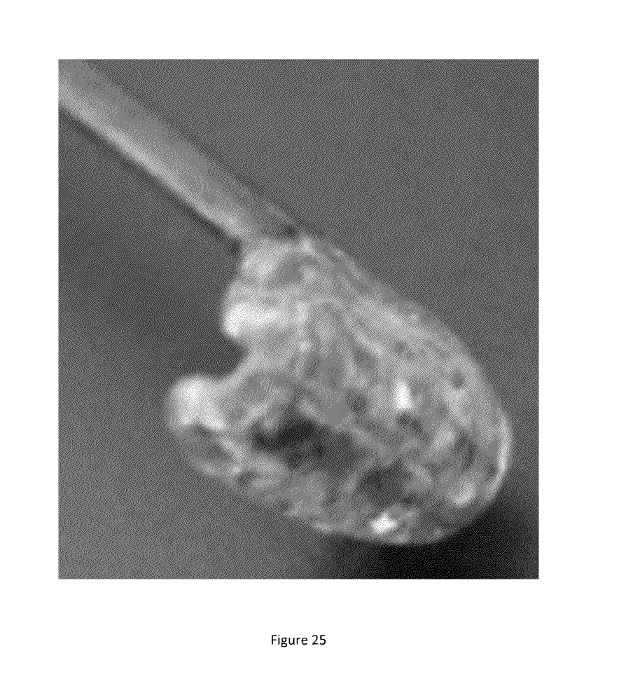

Alternately, an applicator may be fabricated with individually addressable optical source elements to enable adjustment of the intensity and location of the light delivery, as is shown in the embodiment of FIG. 25. Such applicators may be configured to deliver light of a single wavelength to activate or inhibit nerves. Alternately, they may be configured to deliver light of two or more different wavelengths, or output spectra, to provide for both activation and inhibition in a single device, or a plurality of devices.

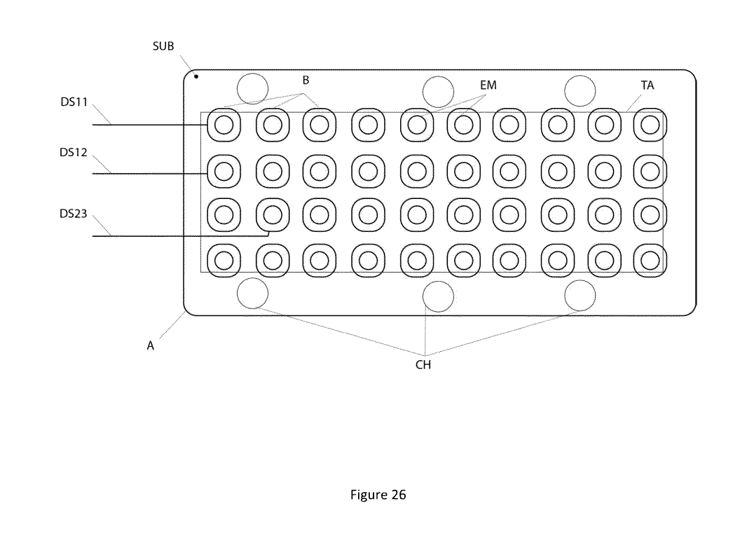

An alternate example of such an applicator is shown in FIG. 26, where Applicator A is comprised of Optical Source Elements, or Emitters (EM). Element “B” is representative of the body of the patient/subject; element “DS”xx represents the pertinent delivery segments as per their coordinates in rows/columns on the applicator (A); element “SUB” represents the substrate, element “CH” represents closure holes, and element “TA” a textured area, as described above.

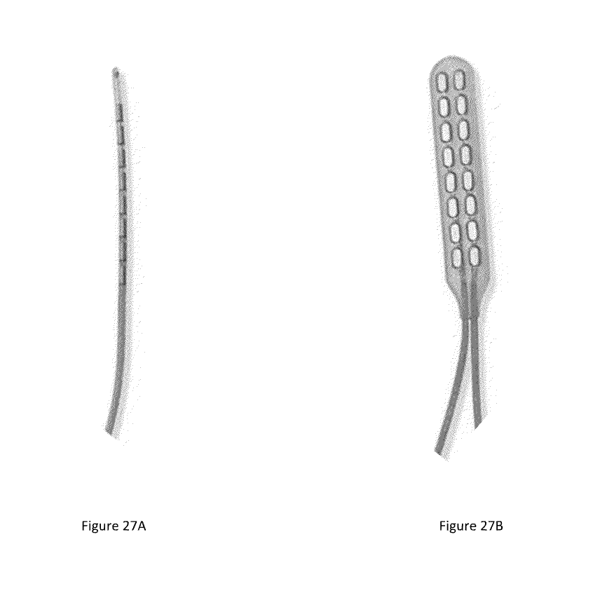

Alternate configurations are shown in FIGS. 27A and 27B, wherein applicators configured as linear and planar arrays of emitters, or alternately output couplers, are shown.

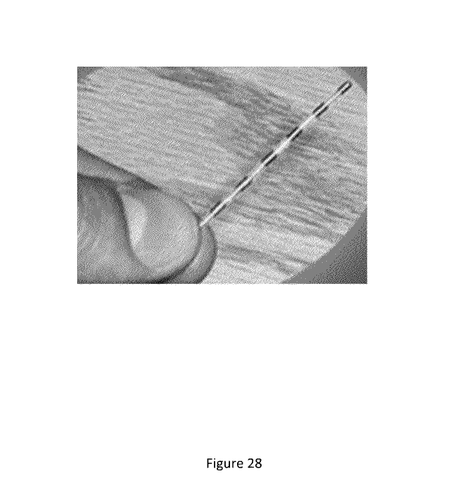

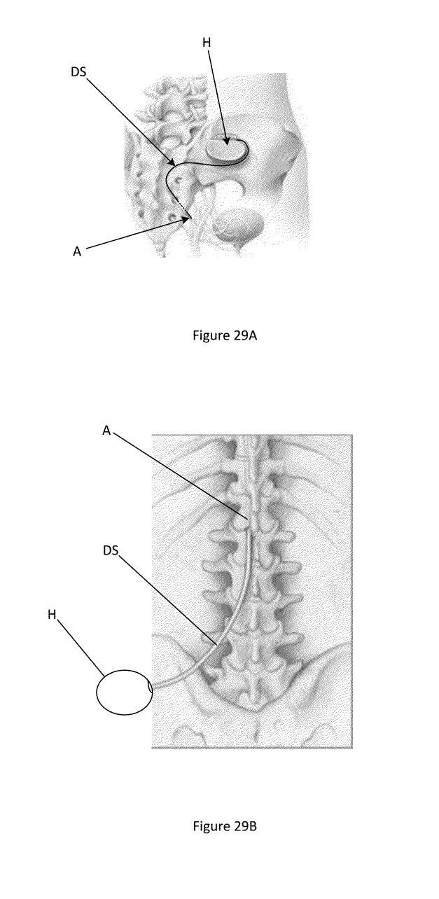

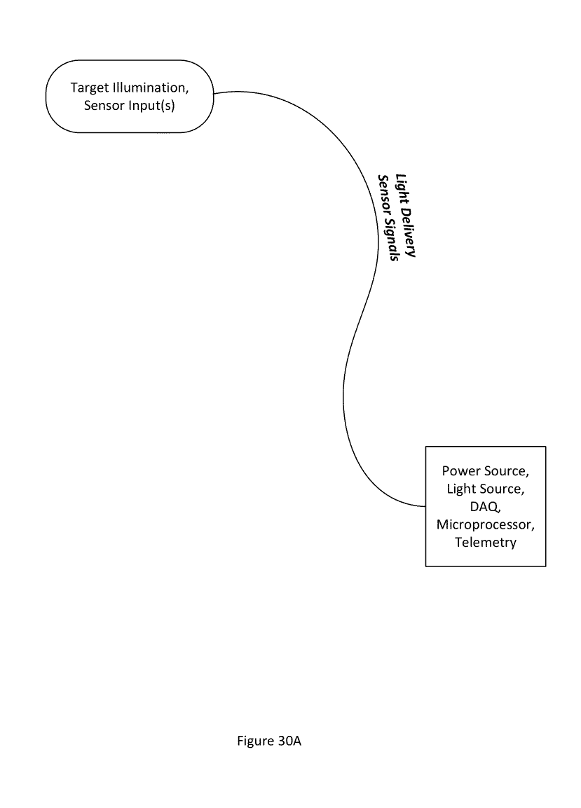

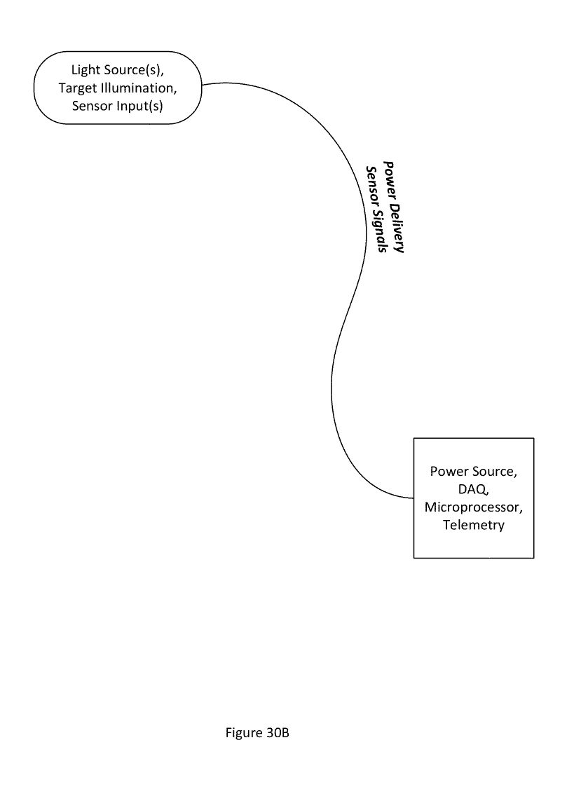

A linear array optogenetic light applicator (A), or “optarray, may be inserted into the intrathecal space to deliver light to the sacral roots for optogenetic modulation of neurons involved in bowel, bladder, and erectile function. Alternately, it may be inserted higher in the spinal column for pain control applications, such as those described elsewhere in this application. Either the linear or matrix array optarray(s) may be inserted into the anterior intrathecal to control motor neurons and/or into the posterior intrathecal to control sensory neurons. A single optical element may be illuminated for greater specificity, or multiple elements may be illuminated. FIG. 28 illustrates an alternative view of an exemplary linear array.

The system may be tested for utility at the time of implantation, or subsequent to it. The tests may provide for system configurations, such as which areas of the applicator are most effective, or efficacious, by triggering different light sources alone, or in combination, to ascertain their effect on the patient. This may be utilized when a multi-element system, such as an array of LEDs, for example, or a multiple output coupling method is used. Such diagnostic measurements may be achieved by using an implanted electrode that resides on, in or near the applicator, or one that was implanted elsewhere, as will be described in another section. Alternately, such measurements maybe made at the time of implantation using a local nerve electrode for induced stimulation, and/or an electrical probe to query the nerve impulses intraoperatively using a device such as the Stimulator sold under the tradename “Checkpoint”® from NDI and Checkpoint Surgical, Inc. to provide electrical stimulation of exposed motor nerves or muscle tissue and in turn locate and identify nerves as well to test their excitability. Once obtained, an applicator illumination configuration may be programmed into the system for optimal therapeutic outcome using an external Programmer/Controller (P/C) via a Telemetry Module (TM) into the Controller, or Processor/CPU of the system Housing (H), as are defined further below.

FIG. 29A illustrates the gross anatomical location of an implantation/installation configuration wherein a controller housing (H) is implanted adjacent the pelvis, and is operatively coupled (via the delivery segment DS) to an applicator (A) positioned to stimulate one or more of the sacral nerve roots.

FIG. 29B illustrates the gross anatomical location of an implantation/installation configuration wherein a controller housing (H) is implanted adjacent the pelvis, and is operatively coupled (via the delivery segment DS) to an applicator (A) positioned to stimulate one or more of the lumbar, thoracic, or cervical nerve roots, such as by threading the delivery segment and applicator into the intrathecal space to reach the pertinent root anatomy.

The electrical connections for devices such as these where the light source is either embedded within, on, or located nearby to the applicator, may be integrated into the applicators described herein. Materials like the product sold by NanoSonics, Inc. under the tradename MetalRubber® and/or mc10's extensible inorganic flexible circuit platform may be used to fabricate an electrical circuit on or within an applicator. Alternately, the product sold by DuPont, Inc., under the tradename Pyralux®, or other such flexible and electrically insulating material, like polyimide, may be used to form a flexible circuit; including one with a copper-clad laminate for connections. Pyralux in sheet form allows for such a circuit to be rolled. More flexibility may be afforded by cutting the circuit material into a shape that contains only the electrodes and a small surrounding area of polyimide.

Such circuits may then be encapsulated for electrical isolation using a conformal coating. A variety of such conformal insulation coatings are available, including by way of non-limiting example, parlene (Poly-Para-Xylylene) and parlene-C (parylene with the addition of one chlorine group per repeat unit), both of which are chemically and biologically inert. Silicones and polyurethanes may also be used, and may be made to comprise the applicator body, or substrate, itself. The coating material can be applied by various methods, including brushing, spraying and dipping. Parylene-C is the most bio-accepted coating for stents, defibrillators, pacemakers and other devices permanently implanted into the body.

In a particular embodiment, biocompatible and bio-inert coatings may be used to reduce foreign body responses, such as that may result in cell growth over or around an applicator and change the optical properties of the system. These coatings may also be made to adhere to the electrodes and to the interface between the array and the hermetic packaging that forms the applicator.

By way of non-limiting example, both parylene-C and poly(ethylene glycol) (PEG, described earlier) have been shown to be biocompatible and may be used as encapsulating materials for an applicator. Bioinert materials non-specifically downregulate, or otherwise ameliorate, biological responses. An example of such a bioinert material for use in an embodiment of the present invention is phosphoryl choline, the hydrophilic head group of phospholipids (lecithin and sphingomyelin), which predominate in the outer envelope of mammalian cell membranes. Another such example is Polyethylene oxide polymers (PEO), which provide some of the properties of natural mucous membrane surfaces. PEO polymers are highly hydrophilic, mobile, long chain molecules, which may trap a large hydration shell. They may enhance resistance to protein and cell spoliation, and may be applied onto a variety of material surfaces, such as PDMS, or other such polymers. An alternate embodiment of a biocompatible and bioinert material combination for use in practicing the present invention is phosphoryl choline (PC) copolymer, which may be coated on a PDMS substrate. Alternately, a metallic coating, such as Gold or Platinum, as were described earlier, may also be used. Such metallic coatings may be further configured to provide for a bioinert outer layer formed of self-assembled monolayers (SAMs) of, for example, D-mannitol-terminated alkanethiols. Such a SAM may be produced by soaking the intended device to be coated in 2 mM alkanethiol solution (in ethanol) overnight at room temperature to allow the SAMs to form upon it. The device may then be taken out and washed with absolute ethanol and dried with nitrogen to clean it.

A variety of embodiments of light applicators are disclosed herein. There are further bifurcations that depend upon where the light is produced (i.e., in or near the applicator vs. in the housing or elsewhere). FIGS. 30A and 30B illustrate these two configurations.

Referring to FIG. 30A, in a first configuration, light is generated in the housing and transported to the applicator via the delivery segment. The delivery segment(s) may be optical waveguides, selected from the group consisting of round fibers, hollow waveguides, holey fibers, photonic bandgap devices, and/or slab configurations, as have described previously. Multiple waveguides may also be employed for different purposes. As a non-limiting example, a traditional circular cross-section optical fiber may be used to transport light from the source to the applicator because such fibers are ubiquitous and may be made to be robust and flexible. Alternately, such a fiber may be used as input to another waveguide, this with a polygonal cross-section providing for regular tiling. Such waveguides have cross-sectional shapes that pack together fully, i.e. they form an edge-to-edge tiling, or tessellation, by means of regular congruent polygons. That is, they have the property that their cross-sectional geometry allows them to completely fill (pack) a two-dimensional space. This geometry yields the optical property that the illumination may be made to spatially homogeneous across the face of such a waveguide. Complete homogeneity is not possible with other geometries, although they may be made to have fairly homogeneous irradiation profiles nonetheless. For the present application, a homogenous irradiation distribution is useful because it may provide for uniform illumination of the target tissue. Thus, such regular-tiling cross-section waveguides may be useful. It is also to be understood that this is a schematic representation and that multiple applicators and their respective delivery segments may be employed. Alternately, a single delivery segment may service multiple applicators. Similarly, a plurality of applicator types may also be employed, based upon the clinical need.

Referring to the configuration of FIG. 30B, light is in the applicator. The power to generate the optical output is contained within the housing and is transported to the applicator via the delivery segment. It is to be understood that this is a schematic representation and that multiple applicators and their respective delivery segments may be employed. Similarly, a plurality of applicator types may also be employed.

The pertinent delivery segments may be optical waveguides, such as optical fibers, in the case where the light is not generated in or near the applicator(s). Alternately, when the light is generated at or near the applicator(s), the delivery segments may be electrical wires. They may be further comprised of fluidic conduits to provide for fluidic control and/or adjustment of the applicator(s). They may also be any combination thereof, as dictated by the specific embodiment utilized, as have been previously described.

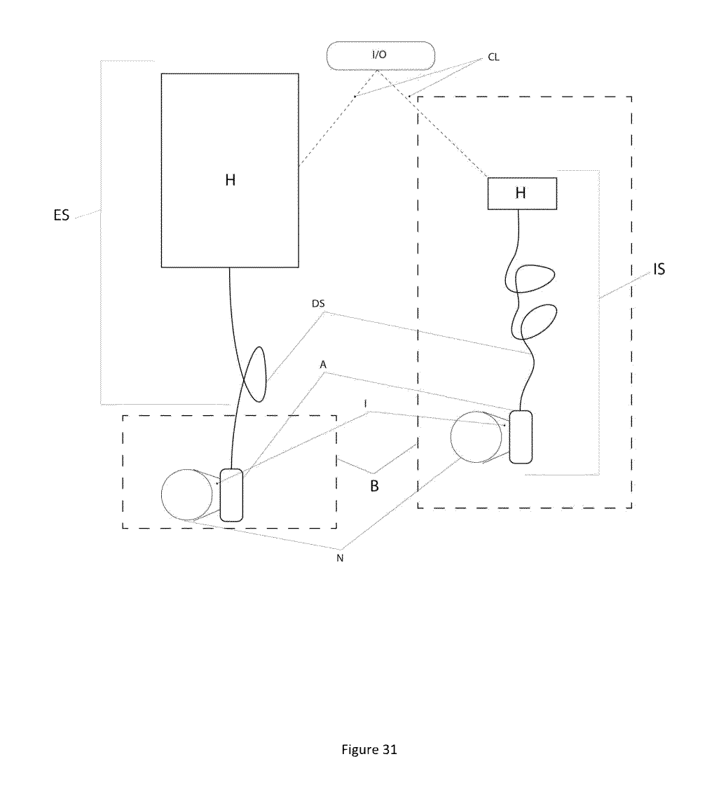

Embodiments of the subject system may be partially, or entirely, implanted in the body of a patient. FIG. 31 illustrates this, wherein the left hand side of the illustration schematically depicts the partially implanted system, and the right hand side of the illustration the fully implanted device. The housing H may be implanted, carried, or worn on the body (B), along with the use of percutaneous feed-throughs or ports for optical and/or electrical conduits that comprise the delivery segments (DSx) that connect to Applicator(s) A.

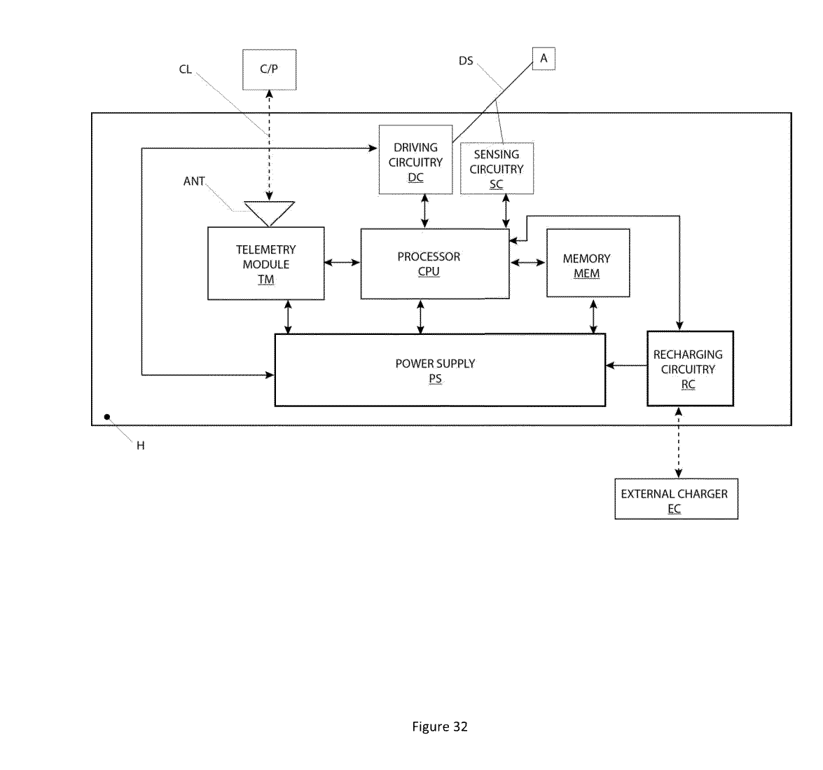

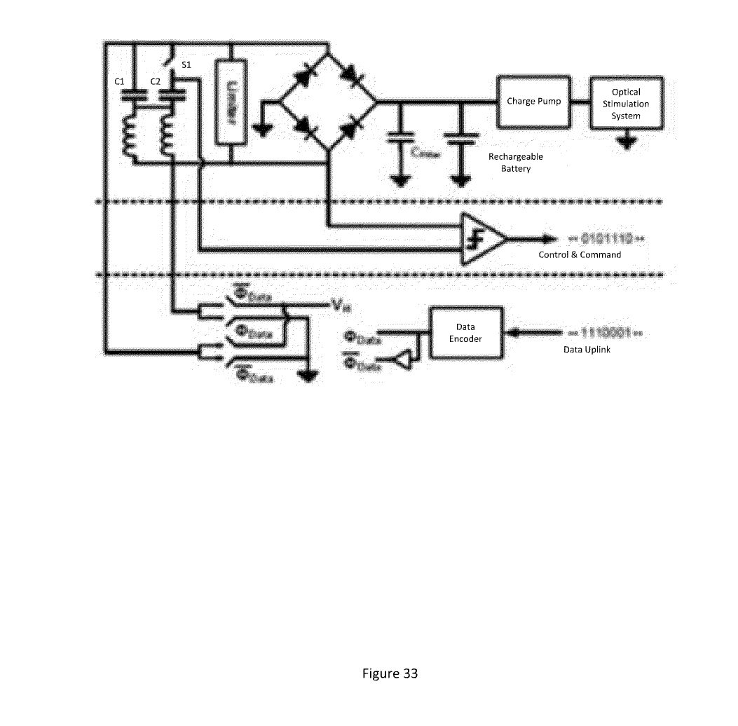

Referring to FIG. 32, a block diagram is depicted illustrating various components of an example implantable housing H. In this example, implantable stimulator includes processor CPU, memory M, power source PS, telemetry module TM, antenna ANT, and the driving circuitry DC for an optical stimulation generator (which may or may not include a light source, as has been previously described). The Housing H is coupled to one Delivery Segments DSx, although it need not be. It may be a multi-channel device in the sense that it may be configured to include multiple optical paths (e.g., multiple light sources and/or optical waveguides or conduits) that may deliver different optical outputs, some of which may have different wavelengths. More or less delivery segments may be used in different implementations, such as, but not limited to, one, two, five or more optical fibers and associated light sources may be provided. The delivery segments may be detachable from the housing, or be fixed.

Memory (MEM) may store instructions for execution by Processor CPU, optical and/or sensor data processed by sensing circuitry SC, and obtained from sensors both within the housing, such as battery level, discharge rate, etc., and those deployed outside of the Housing (H), possibly in Applicator A, such as optical and temperature sensors, and/or other information regarding therapy for the patient. Processor (CPU) may control Driving Circuitry DC to deliver power to the light source (not shown) according to a selected one or more of a plurality of programs or program groups stored in Memory (MEM). The Light Source may be internal to the housing H, or remotely located in or near the applicator (A), as previously described. Memory (MEM) may include any electronic data storage media, such as random access memory (RAM), read-only memory (ROM), electronically-erasable programmable ROM (EEPROM), flash memory, etc. Memory (MEM) may store program instructions that, when executed by Processor (CPU), cause Processor (CPU) to perform various functions ascribed to Processor (CPU) and its subsystems, such as dictate pulsing parameters for the light source.

In accordance with the techniques described in this disclosure, information stored in Memory (MEM) may include information regarding therapy that the patient had previously received. Storing such information may be useful for subsequent treatments such that, for example, a clinician may retrieve the stored information to determine the therapy applied to the patient during his/her last visit, in accordance with this disclosure. Processor CPU may include one or more microprocessors, digital signal processors (DSPs), application-specific integrated circuits (ASICs), field-programmable gate arrays (FPGAs), or other digital logic circuitry. Processor CPU controls operation of implantable stimulator, e.g., controls stimulation generator to deliver stimulation therapy according to a selected program or group of programs retrieved from memory (MEM). For example, processor (CPU) may control Driving Circuitry DC to deliver optical signals, e.g., as stimulation pulses, with intensities, wavelengths, pulse widths (if applicable), and rates specified by one or more stimulation programs. Processor (CPU) may also control Driving Circuitry (DC) to selectively deliver the stimulation via subsets of Delivery Segments (DSx), and with stimulation specified by one or more programs. Different delivery segments (DSx) may be directed to different target tissue sites, as was previously described.

Telemetry module (TM) may include a radio frequency (RF) transceiver to permit bi-directional communication between implantable stimulator and each of clinician programmer and patient programmer (C/P). Telemetry module (TM) may include an Antenna (ANT), of any of a variety of forms. For example, Antenna (ANT) may be formed by a conductive coil or wire embedded in a housing associated with medical device. Alternatively, antenna (ANT) may be mounted on a circuit board carrying other components of implantable stimulator or take the form of a circuit trace on the circuit board. In this way, telemetry module (TM) may permit communication with a controller/programmer (C/P). Given the energy demands and modest data-rate requirements, the Telemetry system may be configured to use inductive coupling to provide both telemetry communications and power for recharging, although a separate recharging circuit (RC) is shown in FIG. 32 for explanatory purposes. An alternate configuration is shown in FIG. 33.

Referring to FIG. 33, a telemetry carrier frequency of 175 kHz aligns with a common ISM band and may use on-off keying at 4.4 kbps to stay well within regulatory limits. Alternate telemetry modalities are discussed elsewhere herein. The uplink may be an H-bridge driver across a resonant tuned coil. The telemetry capacitor, C1, may be placed in parallel with a larger recharge capacitor, C2, to provide a tuning range of 50-130 kHz for optimizing the RF-power recharge frequency. Due to the large dynamic range of the tank voltage, the implementation of the switch, S1, employs a nMOS and pMOS transistor connected in series to avoid any parasitic leakage. When the switch is OFF, the gate of pMOS transistor is connected to battery voltage, VBattery, and the gate of nMOS is at ground. When the switch is ON, the pMOS gate is at negative battery voltage, −VBattery, and the nMOS gate is controlled by charge pump output voltage. The ON resistance of the switch is designed to be less than 5Ω to maintain a proper tank quality factor. A voltage limiter, implemented with a large nMOS transistor, may be incorporated in the circuit to set the full wave rectifier output slightly higher than battery voltage. The output of the rectifier may then charge a rechargeable battery through a regulator.

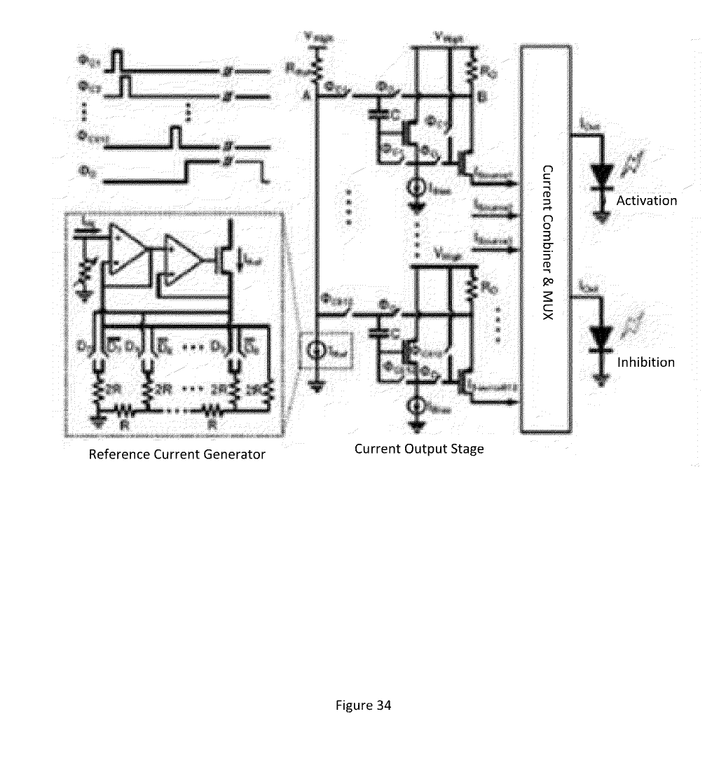

FIG. 34 relates to an embodiment of the Driving Circuitry DC, and may be made to a separate integrated circuit (or “IC”), or application specific integrated circuit (or “ASIC”), or a combination of them.

The control of the output pulse train, or burst, may be managed locally by a state-machine, as shown in this non-limiting example, with parameters passed from the microprocessor. Most of the design constraints are imposed by the output drive DAC. First, a stable current is required to reference for the system. A constant current of 100 nA, generated and trimmed on chip, is used to drive the reference current generator, which consists of an R-2Rbased DAC to generate an 8-bit reference current with a maximum value of 5 A. The reference current is then amplified in the current output stage with the ratio of Rₒ and R_{ref}, designed as a maximum value of 40. An on-chip sense-resistor-based architecture was chosen for the current output stage to eliminate the need to keep output transistors in saturation, reducing voltage headroom requirements to improve power efficiency. The architecture uses thin-film resistors (TFRs) in the output driver mirroring to enhance matching. To achieve accurate mirroring, the nodes X and Y may be forced to be the same by the negative feedback of the amplifier, which results in the same voltage drop on Rₒ and R_{ref}. Therefore, the ratio of output current, I_{O}, and the reference current, I_{ref}, equals to the ratio of and R_{ref} and R_{O}.

The capacitor, C, retains the voltage acquired in the precharge phase. When the voltage at Node Y is exactly equal to the earlier voltage at Node X, the stored voltage on C biases the gate of P2 properly so that it balances I_{bias}. If, for example, the voltage across R_{O} is lower than the original R_{ref} voltage, the gate of P2 is pulled up, allowing I_{bias} to pull down on the gate on P1, resulting in more current to R_{O}. In the design of this embodiment, charge injection is minimized by using a large holding capacitor of 10 pF. The performance may be eventually limited by resistor matching, leakage, and finite amplifier gain. With 512 current output stages, the optical stimulation IC may drive two outputs for activation and inhibition (as shown in the figure) with separate sources, each delivering a maximum current of 51.2 mA.

Alternatively, if the maximum back-bias on the optical element can withstand the drop of the other element, then the devices can be driven in opposite phases (one as sinks, one as sources) and the maximum current exceeds 100 mA. The stimulation rate can be tuned from 0.153 Hz to 1 kHz and the pulse or burst duration(s) can be tuned from 100 s to 12 ms. However, the actual limitation in the stimulation output pulse-train characteristic is ultimately set by the energy transfer of the charge pump, and this must be considered when configuring the therapeutic protocol.

The Housing H (or applicator, or the system via remote placement) may further contain an accelerometer to provide sensor input to the controller resident in the housing. This may be useful for modulation and fine control of a hypertension device, for example, or for regulation of a pacemaker. Remote placement of an accelerometer may be made at or near the anatomical element under optogenetic control, and may reside within the applicator, or nearby it. In times of notable detected motion, the system may alter it programming to accommodate the patient's intentions and provide more or less stimulation and/or inhibition, as is required for the specific case at hand.

The Housing H may still further contain a fluidic pump (not shown) for use with the applicator, as was previously described herein.

External programming devices for patient and/or physician can be used to alter the settings and performance of the implanted housing. Similarly, the implanted apparatus may communicate with the external device to transfer information regarding system status and feedback information. This may be configured to be a PC-based system, or a stand-alone system. In either case, the system must communicate with the housing via the telemetry circuits of Telemetry Module (TM) and Antenna (ANT). Both patient and physician may utilize controller/programmers (C/P) to tailor stimulation parameters such as duration of treatment, optical intensity or amplitude, pulse width, pulse frequency, burst length, and burst rate, as is appropriate.

Once the communications link (CL) is established, data transfer between the MMN programmer/controller and the housing may begin. Examples of such data are:

1. From housing to controller/programmer:
a. Patient usage
b. Battery lifetime
c. Feedback data
   i. Device diagnostics (such as direct optical transmission measurements by an emitter-opposing photosensor)

2. From controller/programmer to housing:
a. Updated illumination level settings based upon device diagnostics
b. Alterations to pulsing scheme
c. Reconfiguration of embedded circuitry
   i. FPGA, etc.

By way of non-limiting examples, near field communications, either low power and/or low frequency; such as ZigBee, may be employed for telemetry. The tissue(s) of the body have a well-defined electromagnetic response(s). For example, the relative permittivity of muscle demonstrates a monotonic log-log frequency response, or dispersion. Therefore, it is advantageous to operate an embedded telemetry device in the frequency range of ≦1 GHz. In 2009 (and then updated in 2011), the US FCC dedicated a portion of the EM Frequency spectrum for the wireless biotelemetry in implantable systems, known as The Medical Device Radiocommunications Service (known as “MedRadio”). Devices employing such telemetry and known as “medical micropower networks” or “MMN” services. The currently reserved spectra are in the 401-406, 413-419, 426-432, 438-444, and 451-457 MHz ranges, and provide for these authorized bandwidths:
401-401.85 MHz: 100 kHz
401.85-402 MHz: 150 kHz
402-405 MHz: 300 kHz
405-406 MHz: 100 kHz
413-419 MHz: 6 MHz
426-432 MHz: 6 MHz
438-444 MHz: 6 MHz
451-457 MHz: 6 MHz

The rules do not specify a channeling scheme for MedRadio devices. However, it should be understood that the FCC stipulates that:
MMNs should not cause harmful interference to other authorized stations operating in the 413-419 MHz, 426-432 MHz, 438-444 MHz, and 451-457 MHz bands.
MMNs must accept interference from other authorized stations operating in the 413-419 MHz, 426-432 MHz, 438-444 MHz, and 451-457 MHz bands.
MMN devices may not be used to relay information to other devices that are not part of the MMN using the 413-419 MHz, 426-432 MHz, 438-444 MHz, and 451-457 MHz frequency bands.
An MMN programmer/controller may communicate with a programmer/controller of another MMN to coordinate sharing of the wireless link.
Implanted MMN devices may only communicate with the programmer/controller for their MMN.
An MMN implanted device may not communicate directly with another MMN implanted device.
An MMN programmer/controller can only control implanted devices within one patient.

Interestingly, these frequency bands are used for other purposes on a primary basis such as Federal government and private land mobile radios, Federal government radars, and remote broadcast of radio stations. It has recently been shown that higher frequency ranges are also applicable and efficient for telemetry and wireless power transfer in implantable medical devices.

An MMN may be made not to interfere or be interfered with by external fields by means of a magnetic switch in the implant itself. Such a switch may be only activated when the MMN programmer/controller is in close proximity to the implant. This also provides for improved electrical efficiency due to the restriction of emission only when triggered by the magnetic switch. Giant Magnetorestrictive (GMR) devices are available with activation field strengths of between 5 and 150 Gauss. This is typically referred to as the magnetic operate point. There is intrinsic hysteresis in GMR devices, and they also exhibit a magnetic release point range that is typically about one-half of the operate point field strength. Thus, a design utilizing a magnetic field that is close to the operate point will suffer from sensitivities to the distance between the housing and the MMN programmer/controller, unless the field is shaped to accommodate this. Alternately, one may increase the field strength of the MMN programmer/controller to provide for reduced sensitivity to position/distance between it and the implant. In a further embodiment, the MMN may be made to require a frequency of the magnetic field to improve the safety profile and electrical efficiency of the device, making it is less susceptible to errant magnetic exposure. This can be accomplished by providing a tuned electrical circuit (such as an L-C or R-C circuit) at the output of the switch.

Alternately, another type of magnetic device may be employed as a switch. By way of non-limiting example, a MEMS device may be used. A cantilevered MEMS switch may be constructed such that one member of the MEMS may be made to physically contact another aspect of the MEMS by virtue of its magnetic susceptibility, similar to a miniaturized magnetic reed switch. The suspended cantilever may be made to be magnetically susceptible by depositing a ferromagnetic material (such as, but not limited to Ni, Fe, Co, NiFe, and NdFeB) atop the end of the supported cantilever member. Such a device may also be tuned by virtue of the cantilever length such that it only makes contact when the oscillations of the cantilever are driven by an oscillating magnetic field at frequencies beyond the natural resonance of the cantilever.

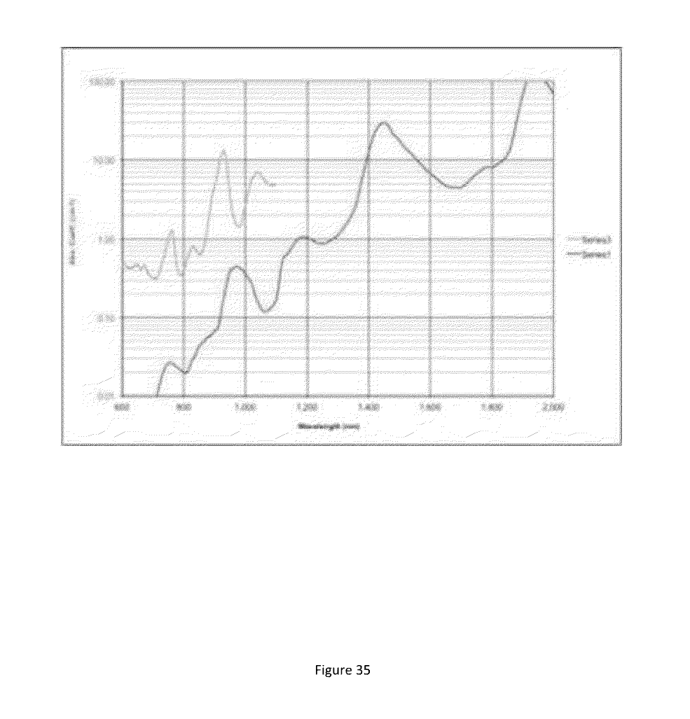

Alternately, an infrared-sensitive switch might be used. In this embodiment of this aspect of the present invention, a photodiode or photoconductor may be exposed to the outer surface of the housing and an infrared light source used to initiate the communications link for the MMN. Infrared light penetrates body tissues more readily than visible light due to its reduced scattering. However, water and other intrinsic chromophores have avid absorption, with peaks at 960, 1180, 1440, and 1950 nm, as are shown in the spectra of FIG. 35, where the water spectrum runs form 700-2000 nm and that of adipose tissue runs from 600-1100 nm.

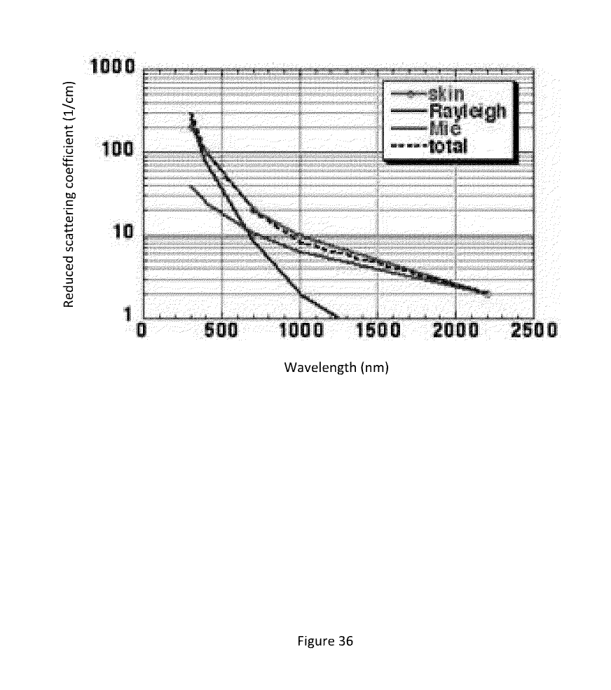

However, the penetration depth in tissue is more influenced by its scattering properties, as shown in the spectrum of FIG. 36, which displays the optical scattering spectrum for human skin, including the individual components from both Mie (elements of similar size to the wavelength of light) and Rayleigh (elements of smaller size than the wavelength of light) scattering effects.

This relatively monotonic reduction in optical scattering far outweighs absorption, when the abovementioned peaks are avoided. Thus, an infrared (or near-infrared) transmitter operating within the range of 800-1300 nm is preferred. This spectral range is known as the skin's “optical window.”

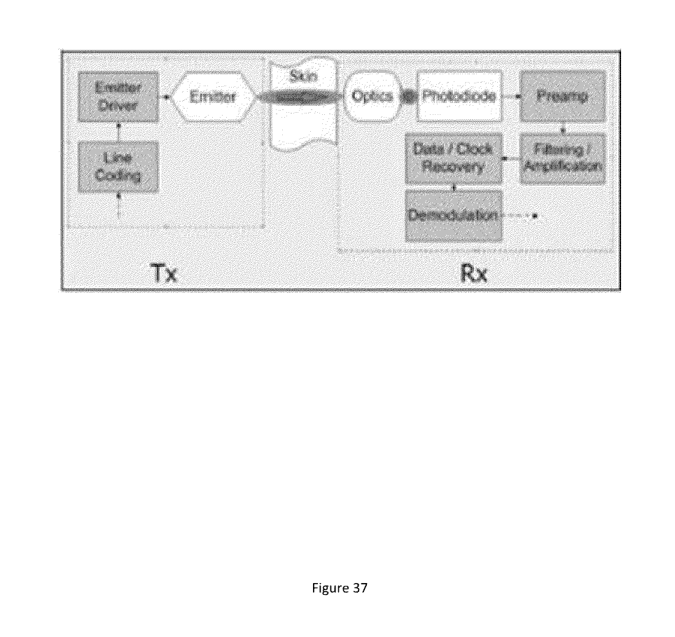

Such a system may further utilize an electronic circuit, such as that shown in FIG. 37, for telemetry, and not just a sensing switch. Based upon optical signaling, such a system may perform at high data throughput rates.

Generically, the SNR of a link is defined as,

$${SNR}_{i} = \frac{{I}_{s}}{{I}_{N}} = \frac{{P}_{s} R}{{I}_{{N}_{elec}} + {P}_{{N}_{amb}} R}$$

where Iₛ and I_{N} are the photocurrents resulting from incident signal optical power and photodiode noise current respectively, Pₛ is the received signal optical power, R is the photodiode responsivity (A/W), I_{Nelec} is the input referred noise for the receiver and P_{Namb} is the incident optical power due to interfering light sources (such as ambient light).
PS can be further defined as

P_{S}=∫_{A}_{_{T}}P_{Tx}J_{Rxλ}η_{λ}dA

where P_{TX} (W) is the optical power of the transmitted pulse, J_{Rxλ} (cm⁻²) is the tissue's optical spatial impulse response flux at wavelength λ, η_{λ} is an efficiency factor (η_{λ}≦1) accounting for any inefficiencies in optics/optical filters at λ and A_{T} represents the tissue area over which the receiver optics integrate the signal.

The abovementioned factors that affect the total signal photocurrent and their relationship to system level design parameters include emitter wavelength, emitter optical power, tissue effects, lens size, transmitter-receiver misalignment, receiver noise, ambient light sources, photodiode responsivity, optical domain filtering, receiver signal domain filtering, line coding and photodiode and emitter selection. Each of these parameters can be independently manipulated to ensure that the proper signal strength for a given design will be achieved.

Most potentially interfering light sources have signal power that consists of relatively low frequencies (e.g. Daylight: DC, Fluorescent lights: frequencies up to tens or hundreds of kilohertz), and can therefore be rejected by using a high-pass filter in the signal domain and using higher frequencies for data transmission.

The emitter may be chosen from the group consisting of, by way of non-limiting example, a VCSEL, an LED, a HCSEL. VCSELs are generally both higher brightness and more energy efficient than the other sources and they are capable of high-frequency modulation. An example of such a light source is the device sold under the model identifier “HFE 4093-342” from Finisar, Inc., which operates at 860 nm and provides ≦5 mW of average power. Other sources are also useful, as are a variety of receivers (detectors). Some non-limiting examples are listed in the following table.

| | | | |
|---|---|---|---|
| 820-850 | nm | Agilent HFBR-1412 | Agilent HFBR-2412 |
| | | Agilent HFBR-1416 | Agilent HFBR-2416 |
| | | Hamamatsu L1915 | Hamamatsu GT4176 |
| | | Hamamatsu L5128 | |
| | | Hamamatsu L5871 | |
| | | Hamamatsu L6486 | |
| 950 | nm | Infineon SFH 4203 | Infineon SFH 203 |
| | | Infineon SFH 4301 | Infineon SFH 5400 |
| | | Infineon SFH 4502 | Infineon SFH 5440 |
| | | Infineon SFH 4503 | Infineon SFH5441 |
| 1300 | nm | Agilent HFBR-1312 | Agilent HFBR-2316 |
| | | Hamamatsu L7866 | |
| | | Hamamatsu L7850 | |

Alignment of the telemetry emitter to receiver may be improved by using a non-contact registration system, such as an array of coordinated magnets with the housing that interact with sensors in the controller/programmer to provide positional information to the user that the units are aligned. In this way, the overall energy consumption of the entire system may be reduced.

Although glycerol and polyethylene glycol (PEG) reduce optical scattering in human skin, their clinical utility has been very limited. Penetration of glycerol and PEG through intact skin is very minimal and extremely slow, because these agents are hydrophilic and penetrate the lipophilic stratum corneum poorly. In order to enhance skin penetration, these agents need to be either injected into the dermis or the stratum corneum has to be removed, mechanically (e.g., tape stripping, light abrasion) or thermally (e.g., erbium: YAG laser ablation), etc. Such methods include tape stripping, ultrasound, iontophoresis, electroporation, microdermabrasion, laser ablation, needle-free injection guns, and photomechanically driven chemical waves (aka “optoporation”). Alternately, microneedles contained in an array or on a roller (such as the Dermaroller) may be used to decrease the penetration barrier. The Dermaroller is configured such that each of its 192 needles has a 70 μm diameter and 500 μm height. These microneedles are distributed uniformly atop a 2 cm wide by 2 cm diameter cylindrical roller. Standard use of the microneedle roller typically results in a perforation density of 240 perforations/cm2 after 10 to 15 applications over the same skin area. While such microneedle approaches are certainly functional and worthwhile, clinical utility would be improved if the clearing agent could simply be applied topically onto intact skin and thereafter migrate across the stratum corneum and epidermis into the dermis. Food and Drug Administration (FDA) approved lipophilic polypropylene glycol-based polymers (PPG) and hydrophilic PEG-based polymers, both with indices of refraction that closely match that of dermal collagen (n=1.47) are available alone and in a combined pre-polymer mixture, such as polydimethylsiloxane (PDMS). PDMS is optically clear, and, in general, is considered to be inert, non-toxic and non-flammable. It is occasionally called dimethicone and is one of several types of silicone oil (polymerized siloxane), as was described in detail in an earlier section. The chemical formula for PDMS is CH₃[Si(CH₃)₂O]ₙSi(CH₃)₃, where n is the number of repeating monomer [SiO(CH₃)₂] units. The penetration of these optical clearing agents into appropriately treated skin takes about 60 minutes to achieve a high degree of scattering reduction and commensurate optical transport efficiency. With that in mind, a system utilizing this approach may be configured to activate its illumination after a time sufficient to establish optical clearing, and in sufficient volume to maintain it nominally throughout or during the treatment exposure. Alternately, the patient/user may be instructed to treat their skin a sufficient time prior to system usage.

Alternately, the microneedle roller may be configured with the addition of central fluid chamber that may contain the tissue clearing agent, which is in communication with the needles. This configuration may provide for enhanced tissue clearing by allowing the tissue clearing agent to be injected directly via the microneedles.

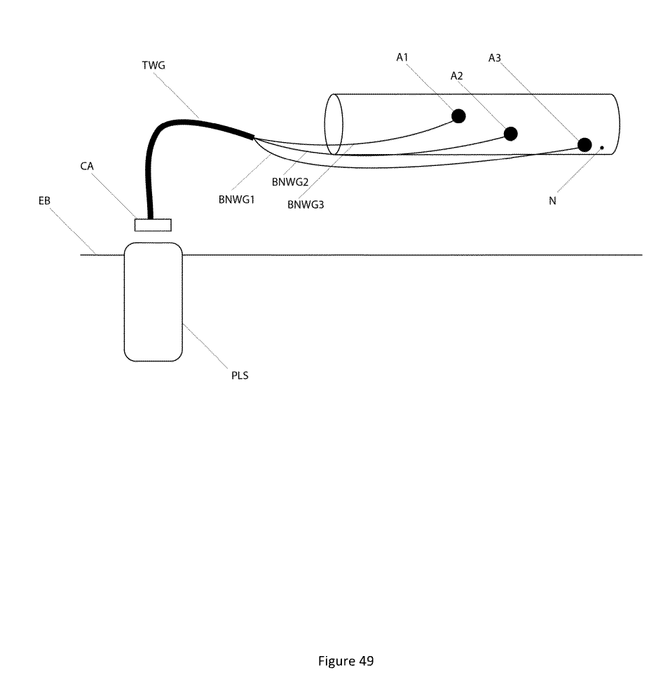

A compression bandage-like system could push exposed emitters and/or applicators into the tissue containing a subsurface optogenetic target to provide enhanced optical penetration via pressure-induced tissue clearing in cases where the applicator is worn on the outside of the body; as might be the case with a few of the clinical indications described herein, like micromastia, erectile dysfunction, and neuropathic pain. This configuration may also be combined with tissue clearing agents for increased effect. The degree of pressure tolerable is certainly a function of the clinical application and the site of its disposition. Alternately, the combination of light source compression into the target area may also be combined with an implanted delivery segment, or delivery segments, that would also serve to collect the light from the external source for delivery to the applicator(s). Such an example is shown in FIG. 49, where External Light Source PLS (which may the distal end of a delivery segment, or the light source itself) is placed into contact with the External Boundary EB of the patient PLS emits light into the body, and it may be collected by Collection Apparatus CA, which may be a lens, a concentrator, or any other means of collecting light, for propagation along Trunk Waveguide TWG, which may a bundle of fibers, or other such configuration, which then bifurcates into separate interim delivery segments BNWGx, that in turn deliver the light to Applicators Ax that are in proximity to Target N.

An electrical synapse is a mechanical and electrically conductive pore between two abutting neurons that is formed at a narrow gap between the pre- and postsynaptic neurons known as a gap junction. At gap junctions, such cells approach within about 3.5 nm of each other, a much shorter distance than the 20 to 40 nm distance that separates cells at a chemical synapse. In many systems, electrical synapse systems co-exist with chemical synapses.

Compared to chemical synapses, electrical synapses conduct nerve impulses faster, but unlike chemical synapses they do not have gain (the signal in the postsynaptic neuron is the same or smaller than that of the originating neuron). Electrical synapses are often found in neural systems that require the fastest possible response, such as defensive reflexes and in cases where a concerted behavior of a subpopulation of cells is required (propagation of calcium waves in astrocytes, etc.). An important characteristic of electrical synapses is that most of the time, they are bidirectional, i.e. they allow impulse transmission in either direction. However, some gap junctions do allow for communication in only one direction.

Normally, current carried by ions could travel in either direction through this type of synapse. However, sometimes the junctions are rectifying synapses, containing voltage-dependent gates that open in response to a depolarization and prevent current from traveling in one of the two directions. Some channels may also close in response to increased calcium (Ca²⁺) or hydrogen (H+) ion concentration so as not to spread damage from one cell to another.

Certain embodiments of the present invention relate to systems, methods and apparatuses that provide for optogenetic control of synaptic rectification in order to offer improved control for both optogenetic and electrical nerve stimulation.

Nerve stimulation, such as electrical stimulation (“e-stim”), causes bidirectional impulses in a neuron, antidromic and orthodromic stimulation. That is, an action potential triggers pulses that propagate in both directions along a neuron. However, the coordinated use of optogenetic inhibition in combination with stimulation to allow only the intended signal to propagate beyond the target location by suppression or cancellation of the errant signal using optogenetic inhibition. This may be achieved in multiple ways using what we will term “multi-applicator devices” or “multi-zone devices”. The function and characteristics of the individual elements utilized in such devices were defined earlier.

In a first embodiment, a multi-applicator device is configured to utilize separate applicators Ax for each interaction zone Zx along the target nerve N, as is shown in FIG. 50A. One example is the use optogenetic applicators on both ends (A1&A3) and an electrical stimulation device (A2) in the middle. This example was chosen to represent a generic situation wherein the desired signal direction may be on either side of the excitatory electrode. The allowed signal direction may be chosen by the selective application of optogenetic inhibition from the applicator on the opposite side of the central Applicator A2. In this non-limiting example, the Errant Impulse EI is on the right hand side, RHS, of the stimulation cuff A2, traveling to the right, as indicated by arrow DIR-EI, and passing through the portion f the target covered by A3 and the Desired Impulse DI is on the left hand side, LHS, of A2, travelling to the left, as indicated by arrow DIR-DI, and, passing through the portion f the target covered by A1. Activation of A3 may serve to disallow transmission of EI via optogenetic inhibition of the signal, suppressing it. Similarly, activation of A1 instead of A3 would serve to suppress the transmission of the Desired Impulse DI and allow the Errant Impulse EI to propagate. Therefore, bi-directionality is maintained in this triple applicator configuration, making it a flexible configuration for Impulse direction control. Such flexibility may not always be clinically required, and simpler designs may be used, as is explained in subsequent paragraphs. This inhibition/suppression signal may accompany or precede the electrical stimulation, as dictated by the specific kinetics of the therapeutic target. Each optical applicator may also be made such that it is capable of providing both optogenetic excitation and inhibition by utilizing two spectrally distinct light sources to activate their respective opsins in the target. In this embodiment, each applicator, Ax, is served by its own Delivery Segment, DSx. These Delivery Segments, DS1, DS2, and DS3, serve as conduits for light and/or electricity, as dictated by the type of applicator present. As previously described, the Delivery Segment(s) connect(s) to a Housing containing the electrical and/or electro-optical components required to provide for power supply, processing, feedback, telemetry, etc. Alternately, Applicator A2 may be an optogenetic applicator and either Applicators A1 or A3 may be used to suppress the errant signal direction.

Alternately, as mentioned above, only a pair of applicators may be required when the therapy dictates that only a single direction is required. Referring to the embodiment of FIG. 50B, the directionality of the Desired Impulse DI and Errant Impulse EI described above is maintained. However, Applicator A3 is absent because the directionality of the Desired Impulse DI is considered to be fixed as leftward, and Applicator A2 is used for optogenetic suppression of the Errant Impulse EI, as previously described.

Alternately, referring to the embodiment of FIG. 50C, a single applicator may be used, wherein the electrical and optical activation zones Z1, Z2, and Z3 are spatially separated, but still contained within a single applicator A.

Furthermore, the combined electrical stimulation and optical stimulation described herein may also be used for intraoperative tests of inhibition in which an electrical stimulation is delivered and inhibited by the application of light to confirm proper functioning of the implant and optogenetic inhibition. This may be performed using the applicators and system previously described for testing during the surgical procedure, or afterwards, depending upon medical constraints and/or idiosyncrasies of the patient and/or condition under treatment. The combination of a multiple-applicator, or multiple-zone applicator, or multiple applicators, may also be define which individual optical source elements within said applicator or applicators may be the most efficacious and/or efficient means by which to inhibit nerve function. That is, an e-stim device may be used as a system diagnostic tool to test the effects of different emitters and/or applicators within a multiple emitter, or distributed emitter, system by suppressing, or attempting to suppress, the induced stimulation via optogenetic inhibition using an emitter, or a set of emitters and ascertaining, or measuring, the patient, or target, response(s) to see the optimal combination for use. That optimal combination may then be used as input to configure the system via the telemetric link to the housing via the external controller/programmer. Alternately, the optimal pulsing characteristics of a single emitter, or set of emitters, may be likewise ascertained and deployed to the implanted system.

Referring to FIGS. 51A-51D, certain aspects of cross section of a nerve bundle (20) are illustrated in the context of injecting genetic material into the nerve in an “intraneural” injection using a needle. Referring to FIG. 51A, a cross section of a nerve bundle (20) is depicted to illustrate that a nerve bundle generally is a composite structure which may comprise thousands of nerve cells which may have various different functions. In certain interventional scenarios, it is desirable to conduct an intraneural injection to target specific portions of the bundle—or at least generally the portion of the bundle that resides within the epineurium. Referring to FIG. 51B, for example, a needle (202) is being advanced (204) toward a nerve bundle (20). FIG. 51C shows the needle inserted across the epineurium and into the nerve bundle (20)—but due to the generally compliant coupling of nerves to nearby tissues, and also due to the compliant and viscoelastic nature of the nerve and other supporting tissues, it may be difficult to determine how far into a given structure the needle has been advanced. Referring to FIG. 51D, to address this, a counterloading member (206) may be utilized to apply a counterload (208) against the nerve bundle (20) while the bundle is being injected from the opposite side. In one embodiment, it may be desirable to understand the geometric relationship between the counterloading member (206) and the needle (202) such that a distance of needle intrusion may be estimated.

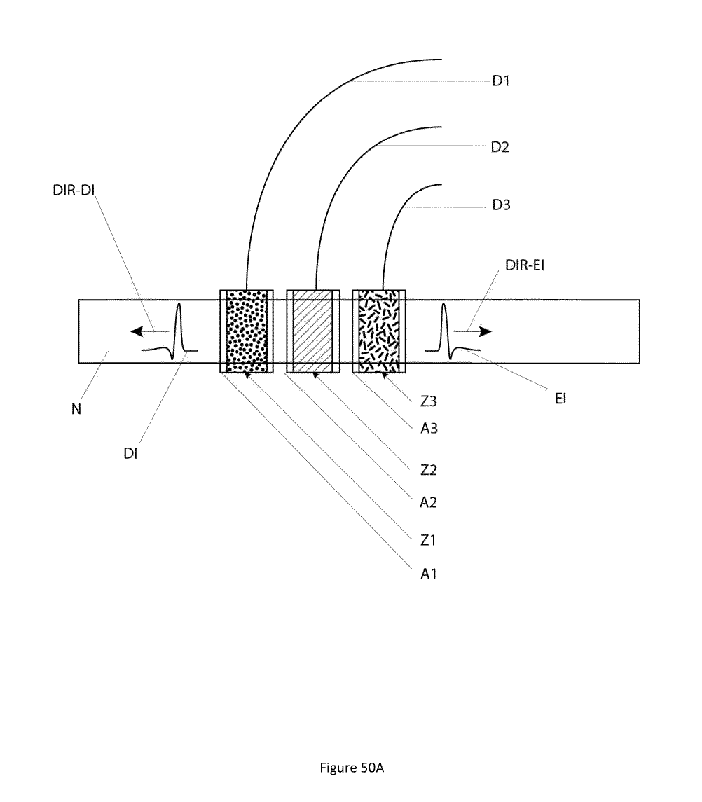

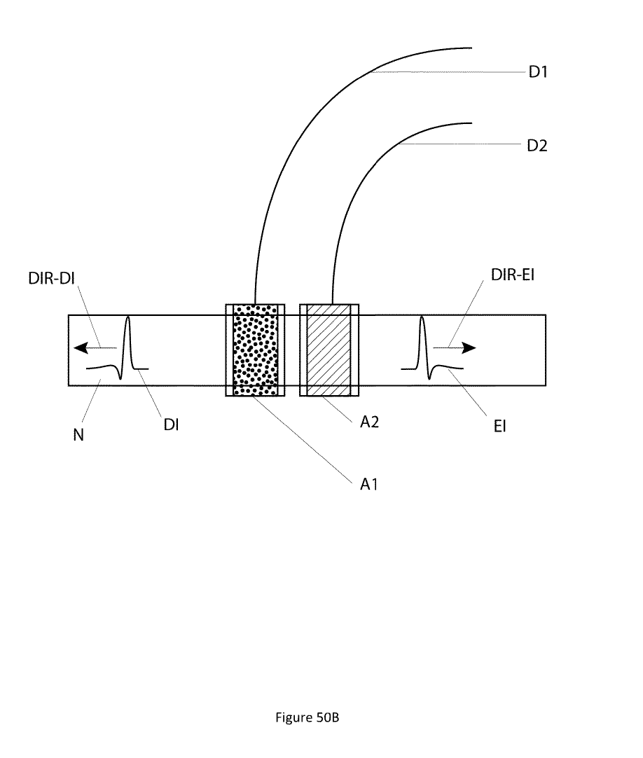

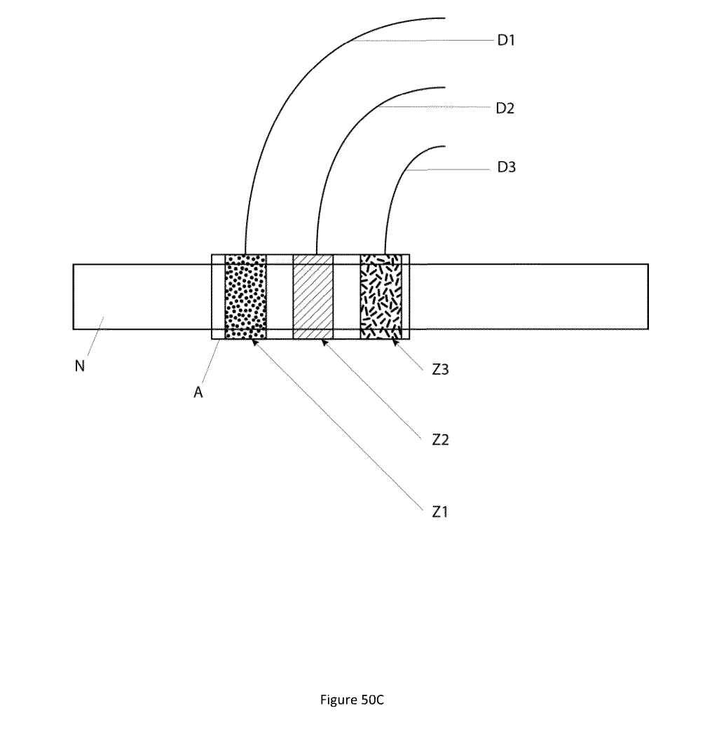

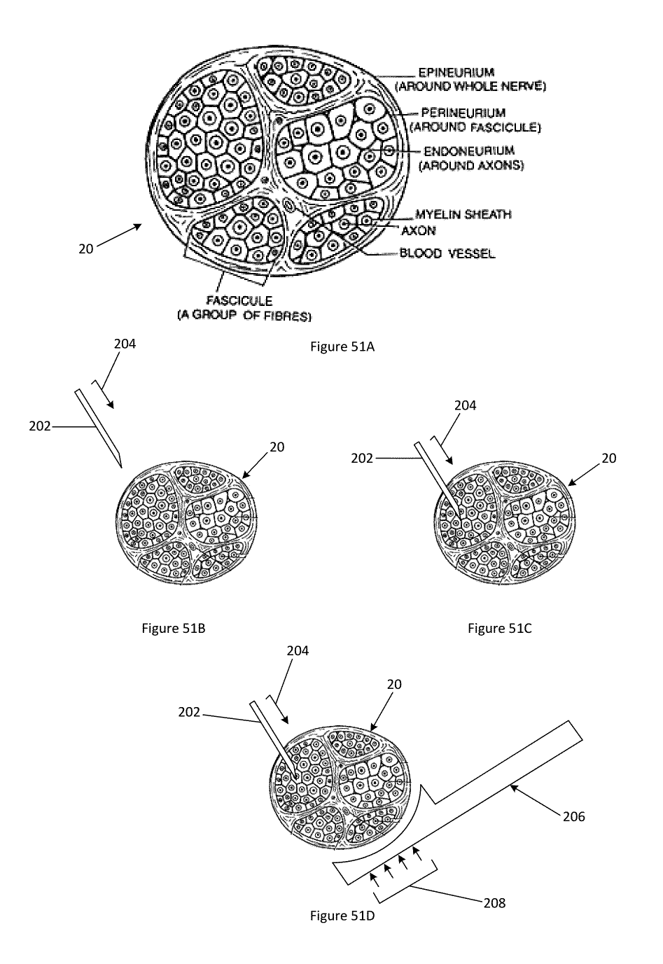

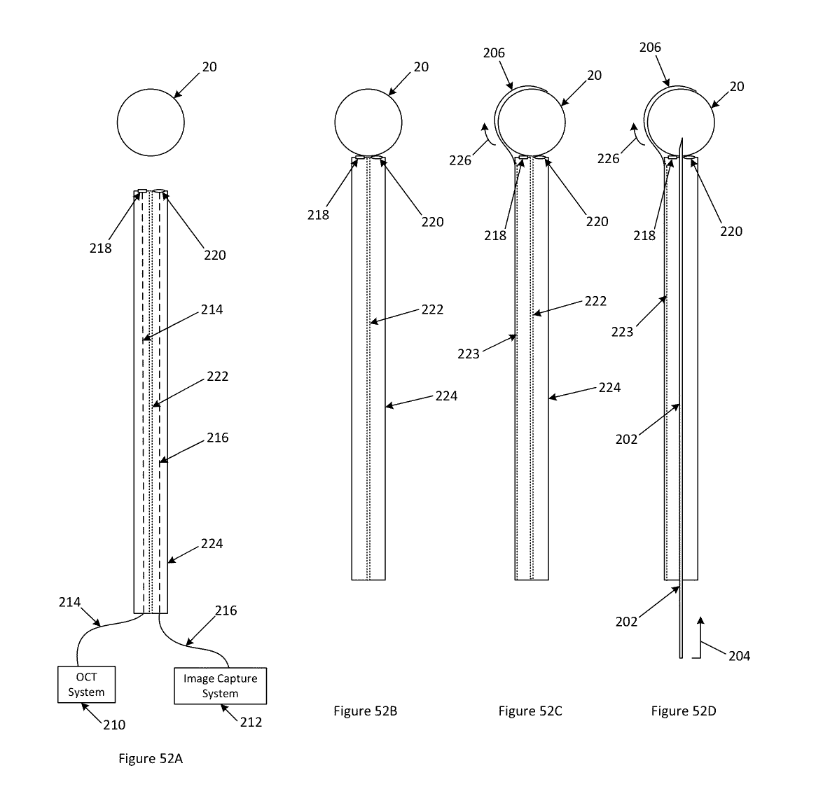

Referring to FIGS. 52A-52D, one embodiment for controllably conducting intraneural injections is depicted. As shown in FIG. 52A, an elongate instrument (224) such as a tube, catheter, manually steerable catheter, robotically steerable catheter, trocar, or the like may be utilized as a platform for controlled intraneural injection. The elongate instrument (224) may comprise a working lumen (222) through which other elongate instruments, such as an injection needle, may be passed. The elongate instrument (224) may also comprise imaging and/or sensing elements configured to assist with finding and interfacing a targeted tissue structure, such as a targeted nerve bundle (20). The embodiment of FIG. 52A features a distally-coupled optical coherence tomography (“OCT”) imaging interface (218), such as a lens, which may be operatively coupled, via a lead (214) which may comprise an optical fiber, to an extracorporeally positioned OCT imaging system which may comprise an interferometer; such systems are available, for example, from ThorLabs, Inc. of Newton, N.J. and may be utilized, for example, to measure the distance between the distal imaging interface (218) and nearby tissue layers or surfaces, such as the layers of a nerve bundle (20). The embodiment of FIG. 52A also features a distal image capture element (220) operatively coupled via a lead element (216) to an extracorporeally positioned image capture system (216) such as a camera. In one embodiment, the distal image capture element (220) may comprise an optical imaging lens, with the lead comprising one or more optical fibers for transmitting image information back to the image capture system (212). In another embodiment, the distal image capture element (220) may comprise an imaging chip, such as a CMOS chip, with the lead electronically transmitting (216) image information back to the image capture system (220) which may comprise an image processor. In another embodiment, the distal image capture element (220) may comprise one or more ultrasound transducers or arrays configured to electronically transmit via an electronic lead (216) image information which may be processed and assembled into ultrasound images by the image capture system (212). For simplicity of illustration, FIGS. 52B-52D do not show the OCT system (210) or image capture system (212), but as shown in FIG. 52B, their functionality may be utilized in practice to assist an operator who may be manually, electromechanically, and/or electromagnetically navigating the elongate instrument (224) to locate the targeted tissue structure, here the nerve bundle (20), and to interface the distal end of the elongate instrument directly against the outer surface of the nerve bundle (20). Radiography, transcutaneous ultrasound, fluoroscopy, and other imaging modalities may be utilized to assist with guidance of the instrumentation to the desired anatomy. Referring to FIG. 52C, in one embodiment, a flexible counterloading member (206), such as a one made from the nickel titanium superalloy known as “Nitinol”, may be movably coupled to the elongate instrument (224) through a working lumen (223) such that the counterloading member (206) may be slidably advanced out of the working lumen and into a configuration wherein it wraps around the nerve bundle (20) and may be utilized to contain and support the nerve bundle (20) while an injection needle (202) is advanced (204) through the central working lumen (222) of the elongate instrument (224) to conduct the intraneural injection, as shown in FIG. 52D. The distal portion or end of the counterloading member may comprise an atraumatic tip geometry to prevent skiving or puncturing into the tissues that it is configured to support.

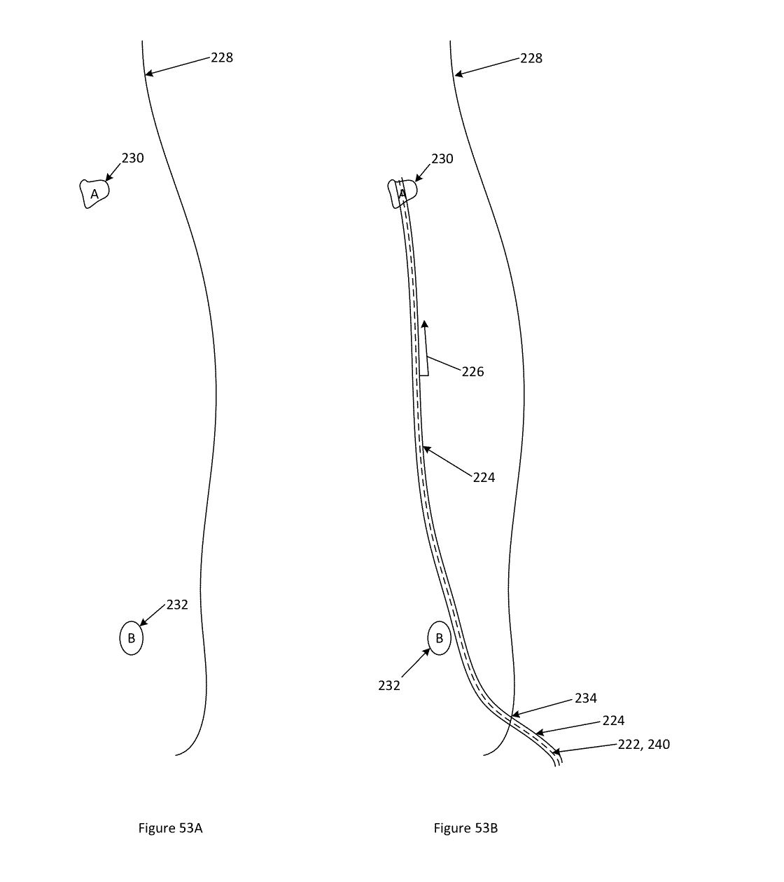

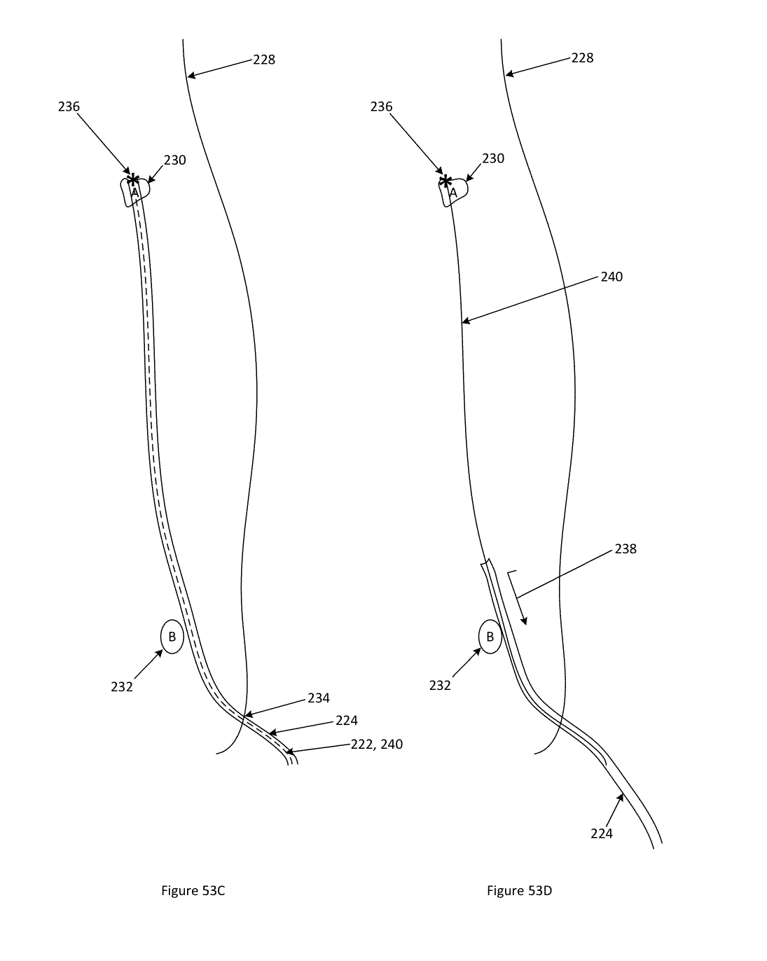

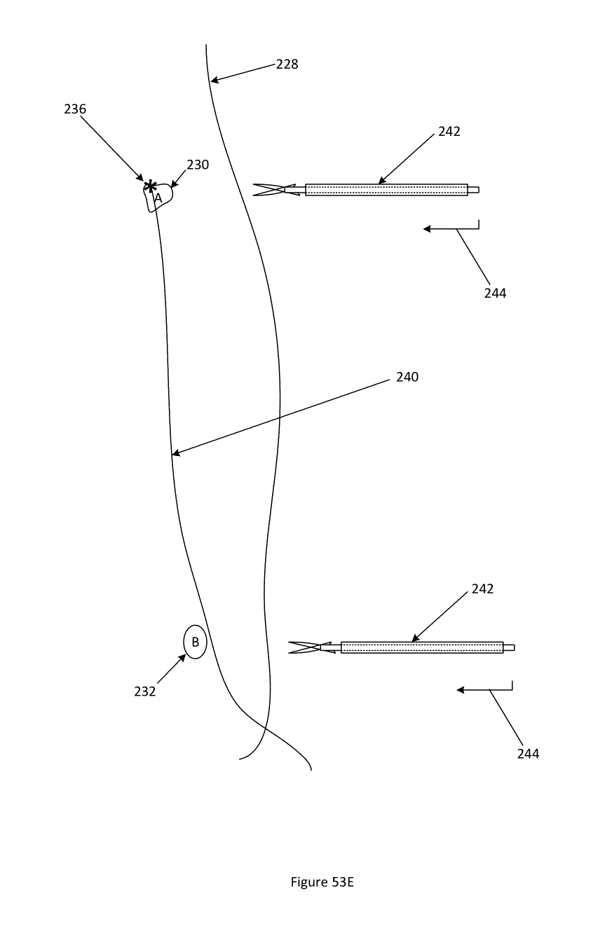

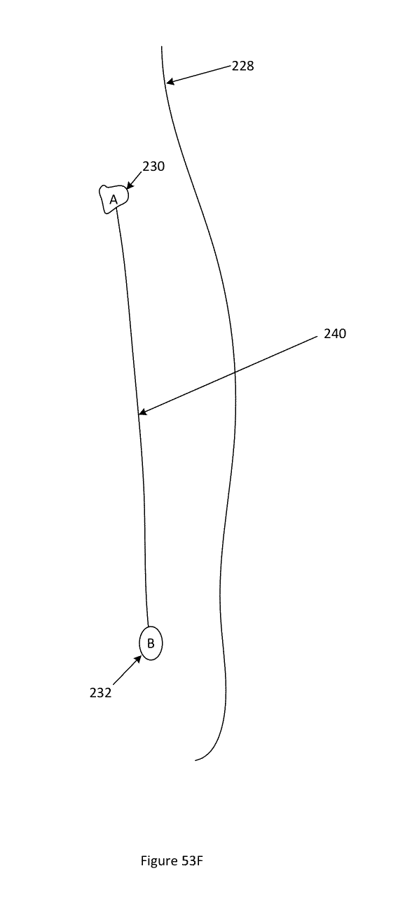

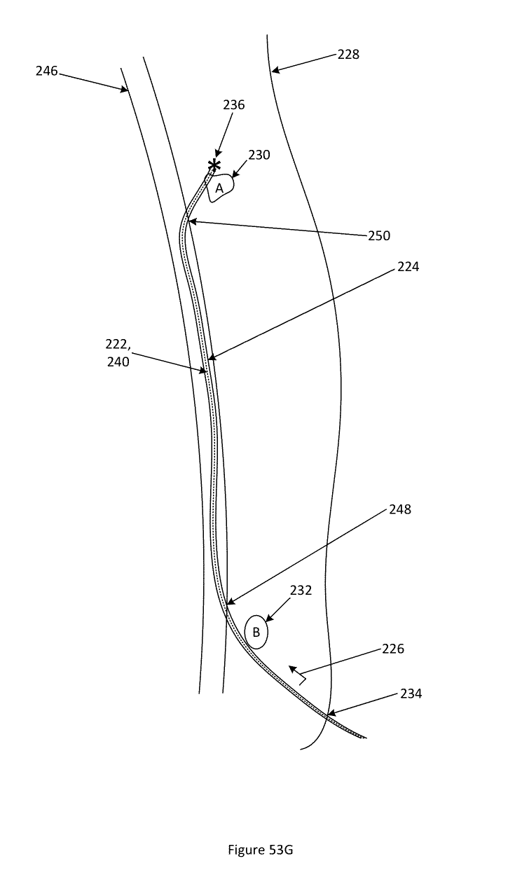

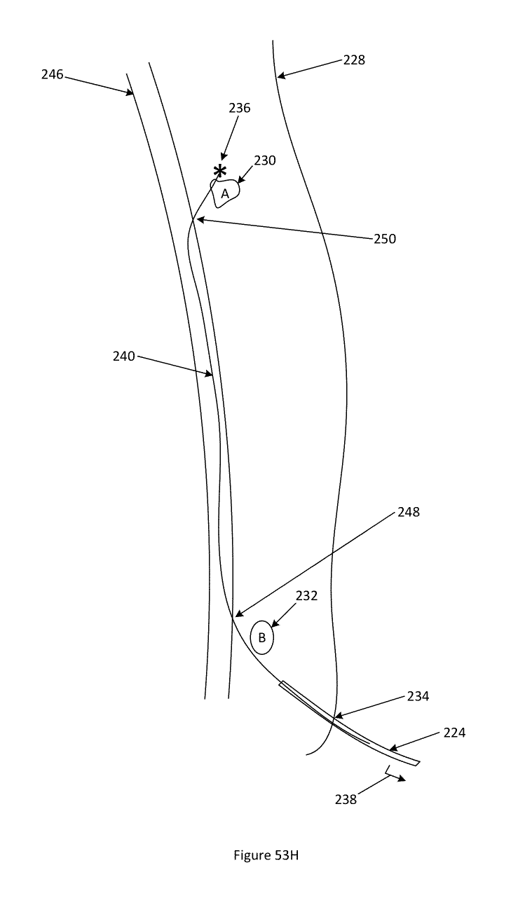

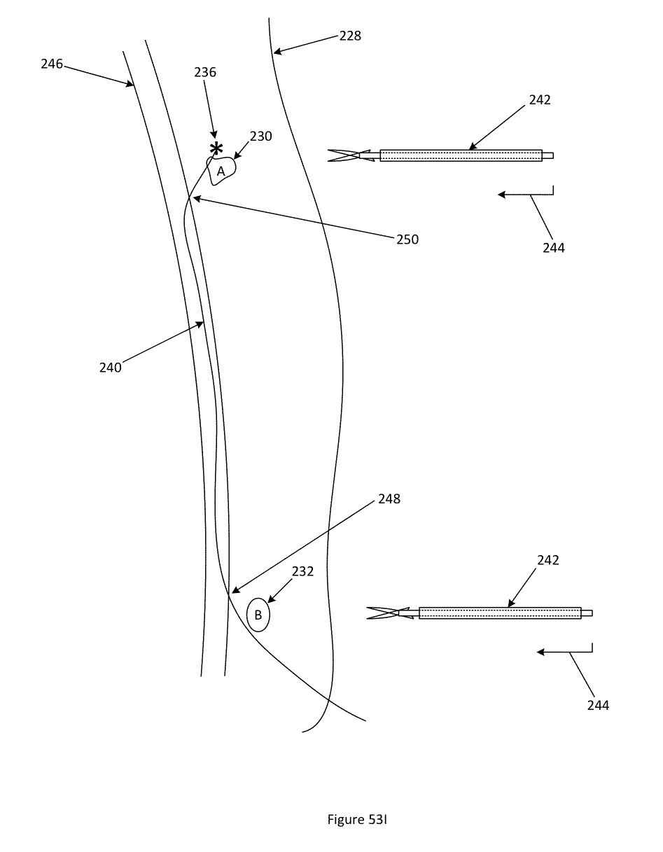

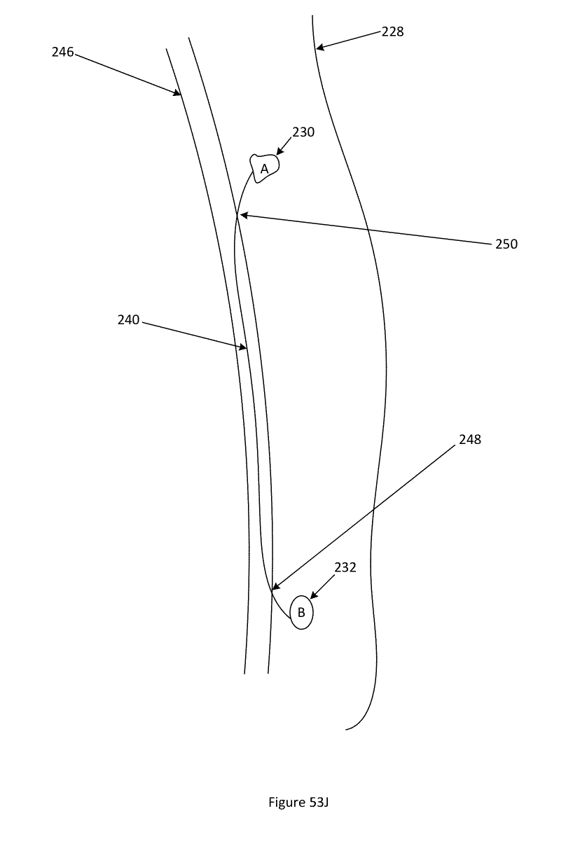

Referring to FIGS. 53A-53J, various aspects of configurations for placing elongate delivery segments (240) are illustrated. Referring to FIG. 53A, should there be a desire to place an electronic or optical lead between tissue structures or locations A (230) and B (232), a conventional surgical approach may involve creating an incision in the skin (228) and other associated layers of tissue to expose a subcutaneous flap, trench, or the like, placing the lead in place, and closing the surgical access. Such a conventional approach involves a large incision, which generally is undesirable. Referring to FIG. 53B, in one embodiment, an elongate instrument, such as those described above, and preferably one comprising a distal cutting tip as well as operator-controlled steerability during insertion (for example, using pull-pull steering tensile members or push push compressive members in a steerable catheter or trocar form, and/or an outer sheath that biases an internally coaxially coupled bent member to a straight configuration, such that relative roll and insertion/retraction of the outer and inner members provides steerability during insertion) may be inserted at a transcutaneous access point (234), inserted (226) past a location near location B (232), and to a location adjacent to location A (230), as shown. A lead (240) may be carried along within the working lumen (222) or inserted later. Referring to FIG. 53C, with the lead inserted past the end of the elongate instrument (224), an anchor member (236), such as a self-expanding Nitinol multifaceted anchor (such as a star or tubular shape), preferably featuring radiopaque markers for subsequent radiography and/or fluoroscopy location, may be utilized to maintain the position of the lead (240) during pullback (238) of the elongate instrument (224), as shown in FIG. 53D. FIG. 53E shows the lead (240) remaining in place between location A (230) and location B (232), with a cutting tool (242) being advanced to create a keyhole or port access directly to both locations (230, 232) to facilitate trimming of the length of the lead (240) and coupling of the resultant ends of the lead to other hardware, such as an applicator, an implantable power source, and the like, as described above. FIG. 53F shows the implanted lead (240) between the two locations (230, 232), as installed using the elongate instrumentation and keyhole or port access type wounds, without a long incision all of the way between the two locations (230, 232).

Referring to FIGS. 53G-53J, a somewhat similar installation is illustrated, with an elongate instrument being utilized to internally pull a lead (240) from one desired location to another—but in this embodiment, a vein is intentionally utilized as a native conduit for at least a portion of the lead pathway. Veins are located throughout the body, have relatively low internal pressure, and may be entered and exited with relatively little or no vascular fluid loss given an appropriate geometry (in one embodiment, a tapered and steerable distal cutting tip may be utilized to carefully manage insertion and exit trajectory of the instrumentation with the venous wall; instrumentation also may be coated with sealant materials, such as Fibrin, to prevent trans-venous pathway leakage). Thus referring to FIG. 53G, the elongate instrument (224) has entered the vein (246) at a location (248) adjacent location B (232), and intentionally exited the vein (246) at a location (250) adjacent location A (230)—thereby using the vein as a convenient conduit for carrying a portion of the lead (240). Referring to FIGS. 53G and 53H, an anchor (236) member is allowed to expand to retain the position of the lead (240) and the elongate instrument (224) is withdrawn (238). FIGS. 531 and 53J illustrate that port access cutting tools (242) may be inserted (244) and utilized as described above, leaving a lead (240) installed between the two desired locations (230, 232).

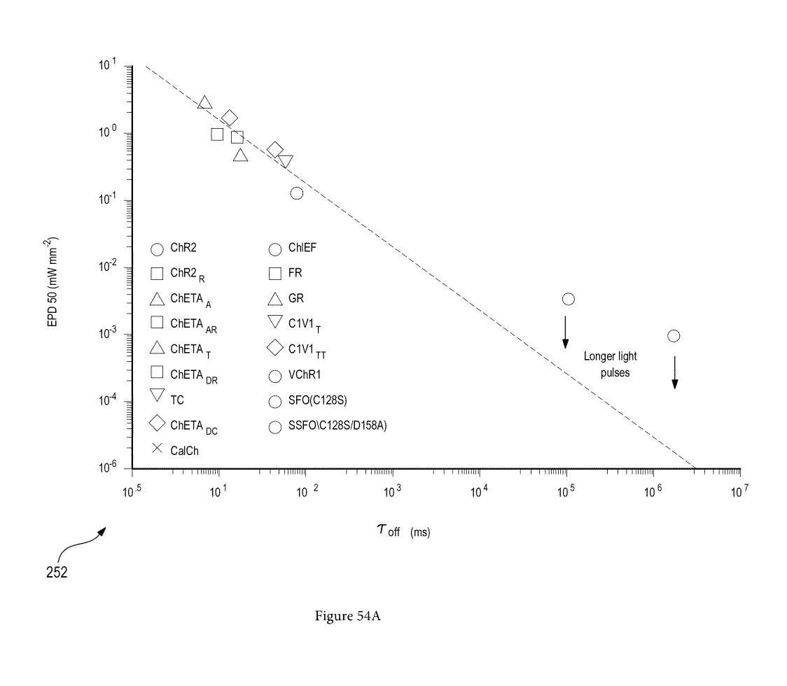

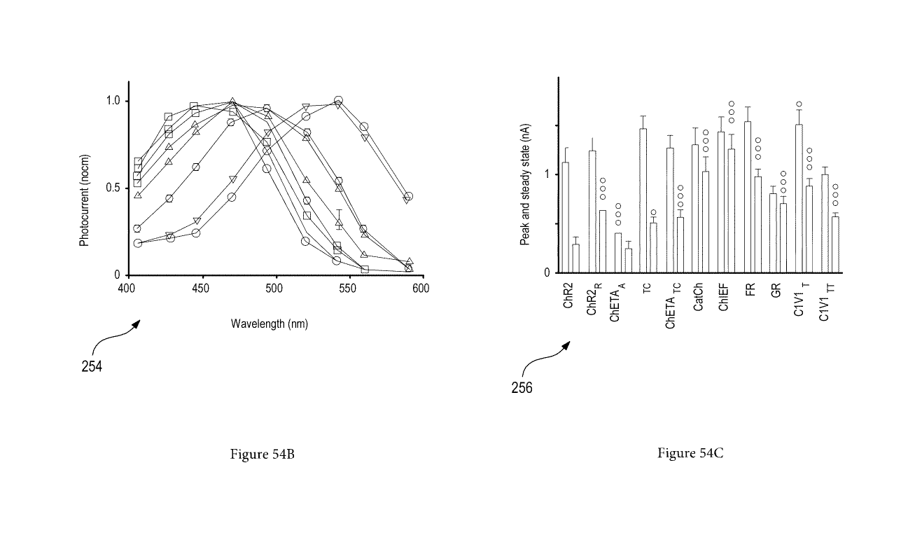

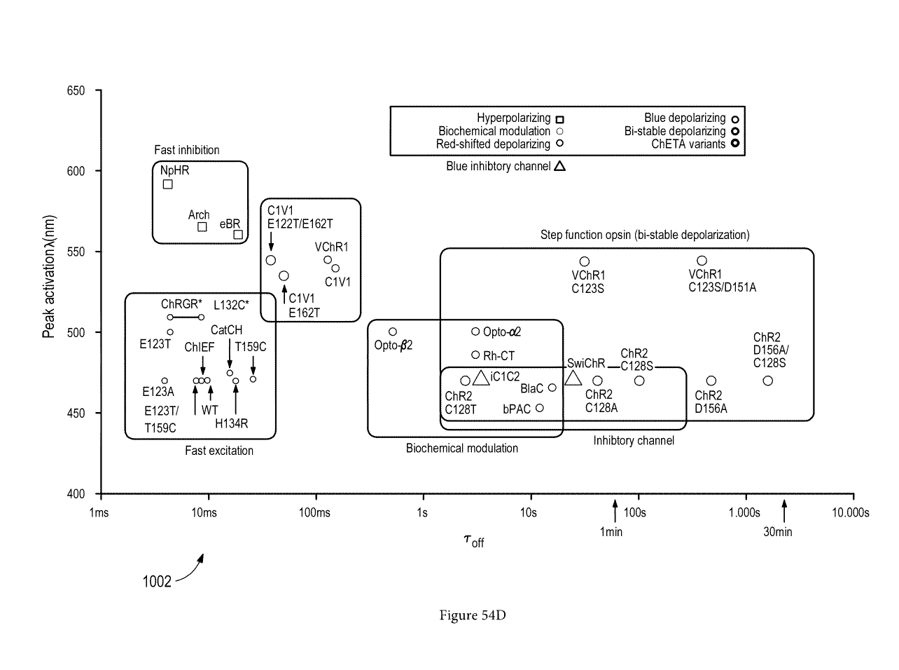

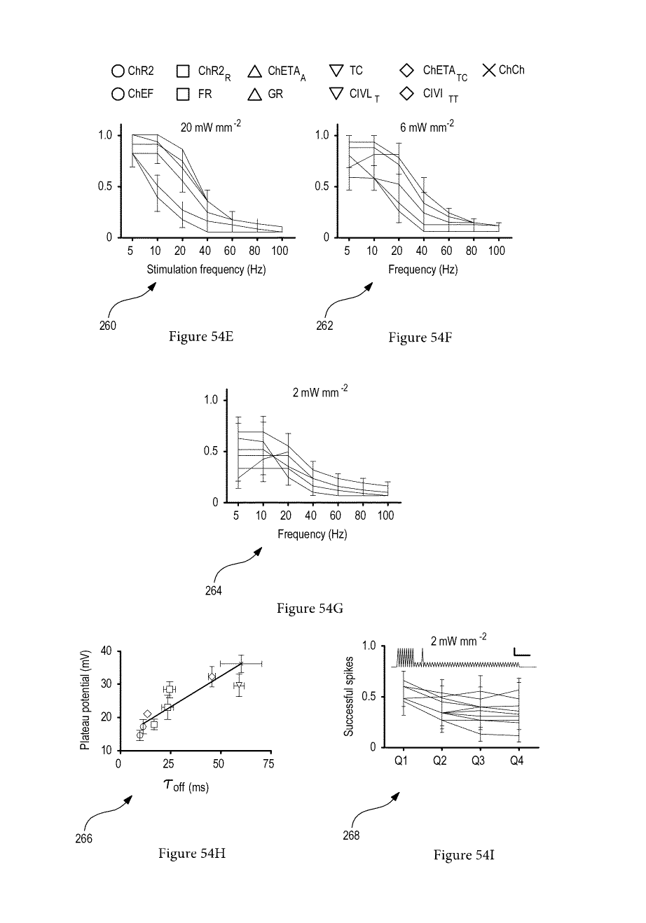

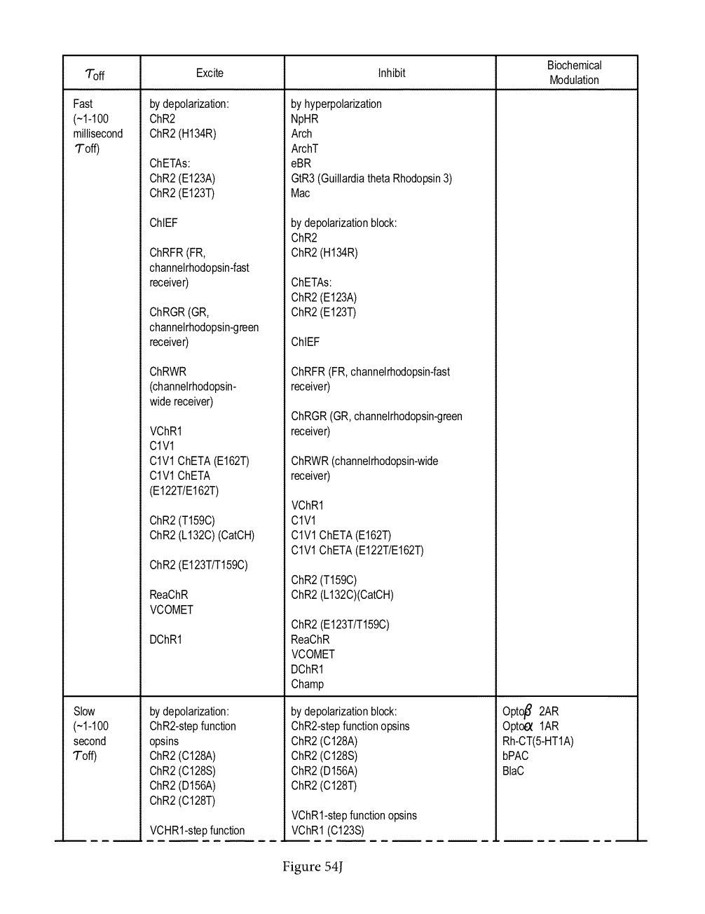

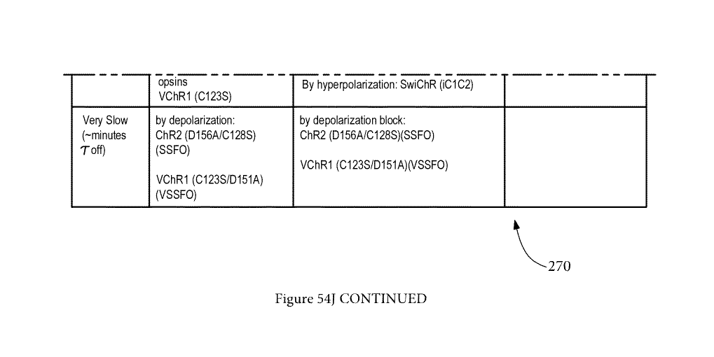

In certain scenarios wherein light sensitivity of opsin genetic material is of paramount importance, it may be desirable to focus less on wavelength (as discussed above, certain “red-shifted” opsins may be advantageous due to the greater permeability of the associated radiation wavelengths through materials such as tissue structures) and more on a tradeoff that has been shown between response time and light sensitivity (or absorption cross-section). In other words, optimal opsin selection in many applications may be a function of system kinetics and light sensitivity. Referring to the plot (252) of FIG. 54A, for example, electrophysiology dose for a 50% response (or “EPD50”; lower EPD50 means more light-sensitive) is plotted versus temporal precision (“tau-off”, which represents the time constant with which an opsin deactivates after the illumination has been discontinued). This data is from Mattis et al, Nat Methods 2011, Dec. 10; 9(2): 159-172, which is incorporated by reference herein in its entirety, and illustrates the aforementioned tradeoff. In addition to EPD50 and tau-off, other important factors playing into opsin selection optimization may include exposure density (“H-thresh”) and photocurrent levels. H-thresh may be assessed by determining the EPD50 dose for an opsin; the longer the channel created by the opsin requires to “reset”, the longer the associated membrane will remain polarized, and thus will block further depolarization. The following table features a few exemplary opsins with characteristics compared.

| | | | | Pentration | | | |
|---|---|---|---|---|---|---|---|
| | | | | Depth | | | |
| | | Tau- | Lambda | [normalized | Peak | SS | Peak |
| | EPD50 | off | Peak | to | Photocurrent | Photocurrent | Potential |
| Opsin | [mW/mm2] | [ms] | [nm] | 475 nm] | [nA] | [nA] | [mV] |
| C1V1t | 0.3 | 75 | 540 | 1.67 | 1.5 | 1 | 30 |
| C1V1tt | 0.4 | 50 | 540 | 1.67 | 1.1 | 0.6 | 32 |
| CatCh | 0.3 | 60 | 475 | 1.00 | 1.25 | 1 | 38 |
| VChR1 | 0.1 | 100 | 550 | 1.80 | | | |

Thus, the combination of low exposure density (H-thresh), long photorecovery time (tau-off), and high photocurrent results in an opsin well-suited for applications that do not require ultra-temporal precision, such as those described herein for addressing satiety, vision restoration, and pain. As described above, a further consideration remains the optical penetration depth of the light or radiation responsible for activating the opsin. Tissue is a turbid medium, and predominantly attenuates the power density of light by Mie (elements of similar size to the wavelength of light) and Rayleigh (elements of smaller size than the wavelength of light) scattering effects. Both effects are inversely proportional to the wavelength, i.e. shorter wavelength is scattered more than a longer wavelength. Thus, a longer opsin excitation wavelength is preferred, but not required, for configurations where there is tissue interposed between the illumination source and the target. A balance may be made between the ultimate irradiance (optical power density and distribution) at the target tissue containing the opsin and the response of the opsin itself. The penetration depth in tissue (assuming a simple lambda⁻⁴ scattering dependence) is listed in the table above. Considering all the abovementioned parameters, both C1V1t and VChR1 are desirable choices in many clinical scenarios, due to combination of low exposure threshold, long photorecovery time, and optical penetration depth. FIGS. 54B-54C and FIGS. 54E-541 feature further plots (254, 256, 260, 262, 264, 266, 268, respectively) containing data from the aforementioned incorporated Mattis et al reference, demonstrating the interplay/relationships of various parameters of candidate opsins. FIG. 54D features a plot (258) similar to that shown in FIG. 3B, which contains data from Yizhar et al, Neuron. 2011 July; 72:9-34, which is incorporated by reference herein in its entirety. The table (270) of FIG. 49J features data from the aforementioned incorporated Yizhar et al reference, in addition to Wang et al, 2009, Journal of Biological Chemistry, 284: 5625-5696 and Gradinaru et al, 2010, Cell: 141:1-12, both of which are incorporated by reference herein in their entirety.

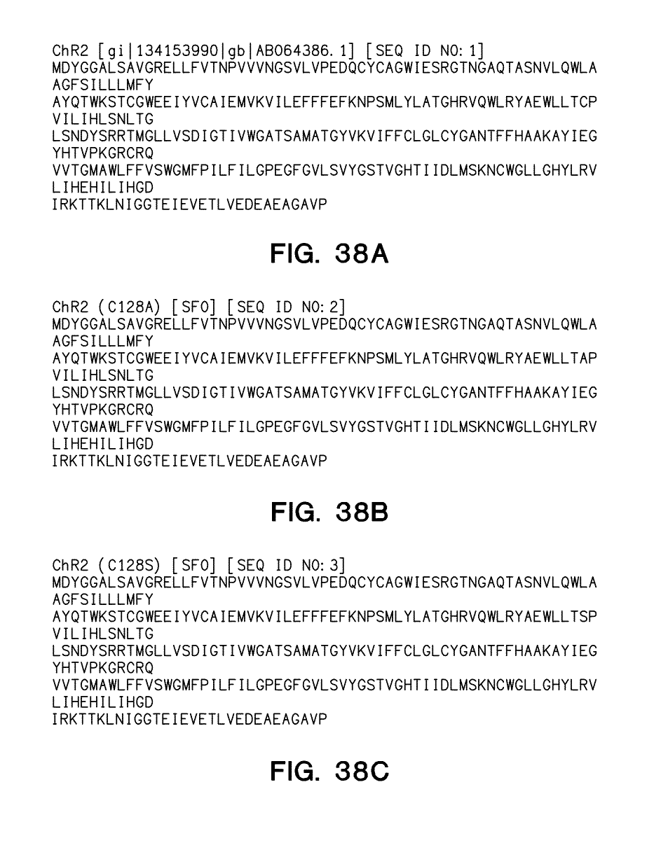

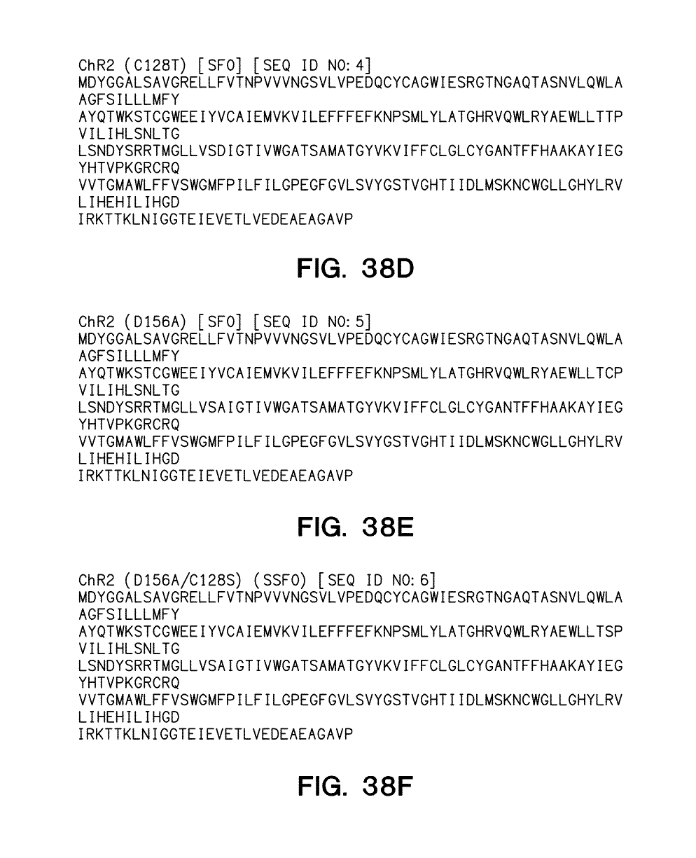

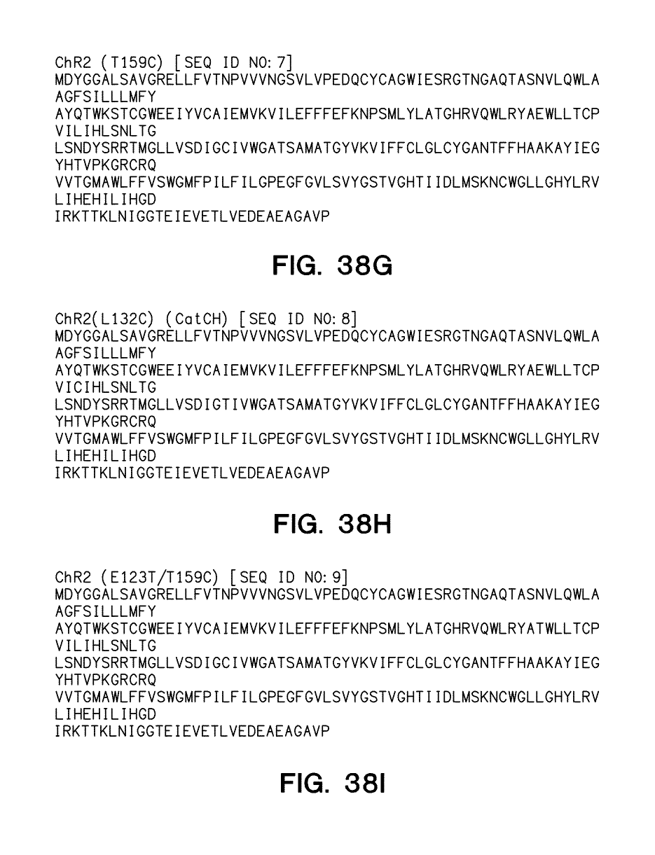

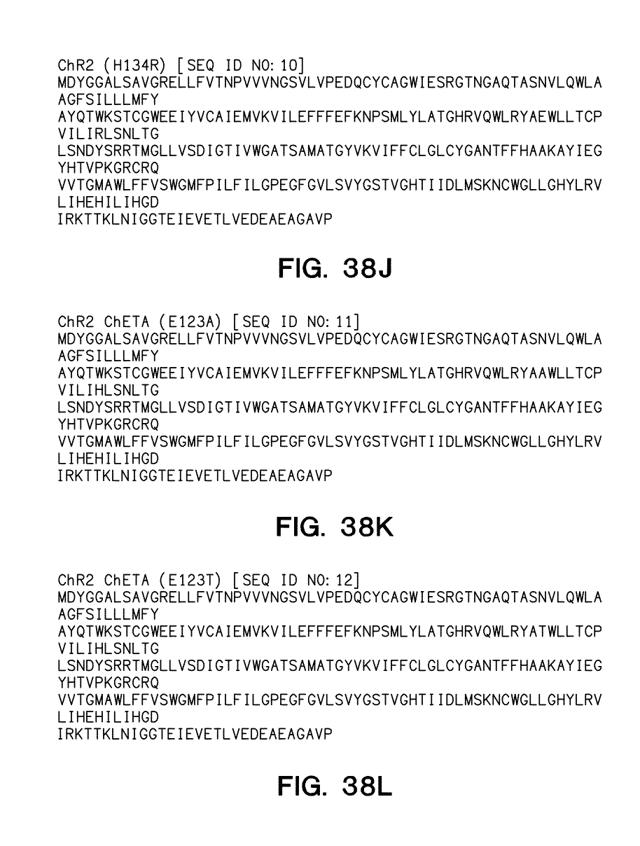

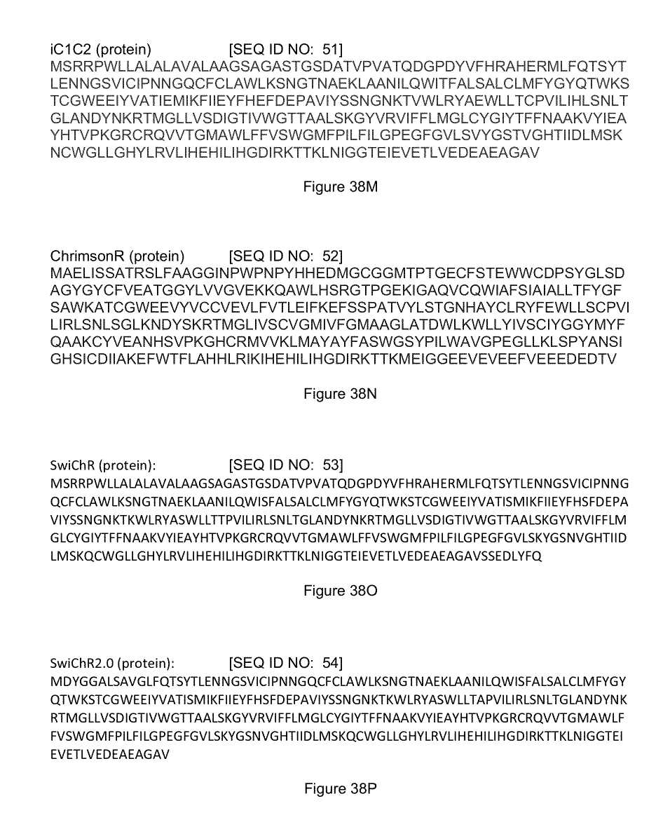

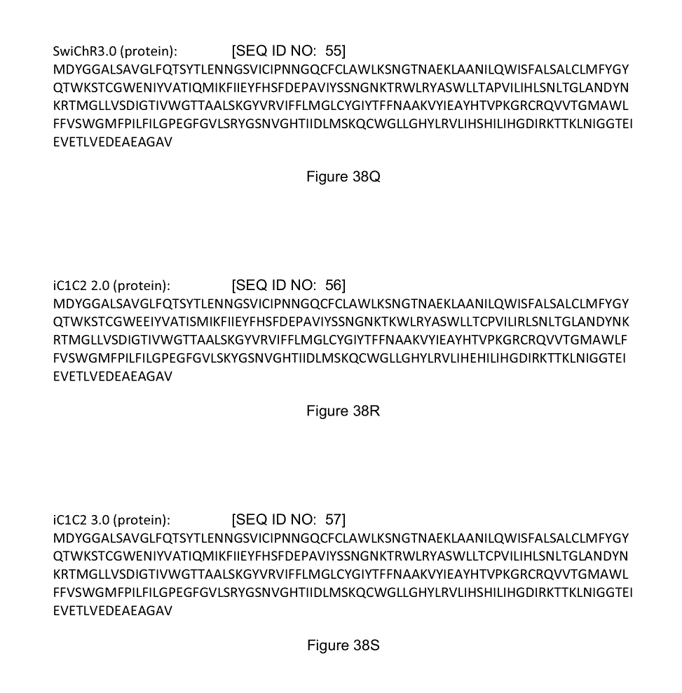

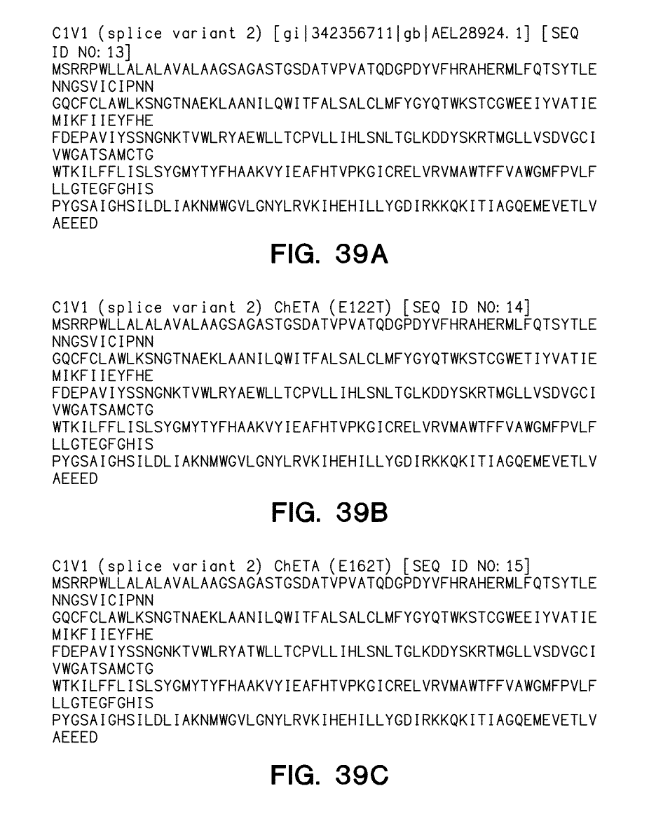

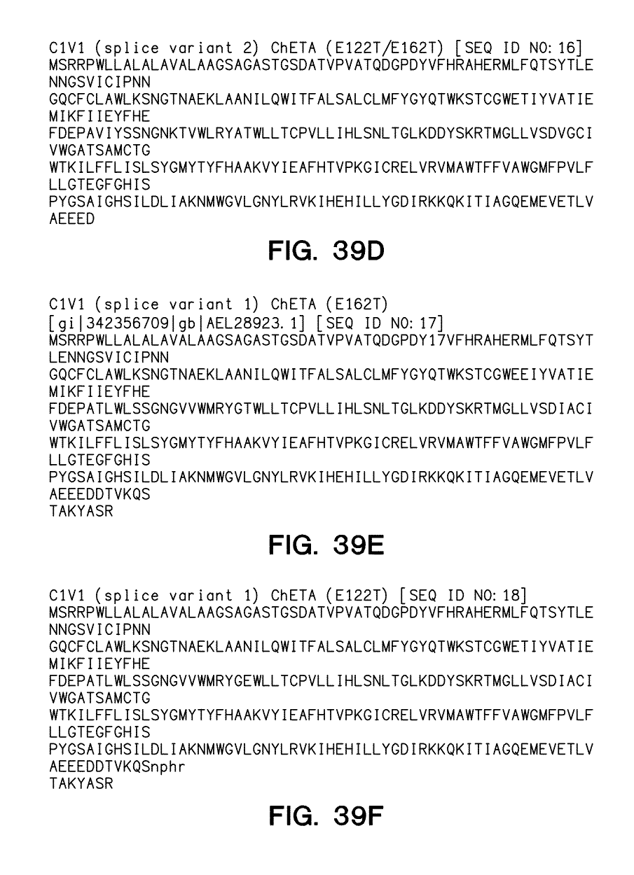

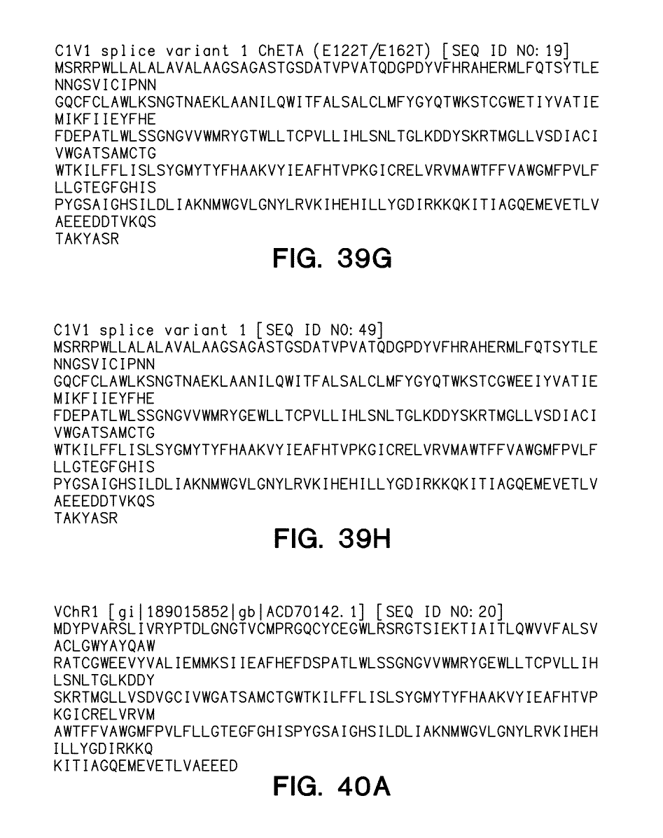

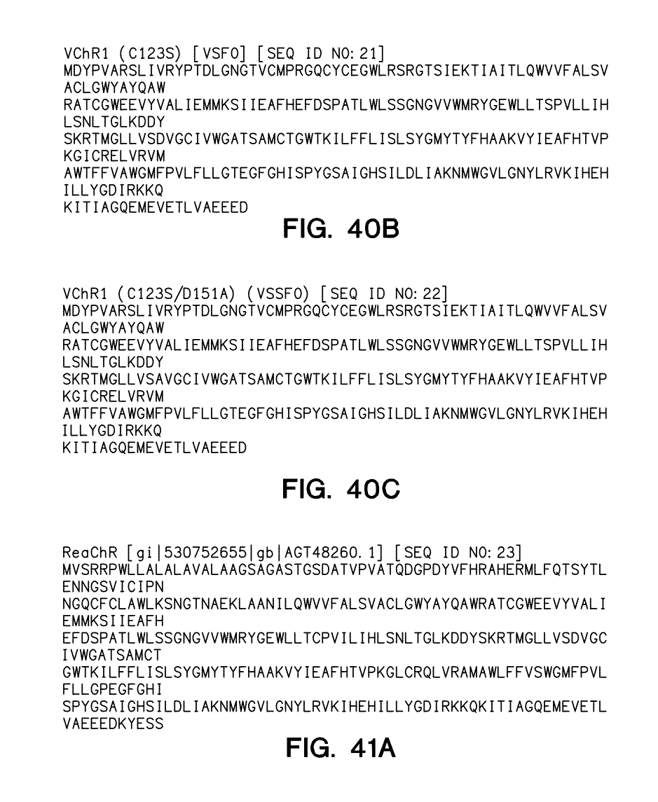

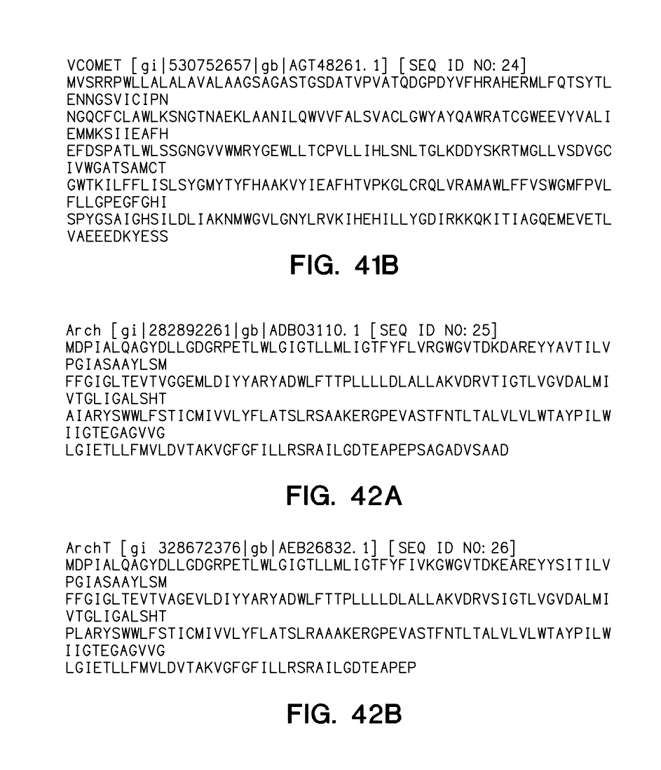

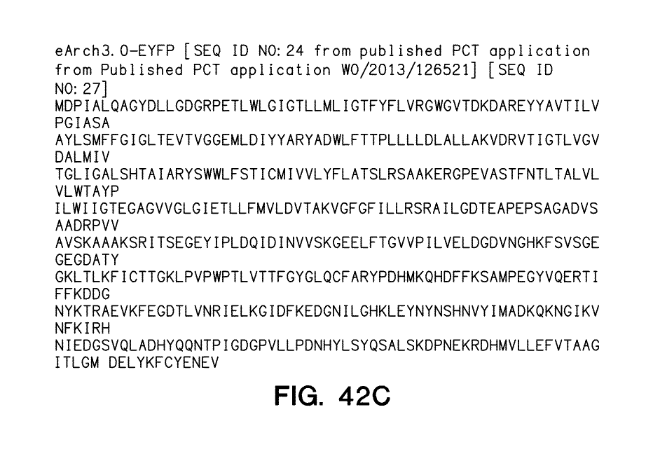

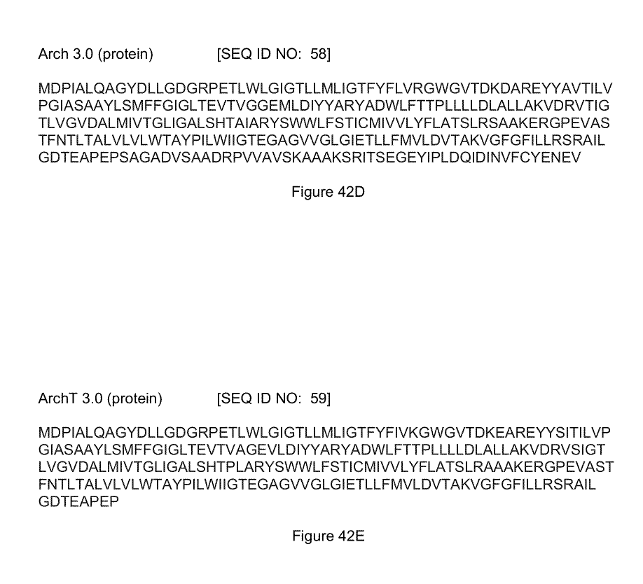

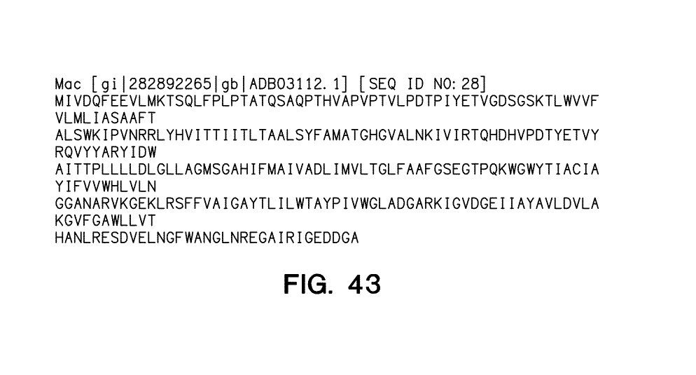

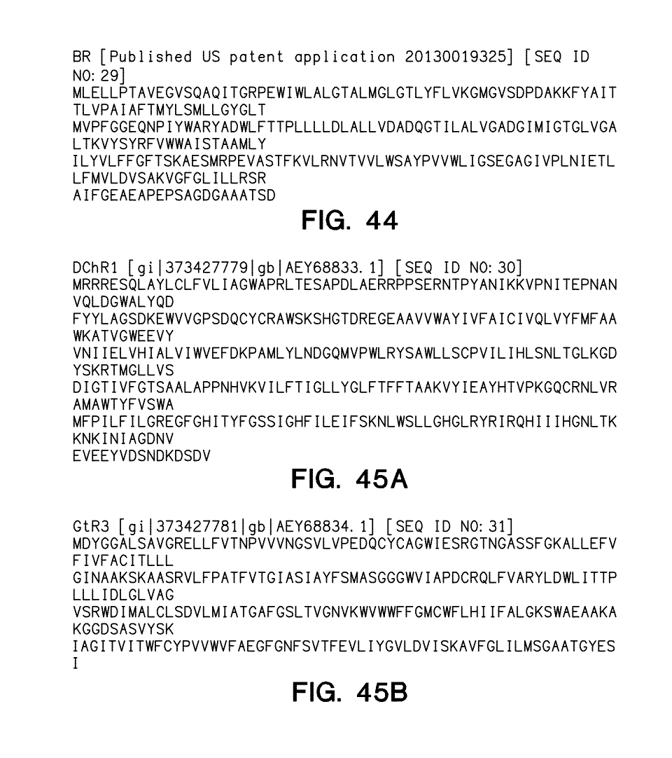

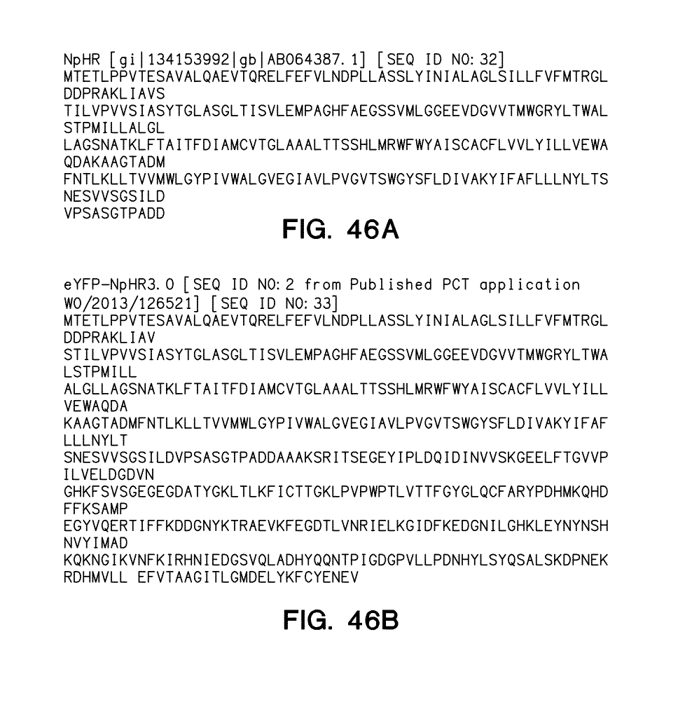

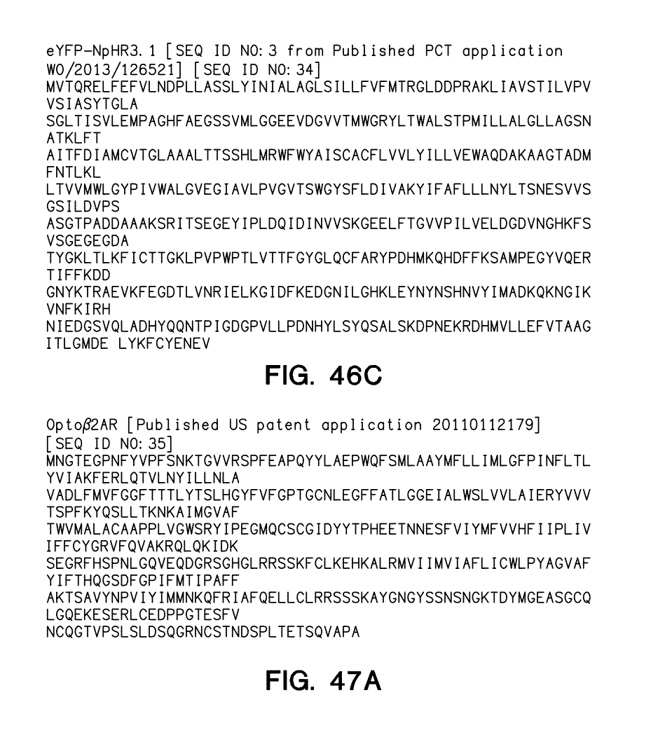

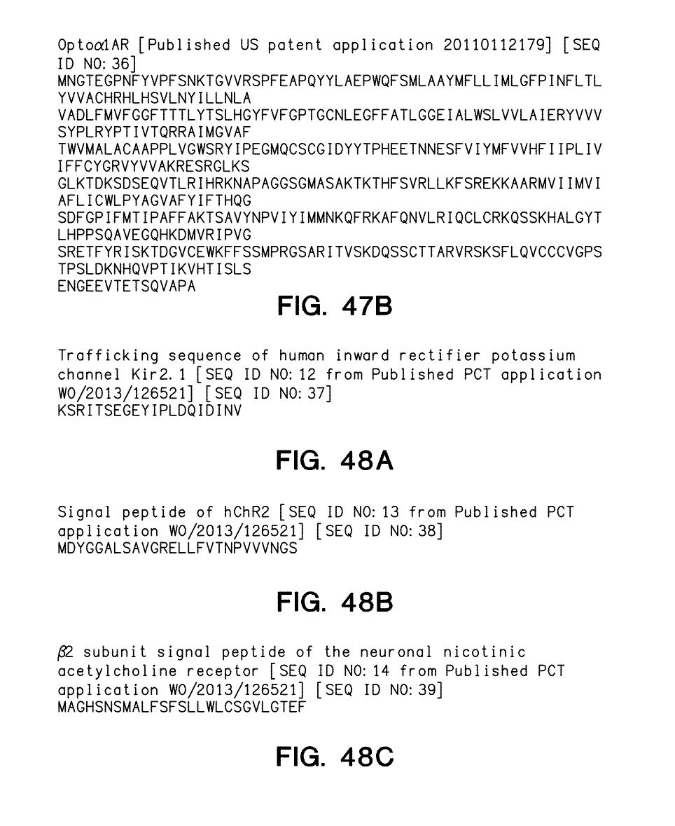

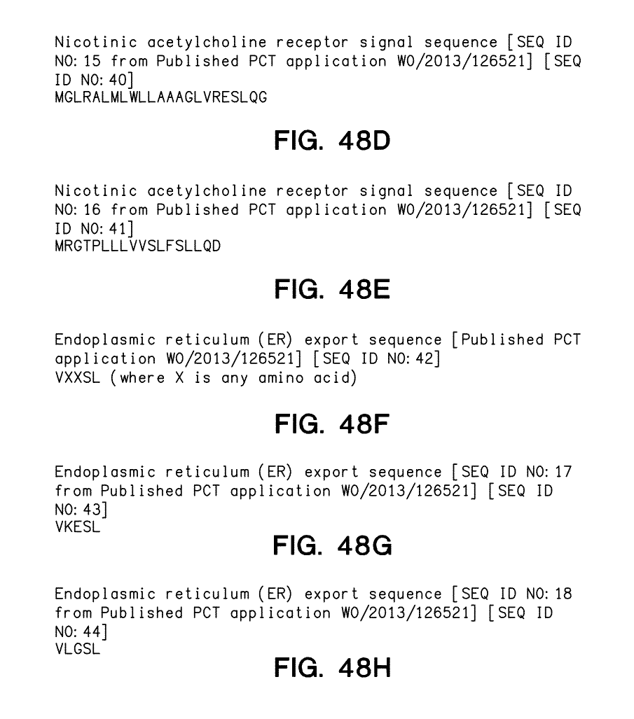

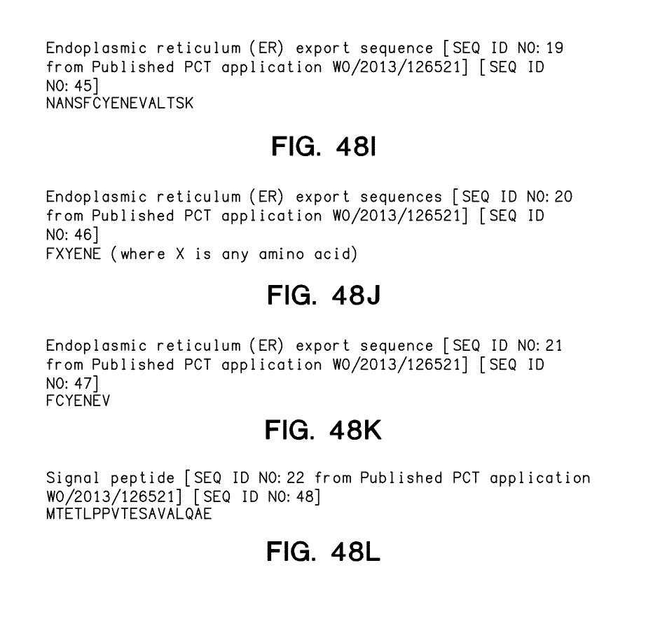

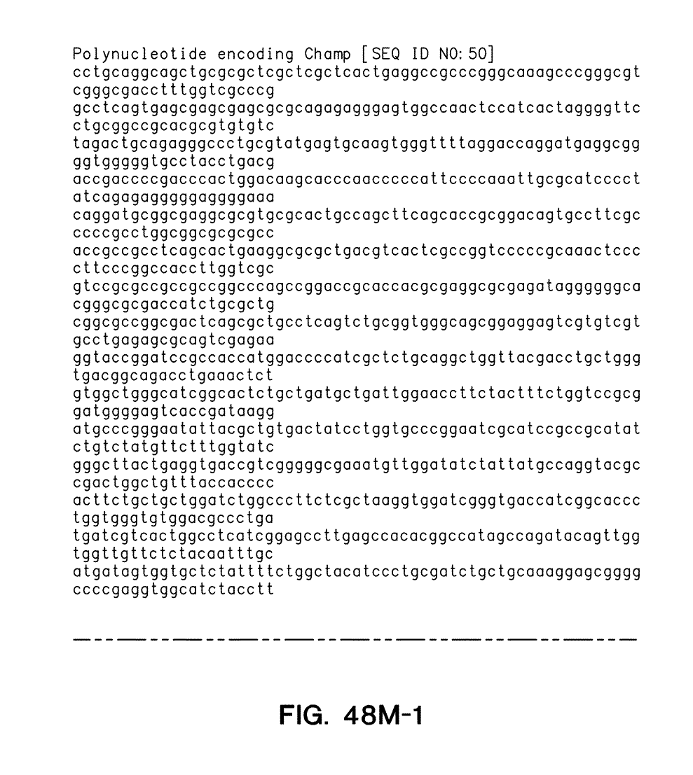

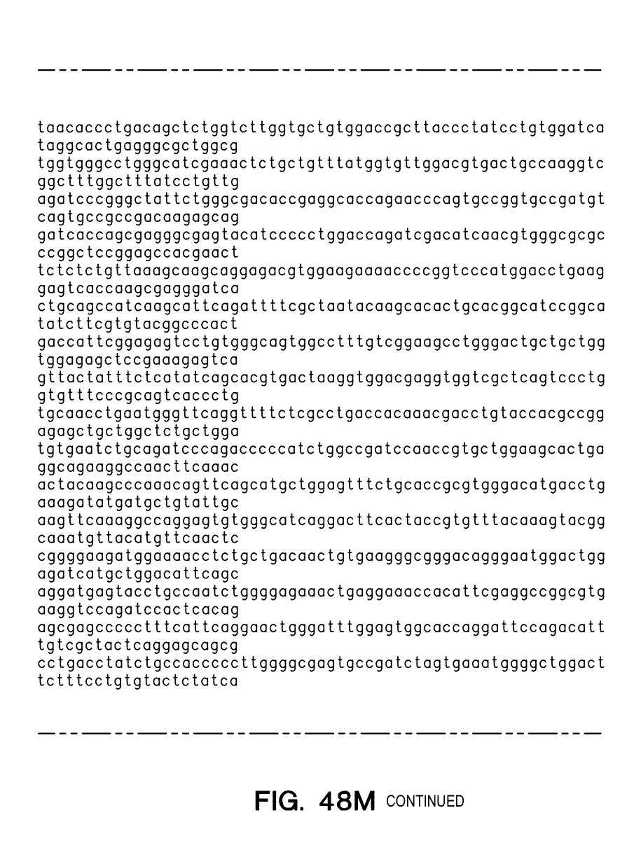

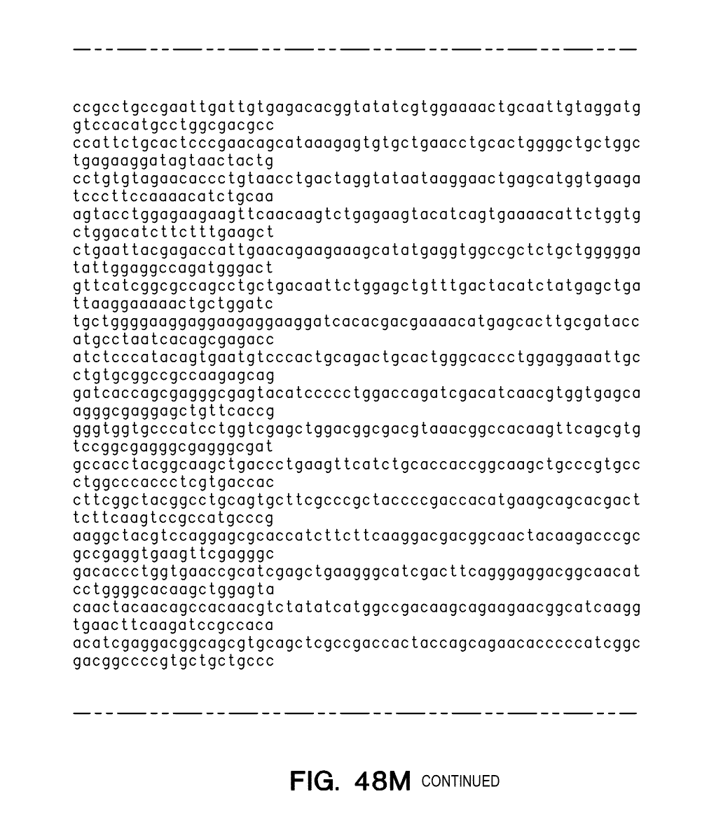

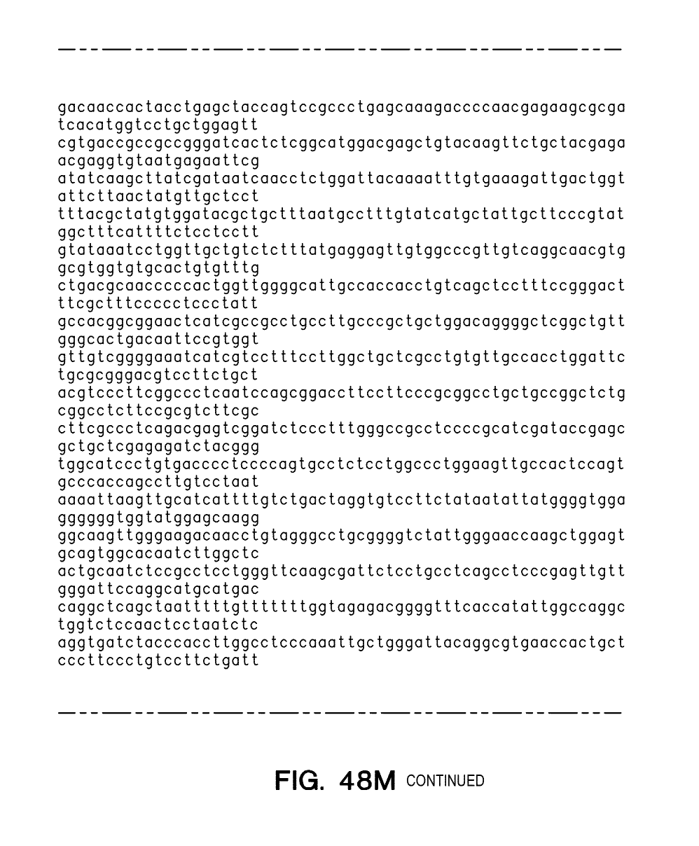

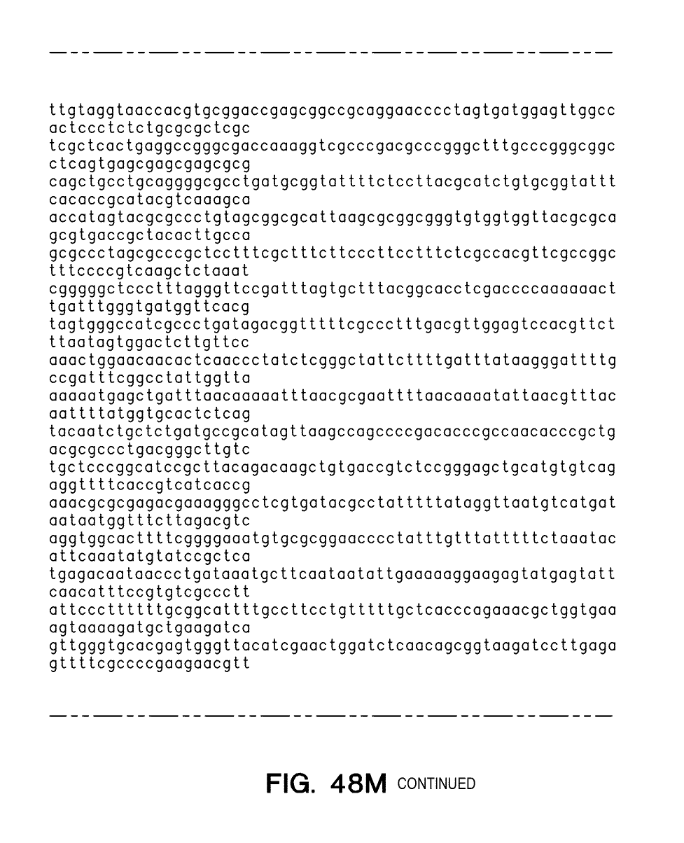

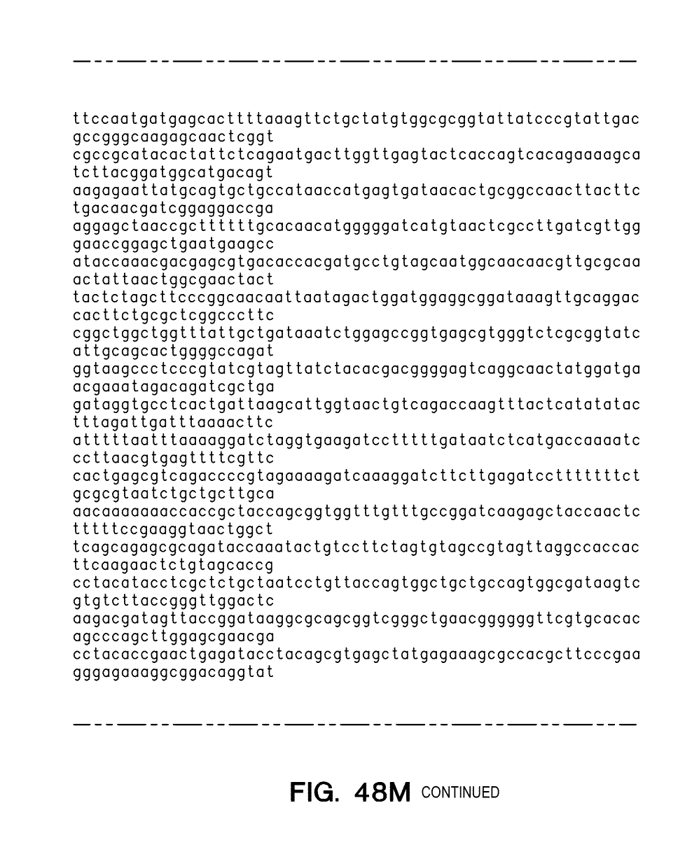

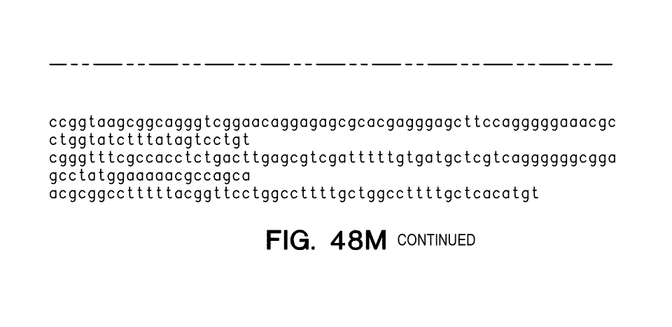

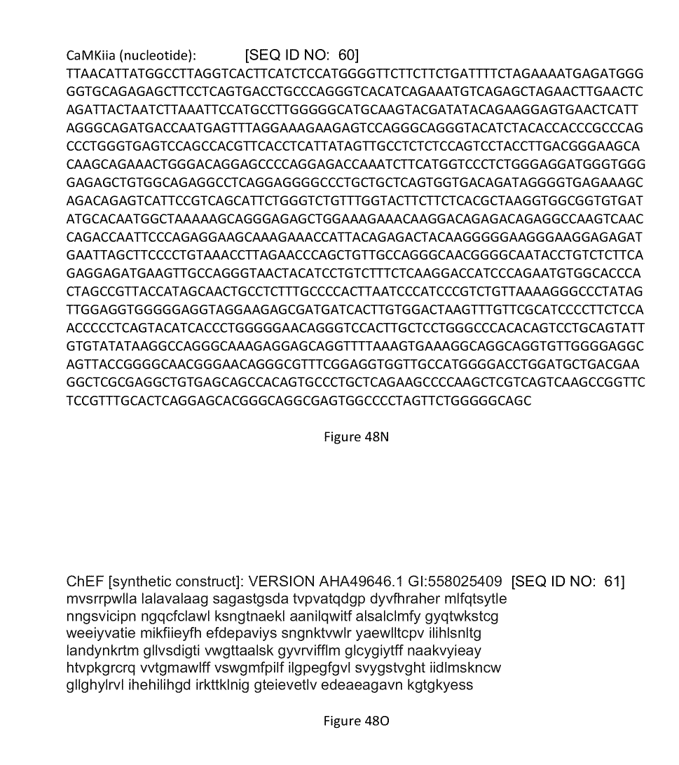

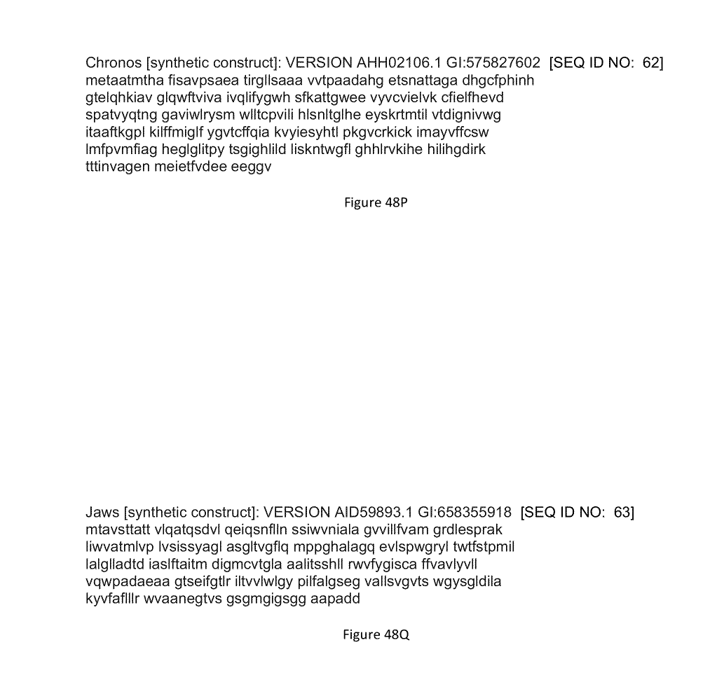

Excitatory opsins useful in the invention may include red-shifted depolarizing opsins including, by way of non-limiting examples, C1V1 and C1V1 variants C1V1/E162T and C1V1/E122T/E162T; blue depolarizing opsins including ChR2/L132C and ChR2/T159C and combinations of these with the ChETA substitutions E123T and E123A; and SFOs including ChR2/C128T, ChR2/C128A, and ChR2/C128S. These opsins may also be useful for inhibition using a depolarization block strategy. Inhibitory opsins useful in the invention may include, by way of non-limiting examples, NpHR, eNpHR 1.0, eNpHR 2.0, eNpHR 3.0, SwiChR, SwiChR 2.0, SwiChR 3.0, Mac, Mac 3.0, Arch, ArchT, Arch 3.0, ArchT 3.0, iChR, ChR2, C1V1-T, C1V1-TT, Chronos, Chrimson, ChrimsonR, CatCh, VChR1-SFO, ChR2-SFO, ChR2-SSFO, ChEF, ChIEF, Jaws, ChloC, Slow ChloC, iC1C2, iC1C2 2.0, and iC1C2 3.0. Opsins including trafficking motifs may be useful. An inhibitory opsin may be selected from those listed in FIG. 54J, by way of non-limiting examples. A stimulatory opsin may be selected from those listed in FIG. 54J, by way of non-limiting examples. An opsin may be selected from the group consisting of Opto-β2AR or Opto-α1AR, by way of non-limiting examples. The sequences illustrated in FIGS. 38A-48Q pertain to opsin proteins, trafficking motifs, and polynucleotides encoding opsin proteins related to configurations described herein. Also included are amino acid variants of the naturally occurring sequences, as determined herein. Preferably, the variants are greater than about 75% homologous to the protein sequence of the selected opsin, more preferably greater than about 80%, even more preferably greater than about 85% and most preferably greater than 90%. In some embodiments the homology will be as high as about 93 to about 95 or about 98%. Homology in this context means sequence similarity or identity, with identity being preferred. This homology will be determined using standard techniques known in the art. The compositions of the present invention include the protein and nucleic acid sequences provided herein including variants which are more than about 50% homologous to the provided sequence, more than about 55% homologous to the provided sequence, more than about 60% homologous to the provided sequence, more than about 65% homologous to the provided sequence, more than about 70% homologous to the provided sequence, more than about 75% homologous to the provided sequence, more than about 80% homologous to the provided sequence, more than about 85% homologous to the provided sequence, more than about 90% homologous to the provided sequence, or more than about 95% homologous to the provided sequence.

In one embodiment, for example, the housing (H) comprises control circuitry and a power supply; the delivery system (DS) comprises an electrical lead to pass power and monitoring signals as the lead operatively couples the housing (H) to the applicator (A); the applicator (A) preferably comprises a single fiber output style applicator, which may be similar to those described elsewhere herein. Generally the opsin configuration will be selected to facilitate controllable inhibitory neuromodulation of the associated neurons within the targeted neuroanatomy in response to light application through the applicator. Thus in one embodiment an inhibitory opsin such as NpHR, eNpHR 1.0, eNpHR 2.0, eNpHR 3.0, SwiChR, SwiChR 2.0, SwiChR 3.0, Mac, Mac 3.0, Arch, ArchT, Arch 3.0, ArchT 3.0, iChR, ChR2, C1V1-T, C1V1-TT, Chronos, Chrimson, ChrimsonR, CatCh, VChR1-SFO, ChR2-SFO, ChR2-SSFO, ChEF, ChIEF, Jaws, ChloC, Slow ChloC, iC1C2, iC1C2 2.0, and iC1C2 3.0 may be utilized. In another embodiment, an inhibitory paradigm may be accomplished by utilizing a stimulatory opsin in a hyper-activation paradigm, as described above. Suitable stimulatory opsins for hyperactivation inhibition may include ChR2, VChR1, certain Step Function Opsins (ChR2 variants, SFO), ChR2/L132C (CatCH), excitatory opsins listed herein, or a red-shifted C1V1 variant (e.g., C1V1) or the Chrimsom family of opsins, which may assist with illumination penetration through fibrous tissues which may tend to creep in or encapsulate the applicator (A) relative to the targeted neuroanatomy. In another embodiment, an SSFO may be utilized. An SFO or an SSFO or an inhibitory channel is differentiated in that it may have a time domain effect for a prolonged period of minutes to hours, which may assist in the downstream therapy in terms of saving battery life (i.e., one light pulse may get a longer-lasting physiological result, resulting in less overall light application through the applicator A). As described above, preferably the associated genetic material is delivered via viral transfection in association with injection paradigm, as described above. An inhibitory opsin may be selected from those listed in FIG. 49J, by way of non-limiting examples. A stimulatory opsin may be selected from those listed in FIG. 49J, by way of non-limiting examples. An opsin may be selected from the group consisting of Opto-β2AR or Opto-α1AR, by way of non-limiting examples. Alternately, an inhibitory channel may also be chosen, and either a single blue light source used for activation, or a combination of blue and red light sources to provide for channel activation and deactivation, as has been described elsewhere herein, such as with regard to FIG. 14.

Alternately, a system may be configured to utilize one or more wireless power transfer inductors/receivers that are implanted within the body of a patient that are configured to supply power to the implantable power supply.

There are a variety of different modalities of inductive coupling and wireless power transfer. For example, there is non-radiative resonant coupling, such as is available from Witricity, or the more conventional inductive (near-field) coupling seen in many consumer devices. All are considered within the scope of the present invention. The proposed inductive receiver may be implanted into a patient for a long period of time. Thus, the mechanical flexibility of the inductors may need to be similar to that of human skin or tissue. Polyimide that is known to be biocompatible was used for a flexible substrate.

By way of non-limiting example, a planar spiral inductor may be fabricated using flexible printed circuit board (FPCB) technologies into a flexible implantable device. There are many kinds of a planar inductor coils including, but not limited to; hoop, spiral, meander, and closed configurations. In order to concentrate a magnetic flux and field between two inductors, the permeability of the core material is the most important parameter. As permeability increases, more magnetic flux and field are concentrated between two inductors. Ferrite has high permeability, but is not compatible with microfabrication technologies, such as evaporation and electroplating. However, electrodeposition techniques may be employed for many alloys that have a high permeability. In particular, Ni (81%) and Fe (19%) composition films combine maximum permeability, minimum coercive force, minimum anisotropy field, and maximum mechanical hardness. An exemplary inductor fabricated using such NiFe material may be configured to include 200 μm width trace line width, 100 μm width trace line space, and have 40 turns, for a resultant self-inductance of about 25 pH in a device comprising a flexible 24 mm square that may be implanted within the tissue of a patient. The power rate is directly proportional to the self-inductance.

The radio-frequency protection guidelines (RFPG) in many countries such as Japan and the USA recommend the limits of current for contact hazard due to an ungrounded metallic object under the electromagnetic field in the frequency range from 10 kHz to 15 MHz. Power transmission generally requires a carrier frequency no higher than tens of MHz for effective penetration into the subcutaneous tissue.

In certain embodiments of the present invention, an implanted power supply may take the form of, or otherwise incorporate, a rechargeable micro-battery, and/or capacitor, and/or super-capacitor to store sufficient electrical energy to operate the light source and/or other circuitry within or associated with the implant when used along with an external wireless power transfer device. Exemplary microbatteries, such as the Rechargeable NiMH button cells available from VARTA, are within the scope of the present invention. Supercapacitors are also known as electrochemical capacitors.

An inhibitory opsin protein may be selected from the group consisting of, by way of non-limiting examples: NpHR, eNpHR 1.0, eNpHR 2.0, eNpHR 3.0, Mac, Mac 3.0, Arch, Arch3.0, ArchT, Jaws, iC1C2, iChR, and SwiChR families. An inhibitory opsin may be selected from those listed in FIG. 54J, by way of non-limiting examples. A stimulatory opsin protein may be selected from the group consisting of, by way of non-limiting examples: ChR2, C1V1-E122T, C1V1-E162T, C1V1-E122T/E162T, CatCh, CheF, ChieF, Chrimson, VChR1-SFO, and ChR2-SFO. A stimulatory opsin may be selected from those listed in FIG. 49J, by way of non-limiting examples. An opsin may be selected from the group consisting of Opto-β2AR or Opto-α1AR, by way of non-limiting examples. The light source may be controlled to deliver a pulse duration between about 0.1 and about 20 milliseconds, a duty cycle between about 0.1 and 100 percent, and a surface irradiance of between about 50 milliwatts per square millimeter to about 2000 milliwatts per square millimeter at the output face of a 100-200 um core diameter optical fiber.

As described above, a light source, such as laser diode, LED or OLED, by way of nonlimiting examples, may be used as the light engine for powering the photo-sensitive ion channel reaction. When multiple wavelengths, each responsible for stimulating a subset of photo-sensitive ion channels, are required in one device, individual emitters with different wavelengths can be grouped together to achieve what we will refer to as “wavelength multiplexing”. As shown in the exemplary two color channel device shown schematically in FIG. 55, the short wavelength (e.g. blue, green) emitters and long wavelength (e.g. yellow, red) emitters are integrated into a single integrated illumination device IIS to form a multi-wavelength emitting device. The individual emitters, labeled as LS1-LS8. In this configuration of this exemplary embodiment, LS1, LS3, LS5, and LS7 are each one of a set of similar light sources that all utilize a nominal output spectrum, and the other light sources (LS2, LS4, LS6, and LS8) form another set of mutually similar light sources sharing an output spectrum distinct form that of the other set. As such, they may be activated as complete sets, or individually as desired.

Other wavelengths and output spectra are also possible and considered to be within the scope of the present invention. The choice of output color, or spectrum, is a function of the target opsin.

Of course, other more complicated patterns, wavelengths, and number of emitters is possible. FIG. 4A illustrates three (3) such examples of opsin absorption spectra that are relevant to the present invention. Other opsins such as, but not limited to, the excitatory opsins; SFOs, SSFOs, ChR1, and VChR1, and the inhibitory opsins; eARCH, eNpHR2.0, eNpHR3.0, Mac, Arch, and eBR may be also used in the biological target and are also within the scope of the present invention.

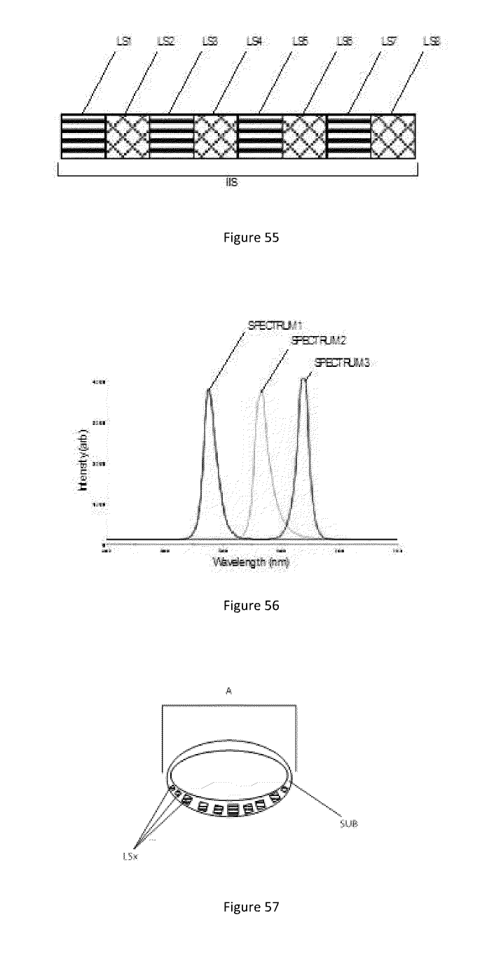

A light-emitting diode (LED, or alternately ILED to denote the distinction between this inorganic system and Organic LEDs, or OLEDs) is a semiconductor light source, and versions are available with emissions across the visible, ultraviolet, and infrared wavelengths, with very high brightness. When a light-emitting diode is forward-biased (switched on), electrons are able to recombine with electron holes within the device, releasing energy in the form of photons. This effect is called electroluminescence and the color of the light (corresponding to the energy of the photon) is determined by the energy gap of the semiconductor. An LED is often small in area (less than 1 mm2), and integrated optical components may be used to shape its radiation pattern, or the radiation pattern of an ensemble of light sources. An example of an LED useful for the present invention is manufactured by Cree Inc., it is a Silicon Carbide device and provides 24 mW of 450±30 nm (blue) light at 20 mA. A table of general LED characteristics is given for reference in FIG. 4c. LEDs such as these typically demonstrate a Lorentzian-like output spectral power distribution, such as those shown in FIG. 56.

Such as is shown in the embodiments of FIGS. 8-11 and 21-26, multiple emitters can be built into one device, either providing higher excitation energy per illumination volume than one individual emitter is capable of, or providing an illumination envelope that covers or conforms to specific neural tissue structure, hence the term “spatial multiplexing”. The following is an example of a 1D emitter array that can be formed into a cylindrical shape that surrounds the target (a.k.a. “cuff”) that illuminates from the circumference of the neural tissue structure that it encircles, as has been described elsewhere herein. Of course, as is common to all embodiments described herein, the applicator may also be configured to be nominally flat (or equivalently, planar, or slab-like applicators as described herein) for deployment upon a tissue surface. This also gives the device spatial control capability by individual control of the emitters. Such an configuration is illustrated in FIG. 57, where light sources LSx are incorporated into Substrate SUB to form Applicator A, the functions and details of which have each been described elsewhere herein.

Alternately, the above two embodiments may be combined to form a system employing both wavelength and spatial multiplexing. As such, each light source may be independently addressable, or made to be addressable in groups that correspond to their output wavelength (i.e. color) and/or position relative to the target tissue. We refer to this configuration as “hybrid multiplexing.”

Optical elements may also be added to the device to deliver light onto a target by means of beam shaping, guiding, concentration, and/or homogenization that shapes, and/or redistributes the optical power from the emitter/light source. The underlying mechanism of such optical elements consists of, but is not limited to, the following four major categories; Diffractive, Refractive, Reflective, and Diffusive.

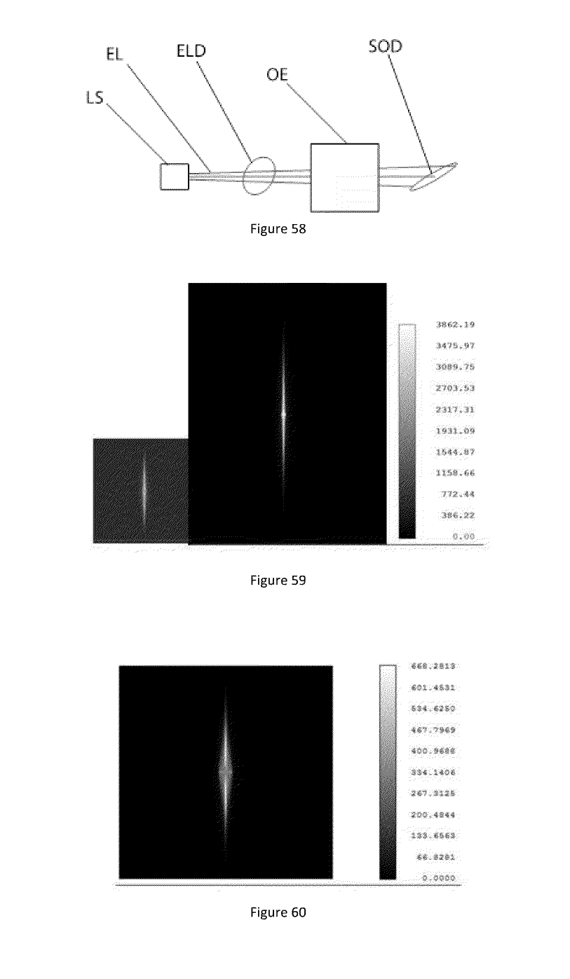

Various illumination profiles (i.e. irradiance distributions, or distributions) may be produced with added diffractive or refractive optical elements to optimize the illumination efficiency for the specific dimension and/or shape of the tissue target, such as, by way of non-limiting example, a nerve cell body or axon. For example, an ellipsoidal or line illumination is more desirable than a Gaussian spot when applying light stimulation on a length of neuron or nerve fiber as this shape is a better match to that of the target and provides for more efficient use of the illumination light than a round spot which “spills” light outside of the nominally linear target when attempting to illuminate along a length of the target. FIG. 58 shows a schematic representation of such a configuration, wherein Light Source LS outputs Emitted Light EL, which is characterized by an Emitted Light Distribution ELD. Optical Element OE intercepts emitted light EL and transforms it to produce Shaped Optical Distribution SOD. A variety of optical element types may be used in this embodiment. For example, a cylindrical lens or prism can convert a Gaussian beam into an elongated beam. A diffraction grating can also transform a single spot into a line by creating a plurality of spots. Alternately, a combination of any or all of these embodiments may be configured and is within the scope of the present invention. Such optics may be made of a size that is on the order of that of the light source itself, and are herein referred to as “micro-optics”.

By way of non-limiting example, prisms can be used to redirect the beam propagation and therefore shaping the output beam profile. The term “prism” here refers broadly to optics and micro-optics that have flat or curved facets that interact an incoming beam and change the beam profile (i.e. the power distribution). For example, a biconic lens that has four curved facets (with radii of curvature of 1 mm) on each side may be made to produce a linear illumination profile SOD when placed at a distance of 2 mm from a 0.2 NA Ø200 μm step-index optical fiber that is transmitting light captured from the Cree LED mentioned earlier. The distribution is shown in FIG. 59.

By way of another non-limiting example, a Ø2 mm cylindrical lens can be used to convert a Gaussian beam into an elongated beam. A cylindrical lens has flat profile in one axis and curved surface in the orthogonal axis, thus comprising optical refractive power only in one direction. The irradiance profile achieved is shown in FIG. 60.

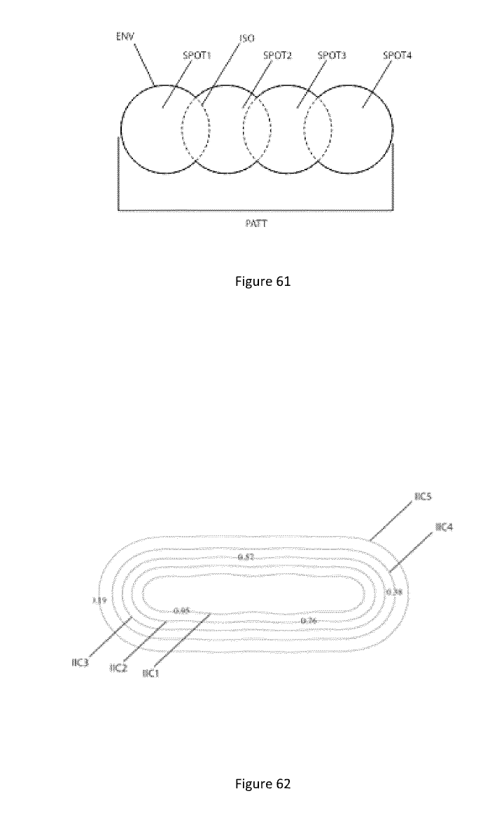

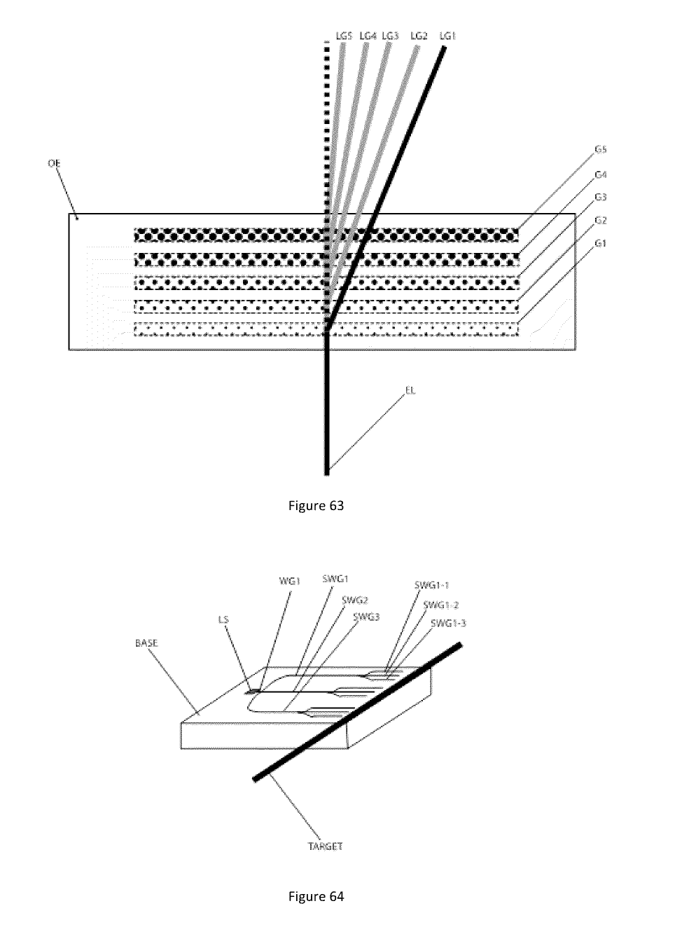

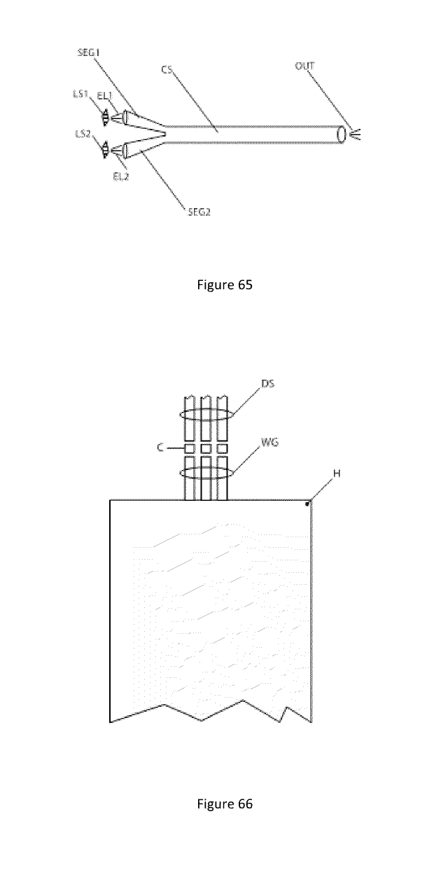

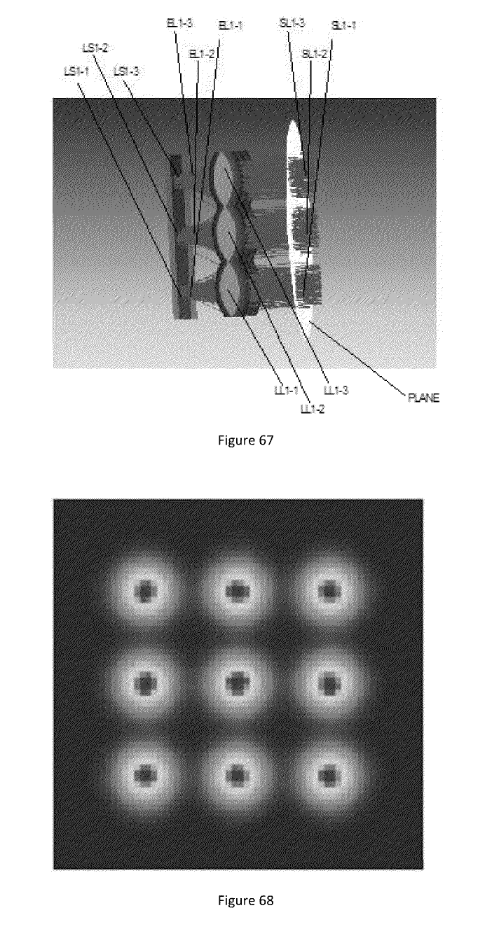

In another non-limiting example, a diffractive optical element, such as a micrograting, may be configured as OE in FIG. 58, such that the separation of the diffraction orders produces an overall irradiation pattern as that shown in FIG. 61, where the dashed lines indicate the outlines ISO of individual spots SPOT1-SPOT4 resulting from diffraction over the first four diffractive orders, and the solid line indicates the envelope ENV of the ensemble. In this example, a distance of ˜1.5R separates the center-points of these spots, where R is the 1/e² Gaussian beam radius. This exemplary 1×4 array configuration produces the cumulative irradiation distribution (profile) shown in FIG. 62, where the numeric labels represent normalized isoirradiance contours (denoted IICx in the figure) for the superposition of four beamlets and IIC5=ENV as represented in FIG. 61.

The diffraction order efficiencies and energy must be balanced to achieve a reasonable overall irradiance profile, as must the anomorphic magnification inherent in grating systems. This dependence is relatively small at small angles and a reasonably uniform overall pattern may be generated as long as the only a few orders are used. For example, the first 3 orders maybe used with an “echellette” grating, or alternately a few of the higher orders may be used with an “echelle” grating. These are diffraction gratings that have been optimized to work at low and high orders, respectively, by “blazing” the periodic corrugations that form the grating, as is well known in the art.

Alternately, a balance between relative diffraction order intensity and spectral bandwidth may be achieved by utilizing a Volume Holographic Grating (VHG) wherein diffraction occurs via phase interaction within a small range of wavelengths and angles around the Bragg-matching condition. This sensitivity may be beneficially exploited by including a plurality of VHGs in a single element such that the angular separation of the different VHGs is nominally balanced across the light source spectrum to provide a nominally uniform cumulative irradiance distribution at the target site, or at an intermediate location in optical communication with the target. The output spectra of light sources such as LEDs, or OLEDs range between 10-100 nm, unlike lasers that have a much narrower output spectrum.

The diffraction efficiency, η, for a VHG is defined as the ratio between the diffracted intensity and the incident intensity. Without considering absorption and Fresnel reflections at the interfaces when using a non-slanted transmission grating with index modulation n₁ and thickness D, and when the Bragg condition is satisfied for wavelength λ_{B}, the diffraction efficiency η_{B} is given as:

$${η}_{B} = {sin}^{2} ( \frac{π {n}_{1} D}{{λ}_{B} cos {θ}_{n}} )$$

where θₙ is the incident angle inside the medium of index n. Furthermore, the spectral and angular efficiencies, η_{λ} and η_{θ}, are further modulated with a sin c function dependence on the spectral and angular bandwidths.

$${η}_{λ} = {η}_{B} sin {c}^{2} ( \frac{{λ}_{B} - λ}{Δ λ} )$$

$${η}_{θ} = {η}_{B} sin {c}^{2} ( \frac{θ - {θ}_{B}}{Δ θ} )$$

where Δλ and Δθ are the deviations at the first spectral and angular nulls respectively.

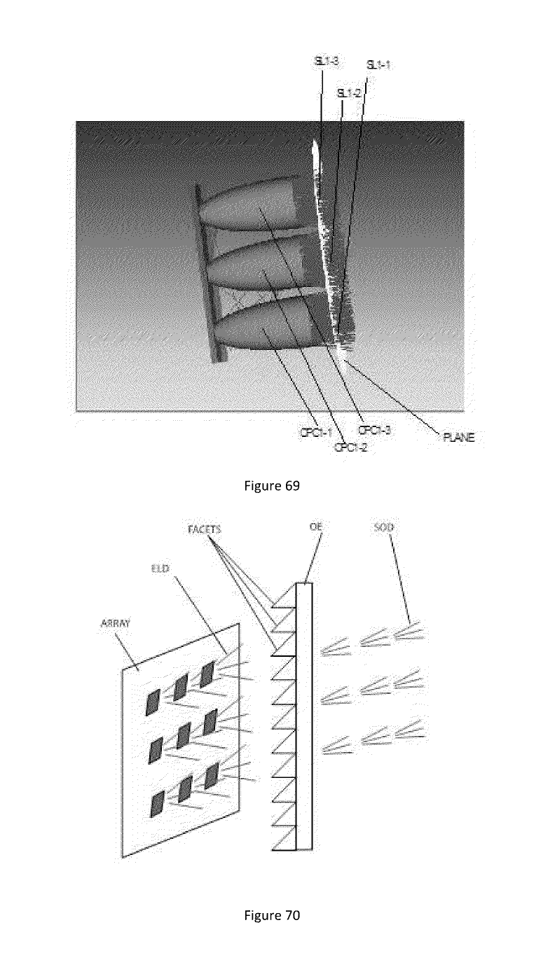

For example, a VHG may be designed such that Δλ, with the additional condition that the λ_{B} be made 5 nm apart for each successive VHG in order to drive different portions of the LED output spectrum into a different location that ultimately spatially overlaps with that of another portion of the LED output spectrum. Because a VHG only functions strongly across a narrow spectral range (Δλ), this approach of spectrally shifting VHGs may be iterated across the LED output spectrum to nominally redistribute all of the LED optical output. Furthermore, the relative intensities of the predominant diffraction orders of different spectral bands that may be produced successive VHGs may be also made to provide a nominally more uniform irradiance distribution by spatially redistributing the diffracted light such that the superposition of all the orders of all the successive VHGs is power balanced across a spatial region. This must also be tailored to the spectral output power distribution to optimize the ultimate uniformity of the resultant irradiation distribution. A schematic representation of successive VHGs for a source with a spectral bandwidth of 25 nm, using the spectral null deviation mentioned above is shown in FIG. 63. In this exemplary embodiment, emitted light EL from a light source (not shown) encounters VHG OE and is subsequently divided by the succession of individual VHGs G1, G2, G3, G4, and G5 such that beamlets LG1-LG5 are produced from interaction with the predominant order of the individual Gratings G1-G5 of the VHG. Furthermore, energy from the higher orders of the individual gratings may be made to overlap with the orders of the other individual gratings. According to the relations given above, the spatial superposition of these beamlets may be made to produce the desired irradiance distribution. This is similar to the underlying scheme in wavelength division multiplexing, but with the added requirement of power balancing across the spectrum to uniformly illuminate a target instead of finely dividing the output spectrum for maximum throughput per channel in order to transmit dense information. Thus, the spatial extent of such an array of “spots” to form an extended irradiation profile (or equivalently, an irradiance distribution) is constrained by the interplay between the light source power and spectrum, the target geometry, and the physical space confining the optical delivery device. Such approaches may be made in 2D, and even 3D by the means described herein.

Thus, it should be understood that the light source emission pattern may be converted into a more desirable pattern for a given target by applying beam shaping as taught herein, and not necessarily for creating a nominally uniform distribution.

When the emitter (light source) is located away from the target tissue, light waveguiding elements can be incorporated into the device to bring light to the proximity of the target. Furthermore, such waveguides may also be built into a monolithic structure to provide for optical power distribution within a single integrated device. An exemplary embodiment of this configuration is shown in FIG. 64. In this example, Light Source LS is a diode grown on a wafer substrate BASE. The emitted light is guided out by slab or channel waveguides WG that are also integrated into the wafer. The light may thus split into multiple channels (9 in this exemplary configuration) by dividing the light conducted within waveguide WG into splitting waveguides SWG1-SWG3. Subsequent to that, the light within splitting waveguides SWG1-SWG3 may be further divided into more waveguide bifurcations, such as SWG1-1-SWG1-3, etc. This configuration allows the light to be distributed along 9 locations on the target tissue TARGET when it is exposed to the output of said waveguides. This output may alternately be used as input for a plurality of delivery segments, as have been described elsewhere herein. The number of channels and their spatial distribution are design parameters that are selected to fit for specific light delivering need. Alternately, the reverse configuration is also within the scope of the present invention. That is, instead of a distribution system, a combining system may be employed. For example, a bifurcated waveguide may be coupled to allow distinct light sources to combine into a common path when more optical power or differing optical spectra are required.

Alternately, along these lines of combining power and or spectra, a light pipe may be used to combine and/or deliver light to the target tissue. A light pipe is a subset of waveguides in that it is a relatively large device—being defined herein as ≧ about 0.5=² in cross sectional area. In the exemplary configuration illustrated in FIG. 65, a bifurcated light pipe, comprised of segments SEG1 & SEG2 that combine into Common Segment CS is used to deliver emitted light EL1 & EL2 from two light sources LS1 & LS2, culminating in output light OUT. The number of emitters to be combined and the light pipe configuration are closely coupled in the design process to aim for optimal efficiency. LS1 & LS2 may share the same nominal output spectrum, or different output spectra. In the former case, LS1 & LS2 may be combined to provide higher irradiance and or irradiation area than achievable with a single light source. In the former configuration, LS1 & LS2 may be used individually to activate a particular set of opsins while leaving another set inactivated. This is a schematic illustration and it should be understood that other approaches, such as 4:1 combiners, etc., are considered as derivative of this basic scheme and understood to be part of the present invention.

The concept of light guiding (or equivalently, waveguiding when used with smaller structures and/or devices) as it has been described herein is applicable both proximal and distal to the biological target. That is, such approaches may be utilized within the optical delivery device, or within the device's power supply and control housing (H), or in between that and the applicator(s), these system components have been described elsewhere herein. In the latter case, the waveguides (WG) may be made to provide for detaching the optical delivery segments (DS). FIG. 66 schematically depicts such a configuration, wherein the light source(s) is(are) contained within the housing H. Their optical output is channeled through waveguides WG, and connected using Connector C to delivery segments DS that supply the applicator A (not shown). This configuration allows for independent replacement of the housing or the delivery segments/optical delivery device. Connector C may be a polymeric sleeve that serves to butt-couple waveguides WG to delivery segments DS, or alternately, it may be an optical element that serves to transmit light between WG and DS.

Optical elements may also be added to change beam width and divergence to help improve the irradiance of the light reaching the target plane. Micro lenses and micro reflectors (micro mirrors) are examples of optical elements that may be used to concentrate light.

One such embodiment utilizes a 3×3 microlens array that is matched to a 3×3 array of LED emitters. This is shown in FIG. 67, where individual light sources LS1-1, LS1-2 and LS1-3 represent the first column of LEDs in the 3×3 array. These sources emit light EL1-1, EL1-2 and EL1-3, respectively. This emitted light reaches the lenslet array comprised of lenses LL1-1, LL1-2, LL1-3, etc. Each lenslet serves to condition the light from a single LED, and create shaped light SL1-1, SL1-2, SL1-3, etc. The irradiance distribution at PLANE is given in FIG. 68. Each LED has an output facet size of 100 um and emission divergence angle of 20°. In the embodiment of this exemplary configuration, each lenslet in the microlens array has front and back radii of curvature of 500 um, and thickness of 300 um. It is placed at a distance of 350 um between the lens apex and the emitter output facet. The material for the microlens array may be BK7, for example, which has a nominal refractive index of n=1.5 throughout the visible portion of the optical spectrum. As shown in Figure K, the micro lens array focuses the divergent diode emission therefore quasi-collimated emission is obtained at the target plane. Although the target tissue is a turbid (i.e. scattering) medium, this biasing of the initial trajectory increases the overall depth of penetration. Using the micro lens array as described, the irradiance at a central axon within a Ø1 mm nerve bundle is ˜2.5× that without, providing a significant overall efficiency improvement.

A similar concentrating effect can be obtained by utilizing a micro-reflector array. FIG. 69 shows such an alternative configuration. This embodiment uses same LED chip array as that of FIG. 67, with the substitution of a 3×3 micro-reflector array in lieu of the microlens array. Each micro-reflector in this embodiment is a compound parabolic concentrator (CPC1-1, CPC1-2, etc.). Each CPC has a top opening aperture of 150 um, slightly larger than the individual LED chip size, over which it fits. The CPC is designed for 20° maximum acceptance angle, which matches with the LED chip divergence angle. Similar to the micro-lens array design, beamlet coming out of the micro-reflector is quasi-collimated with higher irradiance than the unperturbed diode emission and provides an overall irradiance profile at PLANE that is similar to that shown in FIG. 68.

In an alternate embodiment, a beam homogenization element can be added to improve illumination uniformity if the device generates a non-uniform illumination pattern due to factors such as a non-uniform emission profile from individual emitter, or low fill factors from the emitter array. A microlens array, and/or a microreflector array, and/or a diffractive element or array of elements, and/or a diffusive element or array of elements may be used as beam homogenizers. For example, a diffuser with a bulk scattering length of 40 um and scattering angle of 180° and thickness of 100 um may be placed above the emitter array to distribute the light from the individual emitters in order to create a more uniform illumination at the target surface.

Common path optics may also be utilized with an array of light sources to help improve light-tissue interaction rather than utilization of an array of individual optical element that each interacts with an individual emitter. For example, a lens, or Fresnel lens, as shown in FIG. 70, may be used to reduce the divergence of the emission pattern.

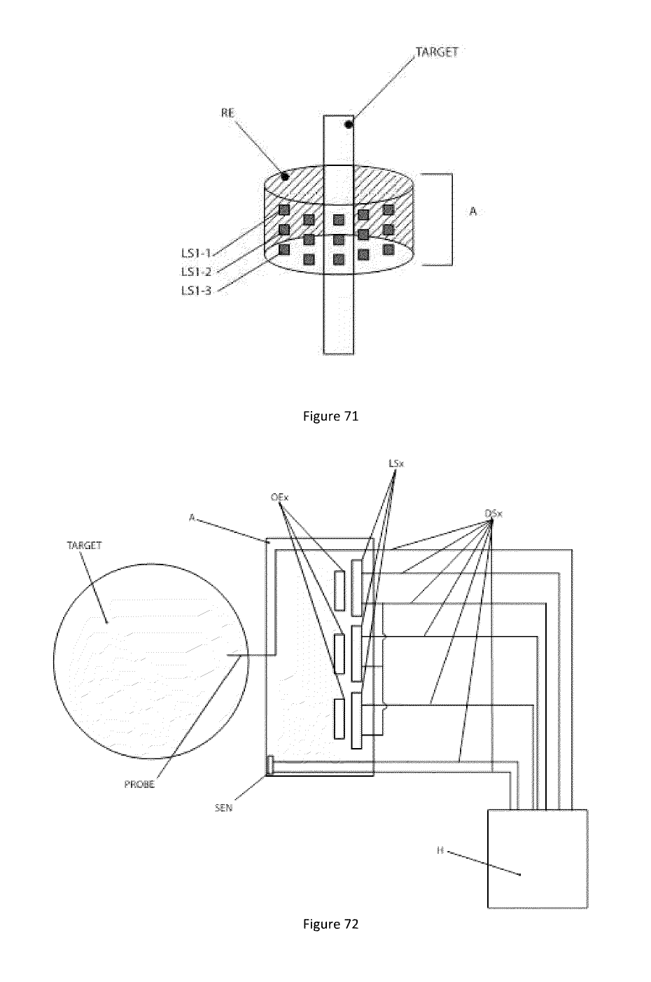

Further improvements maybe made by utilizing a reflective cover over the applicator, as has been described elsewhere herein and also illustrated schematically in FIG. 71 for the configurations described above (which are themselves similar to that of FIGS. 26-27). In this embodiment, Target TARGET is surrounded by Applicator A, which is in turn comprised of an array of light sources (including LS1-1, LS1-2, and LS1-3, etc.) The substrate S (not shown) upon or within which Light Sources LSX-Y are integrated may further comprise Reflective Element RE that serves to redirect light back towards TARGET which would otherwise be lost (as was described especially with respect to Sleeve S of FIG. 10B, Mirror(s) M of FIG. 19, and Reflective Element of FIGS. 21A-21C).

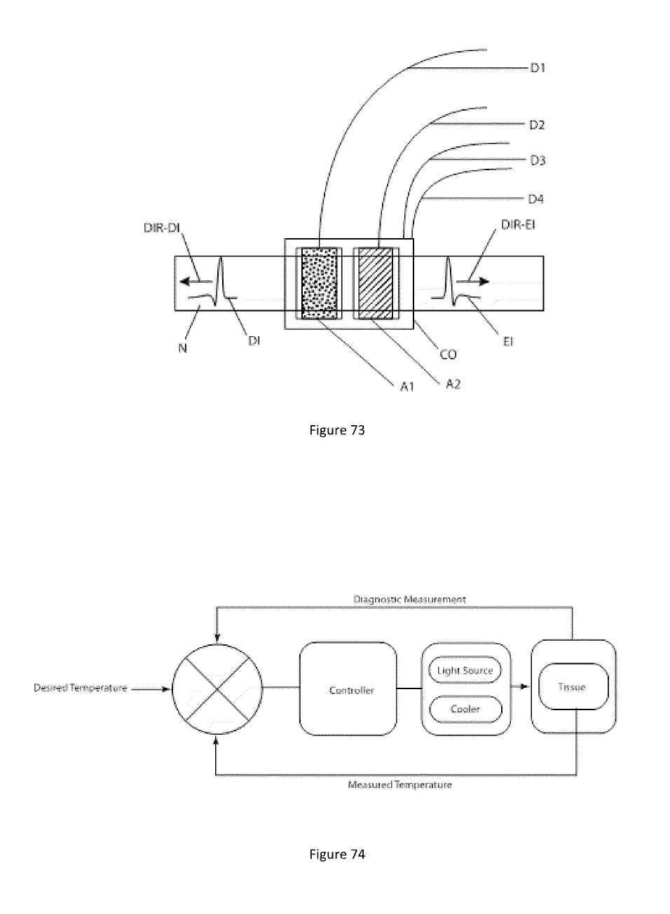

Any or all of the optical applicator and device embodiments described herein may be combined with adjunct technologies to form hybrid systems with enhanced functionality. FIG. 72 is a schematic representation of the generic configuration of such an integrated system where the light sources LSx are located in the optical delivery device portion of an Applicator A along with the (optional) Optical Elements OEx and are electrically connected to the control system and power supply located in Housing H via Delivery Segments DSx. These system components have been described elsewhere herein. However, the embodiment of FIG. 72 includes the additions of Sensor SEN and Probe PROBE which are also connected to Housing H via Delivery Segments DSx. Sensor SEN and Probe PROBE may include measurement and control technology.

The sensor SEN or probe PROBE may be a temperature sensor. Passive devices such as thermistors and thermocouples may be used. Alternately, active digital or analog temperature sensors, such as the ultralow power STLM20 from STMicroelectronics may also be used. The sensor should be placed as close to the target tissue as possible to avoid thermal conduction delays that would occur should it placed well within a insulating polymeric encapsulation. Alternately, the temperature sensor as shown could be a switch that activates an interlock circuit to deactivate the light output once a maximum temperature is reached, and likewise reactivate it once a safe baseline temperature has been established.

Alternately, sensor SEN or probe PROBE may be a electrophysiological probe within or adjacent to the target tissue. Examples of such probes may be a single wire electrode (as shown), a coil located within the optical delivery device, or an array of electrodes to enable recording from multiple locations. These probe configurations are intended for electrophysiological monitoring of the target tissue. Alternately, such probes may be deployed to the ultimate biological target, if not the target tissue for irradiation. Examples of such configurations for measurement of the ultimate desired function rather than the optogenetic target include electromyography (EMG) probes placed in muscles that are innervated by a target motor nerve, or electroneurographic monitoring of a neuron or nerve, or groups/bundles of nerves. Rather than direct implantation of electrodes into the diagnostic target tissues, coils, or antennae may be placed in proximity to the diagnostic target tissues such that they are inductively coupled to them electrically or magnetically and thus able to sense activity.

Alternately, sensor SEN or probe PROBE may be an optical detector that captures remitted light from the target tissue, or its surroundings, including from the light sources LSx themselves. Such detection allows for at least relative or ratiometric measurements that provide information over time about the optical condition of the target and/or illumination device. Such information may be used to adjust the illumination level (light output power) to compensate for degradation of the light source, optical properties of the target and environment, etc.

Alternately, sensor SEN or probe PROBE may be an optical detector that detects fluorescence from the target tissue, and/or its environment. Such a signal may serve to provide information regarding the illumination efficacy, or target tissue condition. An example is background autofluorescence of the target tissue and/or its environment as a means to determine the health of the tissue, or the level of protein expression when a fluorescent probe is co-labeled along with the protein. Such spectrally sensitive detection would further require the use of optical filters to prevent background noise from the illumination light itself.

Alternately, probe PROBE may be an electrical stimulator that is packaged into the applicator, or adjacent to it. In some instances it is valuable to combine electrical stimulation with optical control. Electrical stimulation of a peripheral nerve results in propagation of action potentials in both directions along the nerve. In many cases, propagation of action potentials in only one direction is desired, and propagation in the other direction may produce undesired side effects. To avoid this problem with electrical stimulation, the electrical stimulation may be combined with illumination of an inhibitory opsin (such as NpHR or eARCH by way of non-limiting examples) such that the action potential propagates only in the desired direction along the nerve and is inhibited from propagating in the undesired direction. In other cases, optical stimulation of selective neurons within a neural network may be achieved with an excitatory opsin (such as ChR2 or C1V1 by way of non-limiting examples) and inhibition of this excitatory signal may be achieved with high frequency alternating current electrical stimulation. Other combinations are also possible.

It is often useful to control the temperature of neural tissue to protect the tissue or modulate its properties. Illumination of tissue may raise its temperature due to intrinsic heating and/or from heating of collateral chromophores such as blood and pigment. When the temperature rises it may damage the tissue; thus, it is desirable to control this rise in temperature using a closed loop control circuit in which the temperature of the tissue is measured and used to activate a nerve cooling device that keeps the temperature of the tissue within a specified range, such as the regulatory limit applied to the temperature rise due to electrical stimulation devices, defined as ΔT≦2.0° C. with respect to euthermia. Altering the temperature of the tissue may also change its properties to achieve a desired effect. For example, cooling of nerve tissue changes its conductive properties and can alter the effect of optical stimulation of nerve tissue. For example, at body temperature illumination of a peripheral nerve including ChR2 at 60 Hz causes stimulation of nerve impulses, whereas lowering the temperature of the nerve may cause inhibition of the nerve impulses. Thus, one may achieve activation and inhibition with the same opsin simply by controlling temperature. Rather than using more than one opsin and the requisite spectrally and/or spatially distinct illumination configurations, this allows stimulation and inhibition with a single excitatory opsin using a single illumination applicator by controlling the temperature of the target tissue in which it resides. For example, when ChR2 is expressed in motor neurons the inhibition effects are evident at lower temperatures with a high, whereas excitation would be achieved at physiological temperatures and lower illuminations rates. Temperature and illumination rate can also be manipulated independently to achieve this effect.

As described in the description of FIGS. 50A-50C, nerve stimulation, such as electrical stimulation, causes bidirectional impulses in a neuron. That is, an action potential triggers pulses that propagate in both directions along a neuron. However, the coordinated use of optogenetic inhibition in combination with stimulation to allow only the intended signal to propagate beyond the target location by suppression or cancellation of the unwanted or errant signal using optogenetic inhibition. This may be achieved in multiple ways using what we will term “multi-applicator devices” or “multi-zone devices”. The function and characteristics of the individual elements utilized in such devices are defined elsewhere.

Such a multi-zone device is illustrated in FIG. 73. It is similar to that of FIG. 50B, with the additions of a cooling system comprised of Cooling Object CO that is supplied either electrical power or fluid via Delivery Segments D3&D4 from Housing H (not shown) when either a thermoelectric device or coolant is used, respectively.

In an exemplary embodiment, the system of FIG. 73 is configured to use an optogenetic applicator A2 and an electrical stimulation device A1. This example was chosen to represent a generic situation wherein the desired signal direction may be on either side of the excitatory electrode. The allowed signal direction is defined by the selective application of optogenetic inhibition from the applicator on the opposite side of the central Applicator A2. In this non-limiting example, the Errant Impulse EI is on the RHS of the stimulation cuff A2, traveling to the right, as indicated by arrow DIR-EI, and passing through the portion f the target covered by A3 and the Desired Impulse DI is on the LHS of A2, travelling to the left, as indicated by arrow DIR-DI, and, passing through the portion f the target covered by A1. Activation of A3 may serve to disallow transmission of EI via optogenetic inhibition of the signal, suppressing it. Similarly, activation of A1 instead of A3 would serve to suppress the transmission of the Desired Impulse DI and allow the Errant Impulse EI to propagate. Therefore, bi-directionality is maintained in this triple applicator configuration, making it a flexible configuration for Impulse direction control. Such flexibility may not always be clinically required, and simpler designs may be used, as is explained in subsequent paragraphs. This inhibition/suppression signal may accompany or precede the electrical stimulation, as dictated by the specific kinetics of the therapeutic target. Each optical applicator may also be made such that it capable of providing both optogenetic excitation and inhibition by utilizing two spectrally distinct light sources to activate their respective opsins in the target. In this embodiment, each applicator, Ax, is served by its own Delivery Segment, DSx. These Delivery Segments, DS1, DS2, and DS3, serve as conduits for light and/or electricity, as dictated by the type of applicator present. As previously described, the Delivery Segment(s) connect(s) to a Housing containing the electrical and/or electro-optical components required to provide for power supply, processing, feedback, telemetry, etc. They may also provide coolant flow to Cooling Object CO via a pump. The coolant may be Water, Saline, or other such thermally conductive low viscosity fluid that is bioinert.

The control of target tissue temperature may be accomplished by utilizing thermometers such as thermocouples, RTDs, etc. in conjunction with a feedback loop and a controller, as shown in FIG. 74, wherein the measured temperature may be compared to the desired (setpoint) temperature as input for the controller. The controller may employ a variety of control schemes, such as PID, pseudoderivative, feed-forward, etc. The controller may modulate (either partially or completely) the cooler and/or the light source to maintain the required clinical effect. It may further control the coolant flow and/or temperature. A diagnostic measurement may be obtained via a sensor, such as those described herein, which monitor(s) the function and/or activity of the target tissue, and/or effector tissue, and/or clinical effect. As mentioned earlier, the diagnostic measurement(s) may include, but not be limited to, electromyography (EMG) probes placed in muscles that are innervated by a target motor nerve, or a electroneurographic (ENG) monitoring of a peripheral or central nerve, or groups/bundles of nerves.

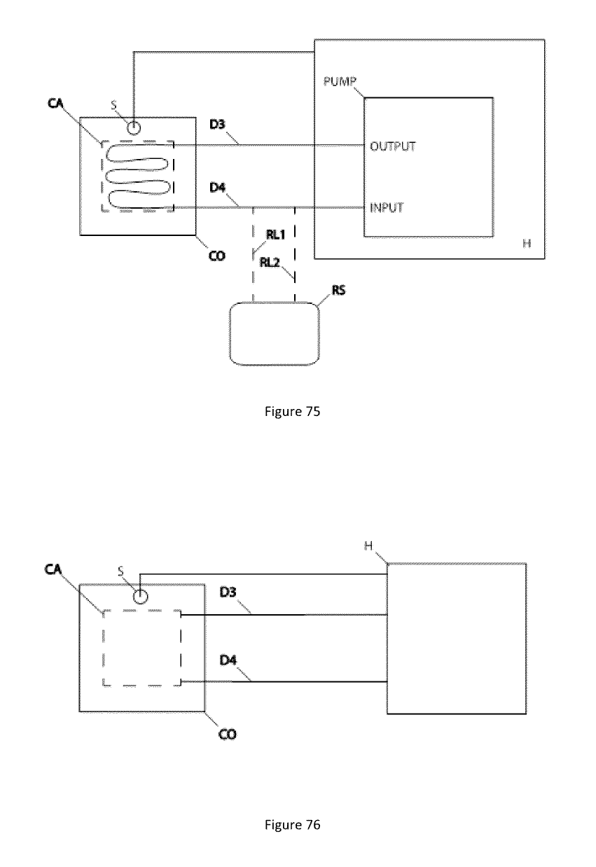

In another alternate exemplary embodiment, Cooling Object CO may be contained within the Applicator A (not shown), as represented in FIG. 75. It contains Cooling Area CA where thermal contact is made with the target tissue, or at a location adjacent to the target tissue that is sufficiently close to the target tissue to provide for good thermal communication (or alternately, low thermal inertia) between it and the target tissue. A PUMP is configured to provide coolant flow to the Cooling Object CO via input line D4 and output line D3. Furthermore, Cooling Object CO may contain a temperature sensor S (or SEN) to sense the measured temperature discussed above that is connected to Housing H and used as input in a sensing circuit (to be described in a subsequent section). The system may alternately be configured to employ a fluid reservoir RS configured to intercept the input (supply) line D4 of the PUMP via reservoir lines RL1&RL2. In this configuration the fluid may be stored at body temperature, rather than at the internal temperature of the housing. Resident within the housing (or elsewhere) may also be a heat exchanger, such as a thermoelectric device (not shown) to cool the coolant.

Alternately, a thermoelectric device may be used to provide the cooling directly to the tissue with out the use of coolant fluid, as is shown in FIG. 76, where D3&D4 are now electrical connections, not fluid connections as before and Cooling Object CO may be imbedded within the Applicator A (not shown). It may also be a plurality of small devices distributed throughout Cooling Area CA as a way to maintain flexibility and size, as would be required for use with small tissue targets, and/or in areas where the applicator will need to flex in-situ to accommodate patient movement.

The system may be tested for utility at the time of implantation, or subsequent to it. The tests may provide for system configurations, such as which areas of the applicator are most effective, or efficacious, by triggering different light sources alone, or in combination, to ascertain their effect on the patient. Furthermore, the effect(s) of cooling may also be queried to discern efficacy via functional or other such testing. Such optical and thermal tests may also be done simultaneously, or in coordination, to determine efficacy and/or overall system efficiency. Such configurations may also utilized a multi-element system, such as an array of LEDs, for example, or a multiple output coupling method is used, as has been described herein by way of non-limiting example. Such diagnostic measurements may be achieved by using an implanted electrode that resides on, in or near the applicator, or one that was implanted elsewhere. Alternately, such measurements maybe made at the time of implantation using a local nerve electrode for induced stimulation, and/or an electrical probe to query the nerve impulses intraoperatively using a device such as the CHECKPOINT Stimulator from NDI and Checkpoint Surgical to provide electrical stimulation of exposed motor nerves or muscle tissue and in turn locate and identify nerves as well to test their excitability. Once obtained, therapeutic configuration may be programmed into the system for optimal clinical outcome using an external Programmer/Controller P/C via a Telemetry Module TM into the Controller, or Processor, CPU of the system Housing H, as has been described above in reference to FIG. 3.

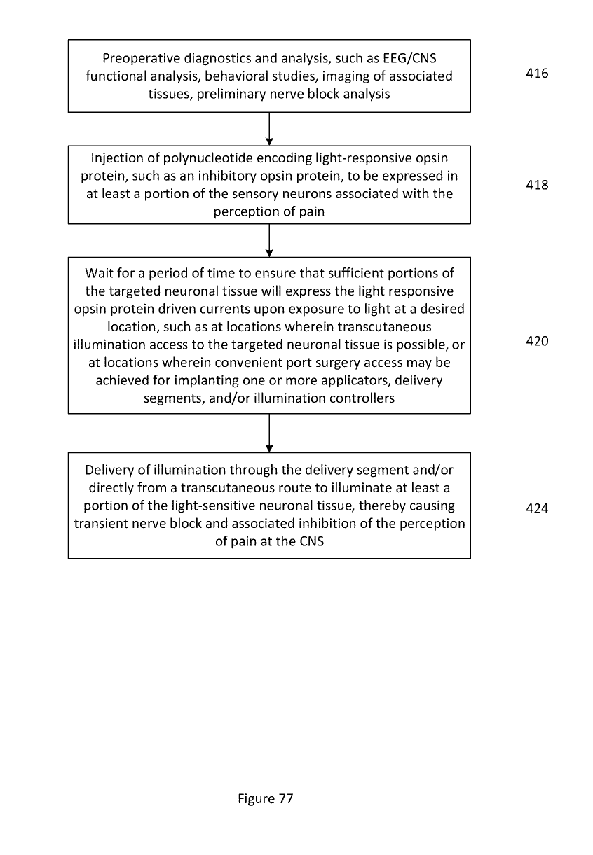

Referring to FIG. 77 and FIGS. 78-80, light-based neural inhibition may be utilized in pain management for neuropathic pain that evolves from peripheral nerves such as the unmyelinated C-fibers that innervate the skin and extremities.

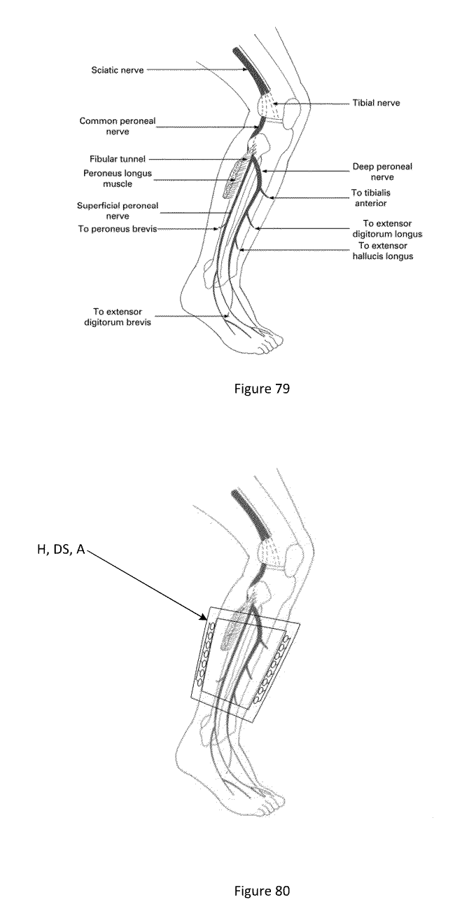

Referring to FIGS. 79 and 80, with successful transfection of targeted sensory neurons, such as the branches of the superficial peroneal nerve and the deep peroneal nerve, using an inhibitory opsin configuration such as NpHR or eARCH, a removable all-in-one external light emitting cuff (H, DS, A) may be applied to the leg to transcutaneously and transiently inhibit the pain sensory functionality of such nerves—thereby avoiding associated pain.

Referring to FIG. 77, after preoperative diagnostics and analysis (416), an inhibitory opsin configuration may be selected and delivered (418), and after expression (420), illumination may mitigate sensation of pain (424). In one embodiment, an inhibitory opsin configuration such as an NpHR, iC1C2, or eARCH is preferred for controllably inhibiting signal conduction along the targeted sensory nerves. SFO and SSFO versions of inhibitory opsins may provide advantageous longer trailing inhibitory effects after stimulation. As described above, the genetic material may be injected into muscles innervated by the targeted nerves for retrograde transport, or the genetic material may be injected directly into the nerves. In one embodiment, an AAV5-Hsyn-iC1C2 (high titer; from a provider such as UNC or Virovek, for example) may be injected intraneurally, intrathecally, or into the DRG related to the nerves of interest; within approximately 3-9 weeks, expression along the nerve, to nociceptors near the skin surface is successful, and robust pain mitigation is observed under transcutaneous illumination with light (e.g., 600 nm wavelength for NpHR, or 470 nm for iC1C2).

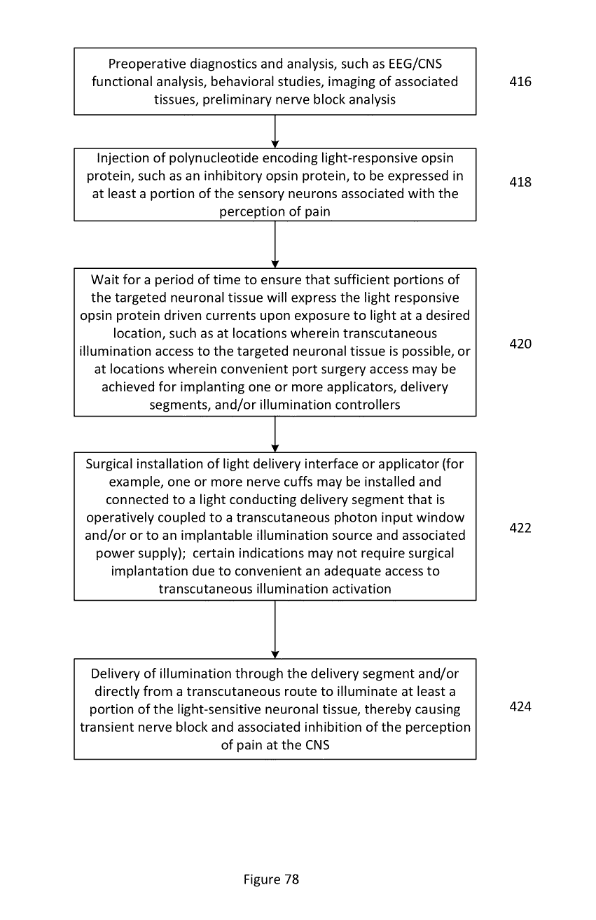

Referring to FIG. 78, after preoperative diagnostics and analysis (416), an inhibitory opsin configuration may be selected and delivered (418), and after expression (420) and hardware installation (422—depending upon the configuration; in transcutaneous illumination configurations implantation of hardware may not be necessary), illumination may mitigate sensation of pain (424). In one embodiment, an inhibitory opsin configuration such as an NpHR, iC1C2, or eARCH is preferred for controllably inhibiting signal conduction along the targeted sensory nerves. SFO and SSFO versions of inhibitory opsins may provide advantageous longer trailing inhibitory effects after stimulation. As described above, the genetic material may be injected into muscles innervated by the targeted nerves for retrograde transport, or the genetic material may be injected directly into the nerves, or intrathecally, or into the DRG related to those nerves. In one embodiment, an AAV5-Hsyn-iC1C2 (high titer; from a provider such as UNC or Virovek, for example) may be intraneurally injected; within approximately 3-9 weeks, expression along the nerve, to the dorsal root, and nociceptors near the skin surface is successful, and robust pain mitigation is observed under transcutaneous illumination with yellow light (e.g., 600 nm wavelength).

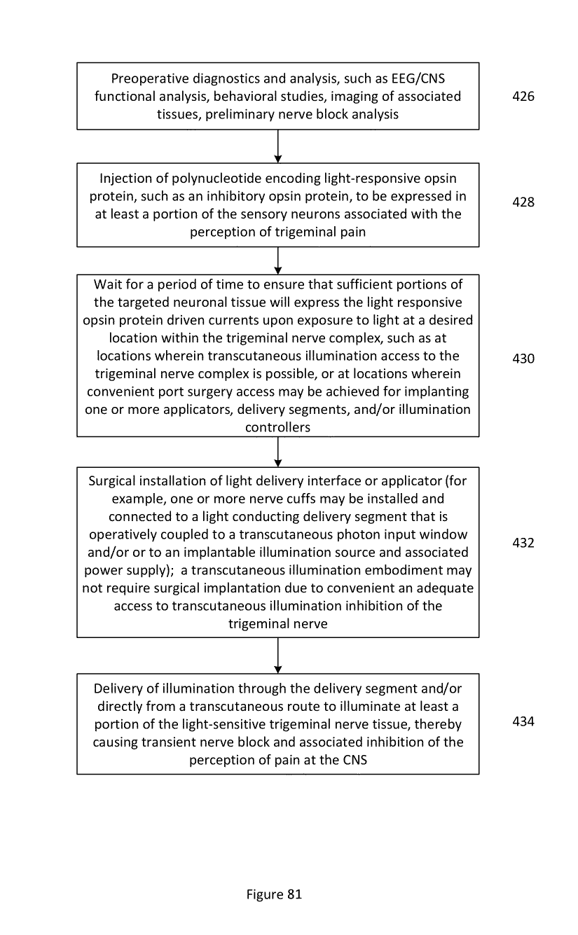

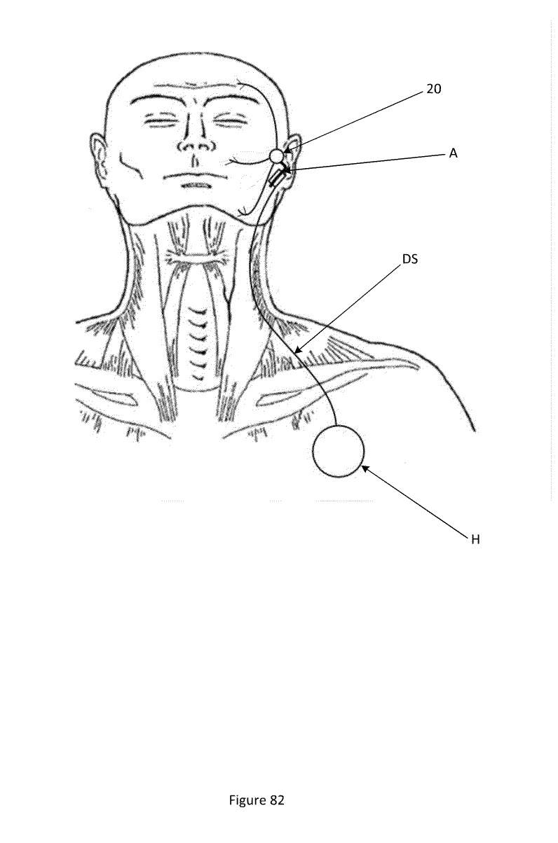

Referring to FIG. 81, a pain mitigation configuration somewhat analogous to that described in reference to FIG. 79 is illustrated, wherein trigeminal pain is mitigated by inhibitory opsin expressing nerve tissue under illumination. After preoperative diagnostics and analysis (426), an opsin configuration, such as the NpHR, iC1C2, or eARCH configurations described above for neuropathic pain, may be selected and delivered (428), such as by direct injection across the skin of the face and into the targeted trigeminal nerve tissue. After expression timing (430), in one embodiment (not shown), the light-sensitive trigeminal nerve tissue may be transcutaneously illuminated to mitigate pain without further implantation of hardware. In another embodiment, hardware such as that featured in FIG. 82 may be installed (432) to facilitate robust illumination of the targeted light-sensitive trigeminal nerve tissue and mitigation of associated pain perception (434). FIG. 82 features a housed illumination controller (H) operatively coupled to a light applicator (A) via a delivery segment (DS), with the applicator (A) positioned to provide robust illumination of the targeted nerve bundle (20) when the controller is commanded to provide illumination. In one embodiment, the controller may be configured to chronically pace the targeted nerve bundle (20) with light to prevent sensory function there. In another embodiment, the controller may be configured to be manually switched on by the patient (e.g., by a remote input device, such as a key fob style device wirelessly coupled to the controller, as described above in reference to fecal incontinence), such that upon sensation of pain, or before an activity known to bring about trigeminal pain, such as tooth brushing, the operator may command the controller to start illuminating. In one embodiment, the controller may be configured to deliver a given time period of illumination; in another embodiment it may be configured to stay on until affirmatively turned off; in another embodiment SFO or SSFO or inhibitory channel functionality may be utilized in the opsin selection process to prolong the effects of each illumination.

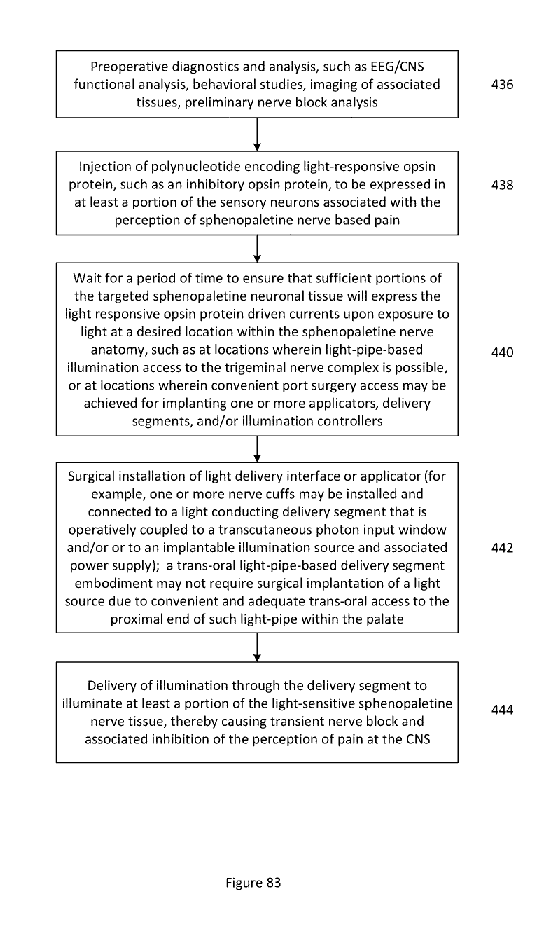

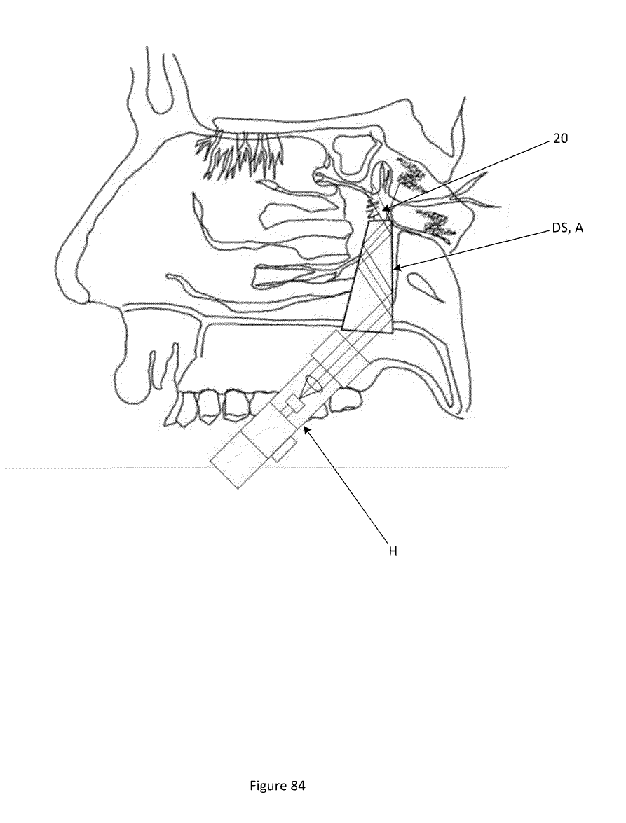

Referring to FIGS. 83 and 84, another pain management embodiment is illustrated wherein the sphenopalatine ganglion, believed to be directly associated with debilitating cluster headaches in some patients, may be inhibitorily light-stimulated to controllably prevent such cluster headaches. Referring to FIG. 84, an external/nonimplantable housed illumination controller (H) may be manually directed/interfaced to a light pipe or waveguide surgically installed across the hard palate of the human mouth, to provide illumination to the sphenopalatine ganglion, which preferably has been directly injected via a precision-guided needle with inhibitory opsin genetic material to make the nerve bundle (20) light sensitive, thus preferably mitigating the associated pain sensation. The optical configuration contained within Housing H may be made similar to that of FIGS. 100A through 100D, described elsewhere herein.

Referring to FIG. 83, after preoperative diagnostics and analysis, an opsin configuration, such as the aforementioned NpHR configuration, may be selected and injected (438). With time for expression (440) and surgical installation of the light delivery hardware (442), such as that shown in FIG. 84, the hardware (H) may be trans-orally illuminated to provide for pain sensation mitigation.

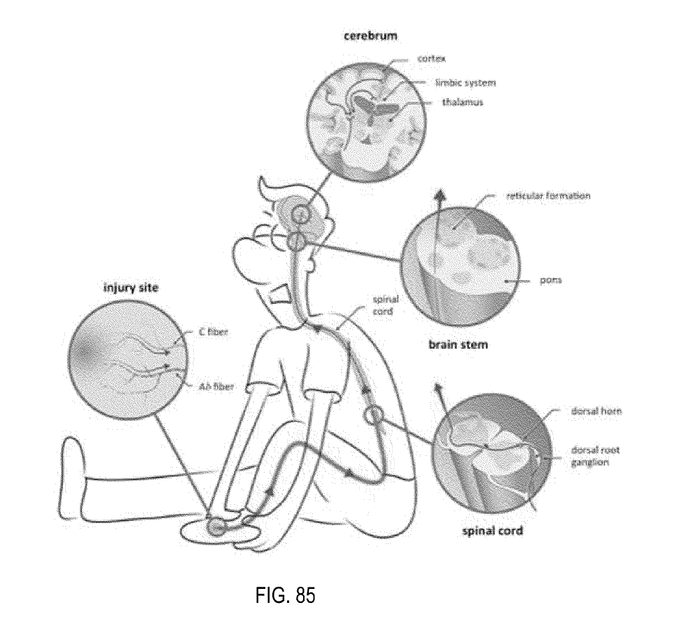

FIG. 85 schematically depicts nervous system involvement in the perception of pain. Receptors of afferent sensory nerves produce signals (action potentials) that travel to the spinal cord, then the brain stem and finally to the cerebrum, where they're processed and pain is perceived. Each of the abovementioned elements in this network may serve as a possible target tissue for optogenetic intervention, as it pertains to the present invention.

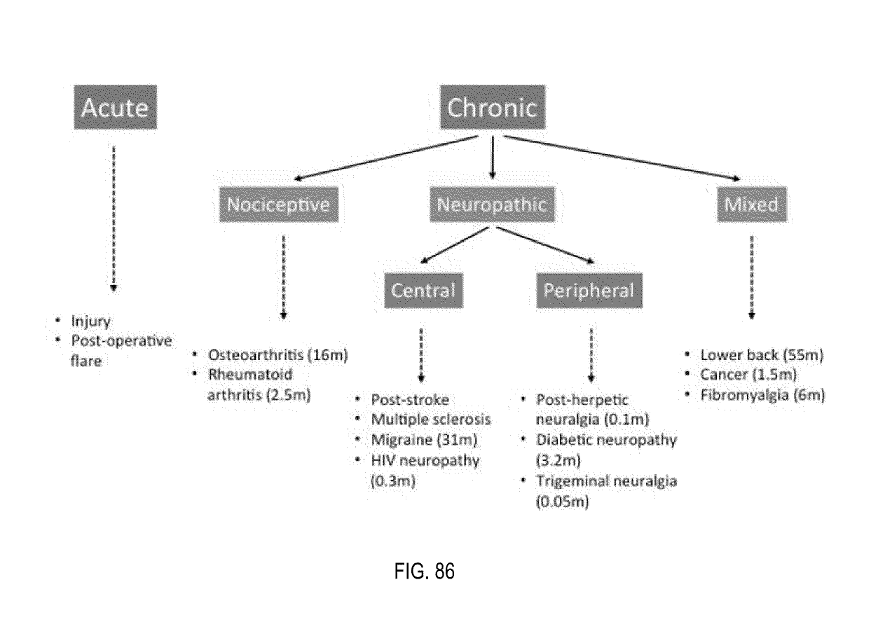

FIG. 86 depicts a listing of a variety of different forms of pain, including chronic and acute, with subdivisions for nociceptive, neuropathic and mixed pain. Each of the abovementioned elements may serve as a possible indication for optogenetic intervention, as it pertains to the present invention.

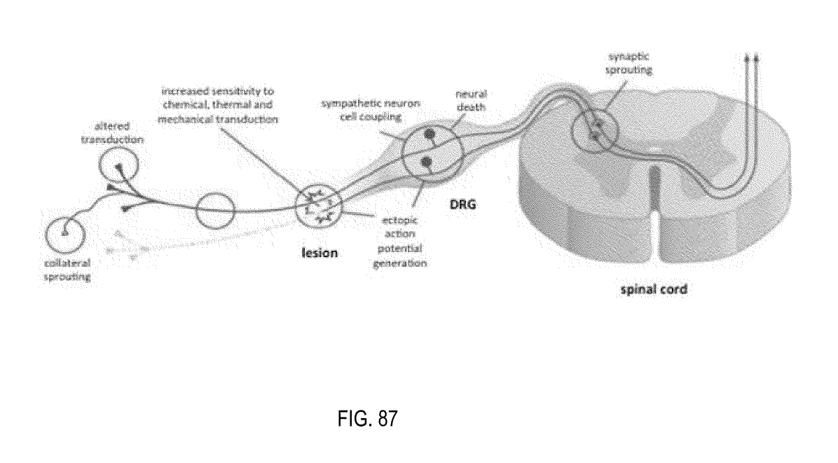

FIG. 87 depicts the involvement of the nervous system in the perception of pain in more detail than FIG. 85, with the addition of possible causes of pain listed at the corresponding anatomical feature or location. Each of the elements in this network may serve as a possible target tissue for optogenetic intervention, as it pertains to the present invention.

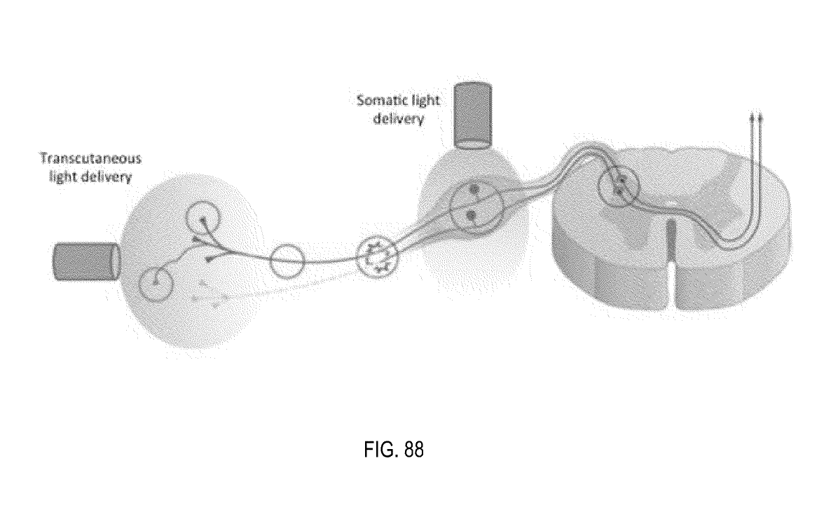

FIG. 88 depicts the same nervous system involvement as FIG. 87, with the addition of possible light delivery routes for treating a DRG (“somatic light delivery”) and nerve endings and/or receptors (“transcutaneous light delivery”), both of which are describe in more detail elsewhere herein.

There are two main approaches to light delivery to the target tissue. The first is Transcutaneous Light Delivery (TLD), in which the light source is extracorporeal and is delivered to the target tissue through the skin, or other epithelial tissue. The other is Somatic Light Delivery (SLD) in which the light source in implanted intracorporeally. A hybrid technique utilizing at least a single lightguide that is at least a partially implanted within the cutis that serves to carry the light it collects from an external light source towards the target tissue. We refer to this as “Percutaneous Light Delivery”, as it involves a configuration in which the otherwise intact skin is disrupted to accommodate the at least partially implanted lightguide(s).

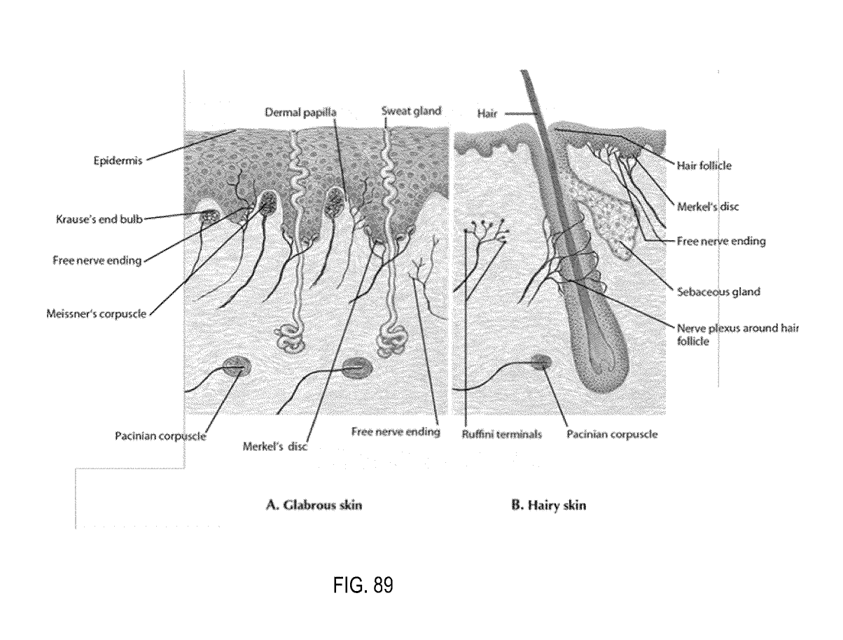

Referring to FIG. 89, in which the typical location and distribution of cutaneous pain receptors is shown, it is to be noted that the free nerve endings (which are composed of A-δ and C fibers) reside within both the dermis and the epidermis for both hairy and glabrous skin. While nociceptive projections are found throughout the skin, they tend to cluster near the dermal-epidermal junction (DEJ) in which the melanin producing keratinocytes of the stratum basale reside. The nominal thickness of the epidermis is typically between 15-100 μm in humans. It varies with anatomical location, and is typically thinner in hairy vs. glabrous skin. Epidermal thickness may not be generally correlated to age or skin type, but certain things, such as smoking and sun damage, tend to cause it to thin. Thus, to target the free nerve endings, the therapeutic light may be made to illuminate through the epidermis and into the superficial dermis to an exemplary depth of approximately 200-300 μm.

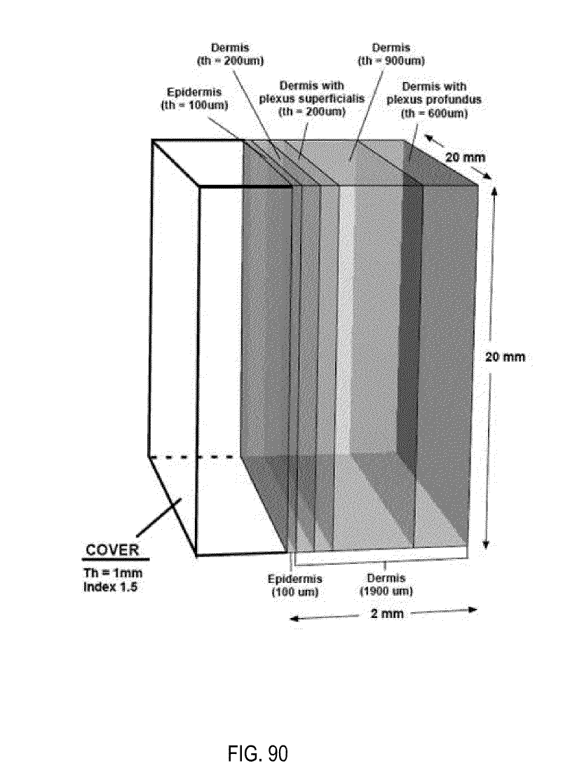

FIG. 90 contains details regarding a 3-dimensional optical model of the skin that may be used to accurately predict photon distributions for transcutaneous delivery and therefore therapeutic dosimetry. The geometry is described by V. Tuchin (Tissue Optics, Light Scattering Methods and Instrumentation for Medical Diagnostics) where he also describes the Monte Carlo technique used herein for simulating light distribution within the tissue given input light conditions. Additionally, S. Jacques from the Oregon Medical Center provides values and formulae for calculating the absorption and tissue properties of human skin as characterized by absorption parameter μₐ and scattering parameters μₛ and g. Models by Jacques decompose human adult skin into three broad categories defined by the melanosome volume fraction in the epidermis as defined in Table 1 below.

TABLE 1

| | Volume fraction of |
|---|---|
| Category | melanosome in epidermis |
| Light-skinned adults | 1.3% to 6.3% |
| Moderately pigmented adults | 11% to 16% |
| Darkly pigmented adults | 18% to 43% |

Specific examples for values at various wavelengths for light-skinned (light pigment at 2% melanosome) and darkly pigmented (dark pigment at 30% melanosome) are given in Tables 2 & 3.

TABLE 2
SUMMARY [Human, Light Pigment]

| | | Tuchin | Wave- | Sacques | Saques | Tuchin |
|---|---|---|---|---|---|---|
| Layer | Th(um) | Index | length | us | us | g |
| epidermis | 100 | 1.5 | 337 | 55.2 | 669 | 0.72 |
| 2% | | | 473 | 17.0 | 778 | 8.75 |
| Melanosome | | | 332 | 11.4 | 179 | 0.77 |
| | | | 577 | 8.7 | 148 | 0.78 |
| | | | 556 | 8.8 | 145 | 0.78 |
| | | | 633 | 6.4 | 119 | 0.79 |
| Dermis | 200 | 1.4 | 337 | 7.41 | 669 | 0.72 |
| 0.2% blood | | | 473 | 1.23 | 233 | 0.75 |
| | | | 532 | 0.58 | 179 | 0.74 |
| | | | 577 | 0.60 | 148 | 0.78 |
| | | | 590 | 0.94 | 145 | 0.79 |
| | | | 633 | 0.35 | 139 | 0.82 |
| Dermis with | 200 | 1.4 | 337 | 50.34 | 669 | 0.72 |
| plexus | | | 473 | 8.39 | 288 | 0.75 |
| superficialis | | | 537 | 10.50 | 179 | 0.77 |
| 5% blood | | | 577 | 7.87 | 148 | 0.78 |
| | | | 390 | 4.84 | 143 | 0.79 |
| | | | 633 | 1.17 | 139 | 0.82 |
| Dermis | 900 | 1.4 | 337 | 7.41 | 669 | 0.72 |
| 0.2% blood | | | 473 | 1.23 | 238 | 0.75 |
| | | | 332 | 0.33 | 179 | 0.77 |
| | | | 577 | 0.69 | 148 | 0.78 |
| | | | 590 | 0.94 | 145 | 0.79 |
| | | | 633 | 0.35 | 139 | 0.82 |
| Dermis with | 600 | 1.4 | 337 | 50.34 | 669 | 0.72 |
| plexus | | | 478 | 8.39 | 288 | 0.75 |
| profundus | | | 532 | 10.50 | 179 | 0.77 |
| 5% blood | | | 577 | 7.87 | 148 | 0.78 |
| | | | 390 | 4.84 | 143 | 0.79 |
| | | | 633 | 1.27 | 139 | 0.82 |

TABLE 3
SUMMARY [Human, Dark Pigment]

| | | Tuchin | Wave- | Sacques | Saques | Tuchin |
|---|---|---|---|---|---|---|
| Layer | Th(um) | Index | length | us | us | g |
| epidermis | 100 | 1.5 | 337 | 748.8 | 669 | 0.72 |
| 30% | | | 473 | 241.3 | 238 | 0.75 |
| melanosome | | | 532 | 568.0 | 179 | 0.77 |
| | | | 577 | 124.3 | 148 | 0.78 |
| | | | 500 | 135.4 | 145 | 0.78 |
| | | | 633 | 91.3 | 119 | 0.79 |
| Dermis | 200 | 1.4 | 337 | 7.41 | 669 | 0.72 |
| 0.2% blood | | | 473 | 1.23 | 233 | 0.75 |
| | | | 537 | 0.93 | 179 | 0.77 |
| | | | 577 | 0.69 | 148 | 0.78 |
| | | | 590 | 0.34 | 145 | 0.79 |
| | | | 633 | 0.35 | 139 | 0.82 |
| Dermis with | 200 | 1.4 | 337 | 50.34 | 669 | 0.72 |
| plexus | | | 473 | 8.39 | 238 | 0.75 |
| superficialis | | | 532 | 10.50 | 179 | 0.77 |
| 5% blood | | | 577 | 7.87 | 148 | 0.78 |
| | | | 590 | 4.84 | 143 | 0.79 |
| | | | 633 | 1.37 | 139 | 0.82 |
| Dermis | 900 | 1.4 | 337 | 7.41 | 669 | 0.72 |
| 0.2% blood | | | 473 | 1.23 | 238 | 0.75 |
| | | | 332 | 0.53 | 179 | 0.77 |
| | | | 577 | 0.69 | 148 | 0.78 |
| | | | 590 | 0.54 | 145 | 0.79 |
| | | | 633 | 0.35 | 139 | 0.82 |
| Dermis with | 600 | 1.4 | 337 | 50.34 | 669 | 0.72 |
| plexus | | | 473 | 8.39 | 288 | 0.75 |
| profundus | | | 532 | 50.56 | 179 | 0.77 |
| 5% blood | | | 577 | 7.87 | 148 | 0.78 |
| | | | 390 | 4.84 | 143 | 0.79 |
| | | | 633 | 1.37 | 139 | 0.82 |

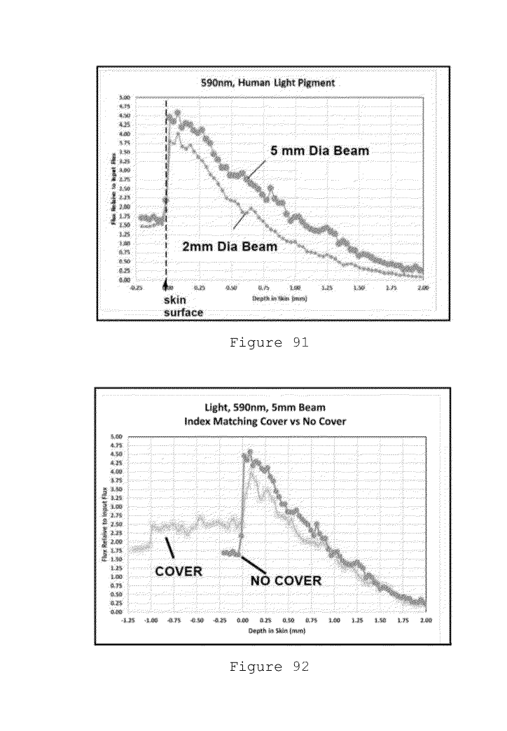

FIG. 91 describes the irradiance along a 590 nm wavelength illumination beam center as it traverses lightly pigmented skin, as defined in Table 2. The subsurface irradiance is higher than the surface irradiance due to refractive index mismatching at the skin surface and the backscattered light from the scattering media.

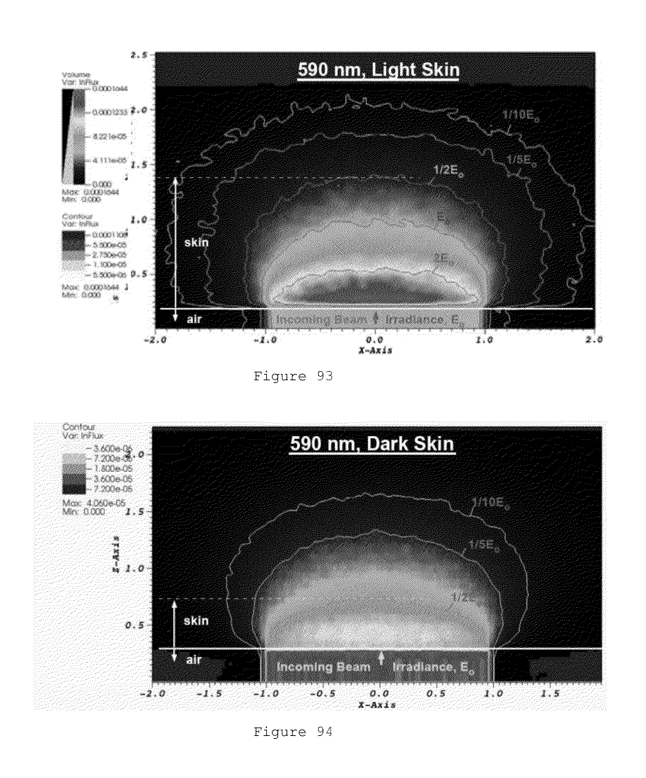

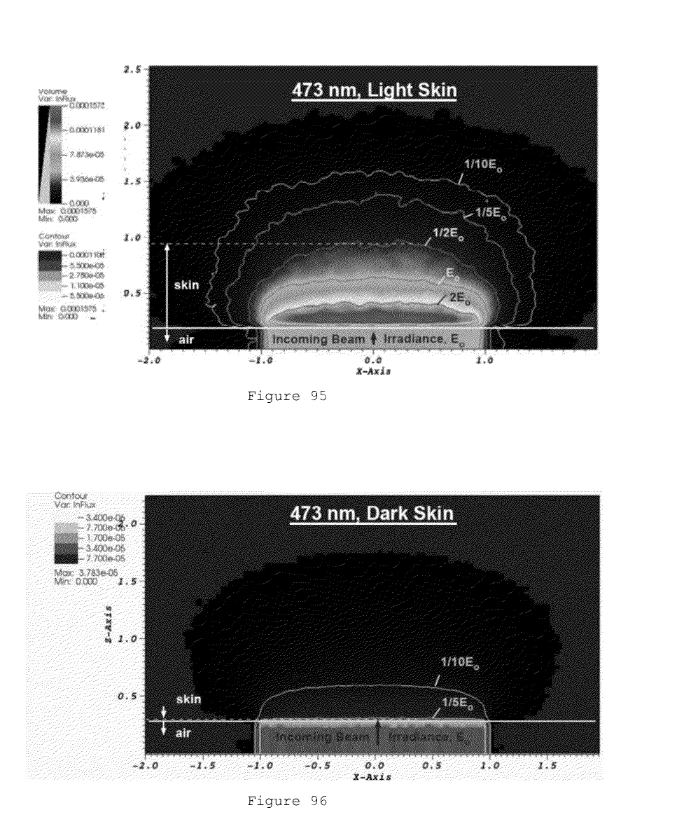

FIG. 92 describes this same configuration with a glass plate placed in contact with the skin surface to improve the index matching and lower the subsurface irradiance by allowing light to be remitted from the tissue. This may be useful in order to avoid overheating the melanin contained in the epidermis, for example, although at the expense of overall system efficiency. It can be seen here that the beam diameter plays a role in the effective penetration depth along the beam center because the edge effects (where light is lost) become proportionately less significant as the beam diameter increases. It can be seen here that light penetrates fairly deeply in this configuration, and is thus able to reach cutaneous sensory nerve endings. This is shown in more detail in FIG. 93.

FIG. 93 shows the 590 nm wavelength beam irradiance in lightly pigmented skin, as defined in Table 2, in cross section through the beam center. Even at depths of 1.8 mm, the exposure is still 10% of that at the surface. This figure is the result of a simulation using 1,000,000 collimated rays at wavelength 590 nm in a uniform beam of 32 mW/mm² of diameter 2 mm and the light skin parameters of Table 2 and is shown in FIG. 93. The power flux distribution is plotted. Contour lines indicate equivalent values of constant irradiance, E. The value of E is normalized to the incident irradiance, Eo. Note the increase in equivalent irradiance just below the surface of the skin. This is a phenomenon that occurs in scattering medium and is well known for lightly pigmented tissue in the biomedical community. Also note the depth of penetration of the light in the tissue. A representative number to use for depth is the value at which the equivalent irradiance drops by a factor of ½ from the incoming irradiance. For this case at wavelength 590 nm, that depth is about 1.2 mm. In general, the deeper or greater this number the better for it increases the likelihood of sufficient light hitting a nerve. But the enhancement or amplification of the light near the surface needs to be managed so that tissue is not damaged.

FIG. 94 shows the 590 nm wavelength beam irradiance in darkly pigmented skin, as defined in Table 3, in cross section through the beam center. Even at depths of approximately 1.3 mm, the exposure is still 10% of that at the surface. The ½Eo penetration depth is reduced to approximately 500 μm.

FIG. 95 shows the 473 nm wavelength beam irradiance in lightly pigmented skin, as defined in Table 2, in cross section through the beam center. Even at depths of approximately 1.5 mm, the exposure is still 10% of that at the surface. The ½Eo penetration depth is reduced to approximately 750 μm.

FIG. 96 shows the 473 nm beam intensity in darkly pigmented skin, as defined in Table 3, in cross section through the beam center. The exposure is still >10% of that at the surface at depths of up to approximately 200 μm. The ½Eo penetration depth is reduced to approximately 50 μm.

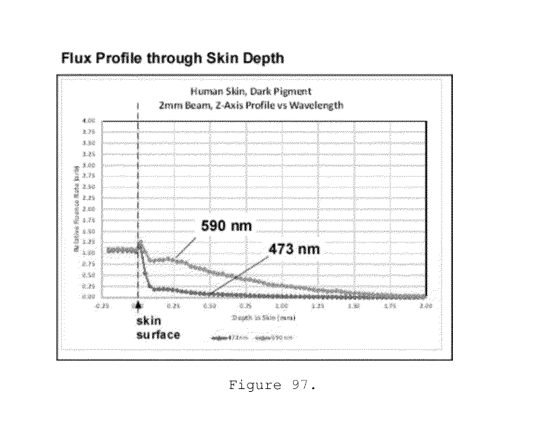

FIG. 97 is plot of the fluence rate vs depth into the skin. It is plot of the flux values down the center of the beam as a function of skin depth. FIG. 14 contains the same results from the simulations used to generate FIGS. 94 and 96. In FIG. 97, the results from the two wavelengths are compared in the same plot and it can be seen that the 590 nm wavelength penetrates deeper than the 473 nm wavelength light. The skin is modeled as described in Tables 2&3.

It's clear that the exposure of cutaneous nerve endings in a variety of skin types is clinically feasible, even with Blue Light.

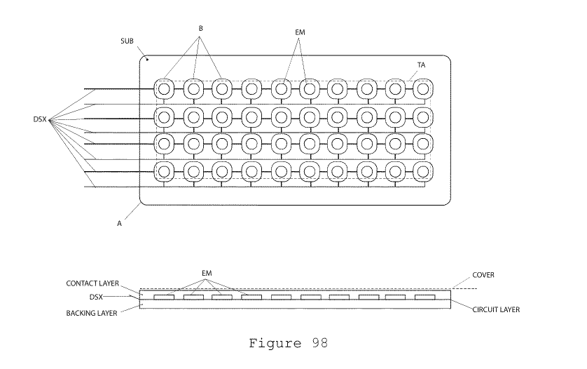

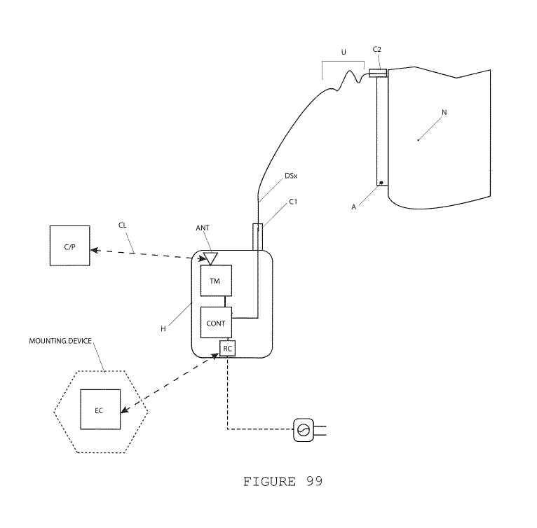

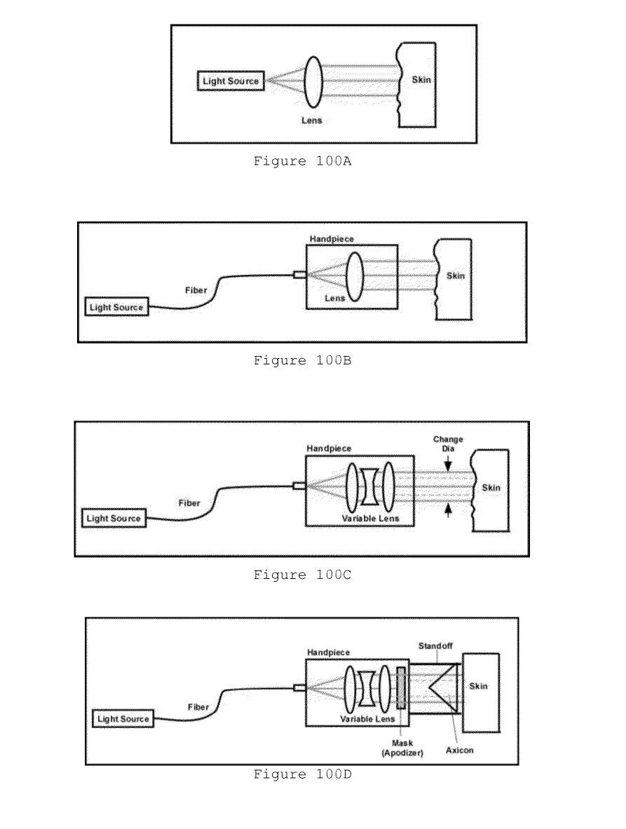

Such as is shown in FIG. 98, an array of LEDs may be used to illuminate the surface of the therapeutic target, such as the skin. In this descriptive exemplary embodiment, a 2-dimensional square array of LEDs composed of emitters EM and bases B is built upon a substrate SUB, which contains a CIRCUIT LAYER with electrical current being provided by Delivery Segments DSx, a CONTACT LAYER and a BACKING LAYER. In this example rows of LEDs are arranged in a serial-parallel configuration, although other configurations are within the scope of the present invention. Emitters EM may be comprised of surface mounting LEDs, such as for example, the LUXEON Z series, or NICHIA 180A, 157X series. Emitters EM may reside on bases B in order to make electrical connections. CONTACT LAYER may be made of a nominally transparent, soft, compliant material, such as silicone, PDMS, or other such material; which may provide a level of comfort for the patient. The thickness of CONTACT LAYER may be configured to provide nominally uniform illumination at the tissue surface. For example, using the LUXEON Z LEDs mentioned above, spaced 4 mm apart (center-to-center), illumination may be uniform to within 10% peak-to-valley using a 2.5 mm thick silicone sheet. CIRCUIT LAYER may be a single layer kapton-based flex circuit with traces configured to carry the current required that is at least in part based up on the topology, number of LEDs, and their peak powers. The number of LEDs may be chosen for a specific treatment area TA. BACKING LAYER may be constructed of a material whose compliance matches that of the CONTACT LAYER, but need not be transparent. Both CONTACT LAYER and BACKING LAYER may be chosen to have improved thermal conductivity to limit tissue heating due to electrical inefficiencies of the LEDs, and photothermal effects due to collateral heating of tissue pigmentation. However, it should be noted that skin cooling is less of an issue for the present optogenetic therapy than for traditional laser dermatologic procedures because the irradiance used is well under those utilized for traditional laser dermatologic procedures; such as tattoo removal, vascular lesion photothermal therapy, and hair removal. These traditional therapies employ exposures of pulses from 5 ns to 500 ms and surface fluences of between 1 and 100 J/cm², which correspond to a large range of peak irradiances of between 50 mW/mm² and 20 MW/mm², albeit for short exposure times and low pulse repetition rates. Furthermore, a cover COVER may be used to keep the optical surface clean prior to use. It may alternately serve to enclose adhesive, like a bandage, for fixation to a tissue surface. Delivery segments DSx may be collected into a ribbon connector for connection to the rest of the therapeutic system, as shown in FIG. 99.

FIG. 99 relates to an exemplary therapeutic device for use with the applicator described above with respect to FIG. 98. Applicator A, slab-type applicator that is 20 mm wide and 40 mm long, such as is described in more detail with respect to FIGS. 18 and 21-23 of International application number PCT/US2013/000262 (publication number WO/2014/081449), which is incorporated by reference herein in its entirety, is deployed about the surface of target tissue N. Electrical power is delivered to Applicator A via Delivery Segment DS to power the LEDs resident in the applicator. The resulting Light Field may be configured to provide illumination of the target tissues within the surface intensity range of 0.1-40 mW/mm², and may be dependent upon one or more of the following factors; the specific opsin used, its concentration distribution within the tissue, the tissue optical properties, and the size of the target structure(s), or its depth within a larger tissue structure. The system may be operated in a pulsed mode, where the pulse duration may be made from between 0.5 ms to 1 s, with a pulse duration of 10 ms being typically effective for inhibitory channels. Furthermore, the pulse repetition frequency (PRF) may be configured from between 0.1 Hz and 200 Hz, with a PRF of 1 Hz being typically effective for inhibitory channels. Consequently, the duty cycle ranges from 0.005% to 100%, with a duty cycle of 1% being typically effective for inhibitory channels. Although not shown for simplicity and clarity in the present figure, multiple applicators and/or delivery segments may be used for a specific target structure if it is a large target structure when compared to the optical penetration depth within that structure. Delivery Segment DS may be configured to be a ribbon cable. Delivery Segment DS may further comprise Undulations U, which may provide strain relief. Delivery Segment DS may be operatively coupled to Housing H via connector C1 and to the applicator via connector C2. The electrical power and/or current may be controlled by controller CONT, and parameters such as optical intensity, exposure time, pulse duration, pulse repetition frequency, and duty cycle may be configured. The Controller CONT shown within Housing H is a simplification, for clarity, of that described in more detail with respect to FIG. 10. External clinician programmer module and/or a patient programmer module C/P may communicate with Controller CONT via Telemetry module TM via Antenna ANT via Communications Link CL. Power Supply PS, not shown for clarity, may be wirelessly recharged using External Charger EC. Furthermore, External Charger EC may be configured to reside within a Mounting Device MOUNTING DEVICE. Mounting Device MOUNTING DEVICE may be a vest, as is especially well configured for this exemplary embodiment. External Charger EC, as well as External clinician programmer module and/or a patient programmer module C/P and Mounting Device MOUNTING DEVICE may be located within the extracorporeal space ESP, while the rest of the system is implanted and may be located within the intracorporeal space ISP. External Charger EC may also be an AC adapter, as shown by the dotted line and universal AC symbol.

A block diagram is depicted in FIG. 32 illustrating various components of an example housing H. In this example, the housing includes processor CPU, memory M, power source PS, telemetry module TM, antenna ANT, and the driving circuitry DC for an optical stimulation generator. The Housing H is shown coupled to one Delivery Segments DSx for simplicity and clarity. It may be a multi-channel device in the sense that it may be configured to include multiple electronic paths (e.g., multiple light sources and/or sensor connections) that may deliver different optical outputs, some of which may have different wavelengths. The delivery segments may be detachable from the housing, or be fixed.

Memory (MEM) may store instructions for execution by Processor CPU, optical and/or sensor data processed by sensing circuitry SC, and obtained from sensors both within the housing, such as battery level, discharge rate, etc., and those deployed outside of the Housing (H), possibly in Applicator A, such as optical and temperature sensors, and/or other information regarding therapy for the patient. Processor (CPU) may control Driving Circuitry DC to deliver power to the light source (not shown) according to a selected one or more of a plurality of programs or program groups stored in Memory (MEM). The Light Source may be internal to the housing H, or remotely located in or near the applicator (A), as previously described. Memory (MEM) may include any electronic data storage media, such as random access memory (RAM), read-only memory (ROM), electronically-erasable programmable ROM (EEPROM), flash memory, etc. Memory (MEM) may store program instructions that, when executed by Processor (CPU), cause Processor (CPU) to perform various functions ascribed to Processor (CPU) and its subsystems, such as dictate pulsing parameters for the light source, as described earlier.

In accordance with the techniques described in this disclosure, information stored in Memory (MEM) may include information regarding therapy that the patient had previously received. Storing such information may be useful for subsequent treatments such that, for example, a clinician may retrieve the stored information to determine the therapy applied to the patient during his/her last visit, in accordance with this disclosure. Processor CPU may include one or more microprocessors, digital signal processors (DSPs), application-specific integrated circuits (ASICs), field-programmable gate arrays (FPGAs), or other digital logic circuitry. Processor CPU controls operation of implantable stimulator, e.g., controls stimulation generator to deliver stimulation therapy according to a selected program or group of programs retrieved from memory (MEM). For example, processor (CPU) may control Driving Circuitry DC to deliver optical signals, e.g., as stimulation pulses, with intensities, wavelengths, pulse widths (if applicable), and rates specified by one or more stimulation programs. Processor (CPU) may also control Driving Circuitry (DC) to selectively deliver the stimulation via subsets of Delivery Segments (DSx), and with stimulation specified by one or more programs. Different delivery segments (DSx) may be directed to different target tissue sites, as was previously described.

Telemetry module (TM) may include a radio frequency (RF) transceiver to permit bi-directional communication between implantable stimulator and each of clinician programmer and patient programmer (C/P). Telemetry module (TM) may include an Antenna (ANT), of any of a variety of forms. For example, Antenna (ANT) may be formed by a conductive coil or wire embedded in a housing associated with medical device. Alternatively, antenna (ANT) may be mounted on a circuit board carrying other components of implantable stimulator or take the form of a circuit trace on the circuit board. In this way, telemetry module (TM) may permit communication with a controller/programmer (C/P). Given the energy demands and modest data-rate requirements, the Telemetry system may be configured to use inductive coupling to provide both telemetry communications and power for recharging, although a separate recharging circuit (RC) is shown in FIG. 10 for explanatory purposes.

External programming devices for patient and/or physician can be used to alter the settings and performance of the implanted housing. Similarly, the implanted apparatus may communicate with the external device to transfer information regarding system status and feedback information. This may be configured to be a PC-based system, or a stand-alone system. In either case, the system must communicate with the housing via the telemetry circuits of Telemetry Module (TM) and Antenna (ANT). Both patient and physician may utilize controller/programmers (C/P) to tailor stimulation parameters such as duration of treatment, optical intensity or amplitude, pulse width, pulse frequency, burst length, and burst rate, as is appropriate.

Once the communications link (CL) is established, data transfer between the MMN programmer/controller and the housing may begin. Examples of such data are:
1. From housing to controller/programmer:
   d. Patient usage
   e. Battery lifetime
   f. Feedback data
      i. Device diagnostics (such as direct optical transmission measurements by an emitter-opposing photosensor)
2. From controller/programmer to housing:
   g. Updated illumination level settings based upon device diagnostics
   h. Alterations to pulsing scheme
   i. Reconfiguration of embedded circuitry
      i. FPGA, etc.

By way of non-limiting examples, near field communications, either low power and/or low frequency; such as is produced by Zarlink/MicroSEMI may be employed for telemetry, as well as Bluetooth, Low Energy Bluetooth, Zigbee, etc.

FIG. 100A is an optical layout for a simple transcutaneous illumination system. It consists of a light source at the corresponding opsin wavelength. A laser or LED can be used for the light source. A lens can be used to deliver light to the skin. A lens placed approximately 1 focal length away from the source (or its beam waist) may serve to collimate the beam, as shown.

FIG. 100B places a fiber optics between the light source and a handpiece that contains the delivery optics. The handpiece can be made compact by remotely locating the light source via the fiber. It also allows easy exchange to other light sources that either differ in form (Laser, LED) or wavelength by achromatizing the optical design.

In FIG. 100C a schematic of a typical variable lens system used for cutaneous photomedicine is shown within the handpiece. This variable optical system can be user operated to change the spot size at the tissue for instance.

In FIG. 100D a cover is added to the system. This cover can be part of the handpiece and attached via a stand-off. The cover can be used to index match the skin as described previously. It can also be used to cool and compress the skin, as described in U.S. Pat. No. 6,273,884; by Altschuler and Anderson. Compression and cooling can be used to decrease the light induced damage and optimize depth of light penetration.

As used herein, “handpiece” may also refer to any external transcutaneous optical delivery system.

Referring to FIG. 3, a suitable light delivery system comprises one or more applicators (A) configured to provide light output to the targeted tissue structures. The light may be generated within the applicator (A) structure itself, or within a housing (H) that is operatively coupled to the applicator (A) via one or more delivery segments (DS), or at a location between the housing (H) and the applicator (A). The one or more delivery segments (DS) serve to transport, or guide, the light to the applicator (A) when the light is not generated in the applicator itself. In an embodiment wherein the light is generated within the applicator (A), the delivery segment (DS) may simply comprise an electrical connector to provide power to the light source and/or other components which may be located distal to, or remote from, the housing (H). The one or more housings (H) preferably are configured to serve power to the light source and operate other electronic circuitry, including, for example, telemetry, communication, control and charging subsystems. External programmer and/or controller (P/C) devices may be configured to be operatively coupled to the housing (H) from outside of the patient via a communications link (CL), which may be configured to facilitate wireless communication or telemetry, such as via transcutaneous inductive coil configurations, between the programmer and/or controller (P/C) devices and the housing (H). The programmer and/or controller (P/C) devices may comprise input/output (I/O) hardware and software, memory, programming interfaces, and the like, and may be at least partially operated by a microcontroller or processor (CPU), which may be housed within a personal computing system which may be a standalone system, or be configured to be operatively coupled to other computing or storage systems. Such systems are described in International application number PCT/US2013/000262 (publication number WO/2014/081449), which is incorporated by reference herein in its entirety.

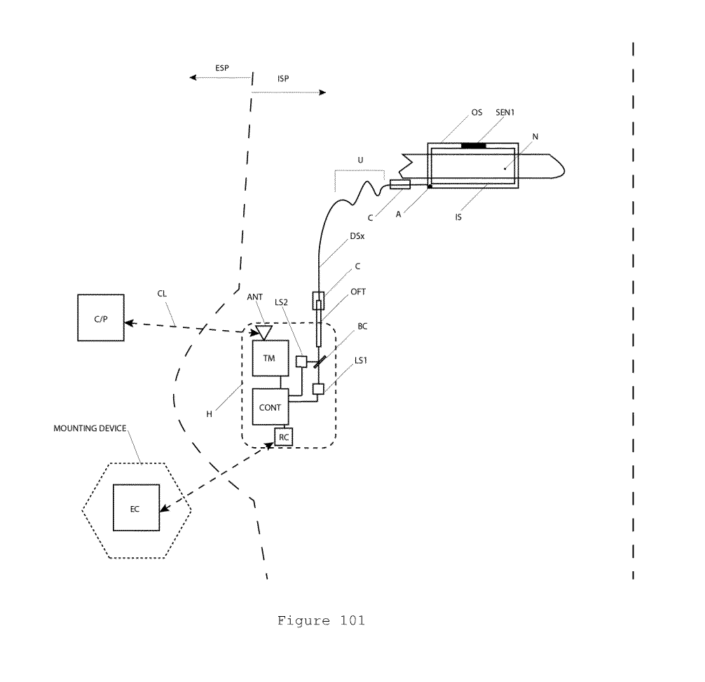

FIG. 101 shows an exemplary embodiment of a system for the treatment of Pain via optogenetic control, configured for therapeutic use as described with respect to FIGS. 6,7A, & 8. Applicator A, a rolled slab-type applicator that is 10 mm wide and 40 mm long when unrolled, such as is described in more detail with respect to FIGS. 18 and 21-23 herein and those of International application number PCT/US2013/000262 (publication number WO/2014/081449), which is incorporated by reference herein in its entirety, is deployed about the target tissue N. Light is delivered to Applicator A via Delivery Segment DS, respectively, to create a Light Field substantially within the applicator. The Light Field may be configured to provide illumination of the target tissues within the intensity range of 0.01-50 mW/mm², and may be dependent upon one or more of the following factors; the specific opsin used, it's concentration distribution within the tissue, the tissue optical properties, and the size of the target structure(s), or its depth within a larger tissue structure. Although not shown for simplicity and clarity in the present figure, multiple applicators and/or delivery segments may be used for a specific target structure if it is a large target structure when compared to the optical penetration depth within that structure. Delivery Segment DS may be configured to be an optical fiber, such as 105 μm core diameter/125 μm cladding diameter/225 μm acrylate coated 0.22 NA step index fiber that is enclosed in a protective sheath, such as a 300 μm OD silicone tube. Connector C may be configured to operatively couple light from Delivery Segment DS to Applicator A. Delivery Segment DS may further comprise Undulations U, which may provide strain relief. Delivery Segment DS may be operatively coupled to Housing H via Optical Feedthrough OFT. Light is provided to Delivery Segment DS from Light Sources LS1 & LS2, within Housing H. Light Sources LS1 & LS2 may be configured to be LEDs, and/or lasers that provide spectrally different output to activate and/or deactivate the opsins resident within target tissue(s), as dictated by the therapeutic paradigm. For example, LS1 may be configured to be a blue laser source, such as the LD-445-20 from Roithner Lasertechnik that produces up to 20 mW of 450 nm light, and is suitable for use in optogenetic intervention using such opsins as ChR2, and/or iC1C2, and/or iChR2, for example. Light Source LS2 may be configured to be a different laser than LS1, such as the QLD0593-9420 from QD Photonics that produces up to 20 mW of 589 nm light, and is suitable for use in optogenetic inhibition using NpHR, or deactivation of iC1C2, for example. Alternately, red light sources of wavelength near 635 nm may be also be used for these purposes. Light Sources LS1 & LS2 may be independently controlled by controller CONT, such that the exposures provided them are configured independently for response of their respective target tissue properties. The Controller CONT shown within Housing H is a simplification, for clarity, of that described in more detail with respect to FIG. 10. External clinician programmer module and/or a patient programmer module C/P may communicate with Controller CONT via Telemetry module TM via Antenna ANT via Communications Link CL. Power Supply PS, not shown for clarity, may be wirelessly recharged using External Charger EC. Furthermore, External Charger EC may be configured to reside within a Mounting Device MOUNTING DEVICE. Mounting Device MOUNTING DEVICE may be a vest, as is especially well configured for this exemplary embodiment. External Charger EC, as well as External clinician programmer module and/or a patient programmer module C/P and Mounting Device MOUNTING DEVICE may be located within the extracorporeal space ESP, while the rest of the system is implanted and may be located within the intracorporeal space ISP. FIGS. 32 through 37, and 99 refer to various components of an example housing H and other system aspects, where at least elements of which are germane to the configuration of this exemplary embodiment.

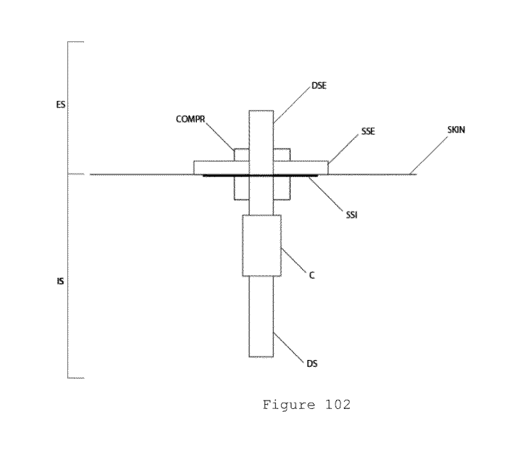

FIG. 102 shows an embodiment of a transcutaneous optical feedthrough, or port, comprising, by way of non-limiting example, an External Delivery Segment DSE, which in turn is routed through a seal, comprised of, External Sealing Element SSE that resides in the extracorporeal space ES, and Internal Sealing Element SSI that resides in the intracorporeal space IS. These sealing elements may held together by means of Compression Element COMPR to substantially maintain an infection-free seal for Transcutaneous Optical Feedthrough COFT. Internal Seal SSI, may comprise a medical fabric sealing surface along with a more rigid member coupled thereto to more substantially impart the compressive force from Compression Element COMPR when forming a percutaneous seal. The medical fabric/textile may be selected from the list consisting of, by way of non-limiting examples; dacron, polyethylene, polypropylene, silicone, nylon, and PTFE. Woven and/or non-woven textiles may be used as a component of Internal Seal SSI. The fabric, or a component thereon, may also be made to elute compounds to modulate wound healing and improve the character of the seal. Such compounds, by way of non-limiting examples, may be selected from the list consisting of; Vascular Endothelial Growth Factor (VEGF), glycosaminoglycans (Gags), and other cytokines. Applicable medical textiles may be available from vendors, such as Dupont and ATEX Technologies, for example. Delivery Segment DS may be connected to the optical and/or electrical connections of Applicator A, not shown for purposes of clarity, not shown. External Delivery Segment DSE may be may be connected to the optical and/or electrical output of Housing H, not shown for purposes of clarity. The surface of the patient, indicated in this example as Skin SKIN, may offer a natural element by way of the epidermis onto which the seal may be formed. Details regarding the means of sealing External Delivery Segment DES, which passes through the Skin SKIN, to Compression Element COMPR are discussed elsewhere herein in regards to optical feedthroughs within Housing H, such as are shown elsewhere herein.

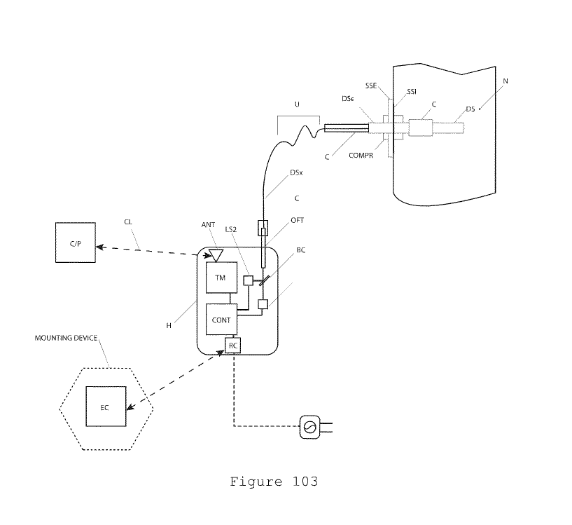

FIG. 103 relates to an exemplary therapeutic device for use with the percutaneous port described above with respect to FIG. 102. FIGS. 32 through 37, 99, and 101 refer to various components of an example housing H and other system aspects, where at least elements of which are germane to the configuration of this exemplary embodiment.

As used herein, the terms “surface intensity” and “intensity” may be used interchangeably, unless otherwise specified.

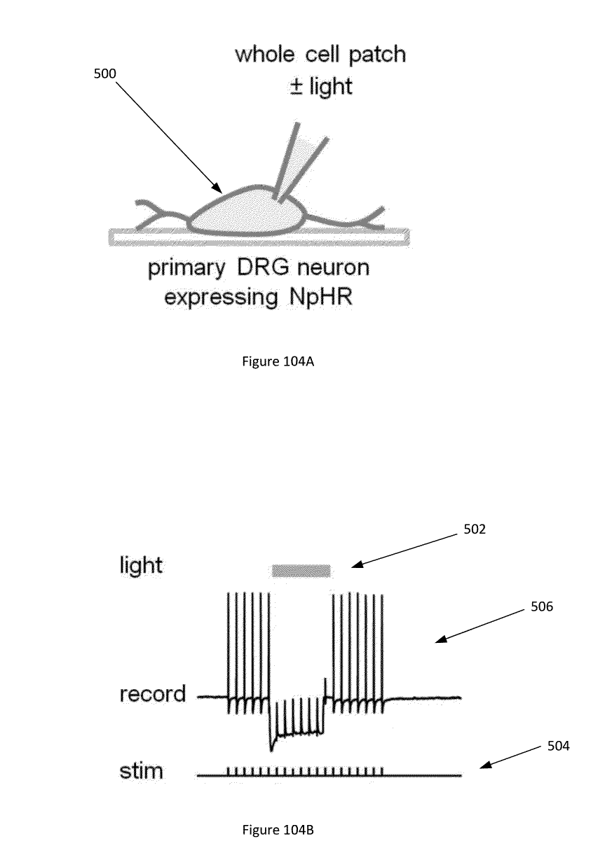

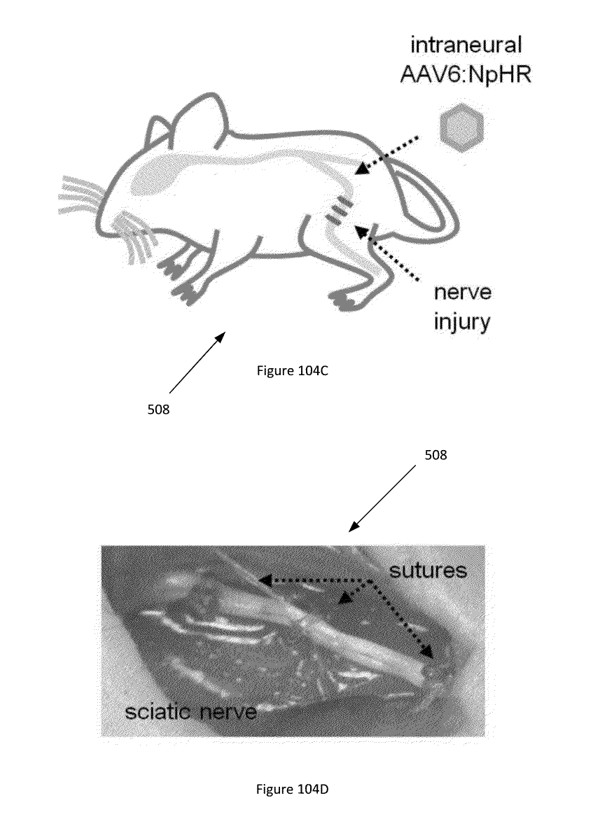

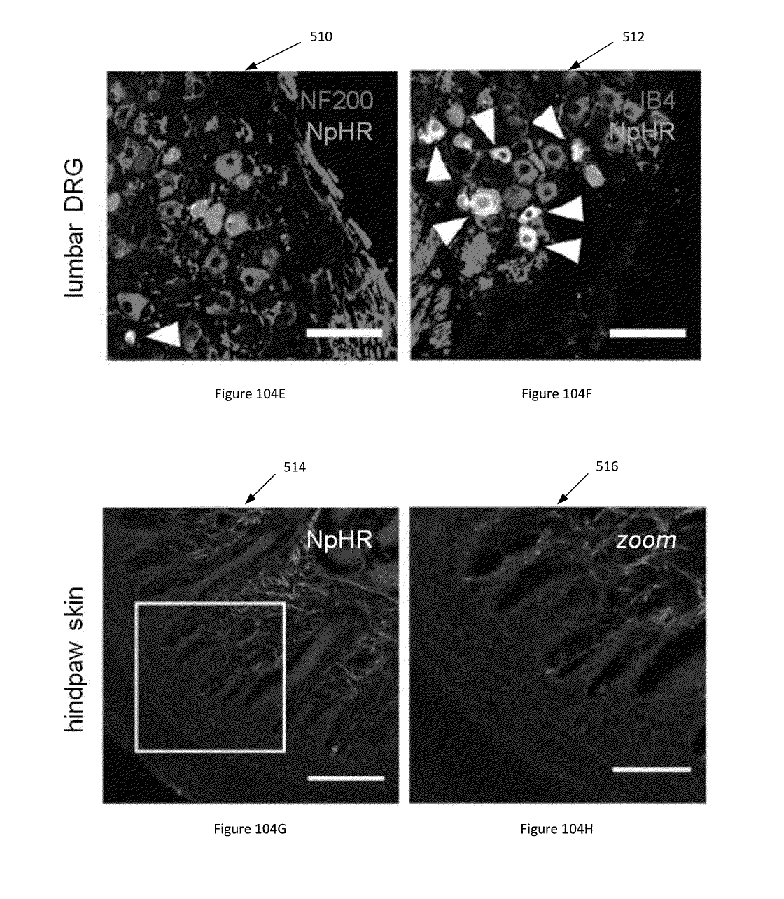

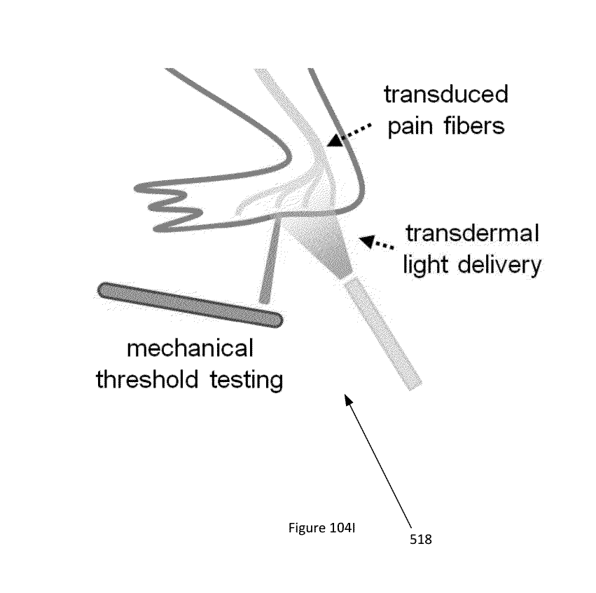

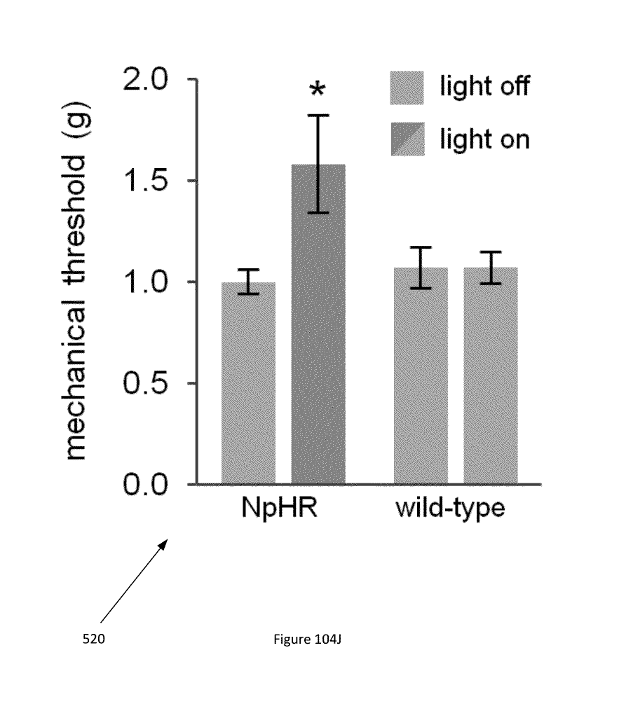

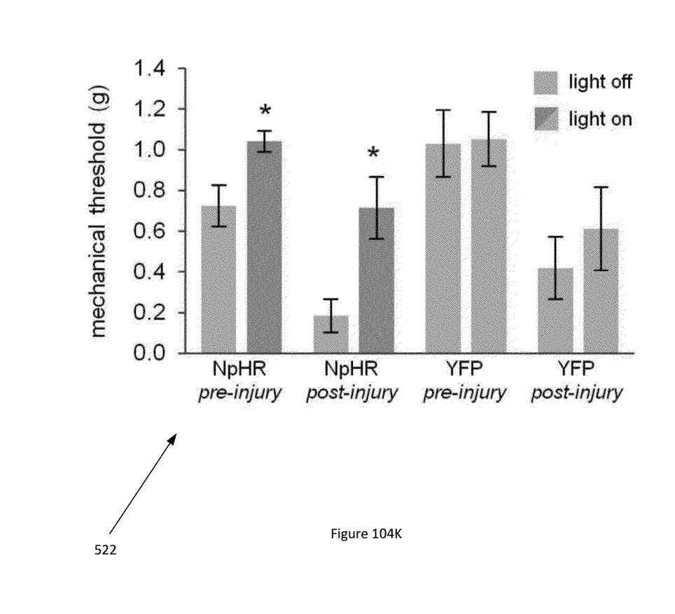

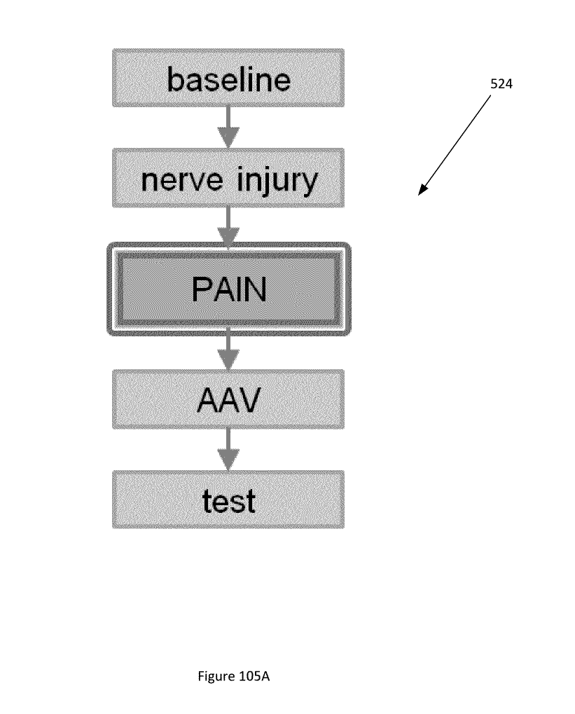

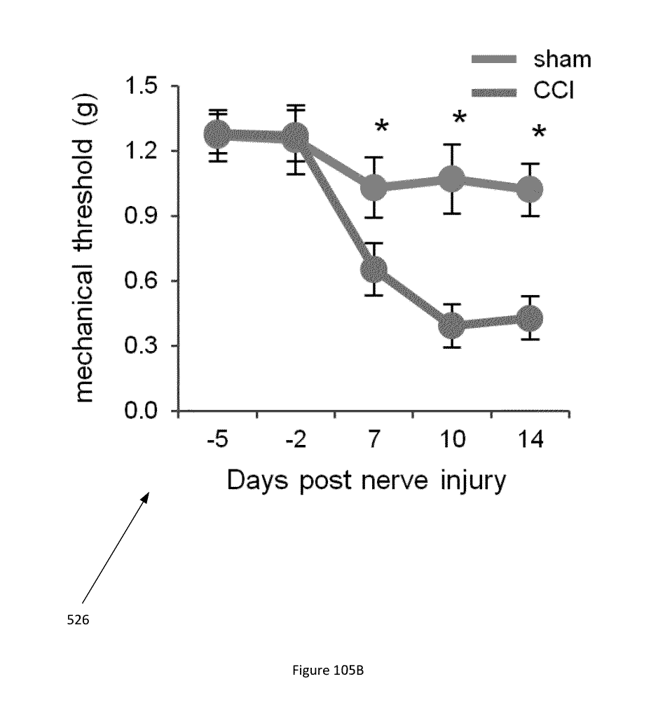

Referring to FIGS. 104A-108G, various aspects of studies and related results are depicted.

Referring to FIGS. 104A-104K, aspects of proof of concept configurations and data are illustrated for inhibition of pain using optogenetic therapy in preclinical models. As shown in FIGS. 104A and 104B, primary dorsal root ganglion (DRG 500) neurons are transfected with NpHR and electrically stimulated (504). Referring to FIG. 104B, the application of yellow light (502) decreases reduces evoked action potentials (506) demonstrating optogenetic inhibition of sensory neuron activity in vitro. Referring to FIGS. 104C and 104D, six week old mice are injected in the sciatic nerve (508) with 1×10¹¹ vg of AAV6:hSyn-NpHR-YFP and sacrificed 3 weeks later. Referring to FIGS. 104E-104H (510, 512, 514, 516), NpHR-YFP expression is observed in pain sensory neurons in DRG (IB4+) but not non-pain sensory neurons (NF200+). White arrows indicate double labeled cells. NpHR-YFP is also trafficked down to nerve endings in skin, where they can then be modulated by transdermal light delivery (518), as shown in FIG. 1041. Referring to the chart (520) of FIG. 104J, application of light decreases mechanical threshold levels in AAV6:NpHR mice but not wild-type mice as determined by von Frey filament testing. Referring to the chart (522) of FIG. 104K, AAV6:NpHR combined with light delivery also blocks acute pain 3 days after nerve injury whereas AAV6:YFP does not display this phenomenon.

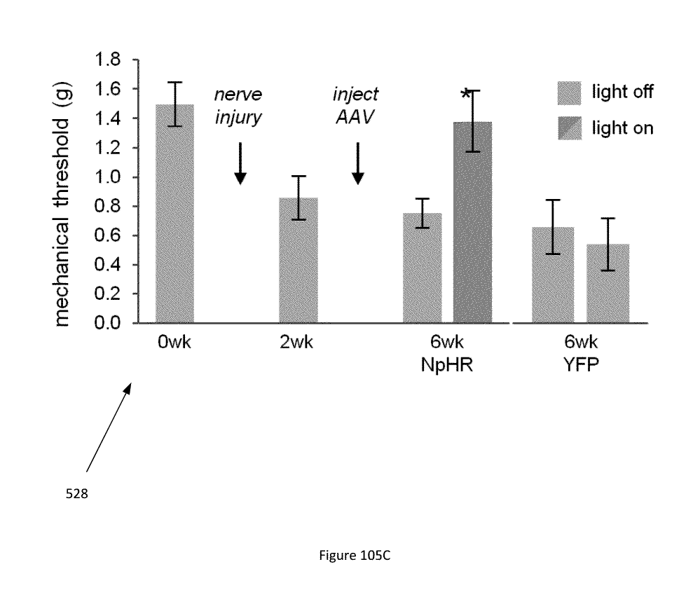

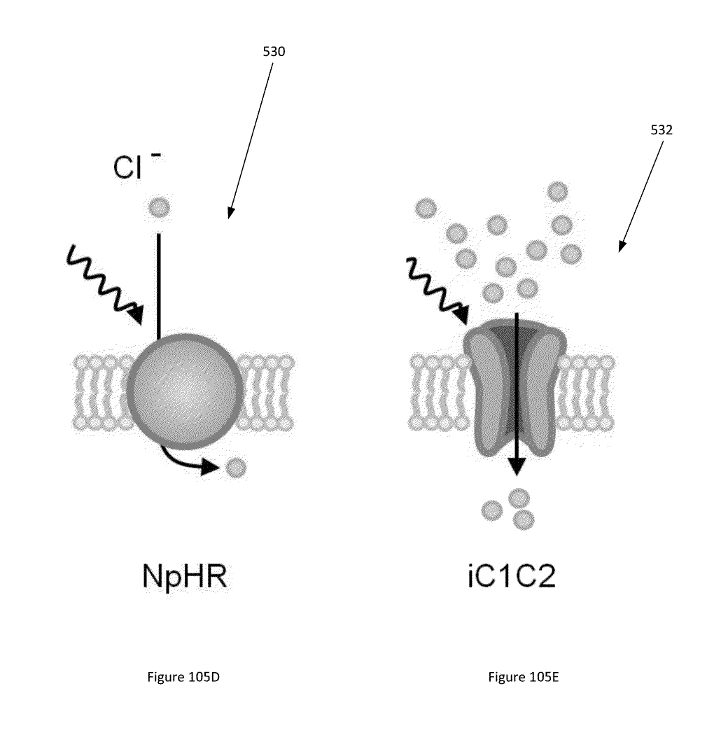

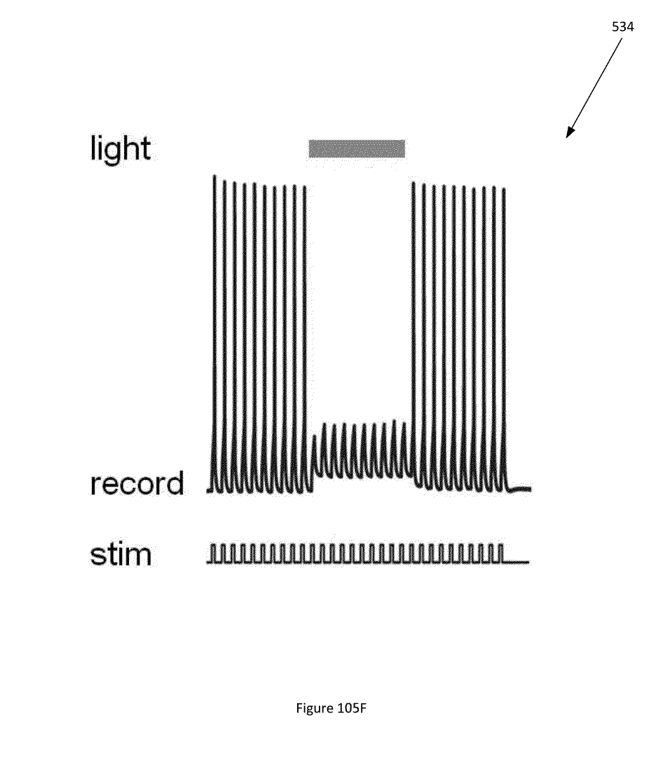

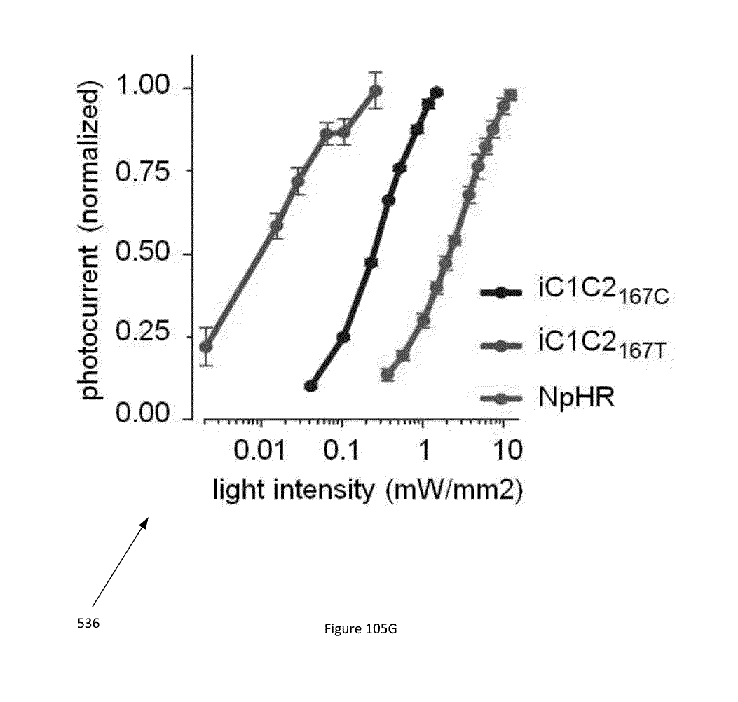

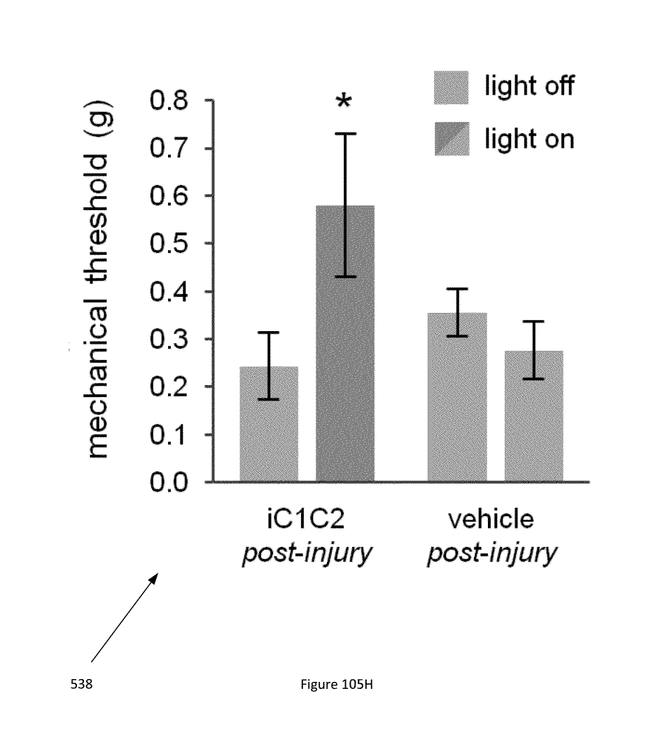

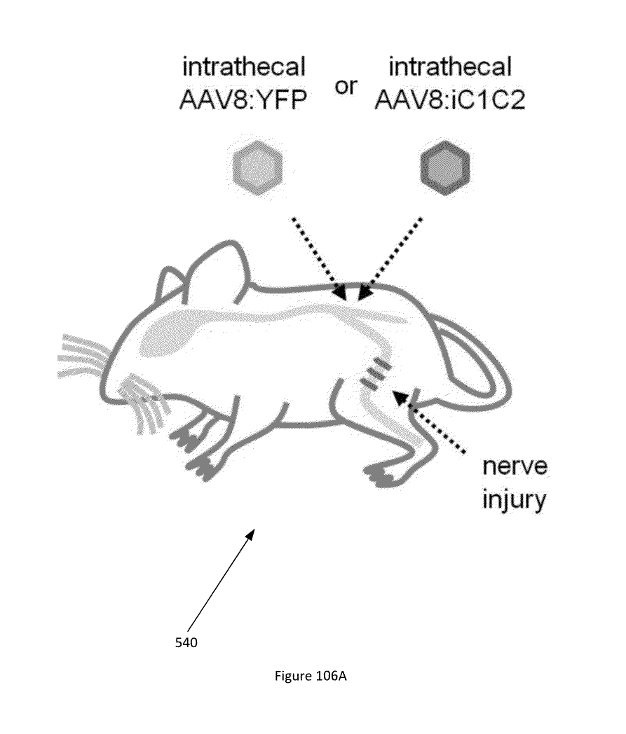

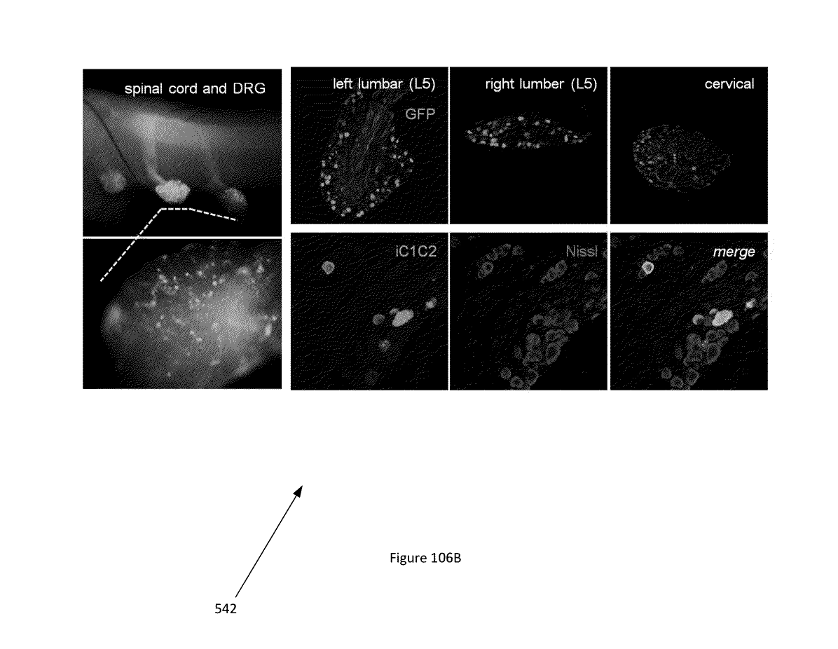

Referring to FIGS. 105A-105H, aspects of preclinical translation of pain inhibition are illustrated. As shown in FIG. 105A, an experimental flow (524) may be set up to determine whether viral delivery following establishment of neuropathic pain can also result in pain inhibition. Referring to the chart (526) of FIG. 105B, mice undergoing chronic constriction injury (“CCI”) have reduced mechanical threshold levels that are stable through time. Referring to the chart (528) of FIG. 105C, AAV6:NpHR is delivered by nerve injection two weeks following induction of mechanical allodynia and results in pain inhibition four weeks later in response to light. This effect was not observed with AAV6:YFP. Referring to FIG. 105D, NpHR is a chloride pump (530) that actively transports one chloride ion per photon of light. Referring to FIG. 105E, iC1C2 is a chloride channel (532) that opens in response to one photon of light and can allow multiple ions to travel across their concentration gradient. Referring to the chart (534) of FIG. 105F, primary neurons expressing iC1C2 are inhibited in response to blue light (e.g. 473 nm) in conditions where extracellular chloride concentrations are high. Referring to the chart (536) of FIG. 105G, two variants of iC1C2 demonstrate higher photocurrents per light intensity than NpHR, presumably due to ability to transport more ions per quantity of light particles. Referring to chart (538) of FIG. 105H, nerve injections of AAV6:iC1C2 into mice with pre-existing CCI result in reduced pain upon application of light.

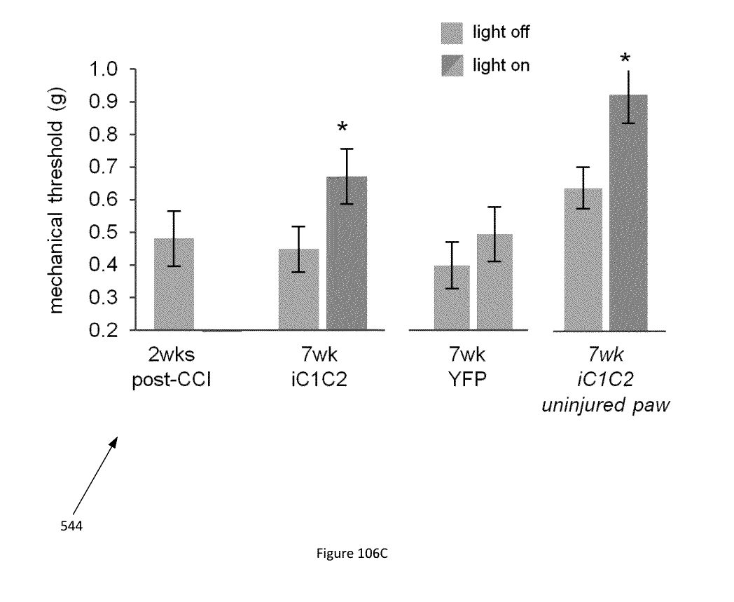

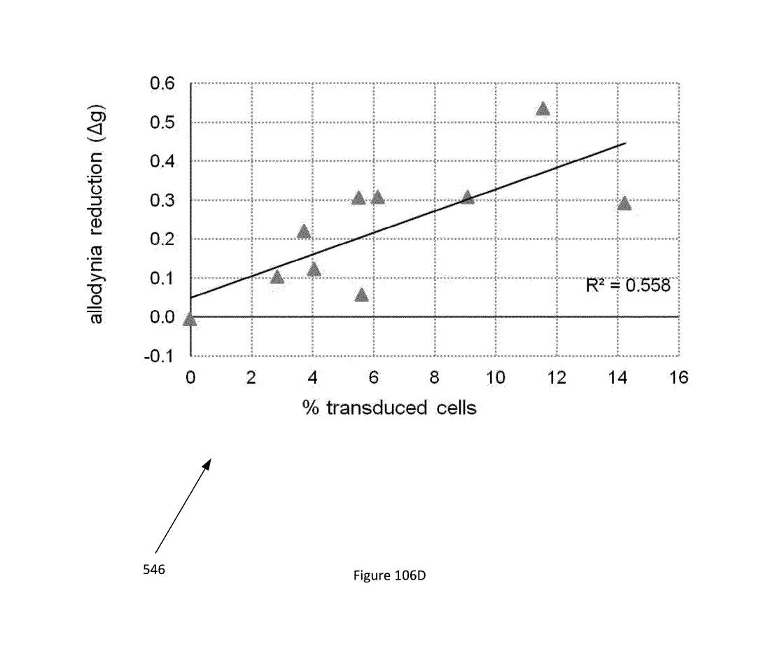

Referring to FIGS. 106A-106D, aspects of intrathecal delivery for optogenetic therapy of neuropathic pain are illustrated. Referring to FIG. 106A, a configuration (540) is illustrated wherein AAV8 expressing either YFP or iC1C2-YFP are injected into the intrathecal space of mice that have undergone CCI using lumbar puncture method. As shown in the tissue fluorescence images (542) of FIG. 106B, four weeks following AAV8:YFP injection animals were sacrificed and gross fluorescence on dissected tissue reveals intense expression in multiple DRGs and spinal cord. Sections reveal transduction in both left and right lumbar DRGs as well as in cervical levels. Expression is also observed following AAV8:iC1C2 in multiple DRG that co-localizes with markers of neurons as expected. Referring to the chart (544) of FIG. 106C, intrathecal injection of AAV8:iC1C2 but not AAV8:YFP reverses mechanical allodynia upon application of light when administered 2 weeks following CCI delivery. Note that this effect is also observed in the uninjured paw. Referring to the chart (546) of FIG. 106D, the magnitude of the reduction in allodynia correlates with percentage of transduced cells.

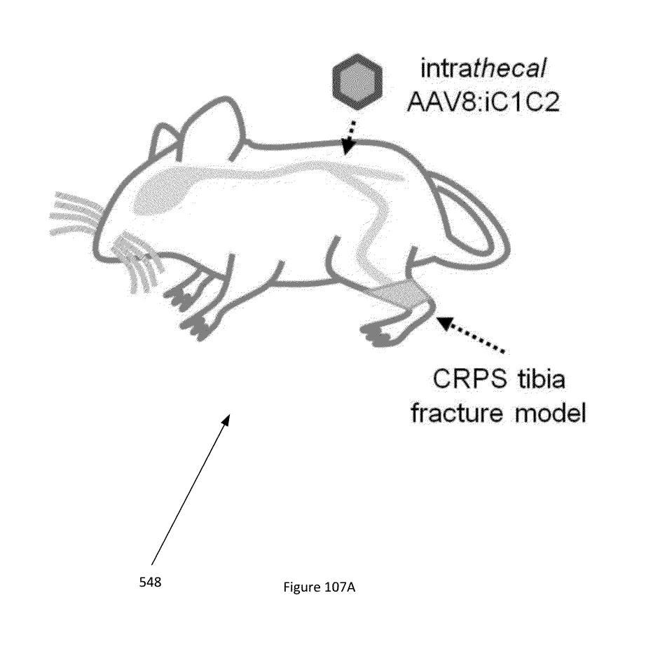

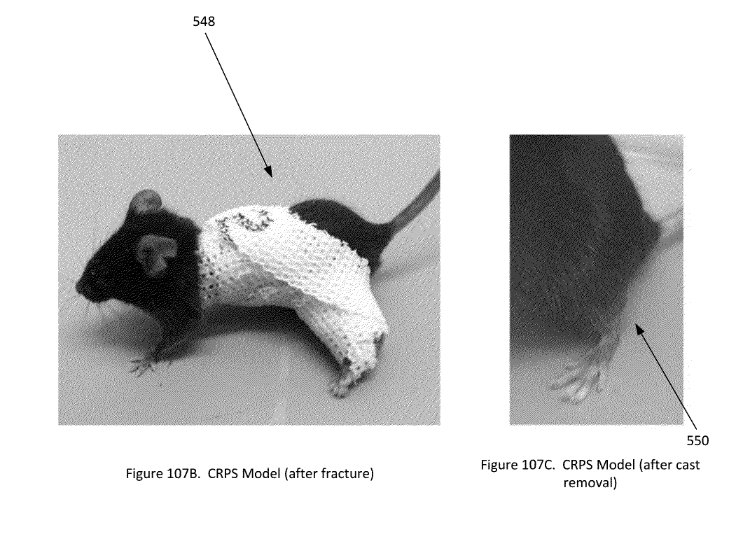

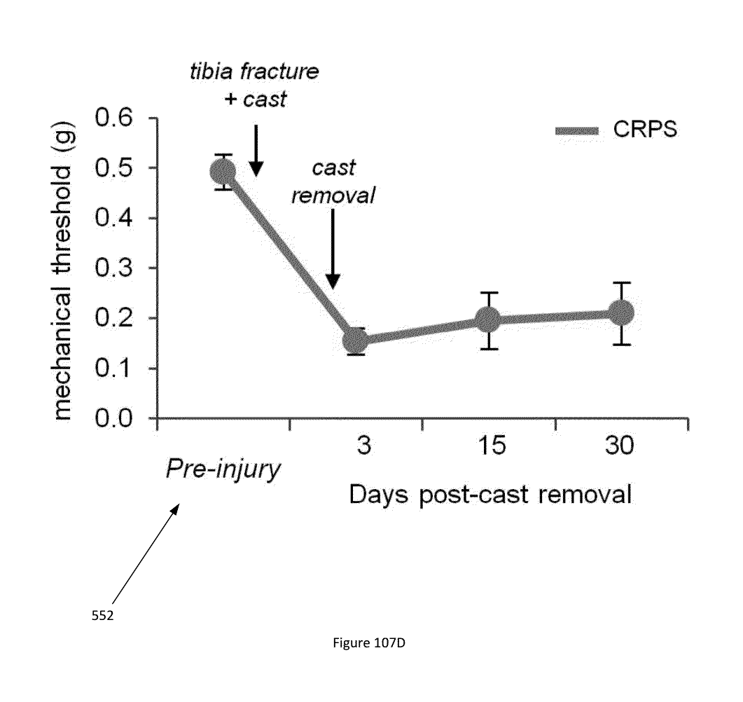

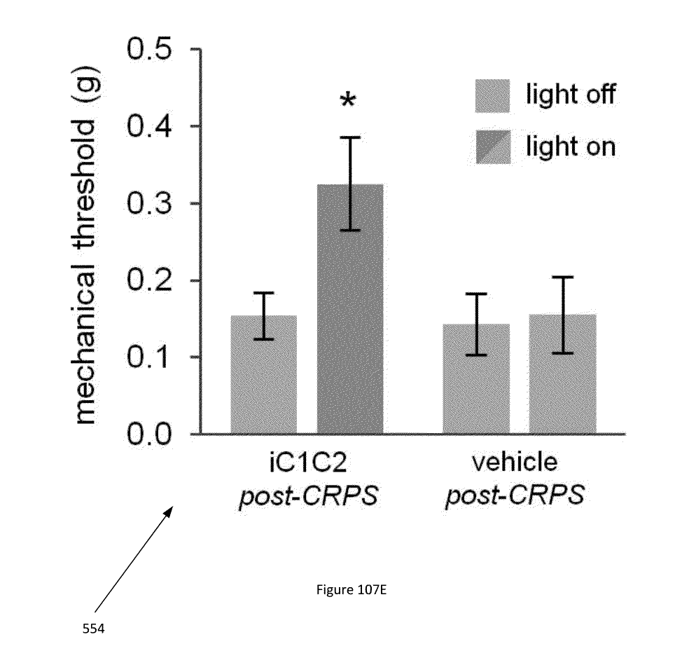

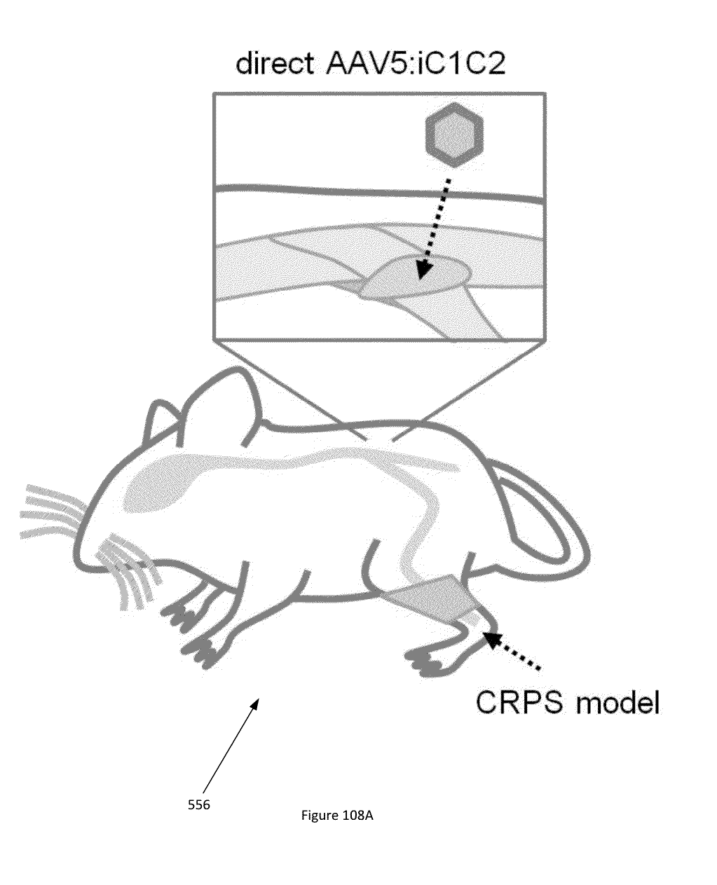

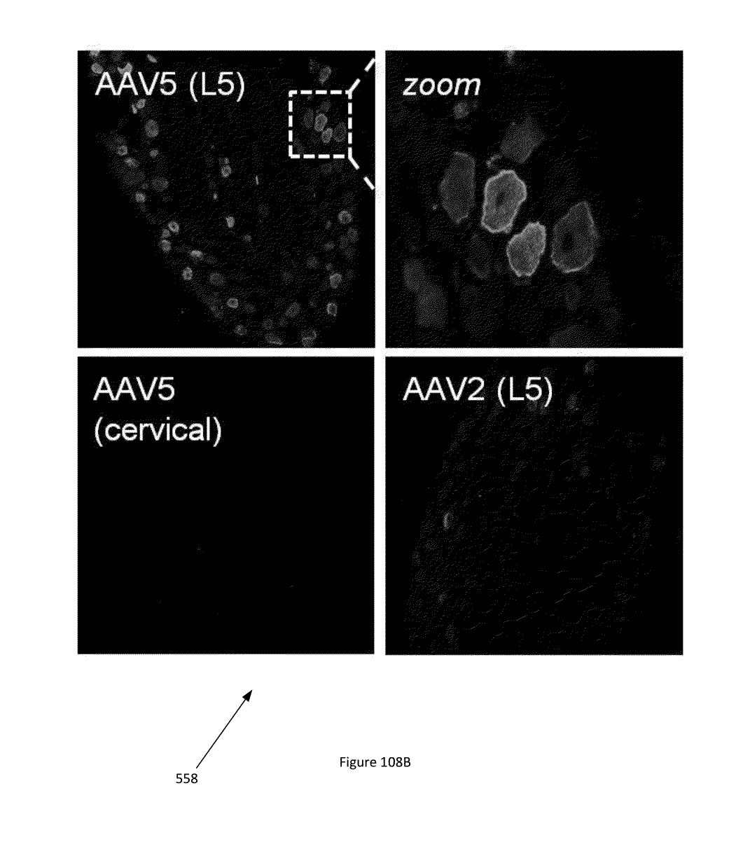

Referring to FIGS. 107A-107E, aspects of pain inhibition in a second model of neuropathic pain are illustrated. Referring to FIG. 107A, to determine whether this approach was amenable to other chronic pain paradigms, intrathecal delivery of AAV8:iC1C2 was performed in a mouse model (548) of complex regional pain syndrome (CRPS). Referring to FIG. 107B, the CRPS mouse model (548) is generated by fracturing the tibia bone and immobilizing the leg (with tibia fracture misaligned) for 4 weeks. Referring to FIG. 107C and the plot (552) of FIG. 107D, upon cast removal (550) there is a significant reduction in mechanical threshold that is stable through time. Referring to the chart (554) of FIG. 107E, the reduction in mechanical thresholds can be reversed following application of light treated with AAV8:iC1C2 but not vehicle. This demonstrates that the optogenetic inhibition of mechanical allodynia can be achieved in different models of neuropathic pain.

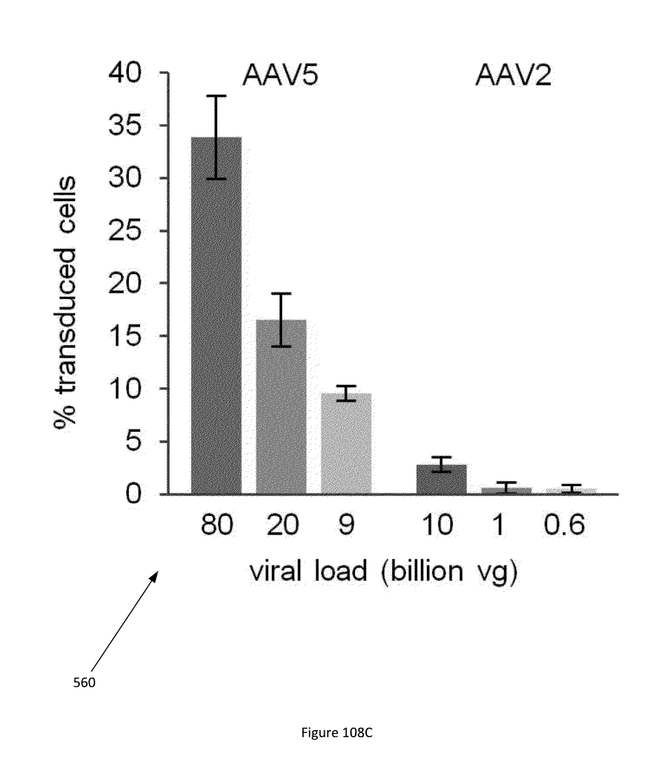

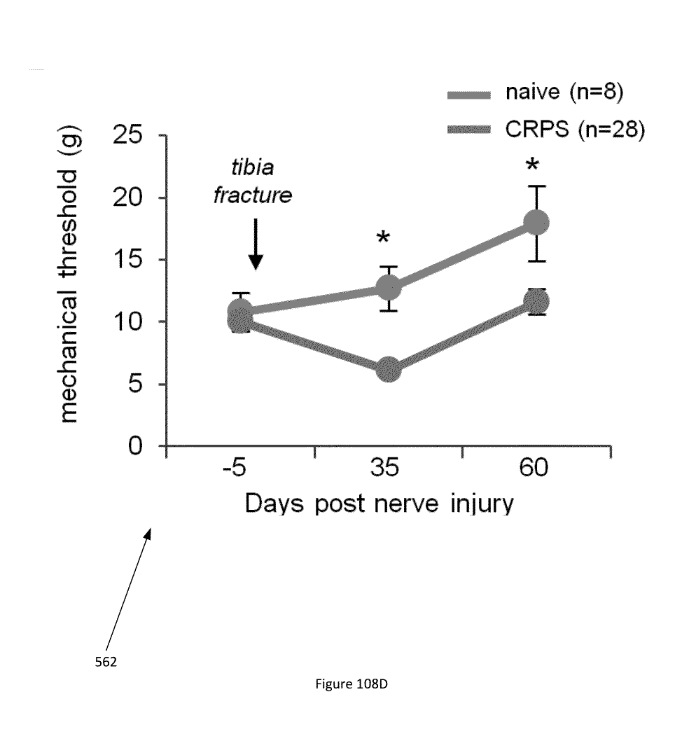

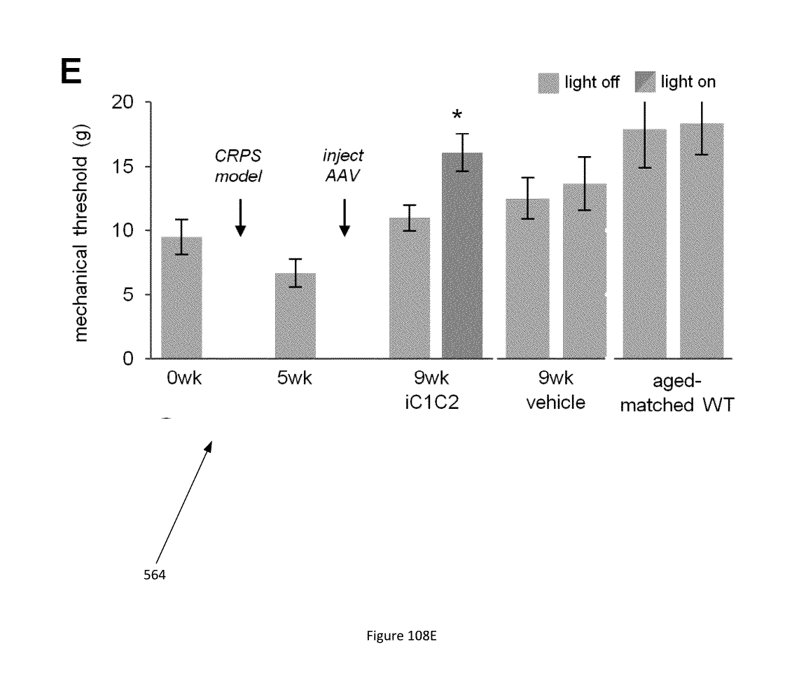

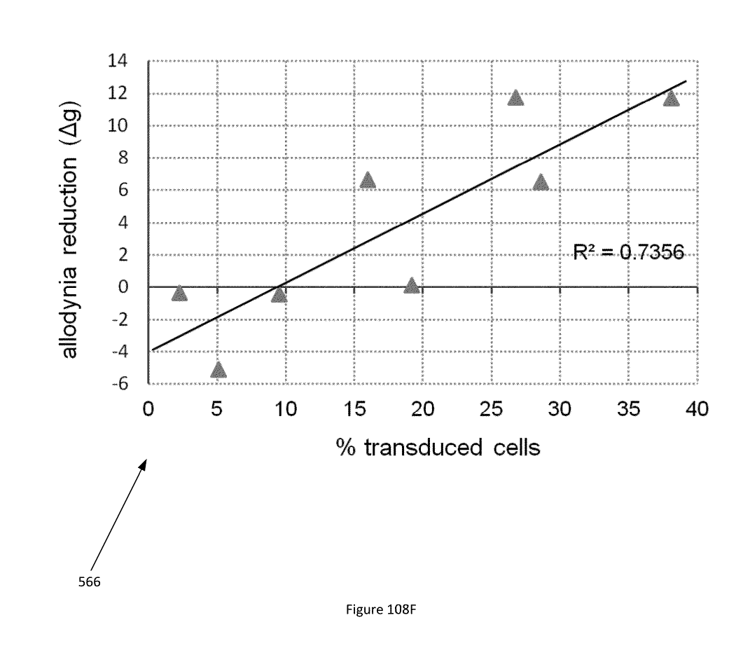

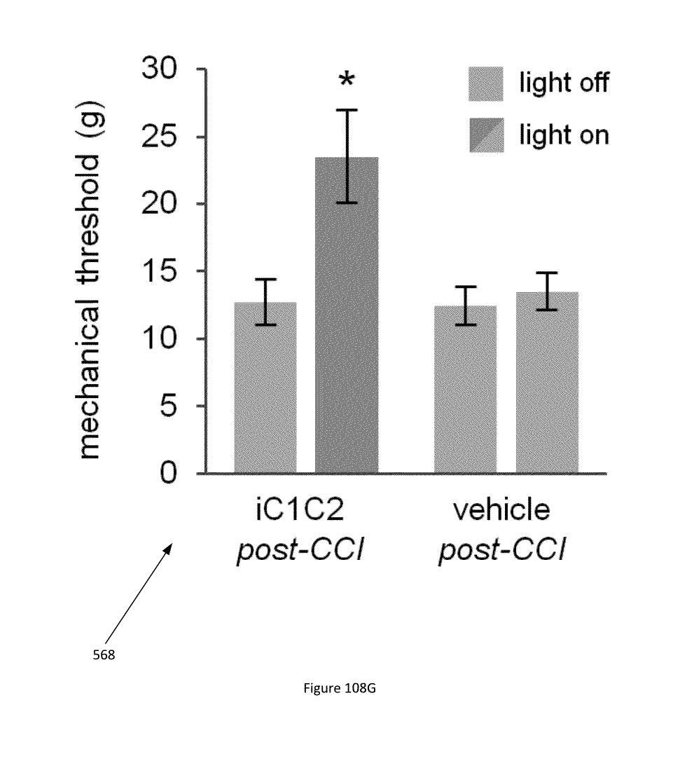

Referring to FIGS. 108A-108G, aspects of direct dorsal root ganglia (“DRG”) delivery for optogenetic therapy of neuropathic pain are illustrated. Referring to FIG. 108A, various doses of AAV5 or AAV2 expressing iC1C2 were injected directly into the lumbar DRG of rats (556). Rats are used as mice generally are too small to precisely target the ganglia. Referring to the images (558) of FIG. 108B, three weeks following injection robust expression was observed with AAV5 with up to 30% of cells observed to express the opsin with the higher dose of the vector, as shown in the chart (560) FIG. 108C. Referring to the chart (562) of FIG. 108D, a rat model of complex regional pain syndrome (“CRPS”) was generated following the change to this species. Note that the tibia fracture/cast immobilization results in mechanical allodynia that is stable through time (despite the actual threshold level increasing through time as a function of the aging rats). Referring to the chart (564) of FIG. 108E, direct DRG injection of AAV5:iC1C2 but not vehicle reverses mechanical allodynia upon application of light when administered 4 weeks following CRPS generation. Note that mechanical thresholds are restored to levels of age-matched wild-type littermates. Referring to the chart (566) of FIG. 108F, the magnitude of the reduction in allodynia correlates with percentage of transduced cells. Referring to the chart (568) of FIG. 108G, direct DRG injection of AAV5:iC1C2 but not vehicle also reverses mechanical allodynia upon application of light when administered 2 weeks following CCI.

These results demonstrate the biological activity and specificity of the present inventive therapy to robustly treat pain.

With regard to construct variations, one construct may comprise a coding sequence for the light activated protein (opsin, channel or pump) driven by a ubiquitous promoter (such as CMV or CAG) or a neuron specific promoter (such as hSyn or NF200) with or without regulatory elements (such as WPRE or beta globin intron) with a poly adenylation signal.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A system for controllably managing pain in the afferent nervous system of a patient having a targeted tissue structure that has been genetically modified to have light sensitive protein, comprising:
a. a light delivery element configured to direct radiation to at least a portion of a targeted tissue structure;
b. a light source configured to provide light to the light delivery element; and
c. a controller operatively coupled to light source;
wherein the targeted tissue structure comprises a sensory neuron of the patient; and wherein the controller is configured to be automatically operated to illuminate the targeted tissue structure with radiation such that a membrane potential of cells comprising the targeted tissue structure is modulated at least in part due to exposure of the light sensitive protein to the radiation.

2. The system of claim 1, wherein the portion of the targeted tissue structure of the patient is selected from the group consisting of: a spinal cord, a nerve cell body, a ganglion, a dorsal root ganglion, an afferent nerve fiber, an afferent nerve bundle, an afferent nerve ending, a sensory nerve fiber, a sensory nerve bundle, a sensory nerve ending, a sensory receptor, a free nerve ending, a mechanoreceptor, and a nociceptor.

3. The system of claim 1, wherein an applicator is disposed to illuminate the target tissue structure, the applicator being comprised of at least a light delivery element and a sensor, wherein the sensor is configured to:
a. produce an electrical signal representative of the state of the target tissue or its environment; and
b. deliver the signal to the controller, wherein the controller is further configured to interpret the signal from the sensor and adjust at least one light source output parameter such that the signal is maintained within a desired range, wherein the light source output parameter may be chosen from the group containing of; current, voltage, optical power, irradiance, pulse duration, pulse interval time, pulse repetition frequency, and duty cycle.

4. The system of claim 3, wherein the sensor is selected from the group consisting of: an optical sensor, a temperature sensor, a chemical sensor, and an electrical sensor.

5. The system of claim 1, wherein the controller is further configured to drive the light source in a pulsatile fashion.

6. The system of claim 5, wherein the current pulses are of a duration within the range of 1 millisecond to 100 seconds.

7. The system of claim 5, wherein the duty cycle of the current pulses is within the range of 99% to 0.1%

8. The system of claim 1, wherein the controller is responsive to a patient input.

9. The system of claim 8, wherein the patient input triggers the delivery of current.

10. The system of claim 5, wherein the current controller is further configured to control one or more variables selected from the group consisting of: the current amplitude, the pulse duration, the duty cycle, and the overall energy delivered.

11. The system of claim 1, wherein the light delivery element is placed about at least 60% of circumference of a nerve or nerve bundle.

12. The system of claim 1, wherein the light delivery element is placed inside the body of a patient.

13. The system of claim 1, wherein the light delivery element is placed outside of the body of a patient.

14. The system of claim 1, wherein the light sensitive protein is an opsin protein.

15. The system of claim 14, wherein the opsin protein is selected from the group consisting of: a depolarizing opsin, a hyperpolarizing opsin, a stimulatory opsin, an inhibitory opsin, a chimeric opsin, and a step-function opsin.

16. The system of claim 14, wherein the opsin protein is selected from the group consisting of: NpHR, eNpHR 1.0, eNpHR 2.0, eNpHR 3.0, SwiChR, SwiChR 2.0, SwiChR 3.0, Mac, Mac 3.0, Arch, ArchT, Arch 3.0, ArchT 3.0, iChR, ChR2, C1V1-T, C1V1-TT, Chronos, Chrimson, ChrimsonR, CatCh, VChR1-SFO, ChR2-SFO, ChR2-SSFO, ChEF, ChIEF, Jaws, ChloC, Slow ChloC, iC1C2, iC1C2 2.0, and iC1C2 3.0.

17. The system of claim 1, wherein the light sensitive protein is delivered to the target tissue using a virus.

18. The system of claim 17, wherein the virus is selected from the group consisting of: AAV1, AAV2, AAV4, AAV5, AAV6, AAV7, AAV8, AAV9, lentivirus, and HSV.

19. The system of claim 17, wherein the virus contains a polynucleotide that encodes for the opsin protein.

20. The system of claim 19, wherein the polynucleotide encodes for a transcription promoter.

21. The system of claim 20, wherein the transcription promoter is selected from the group consisting of: CaMKIIa, hSyn, CAG, CMV, Hb9Hb, Thy1, NF200, and Ef1a.

22. The system of claim 21, wherein the viral construct is selected from the group consisting of: AAV5-hSyn-eNpHR3.0, AAV5-CAG-eNpHR3.0, AAV5-hSyn-Arch3.0, AAV5-CAG-Arch3.0, AAV5-hSyn-iC1C23.0, AAV5-CAG-iC1C23.0, AAV5-hSyn-SwiChR3.0, AAV5-CAG-SwiChR3.0, AAV6-hSyn-eNpHR3.0, AAV6-CAG-eNpHR3.0, AAV6-hSyn-Arch3.0, AAV6-CAG-Arch3.0, AAV6-hSyn-iC1C23.0, AAV6-CAG-iC1C23.0, AAV6-hSyn-SwiChR3.0, AAV6-CAG-SwiChR3.0, AAV8-hSyn-eNpHR3.0, AAV8-CAG-eNpHR3.0, AAV8-hSyn-Arch3.0, AAV8-CAG-Arch3.0, AAV8-hSyn-iC1C23.0, AAV8-CAG-iC1C23.0, AAV8-hSyn-SwiChR3.0, and AAV8-CAG-SwiChR3.0.

23. The system of claim 1, wherein the light source emits light having a wavelength that is within a wavelength range that is selected from the group consisting of: 440 nm to 490 nm, 491 nm to 540 nm, 541 nm to 600 nm, 601 nm to 650 nm, and 651 nm to 700 nm.

24. The system of claim 1, wherein the light delivery element comprises an LED.

25. The system of claim 17, wherein the virus is delivered to an anatomical location that is different than that of the target tissue structure.

26. The system of claim 25, wherein the anatomical location is selected from the group consisting of: a spinal cord, a nerve cell body, a ganglion, a dorsal root ganglion, an afferent nerve fiber, an afferent nerve bundle, an afferent nerve ending, a sensory nerve fiber, a sensory nerve bundle, a sensory nerve ending, and a sensory receptor.
